(19) United States
(12) Patent Application Publication
(10) Pub. No.: US 2025/0059231 A1
(43) Pub. Date: Feb. 20, 2025

(54) Protease inhibitors

(71) Applicant: Protinhi B.V., Nijmegen (NL)

(72) Inventors: Daniel Gironés Delgado-Ureña, Nijmegen (NL); Cindy Elisabeth Johanna Dieteren, Nijmegen (NL); Bernd Nicodemus Maria van Buuren, Nijmegen (NL); Martinus Christiaan Feiters, Nijmegen (NL); Lorenzo Cavina, Nijmegen (NL); Montse Llinas Brunet, Nijmegen (NL); Peter Harold Han Hermkens, Nijmegen (NL)

(73) Assignee: Protinhi B.V., Nijmegen (NL)

(21) Appl. No.: 18/693,180

(22) PCT Filed: Sep. 30, 2022
(86) PCT No.: PCT/EP2022/077408

§ 371 Date: Mar. 19, 2024

(30) Foreign Application Priority Data
Sep. 30, 2021 (EP) 21200242.2
Sep. 9, 2022 (EP) 22194965.4

(51) Int. Cl. C07K 5/103; A61K 38/00; A61P 31/14; C07K 5/087; C07K 5/097; C07K 5/10; C07K 5/107; C07K 5/117

(52) U.S. Cl. CPC C07K 5/101; A61P 31/14; C07K 5/0812; C07K 5/0821; C07K 5/1016; C07K 5/1024; C07K 5/1027; A61K 38/00

(57) ABSTRACT

The present invention relates to a new class of compounds based on alkylated oligopeptides featuring distinct head and tail modifications at their termini. The compounds are useful as inhibitors of viral proteases, particularly of flaviviral proteases, and can therefore be used for treatment of viral infections.

### FIELD OF THE INVENTION

The present invention relates to a new class of compounds based on alkylated oligopeptides featuring distinct head and tail modifications at their termini. The compounds are useful as inhibitors of viral proteases, particularly of flaviviral proteases, and can therefore be used for treatment of viral infections.

### BACKGROUND ART

Flavivirus is a genus of the family Flaviviridae. This genus includes the West Nile virus (WNV), dengue virus (DEN), Tick-borne Encephalitis Virus, Japanese Encephalitis virus, Yellow Fever Virus, and several other viruses that may cause encephalitis.

Flaviviruses are viruses that cause millions of infections each year. Dengue viruses cause a self-limiting disease in humans called dengue fever (DF), which is often resolved in 7-10 days. However, more severe forms of the disease, known as Dengue hemorrhagic fever (DHF) and Dengue shock syndrome (DSS), are common in areas endemic to DEN and lead to considerable morbidity and mortality. According to World Health Organization estimates, 50-100 million cases of DEN infections in tropical and subtropical countries occur each year.

Complicating matters further is the fact that DEN exists as at least four separate serotypes (DEN-1, DEN-2, DEN-3, and DEN-4) with DEN-2 being the most prevalent in many recent epidemics. Unfortunately infection by one serotype does not provide protection from infections by the other serotypes. Furthermore, evidence suggests that subsequent infections by different serotypes may increase the probability of developing the more serious forms of the disease like DHF and DSS.

Every year, it is estimated that there are 50-100 million dengue virus infections with ˜1.5 million documented cases of dengue fever, and “500,000 cases of dengue hemorrhagic fever and shock syndrome. Reported cases increase annually. Approximately 40% of the world's population is at risk of dengue infection from living in regions endemic with the virus.

Symptoms of DENV infection include sudden onset of fever, body pain, headache, joint pain, rashes, and retro-orbital pain. Although usually mild or asymptomatic, the infection can progress into life-threatening serious conditions such as haemorrhagic fever (DHF). The symptoms of mild haemorrhage include petechiae, purpura, ecchymoses, and epistaxis. Up to 2% of the total number of DENV cases (mostly children below 15 years of age) experience progression of the infection to severe DHF, characterized by thrombocytopenia, hemorrhagic manifestations that could affect the skin, nose, gum, and gastrointestinal tract. Dengue shock syndrome (DSS), the most severe form of DHF, is characterized by a weak pulse and sudden drop in blood pressure, which is the result of the collapse of the vascular system owing to hypovolemia caused by vascular leakage. Patients with co-morbidities (e.g. diabetes mellitus, hypertension, cardiac or renal failure, sickle cell anaemia) or the at-risk populations (such as pregnant women, infants, the elderly) are at risk for developing severe disease. Patients with severe dengue have difficulty drinking, which hampers reversal of the effect of shock. Consequently, patients with the more severe forms of dengue may not be able to take oral medications.

West Nile virus (WNV) was introduced into the Western Hemisphere during an outbreak in the United States in 1999. Since this outbreak, WNV has spread throughout much of North America and has become a public health concern. Most WNV infections are asymptomatic; however, about 20% of cases are associated with mild flu-like symptoms. Some of these cases progress to more severe clinical manifestations, including encephalitis and/or flaccid paralysis.

In 1999, WNV emerged in the USA and has successfully spread across the entire country and into Canada, Mexico, and Central and South America. In 2007, the U.S. Centers for Disease Control reported 3630 clinical cases in the USA, with 2350 cases of West Nile fever, 1217 cases of meningitis or encephalitis, and 124 fatalities. Other regions at risk include Asia, Africa, Europe, and the Middle East.

Some members of the genus Flavivirus are transmitted by a vector such as an insect, in many cases the insect is a mosquito.

Flavivirus viruses are enveloped, positive-strand RNA viruses. The viral genome of the Flavivirus genus is translated as a single polyprotein and is subsequently cleaved into mature proteins. Both host signal peptidases and viral NS3 serine protease are involved in processing the polypeptide into viral proteins: structural proteins that form the virion particle, and non-structural proteins, that function in the virus life cycle. The viral NS3 protease has been shown to be required for viral replication, and provides a strategic target for inhibition in the development of flavivirus antivirals.

There is great demand for vaccines or antiviral therapeutics for Flaviviruses, such as Dengue or West Nile viruses. Currently, patients are treated with supportive care to relieve fever, pain, and dehydration. Attempts to treat West Nile disease with Ribavirin have been unsuccessful.

Therefore, there exists a need for improved antiviral therapies, particularly to treat Flavivirus infections such as by dengue virus or West Nile virus. There is a need for compounds that reduce or hinder the progression of such infections. There is a need for compounds that interact with viral machinery. There is a need for compounds that reduce TNF-α or IL-6 levels associated with viral infections.

### SUMMARY OF THE INVENTION

The invention provides a compound of general formula (0) or a salt thereof:

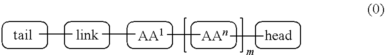

wherein
link is absent or is a linking moiety with a length of 1, 2, 3, 4, 5, or 6 atoms, preferably with a length of 1 or 2 atoms, more preferably 1 atom, wherein the atoms are selected from carbon, nitrogen, oxygen, and sulphur, more preferably at least one carbon atom, preferably at most one nitrogen, oxygen, or sulphur atom, wherein the linking moiety is optionally unsaturated, wherein each atom in the linking moiety is optionally substituted with halogen or with an oxygen atom (either as an —OH moiety or as an ═O moiety) or with a sulphur atom (either as a —SH moiety or as an ═S moiety) or with a nitrogen moiety (either as an —NH₂ moiety or as an ═NH moiety), wherein link connects tail to the N-terminus of AA¹, or wherein link and the N-terminal amine of AA¹ together represent such a linking moiety, wherein link is preferably —C(O)—, —C(═S)—, —C(═NH)—, —(CH₂)₁₋₆—, —O—(CH₂)₂₋₄—C(O)—, —O—(CH₂)₂₋₄—C(═S)—, —O—(CH₂)₂₋₄—C(═NH)—, —S—(CH₂)₂₋₄—C(O)—, —S—(CH₂)₂₋₄—C(═S)—, —S—(CH₂)₂₋₄—C(═NH)—, —NH—(CH₂)₂₋₄—C(O)—, —NH—(CH₂)₂₋₄—C(═S)—, or —NH—(CH₂)₂₋₄—C(═NH)—, more preferably —C(O)—, —C(═S)—, —C(═NH)—, —NH—(CH₂)₂₋₄—C(O)—, —NH—(CH₂)₂₋₄—C(═S)—, or —NH—(CH₂)₂₋₄—C(═NH)—, even more preferably —C(O)—, —C(═S)—, —C(═NH)—, and most preferably —C(O)—,
tail is H, C1-24alkyl, —O—C1-24alkyl, 5-20-membered (hetero)aryl, or 3-20-membered (hetero)cycloalkyl, wherein tail is optionally unsaturated, wherein tail is optionally substituted with halogen, C1-3 (halo)alkyl, or C1-3 (halo)alkoxyl;
head is —H, -h1, —O-h1, —C(O)-h1, —C(O)—N(H)h1, —N(h2) h1, or head is -C1-24alkyl, —(NH)₀₋₁-5-20-membered (hetero)aryl, or —(NH)₀₋₁-3-20-membered (hetero)cycloalkyl, wherein head is optionally unsaturated, wherein head is optionally substituted with halogen, C1-3 (halo)alkyl, or C1-3 (halo)alkoxyl;
h1 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h1 is optionally substituted with halogen, C1-3 (halo)alkyl, or C1-3 (halo)alkoxyl;
h2 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h2 is optionally substituted with halogen, C1-3 (halo)alkyl, or C1-3 (halo)alkoxyl;
AA¹ is an amino acid residue that is connected to its neighbouring AAⁿ via an amide bond to which it contributes a donor carboxylic acid, and that via its amine is connected to link;
AAⁿ is for each instance independently an amino acid residue, wherein the carbonyl moiety in the residue adjacent to head can instead together with head be replaced by —B(OH)₂ or a C1-6alkyl ester thereof, —P(O)(OH)2 or a C1-6alkyl ester thereof, —S(O)₂—C1, or 5-12 membered (hetero)aryl that is optionally substituted with halogen, C1-3 (halo)alkyl, or C1-3 (halo)alkoxy;
m is 1, 2, 3, 4, or 5.

Preferably the compound is of general formula (1) or a salt thereof:

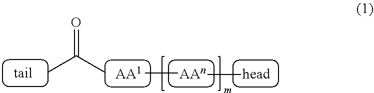

In some embodiments m is 1, 2, or 3, more preferably m is 2 or 3. In some embodiments tail is C1-20alkyl such as methyl or ethyl or hexanyl or heptanyl or nonanyl or undecanyl or tridecanyl or pentadecanyl or nonadecanyl, —O—C1-16alkyl such as —O-butyl, 3-12-membered (hetero)cycloalkyl such as adamantanyl or cyclohexyl, or 5-12-membered (hetero)aryl such as phenyl or biphenyl or bithiophene or substituted indole such as methoxyindole. In some embodiments head is H or —OH; —C(O)—N(H)h1 such as —C(O) NH-Bn or —C(O)—NH₂; 5-10-membered (hetero)aryl such as phenyl, C1-4alkyl such as methyl; —N(h2) h1 such as —N(H)h1 such as —NH₂, —NH(C1-6alkyl) such as —NH (propyl) or —NH (hexyl) or —NH (octyl) or —NH (hexadecyl), —NH—S(O)₂—C1-6 (cyclo)alkyl such as —NH—S(O)₂-cyclopropyl, —NH—CH₂-(5-6-membered aryl) such as —NH—CH₂-methoxyphenyl or —NH—CH₂-trifluoromethylphenyl or —NH-benzyl; or such as —N(C1-6alkyl)₂ such as —N(CH₃)2, or —N(C1-6alkyl)(C1-6alkoxyl) such as —N(CH₃)(OCH₃); or the carbonyl moiety of the AAⁿ adjacent to head together with head is replaced by —B(OH)₂ or together forms optionally substituted 5-membered heteroaryl such as 5-methyl-4-aza-oxazol-2-yl.

In some embodiments amino acid residues are represented by optionally substituted-NH—C(sc1)(sc2)-C(O)—or by optionally substituted and optionally unsaturated proline or pipecolic acid, wherein sc1 is for each instance independently H or —C1-3 (halo)alkyl or —CH₂-(halo)phenyl, sc2 is for each instance independently H or optionally substituted and optionally unsaturated C2-6 (halo)alkyl-N(sc1)₂, C1-6 (halo)alkyl, 5-10-membered (hetero)aryl, C₁-4 (halo)alkyl-[5-10-membered (hetero)aryl], C2-6 (halo)alkyl-N(sc1) C(N (sc1)₂)(═Nsc1), C1-6 (halo)alkyl-C(O)—N(sc1)₂, or C1-4 (halo)alkyl-[3-10-membered (hetero)cycloalkyl]; preferably wherein amino acid residues are optionally substituted and optionally unsaturated alanine, cysteine, aspartic acid, glutamic acid, phenylalanine, glycine, histidine, isoleucine, lysine, leucine, methionine, asparagine, ornithine, pyrrolysine, proline, pipecolic acid, glutamine, arginine, serine, threonine, selenocysteine, valine, tryptophan, or tyrosine, or homo-analogues or nor-analogues thereof; more preferably wherein amino acid residues are optionally substituted and optionally unsaturated lysine, ornithine, arginine, histidine, phenylalanine, alanine, glutamine, tryptophan, proline, or valine, or homo-analogues or nor-analogues thereof such as homophenylalanine, homohistidine, pipecolic acid, and phenylglycine; wherein optional substitutions are preferably halogen, C1-3 (halo)alkyl, C1-3 (halo)alkoxy, guanidinyl, or optionally unsaturated and optionally (halo)methylated-(O)₀₋₁—(CH₂)₀₋₁-5-6-membered (hetero)cycloalkyl such as phenyl or benzyl or imidazolyl or —O- pyridinyl or methyl-dihydropyrrolyl. In some embodiments AA¹ is lysine, arginine, phenylalanine, histidine, alanine, valine, leucine, or tryptophan, preferably it is lysine, arginine, or alanine, even more preferably it is lysine. In some embodiments m is 2 and AA¹ and the two instances of AAⁿ together form a tripeptide represented by KAK, KAH, KAF, KA-homoPhe, KAA, KPA, KPK, FAF, HAH, ROK, RQ-homoPhe, RNF, RAF, or RQF. In some embodiments m is 3 and AA¹ and the three instances of AAⁿ together form a tetrapeptide represented by KAAA, KAAF, KAAH, KAAK, KAAW, KAA-homoHis, KAA-homoPhe, KAA-phenylGly, KAA-(4-OBn-phenylGly), KPAF, KPAH, KPAK, KAPK, KPAW, KPA-homoHis, KPA-homoPhe, KPA-phenylGly, KPA-(4-OBn-phenylGly), AAAK, AAKA, AKAA, AKAK, APAK, A-(4-O-(4-Py)-Pro)-AK, AVAK, AKKK, LKAK, LKKK, VKAK, KPHK, KPH-homoPhe, K-(pipecolic acid)-AK, K-(pipecolic acid)-HK, KP-phenylglycine-K, K-(pipecolic acid)-phenylglycine-K, ARQK, ARQF, ARRK, FKKK, RARK, FAAF, or WAAW.

In some embodiments tail comprises at least 8 carbon atoms, preferably it is pentadecyl; and/or head comprises at most 1-10 total of carbon atoms and heteroatoms, preferably 1-8, more preferably 1-7, or the carbonyl moiety of the AAⁿ adjacent to head together with head is replaced by —B(OH)₂ or together forms optionally methylated 5-membered heteroaryl such as 5-methyl-4-aza-oxazol-2-yl; and/or head is —NH₂. In some embodiments the compound is any one of compounds 1-196.

Also provided is a composition comprising a pharmaceutically acceptable excipient and a compound as defined above, preferably wherein the composition is a pharmaceutical composition.

Also provided is the compound or composition as defined above, for use as a medicament, wherein the medicament is preferably for use in the treatment of a viral infection or a condition related to a viral infection. Also provided is a method for inhibiting a viral protease, the method comprising the step of contacting the viral protease with a compound or a composition as defined above. In some embodiments of the method the viral protease is a flaviviral protease, preferably it is a dengue virus protease, a West Nile virus protease, or a tick-borne encephalitis virus protease. Also provided is a method of treating, preventing, or delaying a viral infection or a condition related to a viral infection in a subject in need thereof, the method comprising the step of administering to the subject an effective amount of a compound as defined above, or a composition as defined above.

### DESCRIPTION OF EMBODIMENTS

### Compounds

The inventors have surprisingly found a family of asymmetric peptide analogues to be potent and specific inhibitors of viral proteases. The invention thus provides a compound of general formula (1) or a salt thereof:

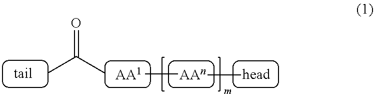

wherein
tail is C1-24alkyl, —O—C1-24alkyl, 5-20-membered (hetero)aryl, or 3-20-membered (hetero)cycloalkyl, wherein tail is optionally unsaturated, wherein tail is optionally substituted with halogen, C1-3 (halo)alkyl, or C1-3 (halo)alkoxyl;
head is —H, -h1, —O-h1, —C(O)-h1, —C(O)—N(H)h1, —N(h2) h1, or head is -C1-24alkyl, —(NH)₀₋₁-5-20-membered (hetero)aryl, or —(NH)₀₋₁-3-20-membered (hetero)cycloalkyl, wherein head is optionally unsaturated, wherein head is optionally substituted with halogen, C1-3 (halo)alkyl, or C1-3 (halo)alkoxyl;
h1 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h1 is optionally substituted with halogen, C1-3 (halo)alkyl, or C1-3 (halo)alkoxyl;
h2 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h2 is optionally substituted with halogen, C1-3 (halo)alkyl, or C1-3 (halo)alkoxyl;
AA¹ is an amino acid residue that is connected to its neighbouring AAⁿ via an amide bond to which it contributes a donor carboxylic acid, and that is connected to tail via an amide bond of a secondary amide to which it contributes a donor amine;
AAⁿ is for each instance independently an amino acid residue, wherein the carbonyl moiety in the residue adjacent to head can instead together with head be replaced by —B(OH)₂ or a C1-6alkyl ester thereof, —P(O)(OH)₂ or a C1-6alkyl ester thereof, —S(O)₂-halogen, or 5-12 membered (hetero)aryl that is optionally substituted with halogen, C1-3 (halo)alkyl, or C1-3 (halo)alkoxy;
m is 1, 2, 3, 4, or 5. Such a compound is referred to hereinafter as a compound according to the invention. A compositions comprising such a compound is referred to as a composition according to the invention.

The compounds generally resemble a short peptide that has a tail and a head attached thereto. The tail is generally a long hydrophobic and/or aliphatic moiety, although it can vary as defined. The head is generally a moiety that can function as a ‘warhead’, or it can be a secondary, shorter tail. However, there are embodiments wherein head is not smaller than tail—it should be noted that names of moieties as used herein do not in themselves imply any property.

### Central Peptide Analogue

The moieties represented by AA¹ and each instance of AAⁿ together form a peptide or an analogue thereof, and together are referred to as “the AAx” herein. The AAx represent a sequence and as represented in general formula (1) they can be considered to depict the traditional representation of peptides, with the N-terminus at the left and the C-terminus at the right. At its N-terminus, AA¹ in general formula (1) has an amide bond that connects it to tail. It is a secondary amide, which is formed of a primary amine and a carboxylic acid. The carbonyl moiety of that amide bond is depicted in general formula (1), as the nitrogen atom of the amide bond is comprised in AA¹, as is common practice for depicting amino acid sequences. For individual reference, the first AAⁿ from the left can be called AA², the second can be called AA³, etc.

m is 1, 2, 3, 4, or 5, and thus determines the length of the central peptide analogue as being 2 to 6. In preferred embodiments m is 1, 2, or 3, preferably m is 2 or 3. In some embodiments m is 1, 2, 3, or 4. In some embodiments m is 2, 3, 4, or 5. In some embodiments m is 2, 3, or 4. In some embodiments m is 3, 4, or 5. In some embodiments m is 1 or 2. In some embodiments m is 3 or 4. In some embodiments m is 4 or 5. In some embodiments m is 1. In some embodiments m is 2. In some embodiments m is 3. In some embodiments m is 4. In some embodiments m is 5.

The AAx are amino acid residues. This term should not be so narrowly construed as to only encompass the canonical 20 amino acids of the genetic code, although these are very suitable for AAⁿ, and except proline are also very suitable for AA¹. What is relevant for amino acid residues of AAx is that the residues when considered in isolation comprise at least an amine and a carboxylic acid, attached to a carbon atom that is often referred to as a backbone atom. AAx are described herein through description of corresponding residues in isolation, for ease of reference (so that reference can be made to their amine, instead of to the donor amine as comprised in the amide bond that connects it to its neighbouring residue; a skilled person is versed in such description). General formula (1-A) below exemplifies a compound of general formula 1 wherein AA¹ is alanine. For reference this alanine residue is enclosed in a box to more precisely indicate it.

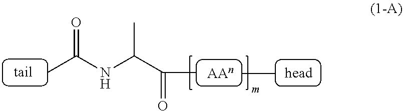

The backbone is considered to be formed by the amine, the carboxylic acid, and the atoms directly connecting those. In preferred embodiments, both the amine and the acid are attached to the same carbon atom, as in the canonical amino acids. In other embodiments the amine and the acid are attached to either end of an ethylene moiety, as in beta-amino acids. Preferably the backbone carbon atoms are comprised in a methylene, ethylene, propylene, or butylene moiety, more preferably methylene, ethylene, or propylene, even more preferably methylene or propylene, most preferably methylene. For reference general formula (1-B) below exemplifies a compound of general formula 1 wherein AA¹ is beta-alanine (3-aminopropanoic acid), and which thus exemplifies a backbone wherein the carbon atoms are comprised in an ethylene moiety. For reference this beta-alanine residue is enclosed in a box to more precisely indicate it.

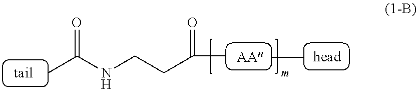

It is preferred that within a compound according to the invention each AAx has the same amount of backbone atoms. In some embodiments, each AAx has three backbone atoms (one of its amine, one of its carbon backbone, and one of its carboxylic acid). In some embodiments, each AAx has four backbone atoms (one of its amine, two of its carbon backbone, and one of its carboxylic acid).

In some embodiments, AA¹ has three backbone atoms while one or more AAⁿ has three or four backbone atoms. In some embodiments, AA¹ has four backbone atoms while one or more AAⁿ has three or four backbone atoms. In some embodiments, AA¹ has three or four backbone atoms while one or more AAⁿ has three or four backbone atoms. In some embodiments, AA¹ has three backbone atoms while one or more AAⁿ has three backbone atoms. In some embodiments, AA¹ has four backbone atoms while one or more AAⁿ has three backbone atoms. In some embodiments, AA¹ has three or four backbone atoms while one or more AAⁿ has three backbone atoms. In some embodiments, AA¹ has three backbone atoms while one or more AAⁿ has four backbone atoms. In some embodiments, AA¹ has four backbone atoms while one or more AAⁿ has four backbone atoms. In some embodiments, AA¹ has three or four backbone atoms while one or more AAⁿ has four backbone atoms. In some embodiments, AA¹ has three backbone atoms while each AAⁿ has three or four backbone atoms. In some embodiments, AA¹ has four backbone atoms while each AAⁿ has three or four backbone atoms. In some embodiments, AA¹ has three or four backbone atoms while each AAⁿ has three or four backbone atoms. In some embodiments, AA¹ has three backbone atoms while each AAⁿ has three backbone atoms. In some embodiments, AA¹ has four backbone atoms while each AAⁿ has three backbone atoms. In some embodiments, AA¹ has three or four backbone atoms while each AAⁿ has three backbone atoms. In some embodiments, AA¹ has three backbone atoms while each AAⁿ has four backbone atoms. In some embodiments, AA¹ has four backbone atoms while each AAⁿ has four backbone atoms. In some embodiments, AA¹ has three or four backbone atoms while each AAⁿ has four backbone atoms.

As for canonical amino acids except glycine, backbone carbon atoms can be further substituted. For instance when a backbone methylene carbon atom is substituted with a further methyl group, the canonical amino acid residue alanine is formed. In addition, the amine of an amino acid can also be further substituted. When an amine is substituted, it is preferred that the backbone of that AAx is not further substituted. Such amine-substituted amino acid residues are known in the art from peptoids, which are oligomers of N-substituted glycines. In some embodiments each AAx is an N-substituted glycine. In some embodiments no AAx is an N-substituted glycine. In some embodiments the amine is not further substituted. For reference general formula (1-P) below exemplifies a compound of general formula 1 wherein AA¹ is N-methylated glycine, and which thus exemplifies a peptoid-type alanine residue (wherein the conventional alanine side chain is now at the residue's backbone nitrogen). For reference this peptoid-type alanine residue is enclosed in a box to more precisely indicate it.

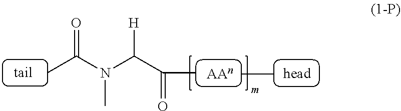

In preferred embodiments the amino acid residues are represented by optionally substituted —NH—C(sc1)(sc2)-C(O)—or —N(sc2)-C(sc1) H—C(O)—or —NH—CH₂—C(sc1)(sc2)-C(O)—or —NH—C(sc1)(sc2)-CH₂—C(O)—or by optionally substituted and optionally unsaturated proline or pipecolic acid; preferably by —NH—C(sc1)(sc2)-C(O)—or by optionally substituted and optionally unsaturated proline or pipecolic acid; wherein
sc1 is for each instance independently H or -C1-3 (halo)alkyl or —CH₂-(halo)phenyl; preferably each sc1 that is not comprised in an instance of sc2 is H, more preferably each sc1 is H; in these cases-NH—C(sc1)(sc2)-C(O)- and —N(sc2)-C(sc1) H—C(O)- and —NH—CH₂—C(sc1)(sc2)-C(O)- and —NH—C(sc1)(sc2)-CH₂—C(O)—are -NH—CH(sc2)-C(O)- and —N(sc2)-CH₂—C(O)- and —NH—CH₂—CH(sc2)-C(O)- and —NH—CH(sc2)-CH₂—C(O)—; preferably —CH₂-(halo)phenyl is —CH₂-phenyl; preferably -C1-3 (halo)alkyl is —CH₃ or —CH₂—CH₃; preferably sc1 is not optionally substituted; preferably each instance of sc1 represents the same moiety;
sc2 is for each instance independently H or optionally substituted and optionally unsaturated C2-6 (halo)alkyl-N(sc1)₂, C1-6 (halo)alkyl, 5-10-membered (hetero)aryl, C₁-4 (halo)alkyl-[5-10-membered (hetero)aryl], C2-6 (halo)alkyl-N(sc1) C(N (sc1)₂)(═Nsc1), C1-6 (halo)alkyl-C(O)—N(sc1)₂, or C1-4 (halo)alkyl-[3-10-membered (hetero)cycloalkyl]; when an instance of sc1 is comprised in an instance of sc2, it is preferably H; in some embodiments sc2 is not optionally substituted and not optionally unsaturated; in some embodiments sc2 is optionally substituted and not optionally unsaturated; in some embodiments sc2 is not optionally substituted and is optionally unsaturated; particularly preferred optional substitutions are halogen, C1-3 (halo)alkyl, or C1-3 (halo)alkoxyl, more preferably halogen, —CH₃, and —O—CH₃; wherein within sc2 optional substitutions are preferably —OH, —SH, —SeH, —S—CH₃, —O—CH₃, and —COOH, more preferably —OH, —SH, and —S—CH₃, most preferably —OH;
wherein in some embodiments sc2 is not optionally substituted; wherein in some embodiments sc2 is not optionally unsaturated; wherein in some embodiments sc2 is not optionally substituted and not optionally unsaturated; wherein sc2 is preferably H, C2-5 (halo)alkyl-N(sc1)₂, C1-4 (halo)alkyl, 5-9-membered (hetero)aryl, C₁-2 (halo)alkyl-[5-9-membered (hetero)aryl], C2-4 (halo)alkyl-N(sc1) C(N (sc1)₂)(═Nsc1), C1-4 (halo)alkyl-C(O)—N(sc1)₂, or C1-2 (halo)alkyl-[3-9-membered (hetero)cycloalkyl];
wherein C2-6 (halo)alkyl-N(sc1)₂ is preferably —CH₂—CH₂—CH₂—NH₂ or —CH₂—CH₂—CH₂—CH₂—NH₂; wherein 5-10-membered (hetero)aryl is preferably phenyl; wherein optionally substituted C₁-4 (halo)alkyl-[5-10-membered (hetero)aryl] is preferably —CH₂-phenyl, —CH₂—CH₂-phenyl, —CH₂-imidazolyl, —CH₂—CH₂-imidazolyl, —CH₂-indolyl, —CH₂—CH₂-indolyl; —CH₂-hydroxyphenyl, —CH₂—CH₂-hydroxyphenyl; wherein optionally substituted C₁-6 (halo)alkyl is preferably —CH₃, —CH(CH₃)₂, -CH₂—CH(CH₃)₂, —CH(CH₃)—CH₂—CH₃, —CH₂—OH, —CH₂—SH, —CH₂—SeH, —CH₂—CH₂—CH₂—S—CH₃, —CH(CH₃)—CH₂—OH, —CH₂—CH₂—COOH, or —CH₂—COOH, more preferably —CH₃, —CH(CH₃)2, —CH₂—CH(CH₃)2, —CH(CH₃)—CH₂—CH₃, —CH₂—OH, —CH₂—SH, —CH₂—SeH, —CH₂—CH₂—CH₂—S—CH₃, or —CH₂ (CH₃)—CH₂—OH; wherein C2-6 (halo)alkyl-N(sc1) C(N (sc1)₂)(═Nsc1) is preferably —CH₂—CH₂—CH₂—N—C(═NH)—NH₂; wherein C1-6 (halo)alkyl-C(O)—N(sc1)₂ is preferably —CH₂—CH₂—C(O) NH₂ or —CH₂—C(O) NH₂; preferably wherein amino acid residues are optionally substituted and optionally unsaturated alanine, cysteine, aspartic acid, glutamic acid, phenylalanine, glycine, histidine, isoleucine, lysine, leucine, methionine, asparagine, ornithine, pyrrolysine, proline, pipecolic acid, glutamine, arginine, serine, threonine, selenocysteine, valine, tryptophan, tyrosine, homophenylalanine, homohistidine, para-guanidinyl-phenylalanine, meta-guanidinyl-phenylalanine, (2-amino-1H-imidazol-4-yl)ethyl-alanine, (2-amino-1H-imidazol-4-yl)methyl-alanine, (2-amino-1H-imidazol-4-yl)-alanine, (pyridin-3-yl)homoalanine, (pyridin-3-yl)alanine, (pyridin-4-yl)alanine, (1H-imidazol-2-yl)aminomethyl-alanine, (1H-imidazol-2-yl)aminoethyl-alanine, (pyridin-4-yl)homoalanine, phenylglycine, (4-O-benzyl)phenylglycine, (4-O-4-pyridinyl) proline, pipecolic acid, or homo-analogues or nor-analogues thereof; more preferably wherein amino acid residues are optionally substituted and optionally unsaturated alanine, cysteine, aspartic acid, glutamic acid, phenylalanine, glycine, histidine, isoleucine, lysine, leucine, methionine, asparagine, ornithine, pyrrolysine, proline, pipecolic acid, glutamine, arginine, serine, threonine, selenocysteine, valine, tryptophan, or tyrosine, or homo-analogues or nor-analogues thereof; in some embodiments these residues are not optionally substituted and not optionally unsaturated; in some embodiments these residues are optionally substituted and not optionally unsaturated; in some embodiments these residues are not optionally substituted and is optionally unsaturated; particularly preferred optional substitutions are halogen, C1-3 (halo)alkyl, or C1-3 (halo)alkoxyl, more preferably halogen, —CH₃, and —O—CH₃;
more preferably wherein amino acid residues are optionally substituted and optionally unsaturated lysine, ornithine, arginine, histidine, phenylalanine, alanine, glutamine, tryptophan, proline, or valine, or homo-analogues or nor-analogues thereof such as homophenylalanine, homohistidine, pipecolic acid, and phenylglycine; as described above;
wherein optional substitutions are preferably halogen, C1-3 (halo)alkyl, C1-3 (halo)alkoxy, guanidinyl (which is —NH—C(═NH)—NH₂), or optionally unsaturated and optionally (halo)methylated —(O)₀₋₁—(CH₂)₀₋₁-5-6-membered (hetero)cycloalkyl such as phenyl or benzyl or imidazolyl or —O-pyridinyl or methyl-dihydropyrrolyl.

AAⁿ can additionally be optionally substituted and optionally unsaturated proline or pipecolic acid, wherein preferred optional substitutions are hydroxyl such as 4-hydroxy, and aryloxy such as 4—O-phenyl or 4-O-pyridinyl. AA¹ is not such a proline or pipecolic acid.

In some embodiments the amino acid residues are represented by optionally substituted-N (sc2)-C(sc1) H—C(O)—or —NH—CH₂—C(sc1)(sc2)-C(O)—or —NH—C(sc1)(sc2)-CH₂—C(O)—or by optionally substituted and optionally unsaturated proline or pipecolic acid; such residues are peptoid-type residues or beta-type residues. Features and conditions apply as described above.

In some embodiments the amino acid residues are represented by optionally substituted-N (sc2)-C(sc1) H—C(O)—or by optionally substituted and optionally unsaturated proline or pipecolic acid; such residues are peptoid-type residues. Features and conditions apply as described above.

In some embodiments the amino acid residues are represented by optionally substituted-NH—CH₂—C(sc1)(sc2)-C(O)—or —NH—C(sc1)(sc2)-CH₂—C(O)—or by optionally substituted and optionally unsaturated proline or pipecolic acid; such residues are beta-type residues. Features and conditions apply as described above.

Optionally the amino acid residues are represented by optionally substituted-N(sc1)-C(sc1)(sc2)-C(O)—or —N(sc2)-C(sc1)₂—C(O)—or —N(sc1)-CH₂—C(sc1)(sc2)-C(O)—or —N(sc1)-C(sc1)(sc2)-CH₂—C(O)—or by optionally substituted and optionally unsaturated proline or pipecolic acid. Each instance of sc1 and sc2 is independently chosen.

AA¹ is an amino acid residue that is connected to its neighbouring AAⁿ via an amide bond to which AA¹ contributes the donor carboxylic acid, and that in general formula (1) is connected to tail via an amide bond to which AA¹ contributes the donor amine. The carbon backbone of AAⁿ is preferably comprised in a methylene moiety. AAⁿ is preferably optionally substituted and optionally unsaturated alanine, cysteine, aspartic acid, glutamic acid, phenylalanine, glycine, histidine, isoleucine, lysine, leucine, methionine, asparagine, ornithine, pyrrolysine, proline, pipecolic acid, glutamine, arginine, serine, threonine, selenocysteine, valine, tryptophan, or tyrosine, or homo-analogues or nor-analogues thereof; in some embodiments these residues are not optionally substituted and not optionally unsaturated; in some embodiments these residues are optionally substituted and not optionally unsaturated; in some embodiments these residues are not optionally substituted and is optionally unsaturated; particularly preferred optional substitutions are halogen, C1-3 (halo)alkyl, or C1-3 (halo)alkoxyl, more preferably halogen, —CH₃, and —O—CH₃. In some embodiments AA¹ is uncharged or positively charged under physiological conditions. More preferably AA¹ is lysine, arginine, phenylalanine, histidine, alanine, valine, leucine, or tryptophan, still more preferably it is alanine, lysine, arginine, or histidine, even more preferably it is alanine, lysine or arginine, still more preferably it is arginine or lysine, most preferably lysine.

AAⁿ is for each instance independently an amino acid residue, wherein the carbonyl moiety in the residue adjacent to head can instead together with head be replaced by —B(OH)₂ or a C1-6alkyl ester thereof, —P(O)(OH)₂ or a C1-6alkyl ester thereof, —S(O)₂-halogen, or 5-12 membered (hetero)aryl that is optionally substituted with halogen, C1-3 (halo)alkyl, or C1-3 (halo)alkoxy. When the carbonyl moiety in the residue adjacent to head together with head replaces a group, this group thus replaces head and the entire amide bond through which it could have been connected to that AAⁿ residue. Such a moiety is referred to herein as a replacing head. For reference general formula (1-H) below exemplifies a compound of general formula 1 wherein m is 1 and AAⁿ is alanine and for this AAⁿ the carbonyl moiety instead together with head is replaced by replacing head. For reference this alanine residue with the replacing head is enclosed in square brackets to more precisely indicate it.

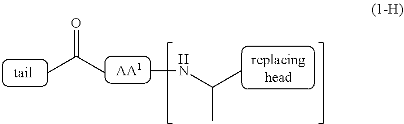

Replacing head can be—B(OH)₂ or a C1-6alkyl ester thereof, —P(O)(OH)₂ or a C1-6alkyl ester thereof, —S(O)₂-halogen (preferably —S(O)₂—Cl), and 5-12 membered (hetero)aryl that is optionally substituted with halogen, C1-3 (halo)alkyl, or C1-3 (halo)alkoxy. Herein, preferred optional substitutions are -F, —CH₃, —CF₃, and —OCH₃, preferred alkyl esters are C2-3 alkyl esters, and preferred 5-12 membered (hetero)aryl is 5-6 membered (hetero)aryl, more preferably 5-6 membered heteroaryl, most preferably 5-membered heteroaryl. For reference general formula (1-H—B) below exemplifies a compound of general formula 1 wherein m is 1 and AAⁿ is alanine and for this AAⁿ the carbonyl moiety instead together with head is replaced by —B(OH)₂ as the replacing head.

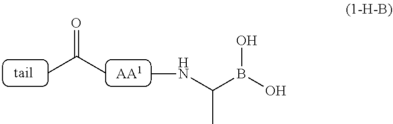

Preferred instances of replacing head are shown below, with a name for reference. Of these, Rh1, Rh2, Rh3, and Rh4 are more preferred. Rh1, Rh2, and Rh3 are even more preferred. Rh1 and Rh2 are most preferred.

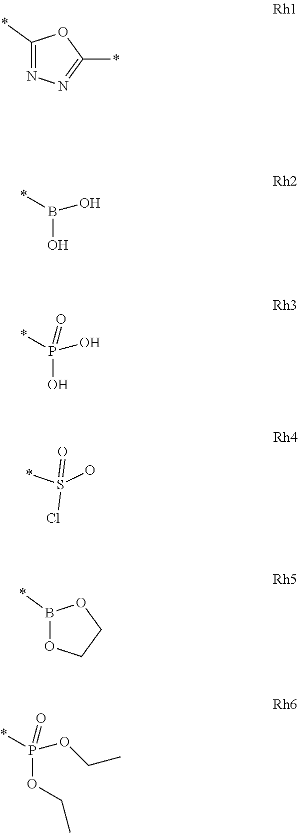

As described above, individual instances of AAⁿ can be referred to as AA², AA³, etc., depending on m. AA² is an amino acid residue that is connected to its neighbouring AA³, when present, via an amide bond to which it contributes the donor carboxylic acid, and that is connected to AA¹ via an amide bond to which it contributes the donor amine which can be a primary amine or a secondary amine such as when AA² is for instance proline. When no AA³ is present, AA² is connected to head via its carbonyl moiety that can be comprise in an amide bond or that can form another moiety, depending on the nature of head. For instance, when head is H, the carbonyl moiety of the relevant AAⁿ contributes to an aldehyde moiety. The same applies to each other instance of AA″, mutatis mutandis.

The carbon backbone of AAⁿ is preferably comprised in a methylene moiety. AAⁿ is for each independent instance preferably optionally substituted and optionally unsaturated alanine, cysteine, aspartic acid, glutamic acid, phenylalanine, glycine, histidine, isoleucine, lysine, leucine, methionine, asparagine, ornithine, pyrrolysine, proline, pipecolic acid, glutamine, arginine, serine, threonine, selenocysteine, valine, tryptophan, or tyrosine, or homo-analogues or nor-analogues thereof; in some embodiments these residues are not optionally substituted and not optionally unsaturated; in some embodiments these residues are optionally substituted and not optionally unsaturated; in some embodiments these residues are not optionally substituted and is optionally unsaturated; particularly preferred optional substitutions are halogen, C1-3 (halo)alkyl, or C1-3 (halo)alkoxyl, more preferably halogen, —CH₃, and —O—CH₃. In some embodiments each individual instance of AAⁿ is independently uncharged or positively charged under physiological conditions. More preferably each individual instance is independently lysine, arginine, phenylalanine, histidine, alanine, valine, leucine, or tryptophan, still more preferably it is lysine, arginine, or histidine, even more preferably it is lysine.

In preferred embodiments, AA² is lysine, arginine, glutamine, asparagine, alanine, phenylalanine, proline, histidine, alanine, valine, leucine, or tryptophan, still more preferably it is lysine, arginine, glutamine, asparagine, alanine, proline or histidine, even more preferably it is lysine, arginine, glutamine, alanine, asparagine, or, proline, even more preferably it is proline, arginine, alanine, or glutamine, even more preferably it is alanine, arginine, glutamine, most preferably it is alanine or glutamine.

In preferred embodiments, AA³, when present, is lysine, arginine, phenylalanine, histidine, alanine, valine, leucine, glutamine, or tryptophan, still more preferably it is lysine, arginine, glutamine, alanine, phenylalanine or histidine, still more preferably it is glutamine, alanine, or phenylalanine.

In preferred embodiments, AA⁴, when present, is lysine, arginine, phenylalanine, histidine, alanine, valine, leucine, or tryptophan, still more preferably it is lysine, alanine or phenylalanine, even more preferably it is lysine.

In preferred embodiments, AA⁵, when present, is lysine, arginine, phenylalanine, histidine, alanine, valine, leucine, or tryptophan, still more preferably it is lysine, arginine, or histidine, even more preferably it is lysine.

In preferred embodiments, AA⁶, when present, is lysine, arginine, phenylalanine, histidine, alanine, valine, leucine, or tryptophan, still more preferably it is lysine, arginine, or histidine, even more preferably it is lysine.

The AAⁿ that is adjacent to head is preferably uncharged or positively charged under physiological conditions, more preferably it is positively charged under physiological conditions. Preferably, none of the AAx are negatively charged under physiological conditions Preferably, at most 4 of the AAx are positively charged under physiological conditions, more preferably at most 3 or the AAx positively charged under physiological conditions, most preferably 2 of the AAx are positively charged under physiological conditions. When one or more of the AAx are positively charged under physiological conditions, it is preferred that AA¹ is positively charged under physiological conditions. When two or more of the AAx are positively charged under physiological conditions, it is preferred that AA¹ and the AAⁿ that is adjacent to head are positively charged under physiological conditions.

Amino acid residues as described herein can be L or D or a mixture thereof. Preferably residues are L, more preferably all residues within an individual embodiment are L. Analogues of residues that are known in the art can also be used, and additional such residues that can be represented by AAx are shown below, where residues can be shown as comprised in a peptide, or as comprised in H-AAx-OH for the free residue, or can be shown as the free residue.

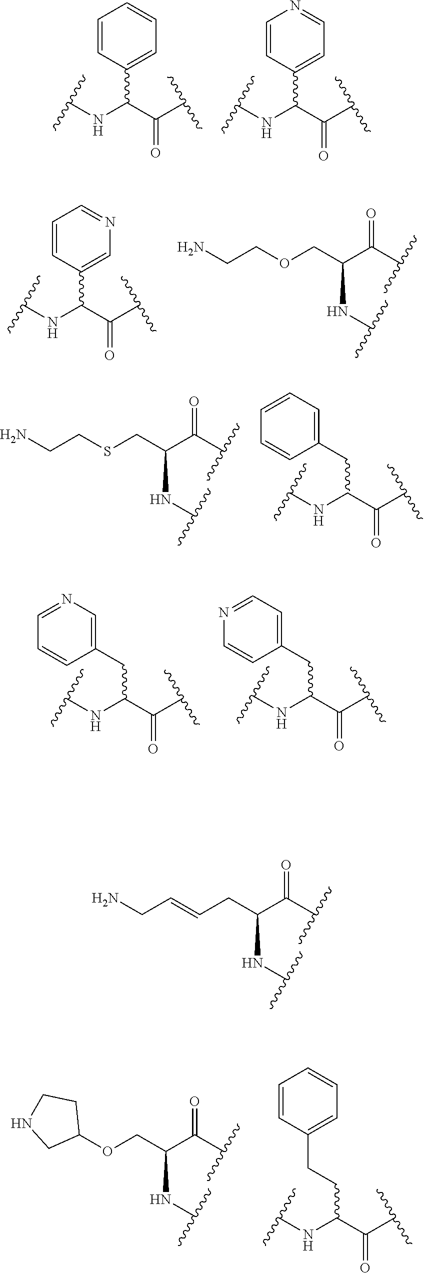

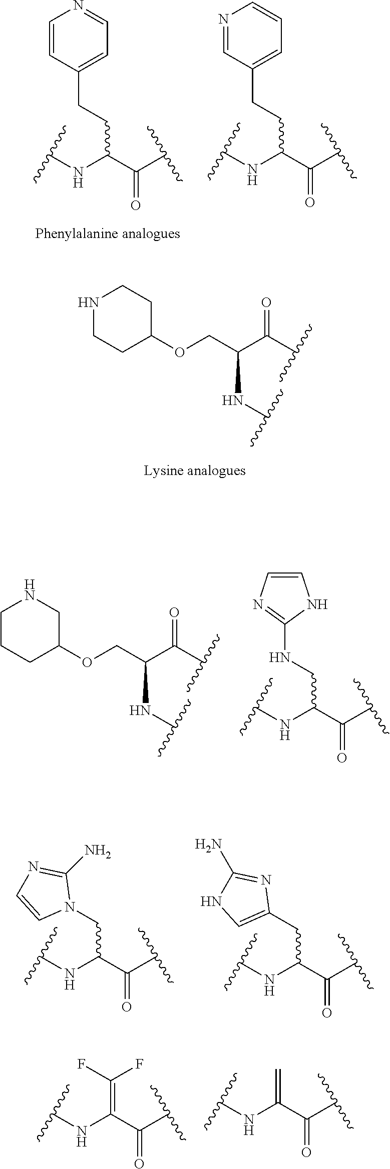

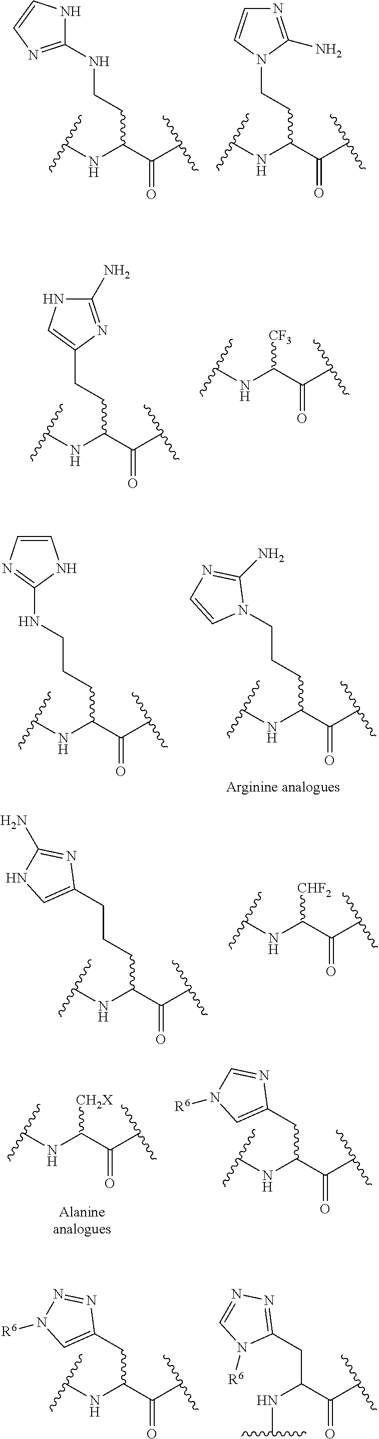

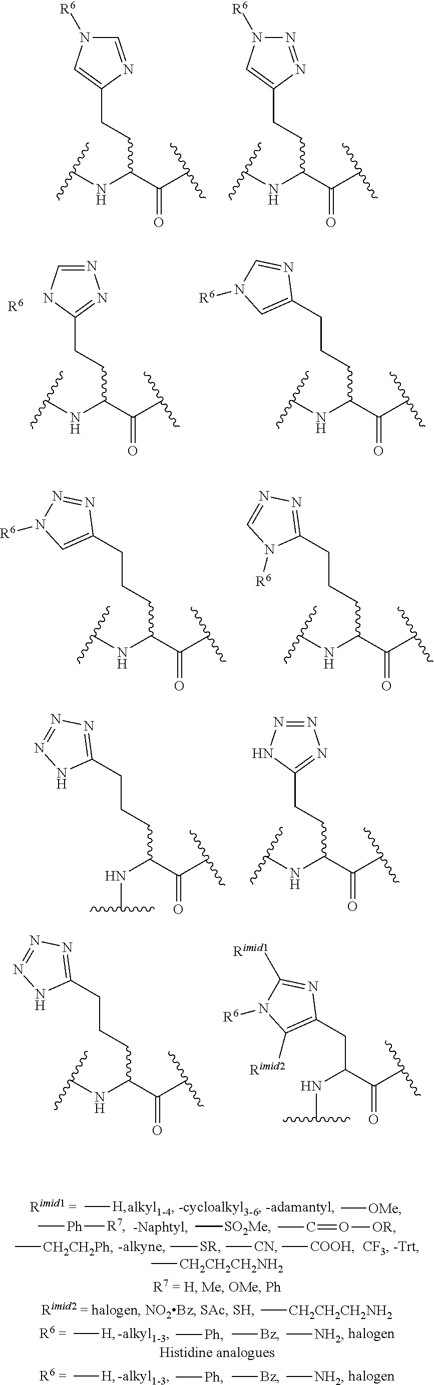

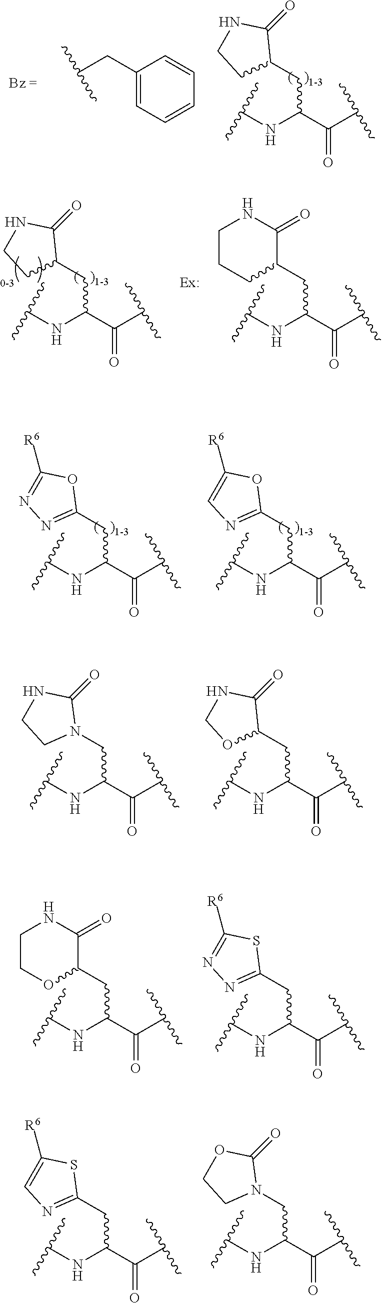

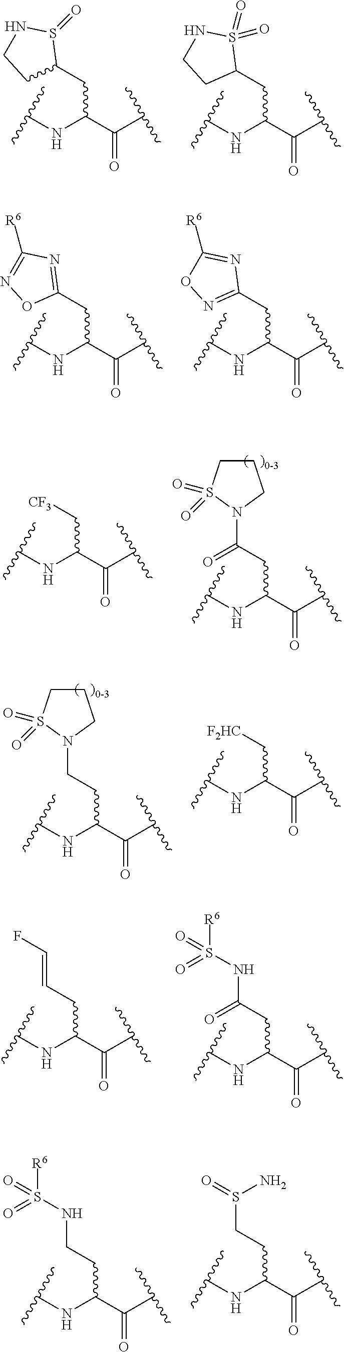

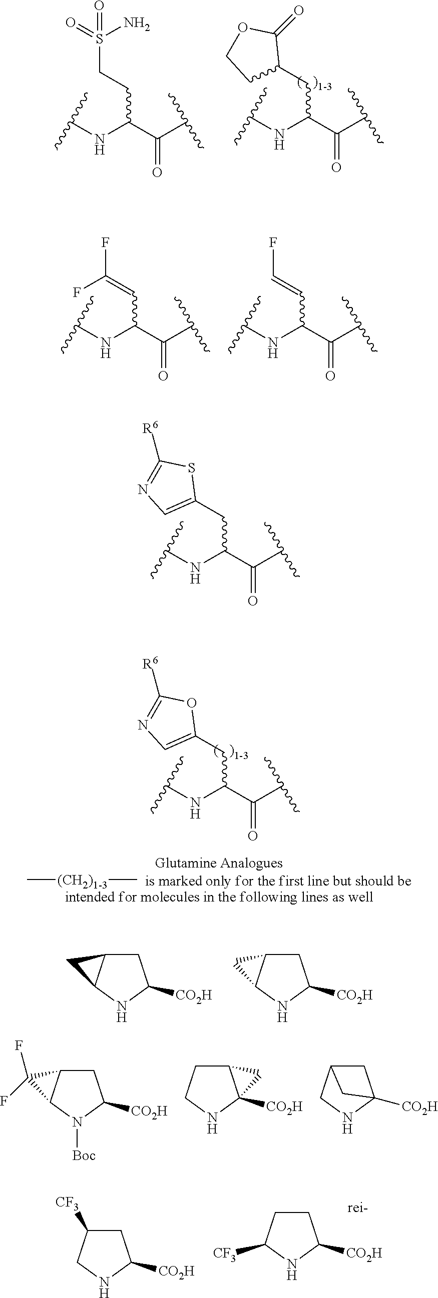

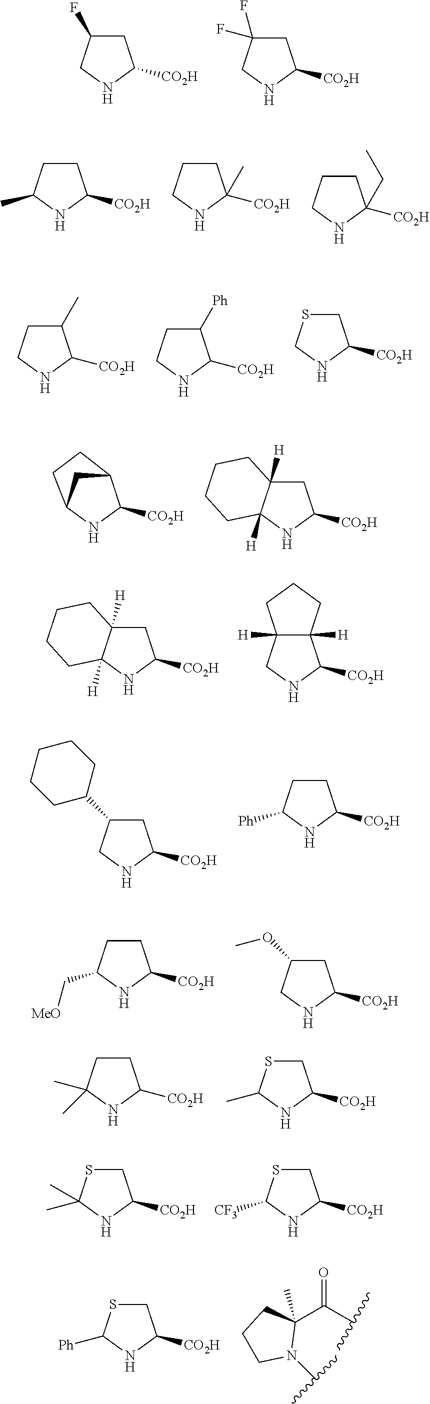

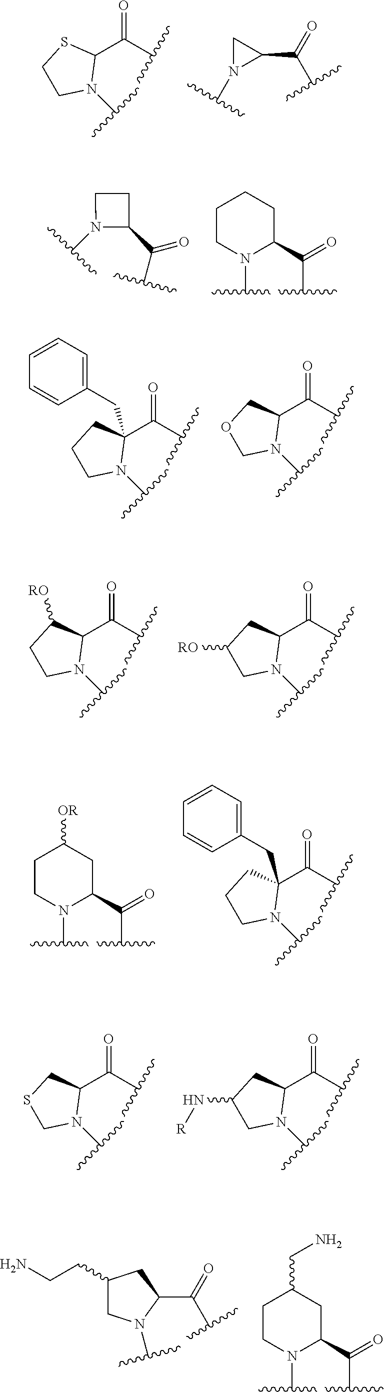

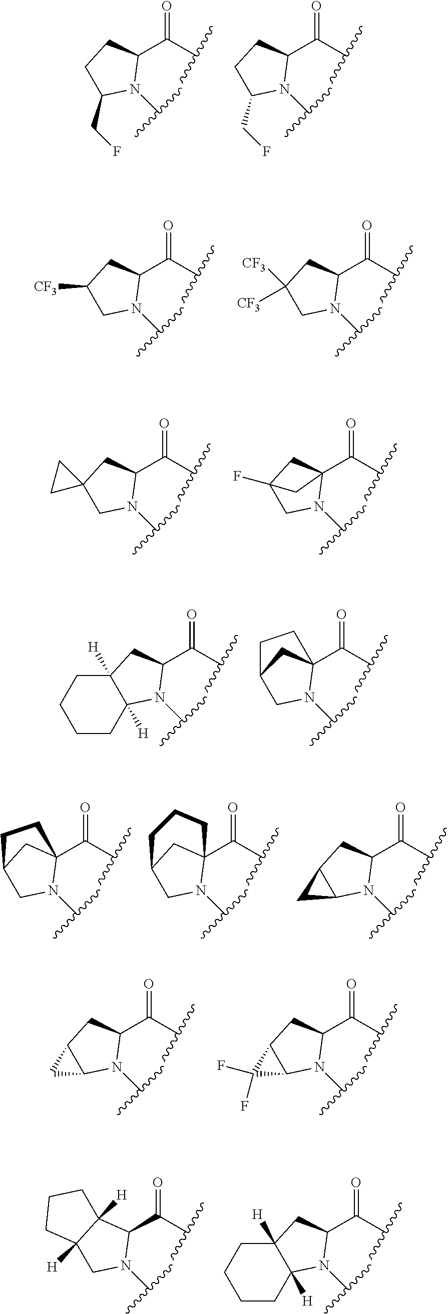

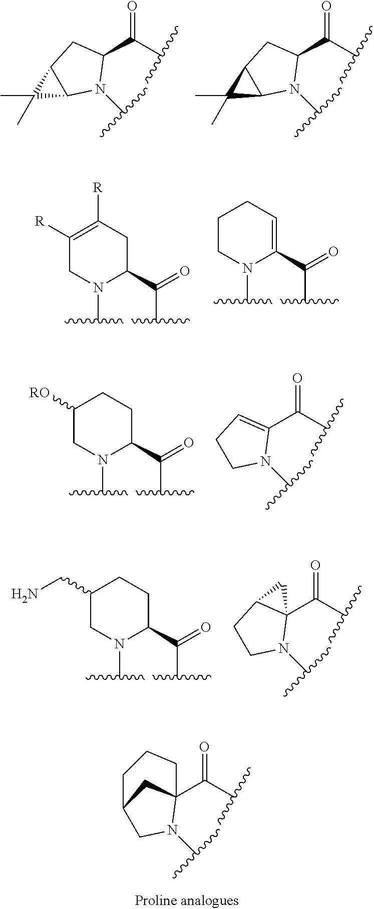

In preferred embodiments m is 2 and AA¹ and the two instances of AAⁿ together form a tripeptide represented by KAK, KAH, KAF, KA-homoPhe, KAA, KPA, KPK, FAF, HAH, ROK, RQF, RAF, RQ-homoPhe, HQF, homoHis-QF, (para-guanidinyl-Phe)-QF, (meta-guanidinyl-Phe)-QF, ((2-amino-1H-imidazol-4-yl)ethyl-Ala)-QF, ((2-amino-1H-imidazol-4-yl)methyl-Ala)-QF, ((2-amino-1H-imidazol-4-yl)-Ala)-QF, ((pyridin-3-yl)homoAla)-QF, ((pyridin-3-yl) Ala)-QF, ((pyridin-4-yl) Ala)-QF, ((1H-imidazol-2-yl)aminomethyl-Ala)-QF, ((1H-imidazol-2-yl)aminoethyl-Ala)-QF, ((pyridin-4-yl)homoAla)-QF, or RNF. In some embodiments the tripeptide is represented by tripeptide represented by KAK, KAH, KAF, KA-homoPhe, KAA, KPA, KPK, FAF, HAH, ROK, RNF, or RQF.

In some embodiments the tripeptide is represented by KAK, KAH, KAF, KA-homoPhe, KAA, KPA, KPK, FAF, HAH, ROK, RQF, RAF, RQ-homoPhe, HQF, homoHis-QF, or RNF. In some embodiments the tripeptide is represented by KAK, KAH, KAF, KAA, KPA, KPK, FAF, HAH, RQK, RQF, RAF, HOF, RQ-homoPhe, or RNF. In some embodiments the tripeptide is represented by KAK, KAH, KAF, KA-homoPhe, KAA, KPA, or KPK. In some embodiments the tripeptide is represented by KAK, KAH, KAF, KA-homoPhe, KAA, FAF, or HAH. In some embodiments the tripeptide is represented by KAK, KAH, KPK, HAH, or RQK. In some embodiments the tripeptide is represented by RQK, RQF, RMF, RAF, KAH, or RQ-homoPhe. In some embodiments the tripeptide is represented by FAF, HAH, RQK, or RQF. In some embodiments the tripeptide is represented by RQK or RQF. Such compounds are shown below, wherein reference is also made to the same structures wherein-C(O)-head are instead-(replacing head). The structures as depicted are more preferred.

| Tripeptide | Structure |
|---|---|
| KAK | 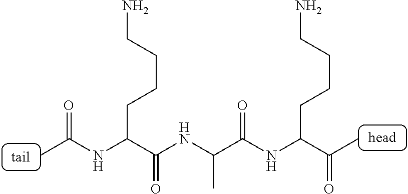 [SMILES: CC(=O)NC(CCCCN)C(=O)NC(C)C(=O)NC(CCCCN)C(C)=O] |
| KAH | 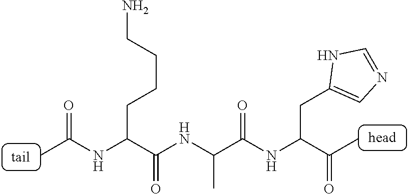 [SMILES: CC(=O)NC(CCCCN)C(=O)NC(C)C(=O)NC(Cc1cnc[nH]1)C(C)=O] |
| KAF | 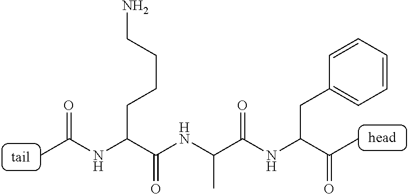 [SMILES: CC(=O)NC(CCCCN)C(=O)NC(C)C(=O)NC(Cc1ccccc1)C(C)=O] |
| KA-homoPhe | 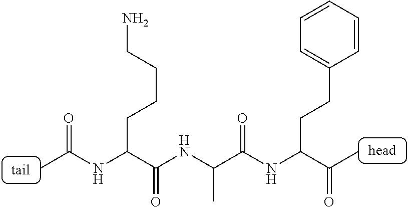 [SMILES: CC(=O)NC(CCCCN)C(=O)NC(C)C(=O)NC(CCc1ccccc1)C(C)=O] |
| KAA | 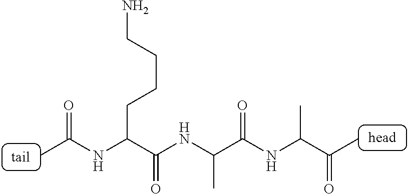 [SMILES: CC(=O)NC(CCCCN)C(=O)NC(C)C(=O)NC(C)C(C)=O] |
| KPA | 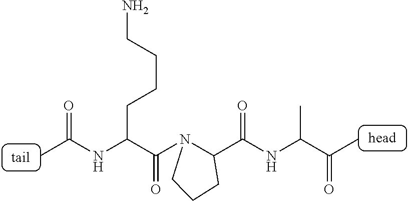 [SMILES: CC(=O)NC(CCCCN)C(=O)N1CCCC1C(=O)NC(C)C(C)=O] |
| KPK | 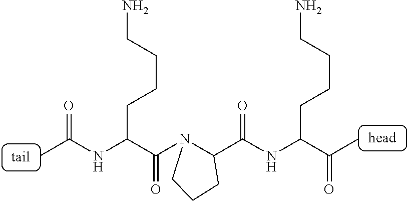 [SMILES: CC(=O)NC(CCCCN)C(=O)N1CCCC1C(=O)NC(CCCCN)C(C)=O] |
| FAF | 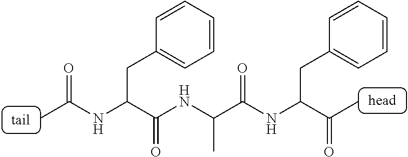 [SMILES: CC(=O)NC(Cc1ccccc1)C(=O)NC(C)C(=O)NC(Cc1ccccc1)C(C)=O] |
| HAH | 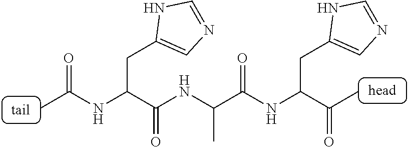 [SMILES: CC(=O)NC(Cc1cnc[nH]1)C(=O)NC(C)C(=O)NC(Cc1cnc[nH]1)C(C)=O] |
| RQK | 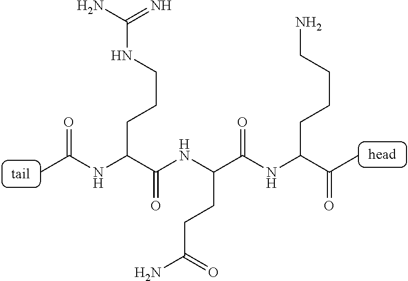 |
| RQF | 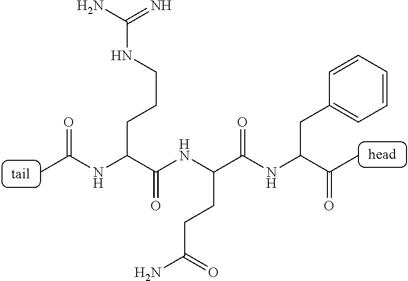 |
| RAF | 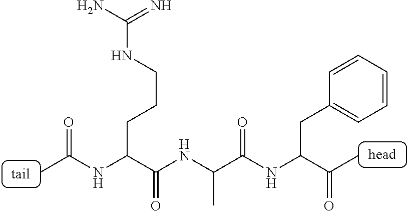 |
| RQ-homoPhe | 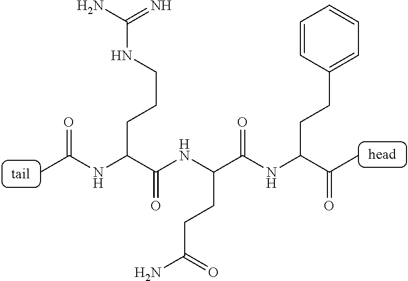 [SMILES: CC(=O)NC(CCCNC(=N)N)C(=O)NC(CCC(N)=O)C(=O)NC(CCc1ccccc1)C(C)=O] |
| HQF | 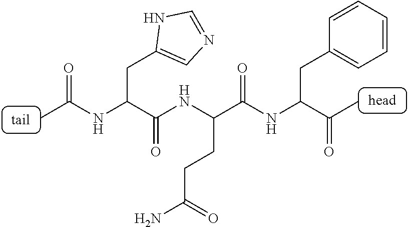 [SMILES: CC(=O)NC(Cc1cnc[nH]1)C(=O)NC(CCC(N)=O)C(=O)NC(Cc1ccccc1)C(C)=O] |
| homoHis-QF | 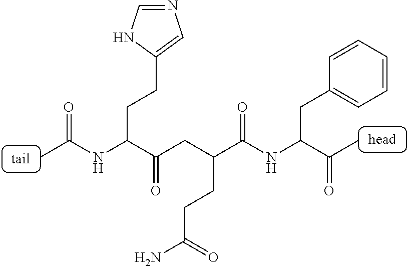 [SMILES: CC(=O)NC(CCc1cnc[nH]1)C(=O)CC(CCC(N)=O)C(=O)NC(Cc1ccccc1)C(C)=O] |
| (para-guanidinyl-Phe)- QF | 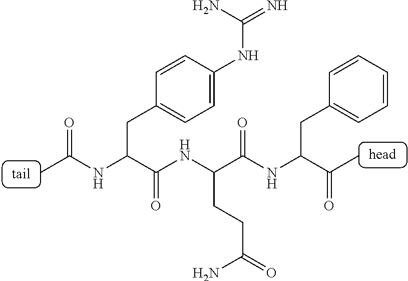 [SMILES: CC(=O)NC(Cc1ccc(NC(=N)N)cc1)C(=O)NC(CCC(N)=O)C(=O)NC(Cc1ccccc1)C(C)=O] |
| (meta-guanidinyl-Phe)- QF | 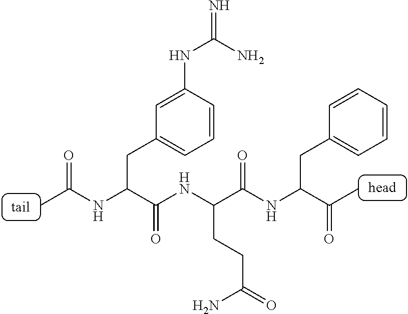 [SMILES: CC(=O)NC(Cc1cccc(NC(=N)N)c1)C(=O)NC(CCC(N)=O)C(=O)NC(Cc1ccccc1)C(C)=O] |
| ((2-amino-1H-imidazol- 4-yl)ethyl-Ala)-QF | 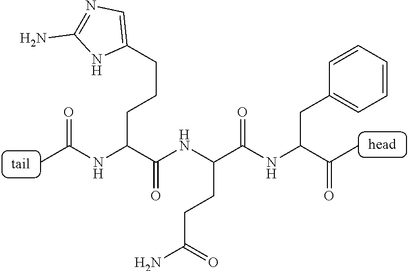 [SMILES: CC(=O)NC(CCCc1cnc(N)[nH]1)C(=O)NC(CCC(N)=O)C(=O)NC(Cc1ccccc1)C(C)=O] |
| ((2-amino-1H-imidazol- 4-yl)methyl-Ala)-QF | 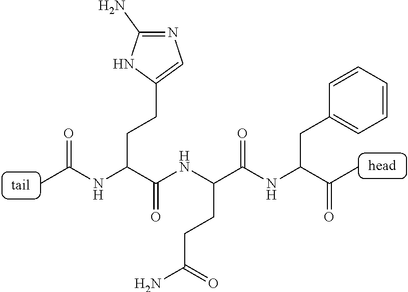 [SMILES: CC(=O)NC(CCc1cnc(N)[nH]1)C(=O)NC(CCC(N)=O)C(=O)NC(Cc1ccccc1)C(C)=O] |
| ((2-amino-1H-imidazol- 4-yl)-Ala)-QF | 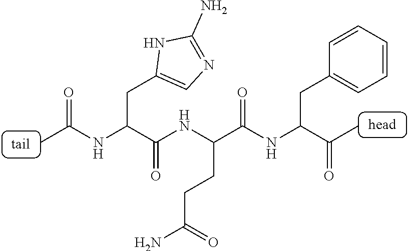 [SMILES: CC(=O)NC(Cc1cnc(N)[nH]1)C(=O)NC(CCC(N)=O)C(=O)NC(Cc1ccccc1)C(C)=O] |
| ((pyridin-3-yl)homoAla)- QF | 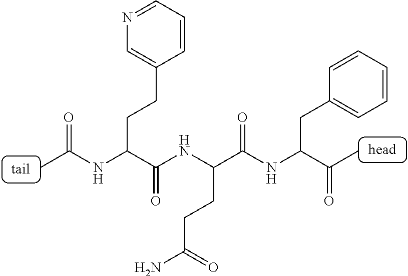 [SMILES: CC(=O)NC(CCc1cccnc1)C(=O)NC(CCC(N)=O)C(=O)NC(Cc1ccccc1)C(C)=O] |
| ((pyridin-3-yl)Ala)-QF | 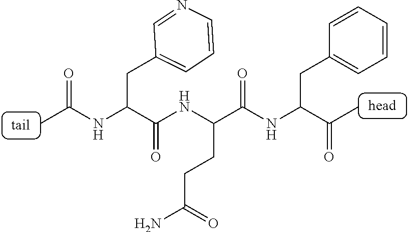 [SMILES: CC(=O)NC(Cc1cccnc1)C(=O)NC(CCC(N)=O)C(=O)NC(Cc1ccccc1)C(C)=O] |
| ((pyridin-4-yl)Ala)-QF | 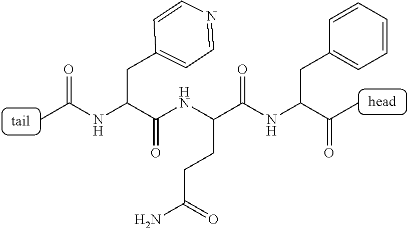 [SMILES: CC(=O)NC(Cc1ccncc1)C(=O)NC(CCC(N)=O)C(=O)NC(Cc1ccccc1)C(C)=O] |
| ((pyridin-4-yl)homoAla)- QF | 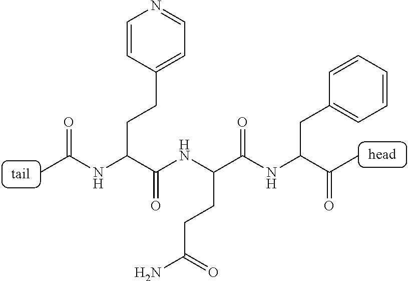 [SMILES: CC(=O)NC(CCc1ccncc1)C(=O)NC(CCC(N)=O)C(=O)NC(Cc1ccccc1)C(C)=O] |
| ((1H-imidazol-2- yl)aminomethyl-Ala)-QF | 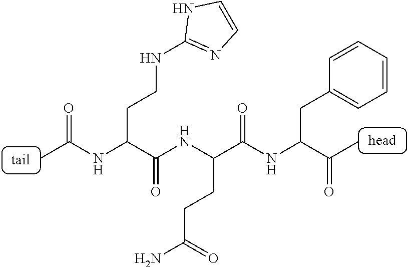 [SMILES: CC(=O)NC(CCNc1ncc[nH]1)C(=O)NC(CCC(N)=O)C(=O)NC(Cc1ccccc1)C(C)=O] |
| ((1H-imidazol-2- yl)aminoethyl-Ala)-QF | 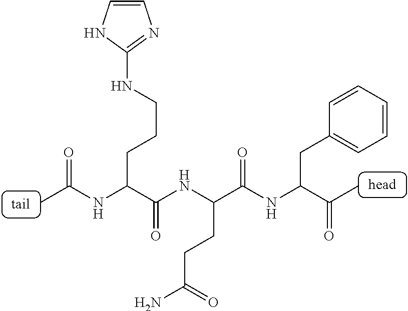 [SMILES: CC(=O)NC(CCCNc1ncc[nH]1)C(=O)NC(CCC(N)=O)C(=O)NC(Cc1ccccc1)C(C)=O] |
| RNF | 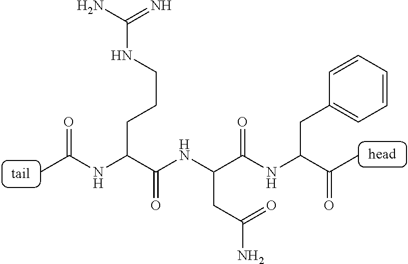 |

In preferred embodiments m is 3 and AA¹ and the three instances of AAⁿ together form a tetrapeptide represented by
KAAA, KAAF, KAAH, KAAK, KAAW, KAA-homoHis, KAA-homoPhe, KAA-phenylGly, KAA-(4-OBn-phenylGly),
KPAF, KPAH, KPAK, KAPK, KPAW, KPA-homoHis, KPA-homoPhe, KPA-phenylGly, KPA-(4-OBn-phenylGly),
AAAK, AAKA, AKAA, AKAK, APAK, A-(4-O-(4-Py)-Pro)-AK, AVAK, AKKK, LKAK, LKKK, VKAK,
KPHK, KPH-homoPhe, K-(pipecolic acid)-AK, K-(pipecolic acid)-HK, KP-phenylglycine-K, K-(pipecolic acid)-phenylglycine-K,
ARQK, ARQF, ARRK, FKKK, RARK, FAAF, or WAAW.

In some embodiments the tetrapeptide is represented by KPAF, KPAH, KPAK, KAPK, KPAW, KPA-homoHis, KPA-homoPhe, KPA-phenylGly, KPA-(4-OBn-phenylGly), AAAK, AAKA, AKAA, AKAK, APAK, A-(4-O-(4-Py)-Pro)-AK, AVAK, AKKK, LKAK, LKKK, VKAK, KPHK, KPH-homoPhe, K-(pipecolic acid)-AK, K-(pipecolic acid)-HK, KP-phenylglycine-K, K-(pipecolic acid)-phenylglycine-K, ARQK, ARQF, ARRK, FKKK, RARK, FAAF, or WAAW. In some embodiments the tetrapeptide is represented by KAAA, KAAF, KAAH, KAAK, KAAW, KAA-homoHis, KAA-homoPhe, KAA-phenylGly, KAA-(4-OBn-phenylGly), AAAK, AAKA, AKAA, AKAK, APAK, A-(4-O-(4-Py)-Pro)-AK, AVAK, AKKK, LKAK, LKKK, VKAK, KPHK, KPH-homoPhe, K-(pipecolic acid)-AK, K-(pipecolic acid)-HK, KP-phenylglycine-K, K-(pipecolic acid)-phenylglycine-K, ARQK, ARQF, ARRK, FKKK, RARK, FAAF, or WAAW. In some embodiments the tetrapeptide is represented by KAAA, KAAF, KAAH, KAAK, KAAW, KAA-homoHis, KAA-homoPhe, KAA-phenylGly, KAA-(4-OBn-phenylGly), KPAF, KPAH, KPAK, KAPK, KPAW, KPA-homoHis, KPA-homoPhe, KPA-phenylGly, KPA-(4-OBn-phenylGly), KPHK, KPH-homoPhe, K-(pipecolic acid)-AK, K-(pipecolic acid)-HK, KP-phenylglycine-K, K-(pipecolic acid)-phenylglycine-K, ARQK, ARQF, ARRK, FKKK, RARK, FAAF, or WAAW. In some embodiments the tetrapeptide is represented by KAAA, KAAF, KAAH, KAAK, KAAW, KAA-homoHis, KAA-homoPhe, KAA-phenylGly, KAA-(4-OBn-phenylGly), KPAF, KPAH, KPAK, KAPK, KPAW, KPA-homoHis, KPA-homoPhe, KPA-phenylGly, KPA-(4-OBn-phenylGly), AAAK, AAKA, AKAA, AKAK, APAK, A-(4-O-(4-Py)-Pro)-AK, AVAK, AKKK, LKAK, LKKK, VKAK, ARQK, ARQF, ARRK, FKKK, RARK, FAAF, or WAAW. In some embodiments the tetrapeptide is represented by KAAA, KAAF, KAAH, KAAK, KAAW, KAA-homoHis, KAA-homoPhe, KAA-phenylGly, KAA-(4-OBn-phenylGly), KPAF, KPAH, KPAK, KAPK, KPAW, KPA-homoHis, KPA-homoPhe, KPA-phenylGly, KPA-(4-OBn-phenylGly), AAAK, AAKA, AKAA, AKAK, APAK, A-(4-O-(4-Py)-Pro)-AK, AVAK, AKKK, LKAK, LKKK, VKAK, KPHK, KPH-homoPhe, K-(pipecolic acid)-AK, K-(pipecolic acid)-HK, KP-phenylglycine-K, or K-(pipecolic acid)-phenylglycine-K.

In some embodiments the tetrapeptide is represented by KAAA, KAAF, KAAH, KAAK, KAAW, KAA-homoHis, KAA-homoPhe, KAA-phenylGly, KAA-(4-OBn-phenylGly), KPAF, KPAH, KPAK, KAPK, KPAW, KPA-homoHis, KPA-homoPhe, KPA-phenylGly, KPA-(4-OBn-phenylGly), KPHK, KPH-homoPhe, K-(pipecolic acid)-AK, K-(pipecolic acid)-HK, KP-phenylglycine-K, or K-(pipecolic acid)-phenylglycine-K. In some embodiments the tetrapeptide is represented by AAAK, AAKA, AKAA, AKAK, APAK, A-(4-O-(4-Py)-Pro)-AK, AVAK, AKKK, LKAK, LKKK, VKAK, ARQK, ARQF, ARRK, FKKK, RARK, FAAF, or WAAW.

In preferred embodiments, when AA¹ is K, AA² is A or P. In preferred embodiments, when AA¹ is K and AA² is A or P, AA³ is A or P. In preferred embodiments, when AA¹ is K, AA² is P. In preferred embodiments, when AA¹ is K, AA² is A. In preferred embodiments, when AA¹ is K and AA² is A, AA³ is A. In preferred embodiments, when AA¹ is K and AA² is P, AA³ is A.

Such compounds are shown below, wherein reference is also made to the same structures wherein-C(O)-head are instead-(replacing head). The structures as depicted are more preferred.

| Tetrapeptide | |
|---|---|
| (SEQ ID NO:) | Structure |
| KAAA (1) | 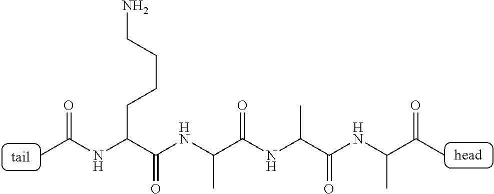 [SMILES: CC(=O)NC(CCCCN)C(=O)NC(C)C(=O)NC(C)C(=O)NC(C)C(C)=O] |
| KAAF (2) | 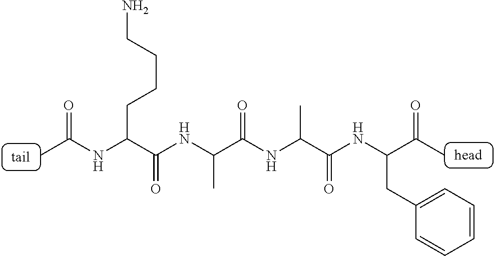 [SMILES: CC(=O)NC(CCCCN)C(=O)NC(C)C(=O)NC(C)C(=O)NC(Cc1ccccc1)C(C)=O] |
| KAAH (3) | 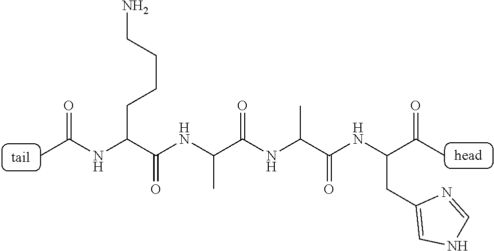 [SMILES: CC(=O)NC(CCCCN)C(=O)NC(C)C(=O)NC(C)C(=O)NC(Cc1c[nH]cn1)C(C)=O] |
| KAAK (4) | 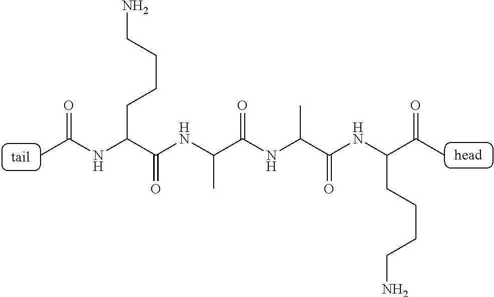 [SMILES: CC(=O)NC(CCCCN)C(=O)NC(C)C(=O)NC(C)C(=O)NC(CCCCN)C(C)=O] |
| KAAW (5) | 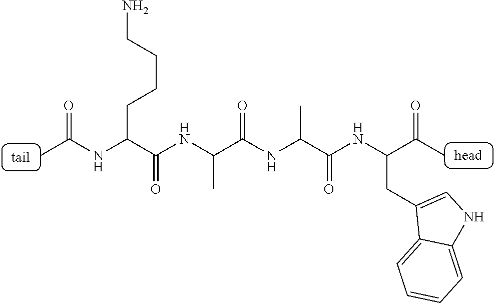 [SMILES: CC(=O)NC(CCCCN)C(=O)NC(C)C(=O)NC(C)C(=O)NC(Cc1c[nH]c2ccccc12)C(C)=O] |
| KAA-homoHis (6) | 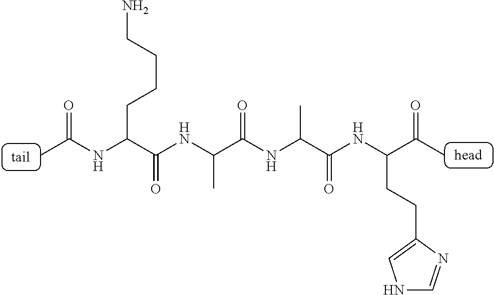 [SMILES: CC(=O)NC(CCCCN)C(=O)NC(C)C(=O)NC(C)C(=O)NC(CCc1c[nH]cn1)C(C)=O] |
| KAA-homoPhe (7) | 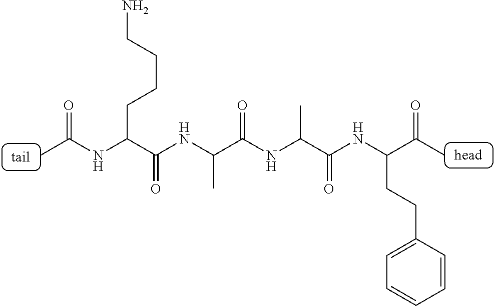 [SMILES: CC(=O)NC(CCCCN)C(=O)NC(C)C(=O)NC(C)C(=O)NC(CCc1ccccc1)C(C)=O] |
| KAA-phenylGly (8) | 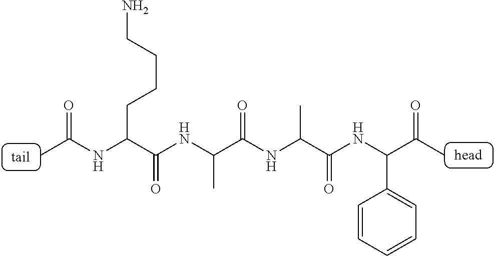 [SMILES: CC(=O)NC(CCCCN)C(=O)NC(C)C(=O)NC(C)C(=O)NC(C(C)=O)c1ccccc1] |
| KAA-(4-OBn- phenylGly) (9) | 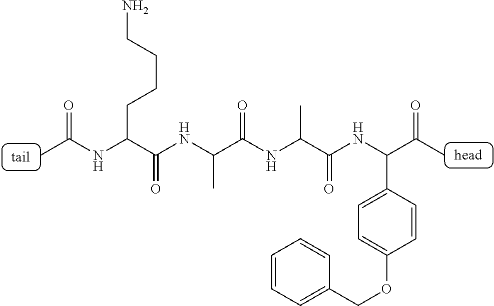 [SMILES: CC(=O)NC(CCCCN)C(=O)NC(C)C(=O)NC(C)C(=O)NC(C(C)=O)c1ccc(OCc2ccccc2)cc1] |
| KPAF (10) | 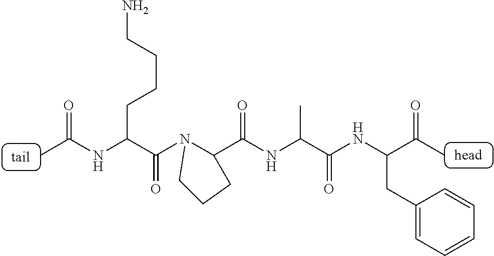 [SMILES: CC(=O)NC(CCCCN)C(=O)N1CCCC1C(=O)NC(C)C(=O)NC(Cc1ccccc1)C(C)=O] |
| KPAH (11) | 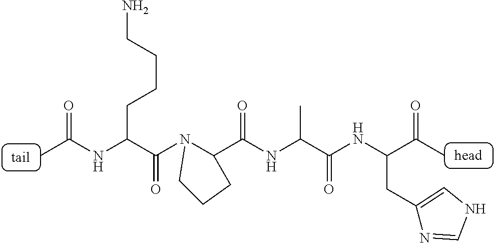 [SMILES: CC(=O)NC(CCCCN)C(=O)N1CCCC1C(=O)NC(C)C(=O)NC(Cc1c[nH]cn1)C(C)=O] |
| KPAK (12) | 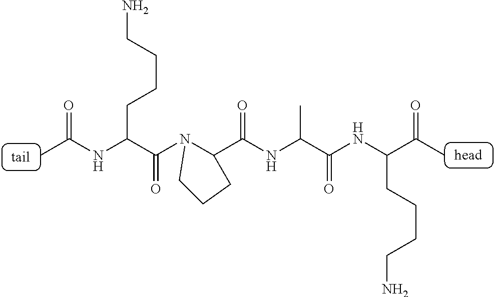 [SMILES: CC(=O)NC(CCCCN)C(=O)N1CCCC1C(=O)NC(C)C(=O)NC(CCCCN)C(C)=O] |
| KAPK (13) | 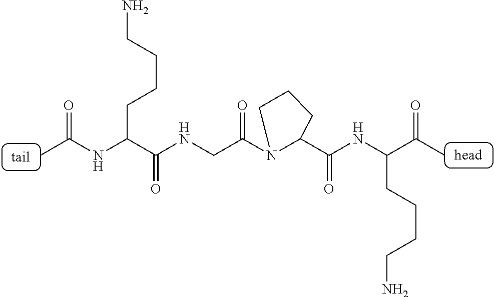 [SMILES: CC(=O)NC(CCCCN)C(=O)NCC(=O)N1CCCC1C(=O)NC(CCCCN)C(C)=O] |
| KPAW (14) | 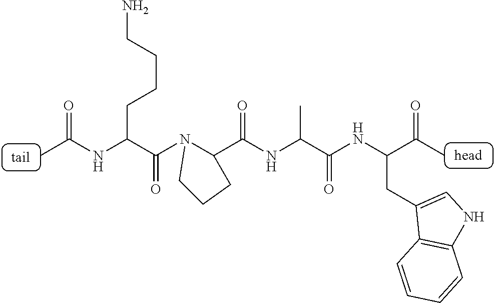 [SMILES: CC(=O)NC(CCCCN)C(=O)N1CCCC1C(=O)NC(C)C(=O)NC(Cc1c[nH]c2ccccc12)C(C)=O] |
| KPA-homoHis (15) | 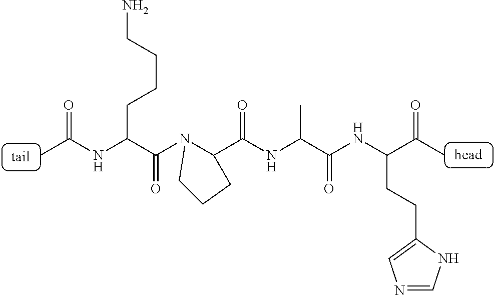 [SMILES: CC(=O)NC(CCCCN)C(=O)N1CCCC1C(=O)NC(C)C(=O)NC(CCc1cnc[nH]1)C(C)=O] |
| KPA-homoPhe (16) | 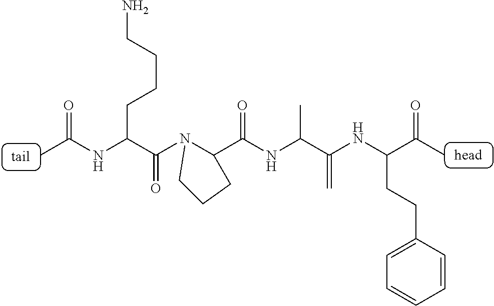 [SMILES: C=C(NC(CCc1ccccc1)C(C)=O)C(C)NC(=O)C1CCCN1C(=O)C(CCCCN)NC(C)=O] |
| KPA-phenylGly (17) | 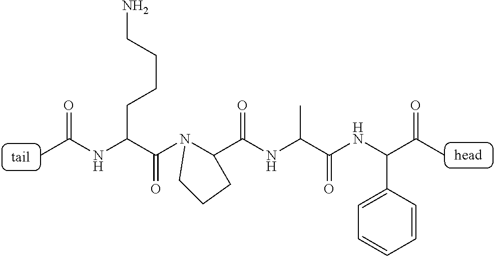 [SMILES: CC(=O)NC(CCCCN)C(=O)N1CCCC1C(=O)NC(C)C(=O)NC(C(C)=O)c1ccccc1] |
| KPA-(4-OBn- phenylGly) (18) | 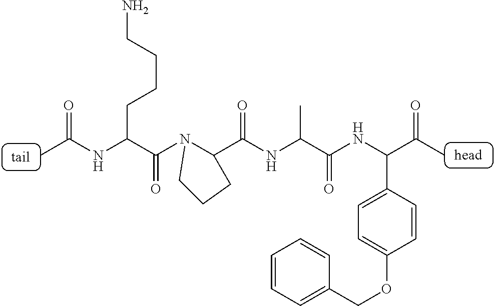 [SMILES: CC(=O)NC(CCCCN)C(=O)N1CCCC1C(=O)NC(C)C(=O)NC(C(C)=O)c1ccc(OCc2ccccc2)cc1] |
| AAAK (19) | 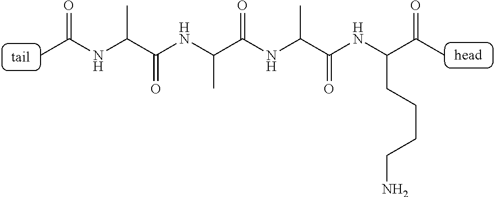 [SMILES: CC(=O)NC(C)C(=O)NC(C)C(=O)NC(C)C(=O)NC(CCCCN)C(C)=O] |
| AAKA (20) | 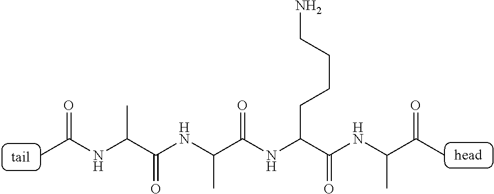 [SMILES: CC(=O)NC(C)C(=O)NC(C)C(=O)NC(CCCCN)C(=O)NC(C)C(C)=O] |
| AKAA (21) | 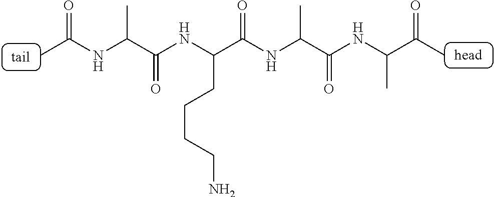 [SMILES: CC(=O)NC(C)C(=O)NC(CCCCN)C(=O)NC(C)C(=O)NC(C)C(C)=O] |
| AKAK (22) | 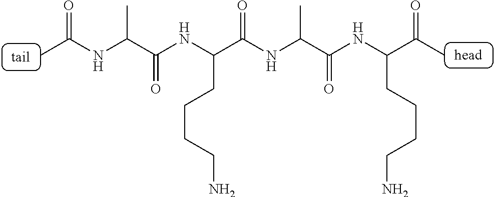 [SMILES: CC(=O)NC(C)C(=O)NC(CCCCN)C(=O)NC(C)C(=O)NC(CCCCN)C(C)=O] |
| APAK (23) | 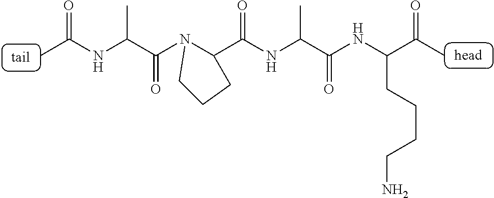 [SMILES: CC(=O)NC(C)C(=O)N1CCCC1C(=O)NC(C)C(=O)NC(CCCCN)C(C)=O] |
| A-(4-O-(4-Py)-Pro)-AK (24) | 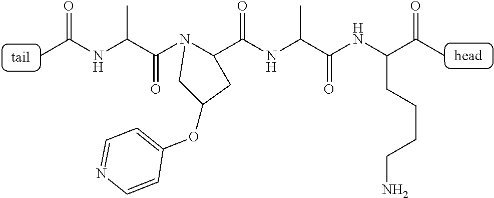 [SMILES: CC(=O)NC(C)C(=O)N1CC(Oc2ccncc2)CC1C(=O)NC(C)C(=O)NC(CCCCN)C(C)=O] |
| AVAK (25) | 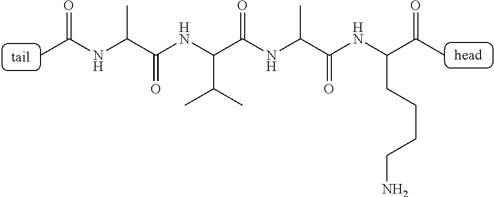 [SMILES: CC(=O)NC(C)C(=O)NC(C(=O)NC(C)C(=O)NC(CCCCN)C(C)=O)C(C)C] |
| AKKK (26) | 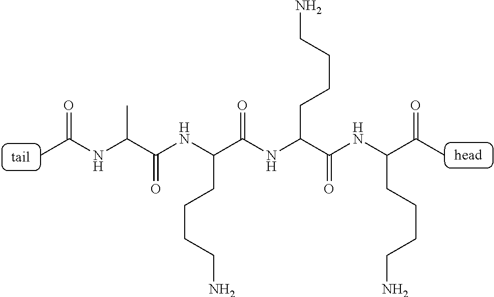 [SMILES: CC(=O)NC(C)C(=O)NC(CCCCN)C(=O)NC(CCCCN)C(=O)NC(CCCCN)C(C)=O] |
| LKAK (27) | 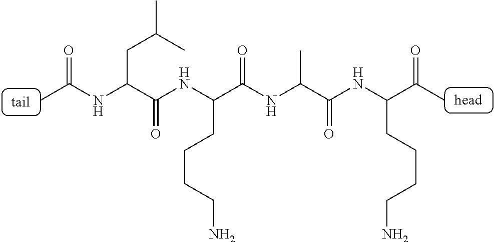 [SMILES: CC(=O)NC(CC(C)C)C(=O)NC(CCCCN)C(=O)NC(C)C(=O)NC(CCCCN)C(C)=O] |
| LKKK (28) | 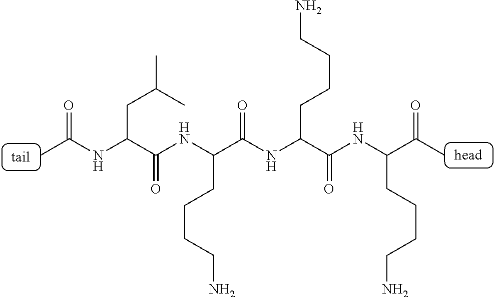 [SMILES: CC(=O)NC(CC(C)C)C(=O)NC(CCCCN)C(=O)NC(CCCCN)C(=O)NC(CCCCN)C(C)=O] |
| VKAK (29 | 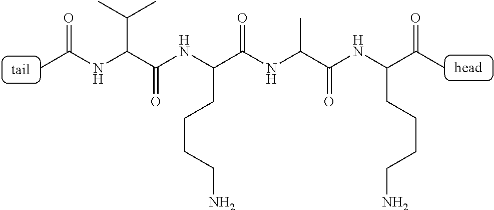 [SMILES: CC(=O)NC(C(=O)NC(CCCCN)C(=O)NC(C)C(=O)NC(CCCCN)C(C)=O)C(C)C] |
| KPHK (30) | 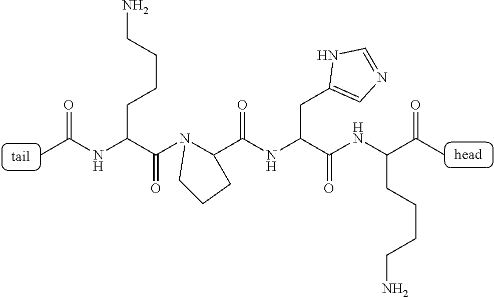 [SMILES: CC(=O)NC(CCCCN)C(=O)N1CCCC1C(=O)NC(Cc1cnc[nH]1)C(=O)NC(CCCCN)C(C)=O] |
| KPH-homoPhe (31) | 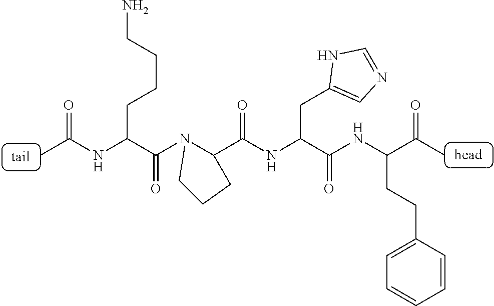 [SMILES: CC(=O)NC(CCCCN)C(=O)N1CCCC1C(=O)NC(Cc1cnc[nH]1)C(=O)NC(CCc1ccccc1)C(C)=O] |
| K-(pipecolic acid)-AK (32) | 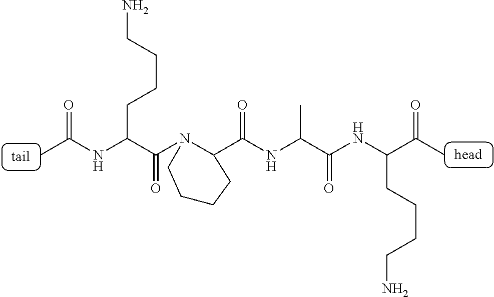 [SMILES: CC(=O)NC(CCCCN)C(=O)N1CCCCC1C(=O)NC(C)C(=O)NC(CCCCN)C(C)=O] |
| K-(pipecolic acid)-HK (33) | 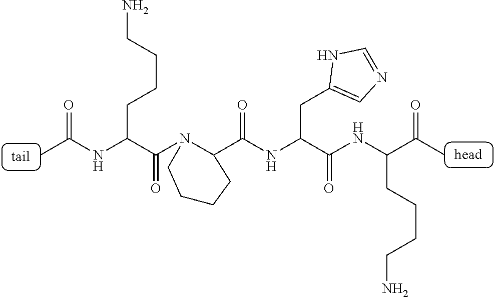 [SMILES: CC(=O)NC(CCCCN)C(=O)N1CCCCC1C(=O)NC(Cc1cnc[nH]1)C(=O)NC(CCCCN)C(C)=O] |
| KP-phenylglycine-K (34) | 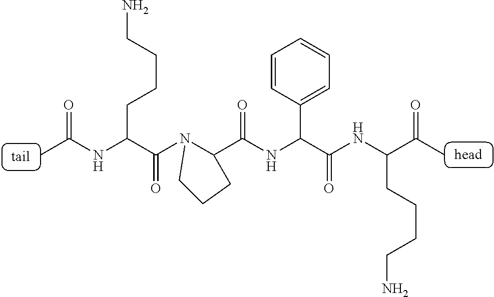 [SMILES: CC(=O)NC(CCCCN)C(=O)N1CCCC1C(=O)NC(C(=O)NC(CCCCN)C(C)=O)c1ccccc1] |
| K-(pipecolic acid)- phenylglycine-K (35) | 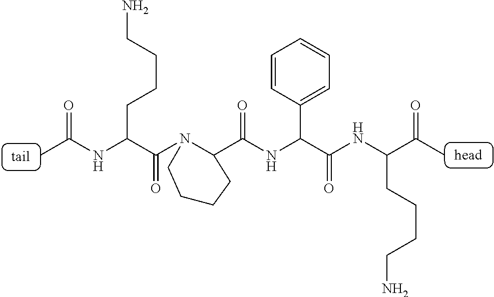 [SMILES: CC(=O)NC(CCCCN)C(=O)N1CCCCC1C(=O)NC(C(=O)NC(CCCCN)C(C)=O)c1ccccc1] |
| ARQK (36) | 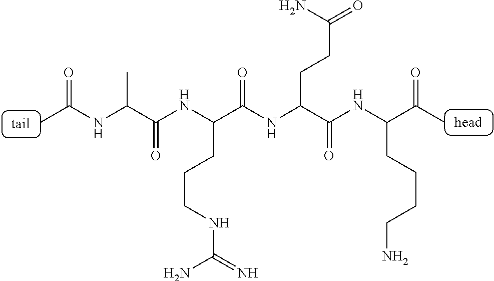 [SMILES: CC(=O)NC(C)C(=O)NC(CCCNC(=N)N)C(=O)NC(CCC(N)=O)C(=O)NC(CCCCN)C(C)=O] |
| ARQF (37) | 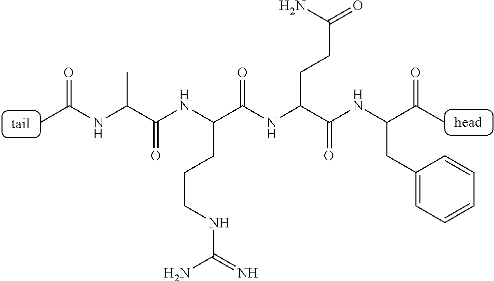 [SMILES: CC(=O)NC(C)C(=O)NC(CCCNC(=N)N)C(=O)NC(CCC(N)=O)C(=O)NC(Cc1ccccc1)C(C)=O] |
| ARRK (38) | 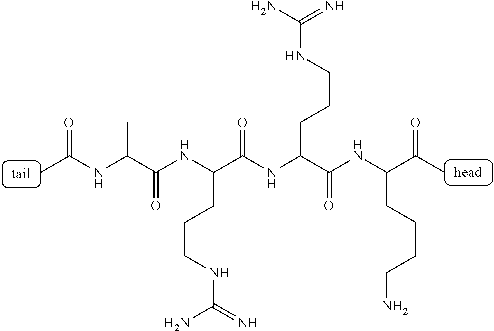 [SMILES: CC(=O)NC(C)C(=O)NC(CCCNC(=N)N)C(=O)NC(CCCNC(=N)N)C(=O)NC(CCCCN)C(C)=O] |
| FKKK (39) | 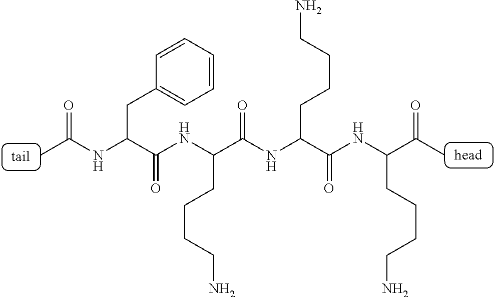 [SMILES: CC(=O)NC(Cc1ccccc1)C(=O)NC(CCCCN)C(=O)NC(CCCCN)C(=O)NC(CCCCN)C(C)=O] |
| RARK (40) | 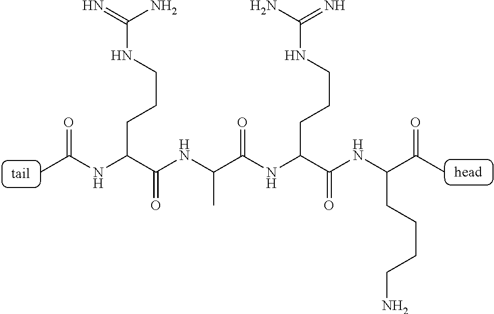 [SMILES: CC(=O)NC(CCCNC(=N)N)C(=O)NC(C)C(=O)NC(CCCNC(=N)N)C(=O)NC(CCCCN)C(C)=O] |
| FAAF (41) | 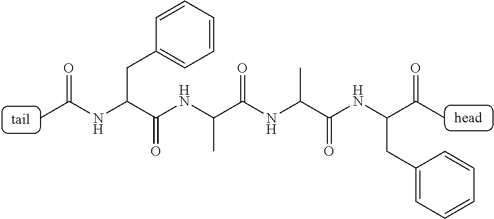 [SMILES: CC(=O)NC(Cc1ccccc1)C(=O)NC(C)C(=O)NC(C)C(=O)NC(Cc1ccccc1)C(C)=O] |
| WAAW (42) | 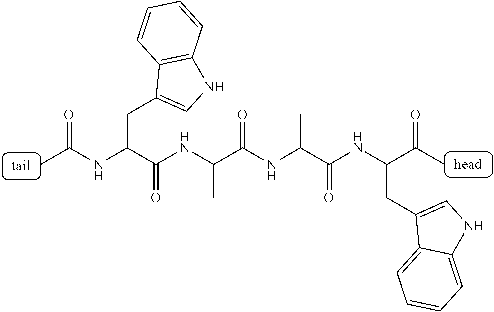 [SMILES: CC(=O)NC(Cc1c[nH]c2ccccc12)C(=O)NC(C)C(=O)NC(C)C(=O)NC(Cc1c[nH]c2ccccc12)C(C)=O] |

### Tail

The tail moiety of the compounds is found at the N-terminus of the central peptide analogues as defined above. The tail is generally connected via a carbonyl moiety as depicted in general formula (1), which thus forms an amide bond with the backbone amine of AA¹. The tail is generally hydrophobic and can be a long aliphatic moiety, although it can vary as defined.
tail is C1-24alkyl, —O—C1-24alkyl, 5-20-membered (hetero)aryl, or 3-20-membered (hetero)cycloalkyl, wherein tail is optionally unsaturated, wherein tail is optionally substituted with halogen, C1-3 (halo)alkyl, or C1-3 (halo)alkoxyl; in some embodiments tail is not optionally unsaturated and not optionally substituted; in some embodiments tail is not optionally unsaturated; in some embodiments tail is not optionally substituted; preferred optional substitutions are -F, -Cl, —CH₃, and —OCH₃, more preferably —CH₃ and —OCH₃, most preferably —OCH₃, tail preferably comprises at least 8 carbon atoms, more preferably at least 10, even more preferably at least 13, most preferably at least 15. A highly preferred tail is pentadecyl (which is —(CH₂)₁₄CH₃) thus forming a palmitic acid amide with the carbonyl moiety of general formula 1 and the N-terminus of AA¹.

In tail C1-24alkyl is preferably C1-20 alkyl, more preferably C2-19 alkyl, even more preferably C6-C15 alkyl, wherein alkyl is preferably straight and unbranched. The alkyl preferably comprises at least 2 carbon atoms, more preferably at least 6, even more preferably at least 9, more preferably at least 13, most preferably at least 15. The alkyl preferably comprises at most 21 carbon atoms, more preferably at most 19, even more preferably at most 17, more preferably at most 15. Another preferred embodiment is where tail is CH₃.

In tail-O-C1-24alkyl is preferably —O-C1-20 alkyl, more preferably —O-C2-19 alkyl, even more preferably —O-C2-C8 alkyl, wherein alkyl is preferably branched. The —O-alkyl preferably comprises at least 2 carbon atoms, more preferably at least 3, even more preferably at least 4. The —O-alkyl preferably comprises at most 20 carbon atoms, more preferably at most 16, even more preferably at most 10, more preferably at most 4. Highly preferred-O-alkyl are -O—CH₃ and —O—C(CH₃)₃, or which the latter is most preferred.

In tail 5-20-membered (hetero)aryl is preferably 5-12 membered, or 5-10 membered. It preferably comprises at least one 5-6 membered (hetero)aryl ring, and optionally it further comprises a second 5-6 membered (hetero)aryl ring. In some embodiments the (hetero)aryl is aryl. In some embodiments the (hetero)aryl is heteroaryl. Preferred 5-20-membered (hetero)aryl are optionally substituted phenyl, optionally substituted thiophene, and optionally substituted indole. A preferred substitution of phenyl herein is a second phenyl, preferably forming biphenyl, which is preferably linked to the compound of general formula (1) at a 4-position. A preferred substitution of thiophene herein is a second thiophene, preferably forming 2-2′-thiophene, which is preferably linked to the compound of general formula (1) at a 5-position. Indole is preferably linked to the compound of general formula (1) at its 2-position, and a preferred substitution of indole is —OCH₃, preferably forming-[4-methoxy-1H-indole-2-yl].

In tail 3-20-membered (hetero)cycloalkyl is preferably 3-15 membered, more preferably 3-membered, even more preferably 5-10 membered, most preferably 5-6 membered or 10-membered. Preferred examples of 3-20-membered (hetero)cycloalkyl are cyclohexyl and adamantanyl, preferably adamantan -2-yl.

In preferred embodiments tail is C1-20alkyl such as methyl or ethyl or hexanyl or heptanyl or nonanyl or undecanyl or tridecanyl or pentadecanyl or nonadecanyl, —O—C1-16alkyl such as —O-butyl, 3-12-membered (hetero)cycloalkyl such as adamantanyl or cyclohexyl, or 5-12-membered (hetero)aryl such as phenyl or biphenyl or bithiophene or substituted indole such as methoxyindole. More preferably tail is C1-20alkyl such as methyl or ethyl or hexanyl or heptanyl or nonanyl or undecanyl or tridecanyl or pentadecanyl or nonadecanyl, 3-12-membered (hetero)cycloalkyl such as adamantanyl or cyclohexyl, or 5-12-membered (hetero)aryl such as phenyl or biphenyl or bithiophene or substituted indole such as methoxyindole. Even more preferably tail is C1-20alkyl such as methyl or ethyl or hexanyl or heptanyl or nonanyl or undecanyl or tridecanyl or pentadecanyl or nonadecanyl or 5-12-membered (hetero)aryl such as phenyl or biphenyl or bithiophene or substituted indole such as methoxyindole.

In preferred embodiments, tail is —CH₃, —CH₂CH₃, —(CH₂)₅CH₃, —(CH₂)₆CH₃, —(CH₂)₈CH₃, —(CH₂)₁₀CH₃, —(CH₂)₁₂CH₃, —(CH₂)₁₄CH₃, —(CH₂)₁₉CH₃, -cyclohexanyl, -adamantan -2-yl, -phenyl, -biphen-4-yl, -[2,2′-bithiophen-5-yl], -[4-methoxy-1H-indole-2-yl], or —O—C(CH₃)₃. In other preferred embodiments, tail is —CH₃, —CH₂CH₃, —(CH₂)₅CH₃, —(CH₂)₆CH₃, —(CH₂)₈CH₃, —(CH₂)₁₀CH₃, —(CH₂)₁₂CH₃, —(CH₂)₁₄CH₃, —(CH₂)₁₉CH₃, -cyclohexanyl, -adamantan -2-yl, -phenyl, or -biphen-4-yl. In other preferred embodiments, tail is —CH₃, —CH₂CH₃, —(CH₂)₅CH₃, —(CH₂)₆CH₃, —(CH₂)₈CH₃, —(CH₂)₁₀CH₃, —(CH₂)₁₂CH₃, —(CH₂)₁₄CH₃, or —(CH₂)₁₉CH₃. In other preferred embodiments, tail is —(CH₂)₈CH₃, —(CH₂)₁₀CH₃, —(CH₂)₁₂CH₃, —(CH₂)₁₄CH₃, or —(CH₂)₁₉CH₃. In other preferred embodiments, tail is -cyclohexanyl, -adamantan -2-yl, -phenyl, -biphen-4-yl, -[2,2′-bithiophen-5-yl], [4-methoxy-1H-indole-2-yl], or —O—C(CH₃)₃. In other preferred embodiments, tail is -phenyl, -biphen-4-yl, -[2,2′-bithiophen-5-yl], or -[4-methoxy-1H-indole-2-yl]. In other preferred embodiments, tail-phenyl, -biphen-4-yl, or -[2,2′-bithiophen-5-yl]. In other preferred embodiments, tail is -biphen-4-yl or -[2,2′-bithiophen-5-yl]. Representative embodiments of tail are shown below.

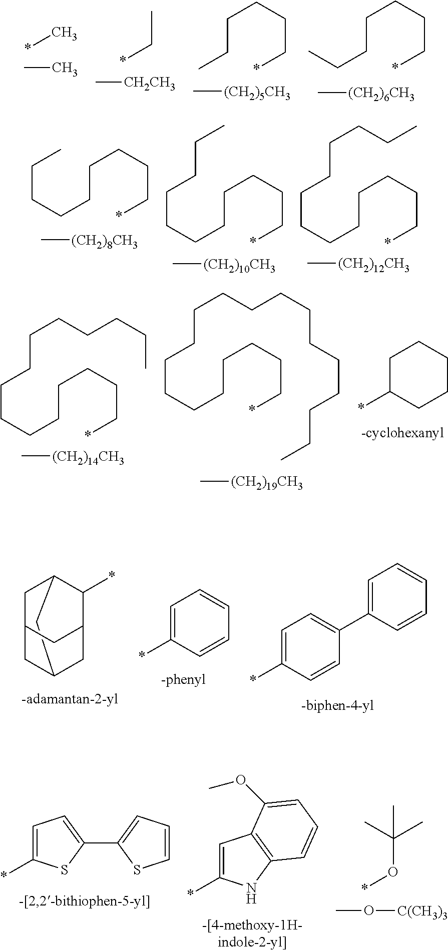

### Head

The head moiety of the compounds is found at the C-terminus of the central peptide analogues as defined above. It is generally a moiety that can help improve the potency of the compounds, or it can be a secondary, shorter tail. However, there are embodiments wherein head is not smaller than tail. head is connected to the carbonyl moiety of the AAⁿ that is adjacent to head, and depending on the nature of head it can thus form moieties ranging from an aldehyde (head is H) or a free carboxylic acid (head is —OH) to an octyl ester (head is —O—(CH₂)₇—CH₃) or a ketoamide (head is —C(O)—NH₂). As described earlier herein, head can also together with the carbonyl moiety of its adjacent AAⁿ form replacing head. In some embodiments replacing head is not formed. In other embodiments replacing head is formed.
head is —H, -h1, —O-h1, —C(O)-h1, —C(O)—N(H)h1, —N(h2) h1, or head is —C1-24alkyl, —(NH)₀₋₁-5-20-membered (hetero)aryl, or —(NH)₀₋₁-3-20-membered (hetero)cycloalkyl, wherein head is optionally unsaturated, wherein head is optionally substituted with halogen, C1-3 (halo)alkyl, or C1-3 (halo)alkoxyl; in some embodiments head is not optionally unsaturated; in some embodiments head is not optionally substituted; in some embodiments head is not optionally unsaturated and not optionally substituted; preferred optional substitutions of head are halogen, C1-2 (halo)alkyl, or C1-2 (halo)alkoxyl, more preferably halogen, —CF₃, —CH₃, or —OCH₃, wherein halogen is preferably F.

When head is —H, the C-terminus of the central peptide forms an aldehyde. When head is —OH it forms a carboxylic acid, and when it is —O-h1 it forms an ester. When head is —NH₂ it forms a terminal amide, and when head is —NH-h1 it forms an amide moiety that is further substituted with h1. When head is —C(O)-h1 it forms an 1,2-diketo moiety that is further substituted with h1, and when head is —C(O)—N(H)h1 is forms a ketoamide that is further substituted with h1.

In preferred embodiments head is -h1, —O-h1, —C(O)-h1, —C(O)—N(H)h1, —N(h2) h1, -C1-14alkyl, -5-6-membered (hetero)aryl, -3-8-membered cycloalkyl, —(NH)-5-6-membered (hetero)aryl, or —NH-3-8-membered cycloalkyl. More preferably head is -h1, —OH, —OCH₃, —C(O)—N(H)h1, —N (H)h1, —CH₃, or -5-6-membered aryl. Most preferably head is —N(h2) h1, of which-N(H)h1 and —N(CH₃) h1 are preferred, and —N(H)h1 is most preferred.

h1 and h2 can be comprised in head. h1 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h1 is optionally substituted with halogen, C1-3 (halo)alkyl, or C1-3 (halo)alkoxyl. h2 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h2 is optionally substituted with halogen, C1-3 (halo)alkyl, or C1-3 (halo)alkoxyl. When both h1 and h2 are present, it is preferred that at least one of h1 and h2 is —H or -CH₃, more preferably —H. In preferred embodiments it is h2 that is then-H or —CH₃, more preferably —H.

h1 is preferably —H, —OH, C1-8 (halo)alkyl, C1-8 (halo)alkoxyl, —S(O)₀₋₂-C1-8 (halo)alkyl, —S(O)₀₋₂-[3-8-membered (hetero)cycloalkyl], -C1-4alkyl-[3-8-membered (hetero)cycloalkyl], 5-6-membered (hetero)aryl, or C1-4alkyl[5-6-membered (hetero)aryl], wherein h1 is optionally substituted with halogen, C1-3 (halo)alkyl, or C1-3 (halo)alkoxyl. h1 is more preferably preferably —H, C1-8 (halo)alkyl, —S(O)₀₋₂-[3-8-membered (hetero)cycloalkyl], 5-6-membered (hetero)aryl, or C1-2alkyl[5-6-membered (hetero)aryl], wherein h1 is optionally substituted with halogen, C1-3 (halo)alkyl, or C1-3 (halo)alkoxyl.

Within h1, C1-8 (halo)alkyl is preferably C1-8alkyl, more preferably C1-6 alkyl; C1-8 (halo)alkoxyl is preferably C1-8alkoxyl, more preferably —O—CH₃; —S(O)₀₋₂-C1-8 (halo)alkyl is preferably —S(O)₂—C1-8alkyl; 3-8-membered (hetero)cycloalkyl is preferably 3-6-membered cycloalkyl; —S(O)₀₋₂-[3-8-membered (hetero)cycloalkyl] is preferably —S(O)₂-[3-6-membered cycloalkyl]; —C1.4alkyl-[3-8-membered (hetero)cycloalkyl] is preferably —CH₂-[3-6-membered cycloalkyl]; 5-6-membered (hetero)aryl is preferably phenyl or 5-methyl-1,3,4-oxadiazol-2-yl; —S(O)₀-2-[5-6-membered (hetero)aryl] is preferably —S(O)₂-[5-6-membered aryl]; and C1-4alkyl[5-6-membered (hetero)aryl] is preferably —CH₂-[5-6-membered (hetero)aryl].

h1 is preferably —H, —CH₃, -phenyl, —CH₂-phenyl, —OCH₃, —SO₂-cyclopropyl, —CH₂-methoxyphenyl, —CH₂-trifluoromethylphenyl, —N—(CH₂)₁₅CH₃, —N—(CH₂)₇, —N—(CH₂)₅CH₃, or —N—(CH₂)₂CH₃. More preferably it is —H, —CH₃, —CH₂-phenyl, even more preferably —H or —CH₃.

When head is h1 then h1 is preferably —H, —CH₃, or -phenyl. When head is —O-h1 then h1 is preferably —H or —CH₃. When head is —C(O)-h1 then h1 is preferably —CH₃. When head is —C(O)—N(H)h1 then h1 is preferably —H or —CH₂-phenyl. When head is —N(h2) h1 then h2 is preferably —H or -CH₃, most preferably —H, and h1 is preferably —H, —CH₃, —OCH₃, —SO₂-cyclopropyl, —CH₂-methoxyphenyl, —CH₂-trifluoromethylphenyl, —CH₂-phenyl, —N—(CH₂)₁₅CH₃, —N—(CH₂)₇, —N—(CH₂)₅CH₃, or —N—(CH₂)₂CH₃.

In preferred embodiments head is H or —OH; —C(O)—N(H)h1 such as —C(O) NH-Bn or —C(O)—NH₂; 5-10-membered (hetero)aryl such as phenyl, C1-4alkyl such as methyl (—CH₃), —N(h2) h1 such as—N(H)h1 such as —NH₂, —NH(C1-6alkyl) such as —NH (propyl) or —NH (hexyl), —NH—S(O)₂-C1-6 (cyclo)alkyl such as —NH—S(O)₂-cyclopropyl, —NH—CH₂-(5-6-membered aryl) such as —NH—CH₂-methoxyphenyl or —NH—CH₂-trifluoromethylphenyl or —NH-benzyl; or such as —N(C1-6alkyl)₂ such as—N(CH₃)₂, or such as —N(C1-6alkyl)(C1-6alkoxyl) such as —N(CH₃)(OCH₃); or the carbonyl moiety of the AAⁿ adjacent to head together with head is replaced by —B(OH)₂ or together forms optionally substituted 5-membered heteroaryl such as 5-methyl-4-aza-oxazol-2-yl. In other embodiments-N(H)h1 is —NH(C1-24alkyl) such as —NH (propyl) or —NH (hexyl) or —NH (octyl) or —NH (hexadecyl).

In other preferred embodiments head comprises at most 1-10 total of carbon atoms and heteroatoms, preferably 1-8, more preferably 1-7, or the carbonyl moiety of the AAⁿ adjacent to head together with head is replaced by —B(OH)₂ or together forms optionally methylated 5-membered heteroaryl such as 5-methyl-4-aza-oxazol-2-yl. More preferably head comprises at most 1-7 total of carbon atoms and heteroatoms, or the carbonyl moiety of the AAⁿ adjacent to head together with head is replaced by —B(OH)₂ or together forms optionally methylated 5-membered heteroaryl such as 5-methyl-4-aza-oxazol-2-yl. Highly preferably head is —NH₂, phenyl, or —N—SO₂-cyclopropyl; or forms replacing heads-(5-methyl-1,3,4-oxadiazol-2-yl) or —B(OH)₂. Most preferably head is —NH₂, phenyl or forms replacing head-B(OH)₂.

Preferred embodiments of head are -H, —CH₃, -phenyl, —OH, —OCH₃, —C(O)—NH₂, —C(O)—NH—CH₂-phenyl, —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₁₅CH₃, —N—(CH₂)₇CH₃, —N—(CH₂)₅CH₃, —N—(CH₂)₂CH₃, replacing head-(5-methyl-1,3,4-oxadiazol-2-yl), and replacing head-B(OH)₂. In some embodiments head is —C(O)—NH₂, —C(O)—NH—CH₂-phenyl, —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₁₅CH₃, —N—(CH₂)₇CH₃, —N—(CH₂)₅CH₃, —N—(CH₂)₂CH₃, replacing head (5-methyl-1,3,4-oxadiazol-2-yl), and replacing head-B(OH)₂. In some embodiments head is —H, —CH₃, -phenyl, —OH, —OCH₃, —C(O)—NH₂, —C(O)—NH—CH₂-phenyl, —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₁₅CH₃, —N—(CH₂)₇CH₃, —N—(CH₂)₅CH₃, or —N—(CH₂)₂CH₃. In some embodiments head is-NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₁₅CH₃, —N—(CH₂)₇CH₃, —N—(CH₂)₅CH₃, or —N—(CH₂)₂CH₃. In some embodiments head is —H, —CH₃, -phenyl, —OH, —OCH₃, —C(O)—NH₂, —C(O)—NH—CH₂-phenyl, —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₇CH₃, —N—(CH₂)₅CH₃, —N—(CH₂)₂CH₃, replacing head-(5-methyl-1,3,4-oxadiazol-2-yl), and replacing head-B(OH)₂. In some embodiments head is —C(O)—NH₂, —C(O)—NH—CH₂-phenyl, —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₇CH₃, —N—(CH₂)₅CH₃, —N—(CH₂)₂CH₃, replacing head-(5-methyl-1,3,4-oxadiazol-2-yl), and replacing head-B(OH)₂. In some embodiments head is —H, —CH₃, -phenyl, —OH, —OCH₃, —C(O)—NH₂, —C(O)—NH—CH₂-phenyl, —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₇CH₃, —N—(CH₂)₅CH₃, or —N—(CH₂)₂CH₃. In some embodiments head is —NH₂, —N(CH₃)₂, —N(—OCH₃)(CH₃), —N—SO₂-cyclopropyl, —NH—CH₂-(methoxyphen-3-yl), —NH—CH₂-(trifluoromethylphen-4-yl), —NH—CH₂-phenyl, —N—(CH₂)₇CH₃, —N—(CH₂)₅CH₃, or —N—(CH₂)₂CH₃. In some embodiments head-NH₂ or —N—SO₂-cyclopropyl. In some embodiments head is —C(O)—NH₂, —C(O)—NH—CH₂-phenyl, —NH₂, —N—SO₂-cyclopropyl, —N—(CH₂)₇CH₃, —N—(CH₂)₅CH₃, —N—(CH₂)₂CH₃, replacing head-(5-methyl-1,3,4-oxadiazol-2-yl), or replacing head-B(OH)₂. In some embodiments head-NH₂ or —N—SO₂-cyclopropyl. In some embodiments head is —C(O)—NH₂, —C(O)—NH—CH₂-phenyl, —NH₂, —N—SO₂-cyclopropyl, —N—(CH₂)₇CH₃, —N—(CH₂)₅CH₃, or —N—(CH₂)₂CH₃. Representative embodiments of head are shown below

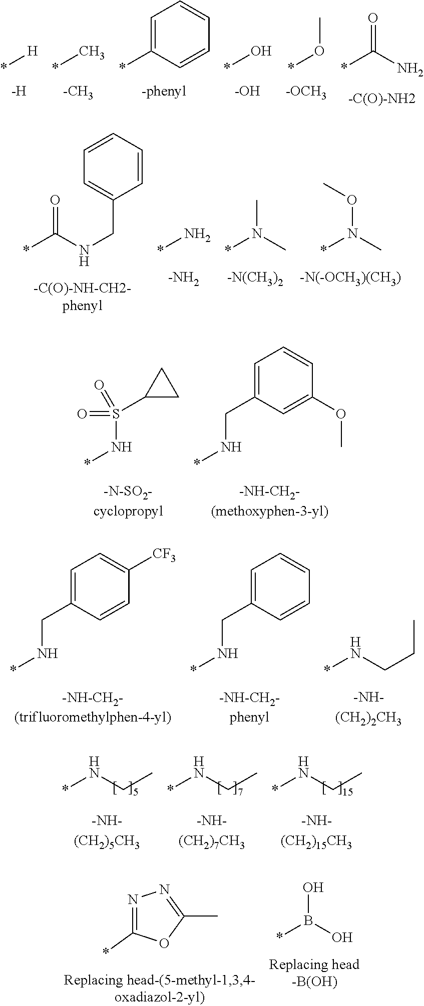

### Further Definitions

In preferred embodiments of the compound according to the invention, tail comprises at least 8 carbon atoms, preferably it is pentadecyl; and/or head comprises at most 1-10 total of carbon atoms and heteroatoms, preferably 1-8, more preferably 1-7, or the carbonyl moiety of the AAⁿ adjacent to head together with head is replaced by —B(OH)₂ or together forms optionally methylated 5-membered heteroaryl such as 5-methyl-4-aza-oxazol-2-yl. When tail is pentadecyl, it can contribute to the formation of a palmitic acid amide, which was found to provide good results. Other attractive amides are C14 and C18, wherein tail is respectively tridecyl or heptadecyl.

In some embodiments, tail comprises at least 8 carbon atoms, preferably it is pentadecyl. In some embodiments, tail comprises at least 10 carbon atoms. In some embodiments, tail comprises at least 11, 12, or preferably 13 carbon atoms. In some embodiments head comprises at most 1-10 total of carbon atoms and heteroatoms, preferably 1-8, more preferably 1-7, or the carbonyl moiety of the AAⁿ adjacent to head together with head is replaced by —B(OH)₂ or together forms optionally methylated 5-membered heteroaryl such as 5-methyl-4-aza-oxazol-2-yl. In some embodiments head comprises at most 1-10 total of carbon atoms and heteroatoms, preferably 1-8, more preferably 1-7. In some embodiments the carbonyl moiety of the AAⁿ adjacent to head together with head is replaced by —B(OH)₂ or together forms optionally methylated 5-membered heteroaryl such as 5-methyl-4-aza-oxazol-2-yl.

In some preferred embodiments tail is —(CH₂)₁₄CH₃, head is —NH₂, phenyl, or forms replacing head-B(OH)₂, and m is 2 and AA¹ and the two instances of AAⁿ together form a tripeptide represented by KAK, KAH, KAF, KA-homoPhe, KAA, KPA, KPK, FAF, HAH, ROK, RQF, RAF, RQ-homoPhe, HQF, homoHis-QF, (para-guanidinyl-Phe)-QF, (meta-guanidinyl-Phe)-QF, ((2-amino-((2-amino-1H-imidazol-4-yl)methyl-Ala)-QF, ((2-amino-1H-1H-imidazol-4-yl)ethyl-Ala)-QF, imidazol-4-yl)-Ala)-QF, ((pyridin-3-yl)homoAla)-QF, ((pyridin-3-yl) Ala)-QF, ((pyridin-4-yl) Ala)-QF, ((1H-imidazol-2-yl)aminomethyl-Ala)-QF, ((1H-imidazol-2-yl)aminoethyl-Ala)-QF, ((pyridin-4-yl)homoAla)-QF, or RNF, or m is 3 and AA¹ and the three instances of AAⁿ together form a tetrapeptide represented by KAAA, KAAF, KAAH, KAAK, KAAW, KAA-homoHis, KAA-homoPhe, KAA-phenylGly, KAA-(4-OBn-phenylGly), KPAF, KPAH, KPAK, KAPK, KPAW, KPA-homoHis, KPA-homoPhe, KPA-phenylGly, KPA-(4-OBn-phenylGly), AAAK, AAKA, AKAA, AKAK, APAK, A-(4-O-(4-Py)-Pro)-AK, AVAK, AKKK, LKAK, LKKK, VKAK, KPHK, KPH-homoPhe, K-(pipecolic acid)-AK, K-(pipecolic acid)-HK, KP-phenylglycine-K, K-(pipecolic acid)-phenylglycine-K, ARQK, ARQF, ARRK, FKKK, RARK, FAAF, or WAAW.

In some embodiments the compounds according to the invention are compounds 1-196. In preferred embodiments the compounds are 1-17, 19, 24, 25, 32, 39-44, 48-54, 72, 82, 83, 90-93, 97, 98, 110-123, 125-130, 132, 133-139, 143, 146-151, 156, 158-190, 192, 193, 196, more preferably 1, 2, 4, 7, 9-17, 19, 24, 25, 32, 39-41, 43, 44, 48-51, 53, 54, 72, 82, 83, 90, 92, 97, 98, 110-123, 125, 128, 129, 132, 133, 134, 136, 137, 139, 146-150, 156, 158-161, 167, 168, 170-177, 180-183, 186, 187, 189, 190, 192, 193, 196, more preferably 9, 11, 32, 38, 43, 44, 69, 70, 71, 72, 73, 75, 77, 79, 81, 82, 83, 86, 87, 88, 89, 92, 94, 98, 113, 114, 115, 116, 117, 118, 122, 123, 129, 136, 137, 156, 158, 162, 167, 168, 170, 171, 174, 175, 176, 190, 192, 196. In other preferred embodiments the compounds are 5, 6, 8, 9, 10, 11, 13, 16, 17, 19, 24, 25, 32, 39, 41, 43, 44, 48, 90, 92, 113, 114, 115, 116, 117, 118, 122, 123, 125, 129, 132, 136, 137, 156, 167, 168, 170, 171, 174, 175, 176, 180, 181, 182, 183, 186, 189, 190, 193, 196, more preferably 9, 10, 43, 44, 50, 51, 53, 54, 113, 114, 118, 136, 170, 189, 196. In other preferred embodiments the compounds are 5, 6, 8, 9, 10, 11, 39, 43, 44, 48, 49, 50, 51, 53, 54, 92, 113, 114, 115, 116, 117, 118, 122, 123, 125, 129, 136, 137, 167, 168, 170, 171, 175, 176, 189, 196, more preferably 44, 49, 50, 51, 53, 54, 113, 114, 118, 136, 137, 170, 189, 196. In other preferred embodiments the compounds are 19, 24, 25, 32, 39, 41, 43, 48, 49, 50, 51, 53, 72, 82, 83, 97, 98, 110, 113, 114, 121, 125, 129, 132, 133, 136, 137, 146, 148, 149, 156, 158, 159, 167, 172, 173.

| Number | |
|---|---|
| (observed | |
| mass) | Structure |
| 1 (719.55) | 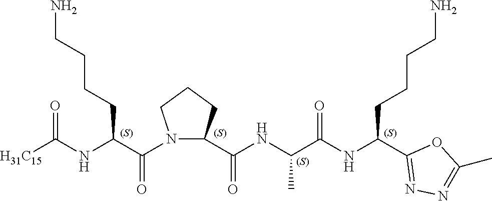 |
| 2 (693.54) | 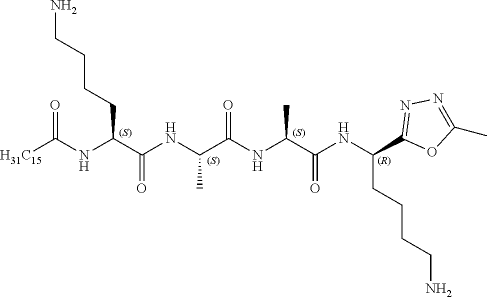 |
| 3 (726.52) | 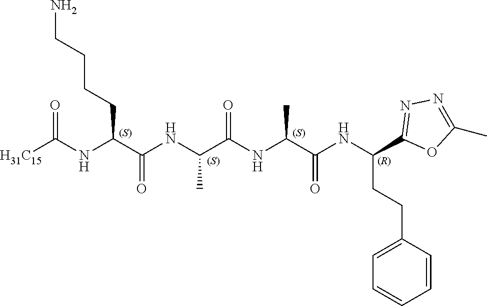 |
| 4 (752.54) | 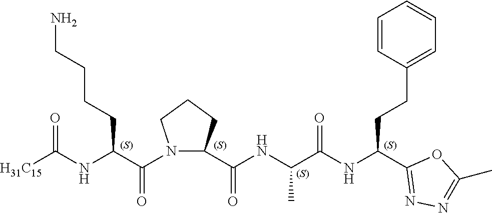 |
| 5 (726.52) | 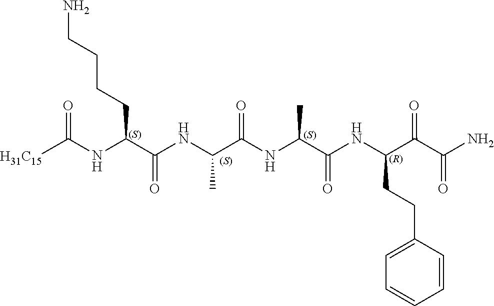 |
| 6 (741.8) | 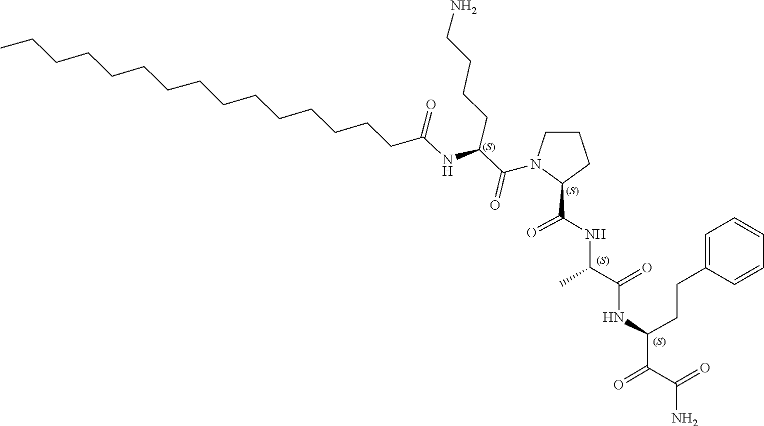 |
| 7 (716.64) | 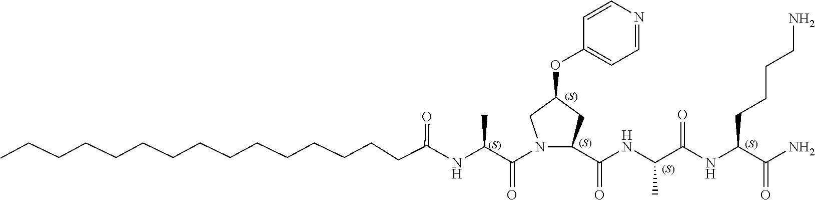 |
| 8 (716.6) | 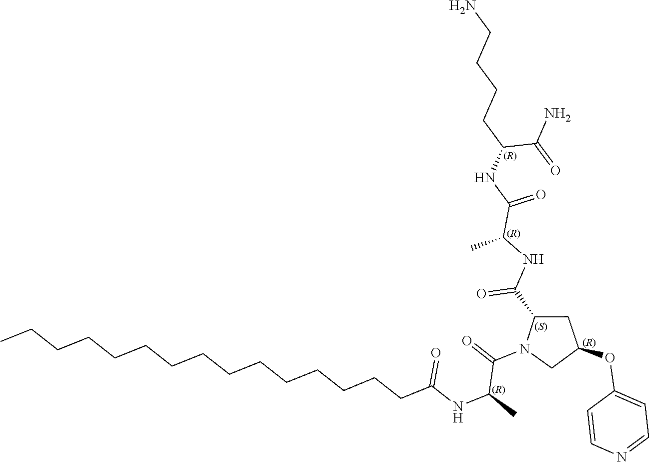 |
| 9 (664.51) | 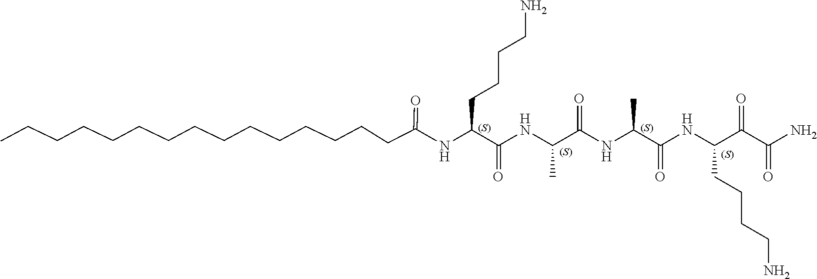 |
| 10 (690.53) | 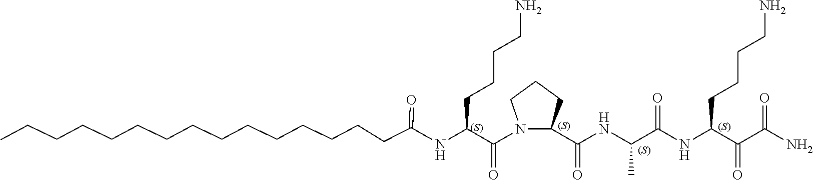 |
| 11 (779.55) | 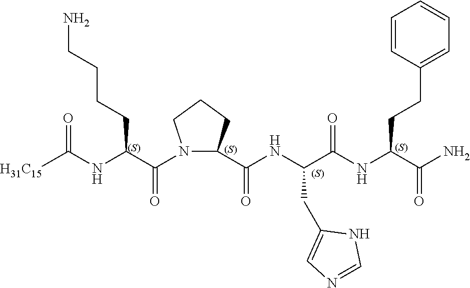 |
| 12 (746.57) | 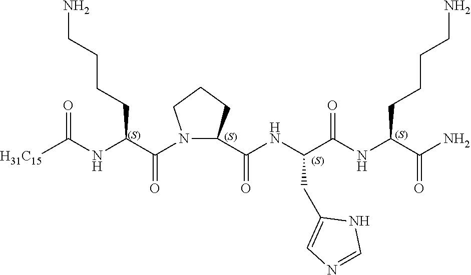 |
| 13 (764.54) | 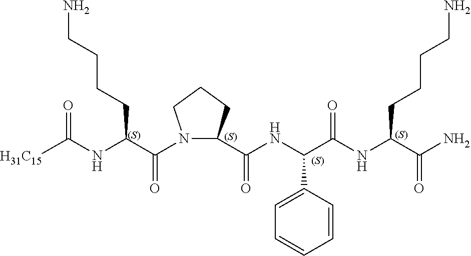 |
| 14 (756.58) | 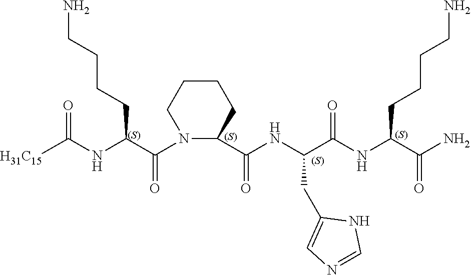 |
| 15 (694.56) | 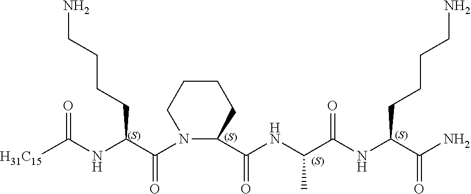 |
| 16 (760.58) | 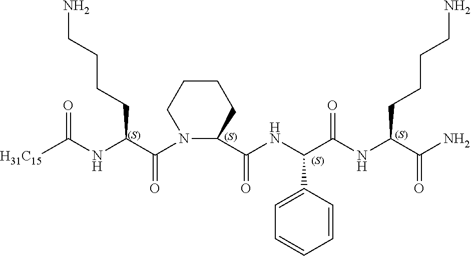 |
| 17 (673.5) | 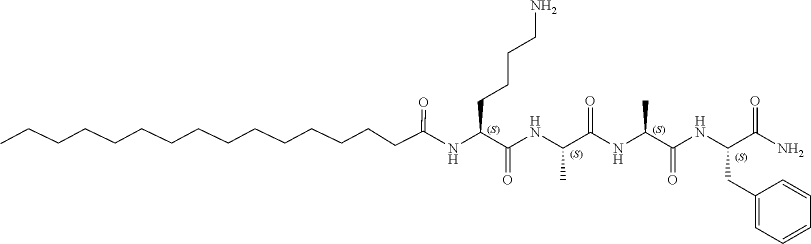 |
| 18 (711.58) | 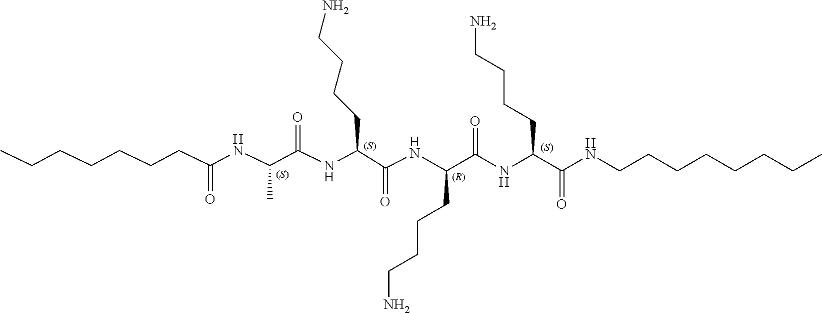 |
| 19 (753.63) | 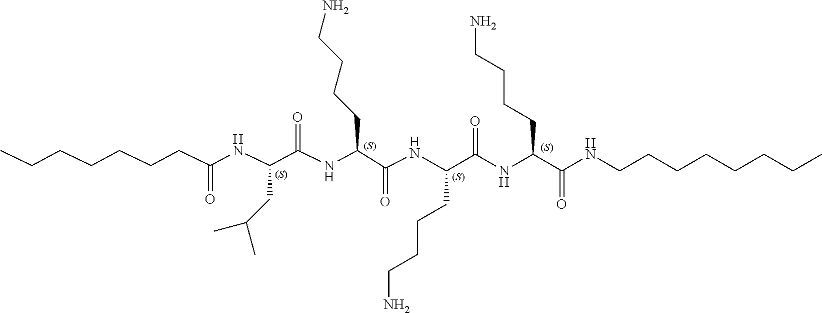 |
| 20 (787.61) | 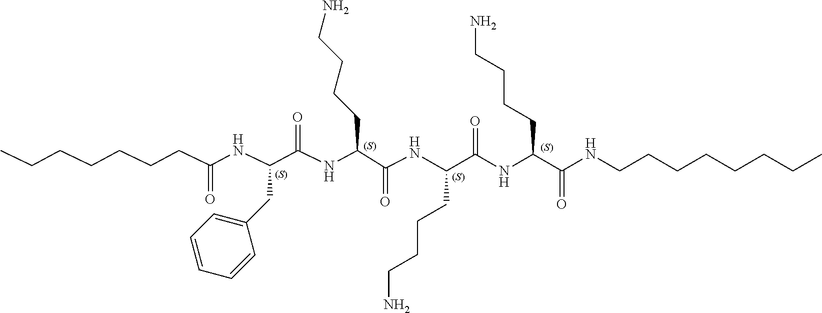 |
| 21 (571.43) | 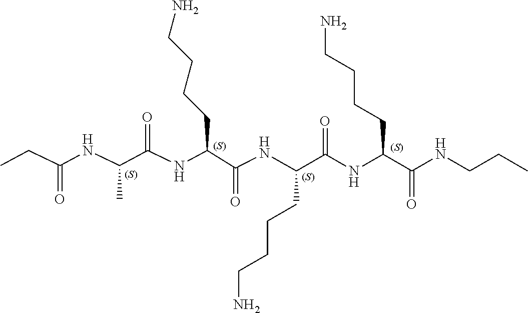 |
| 22 (613.48) | 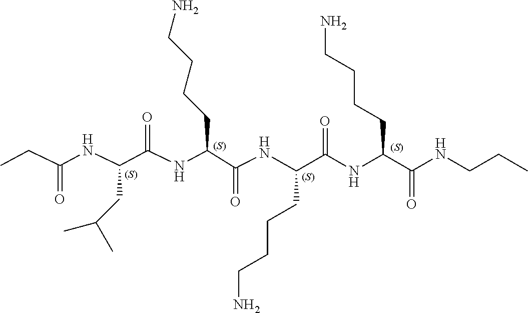 |
| 23 (647.46) | 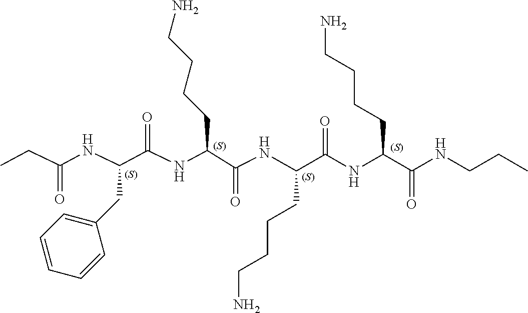 |
| 24 (654.53) | 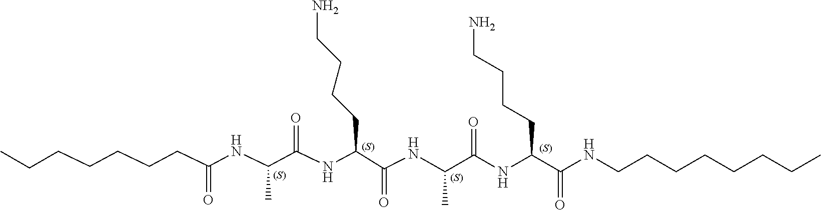 |
| 25 (696.58) | 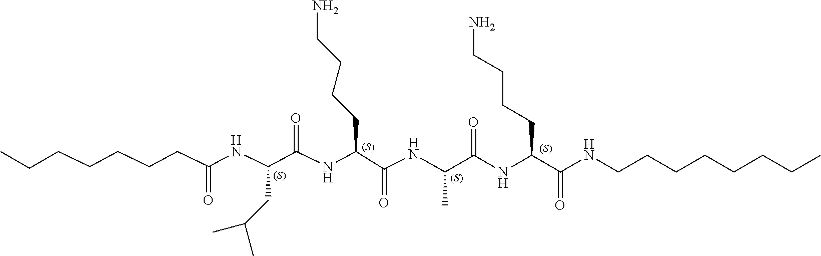 |
| 26 (500.36) | 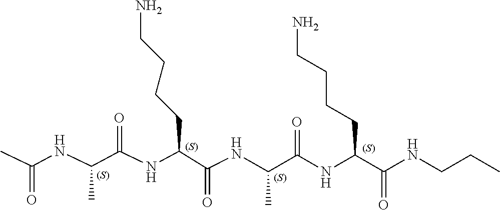 |
| 27 (542.4) | 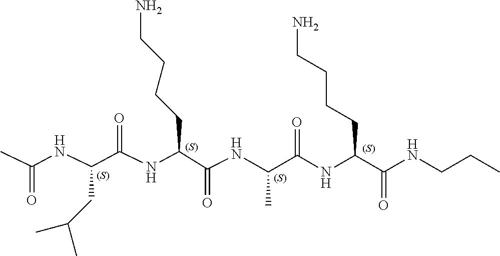 |
| 28 (767.6) | 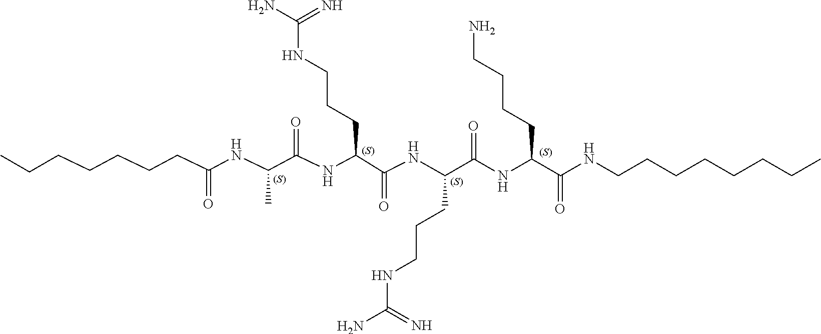 |
| 29 (767.6) | 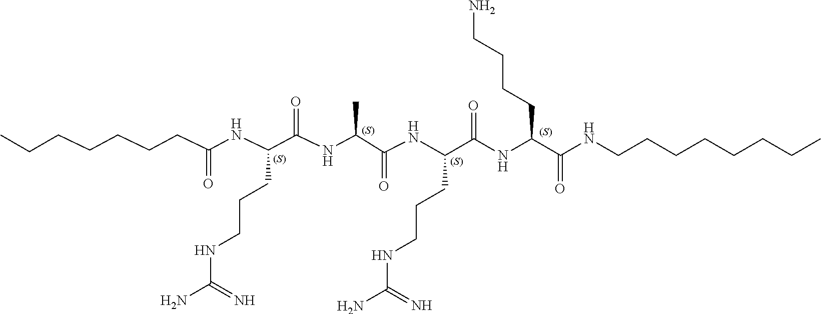 |
| 30 (627.44) | 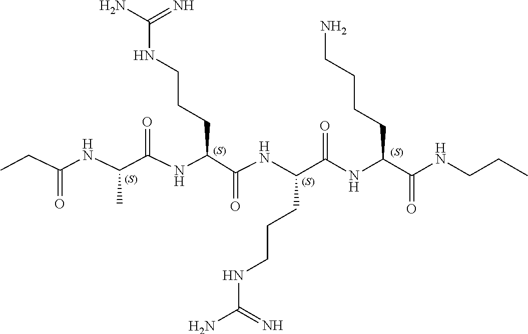 |
| 31 () | 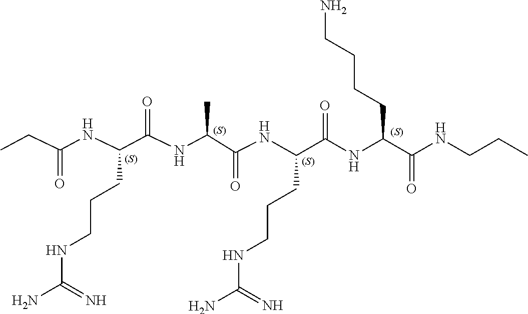 |
| 32 (725.81) | 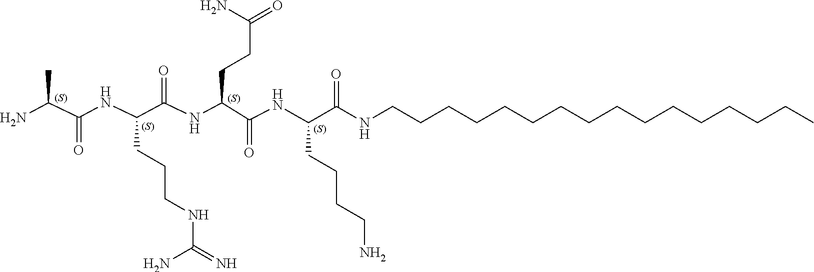 |
| 33 (605.45) | 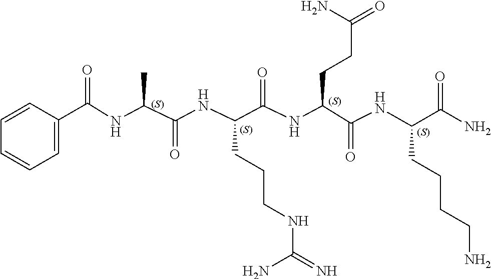 |
| 34 (695.5) | 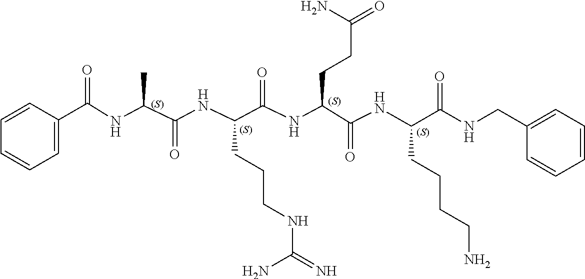 |
| 35 (693.52) | 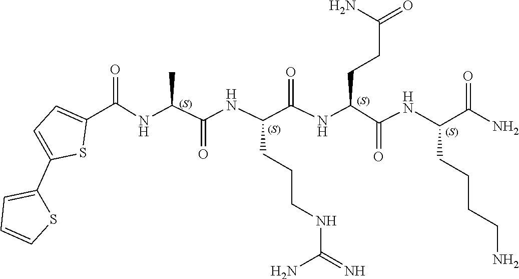 |
| 36 (813.61) | 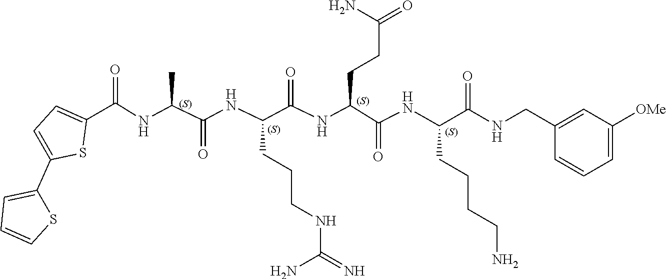 |
| 37 (681.51) | 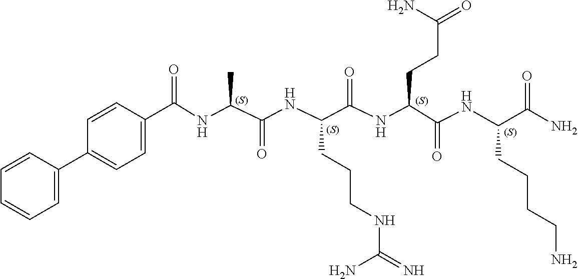 |
| 38 (839.62) | 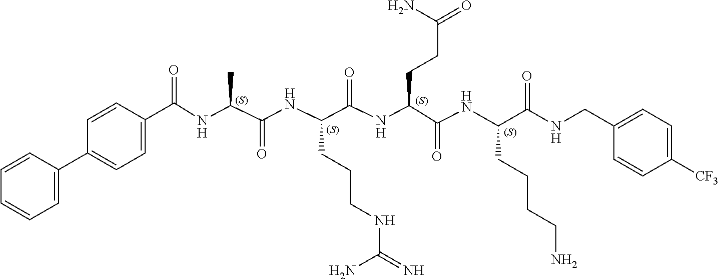 |
| 39 (740.39) | 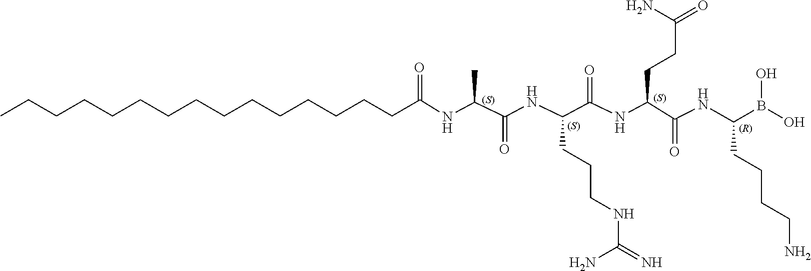 |
| 40 (668.14) | 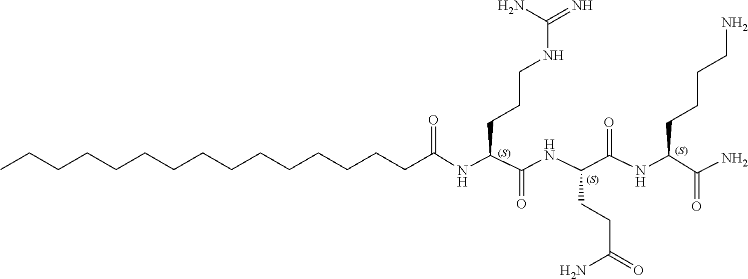 |
| 41 (669.36) | 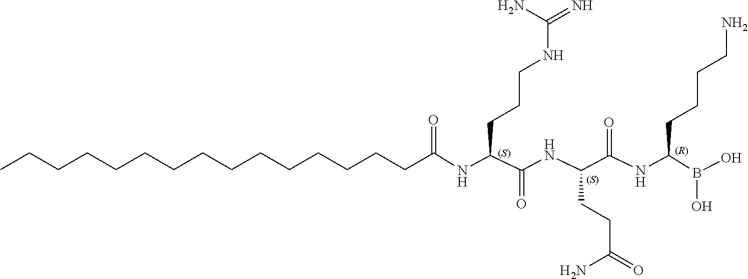 |
| 42 (729.5) | 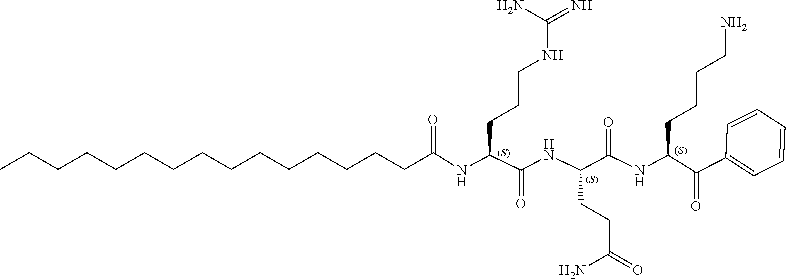 |
| 43 (687.49) | 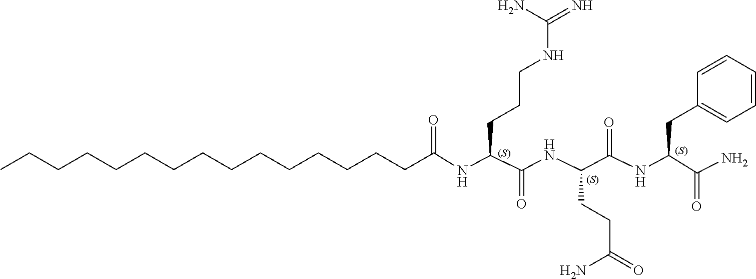 |
| 44 (687.2) | 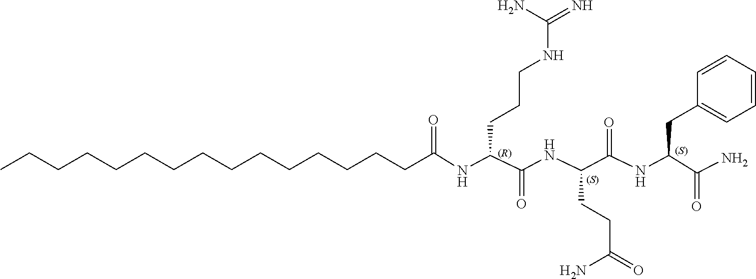 |
| 45 (688.2) | 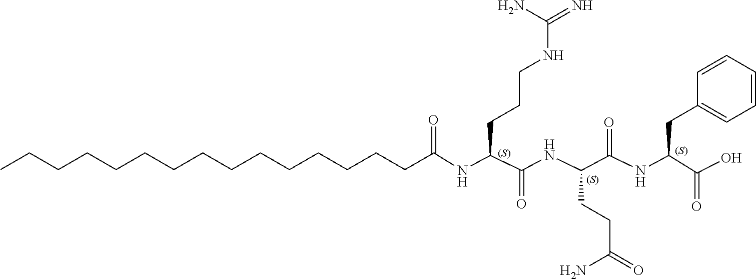 |
| 46 (688.2) | 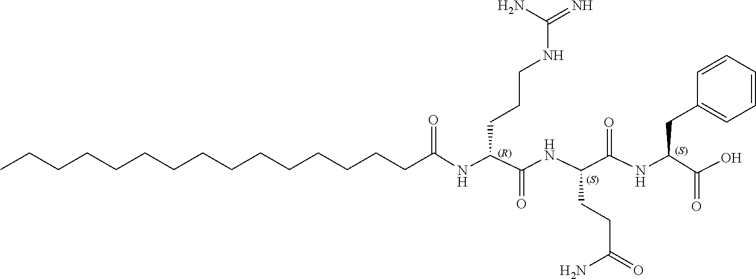 |
| 47 (543.34) | 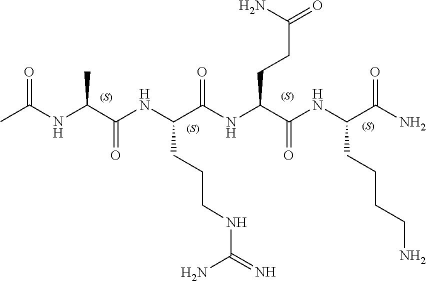 |
| 48 (458.31) | 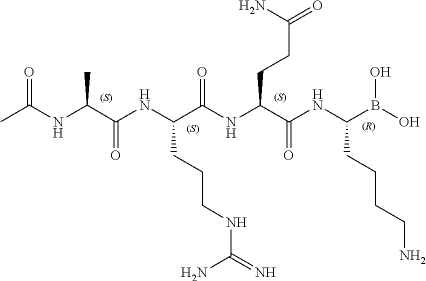 |
| 49 (687.49) | 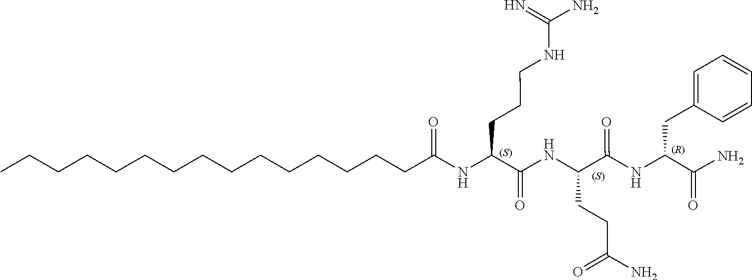 |
| 50 (673.47) | 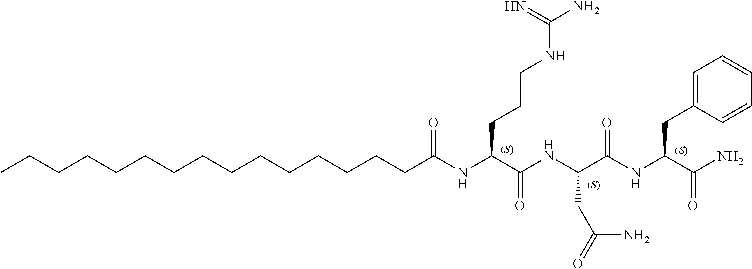 |
| 51 (630.47) | 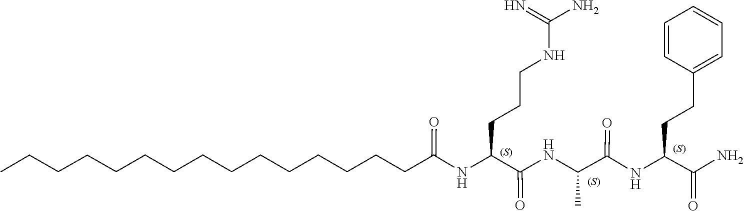 |
| 52 (668.45) | 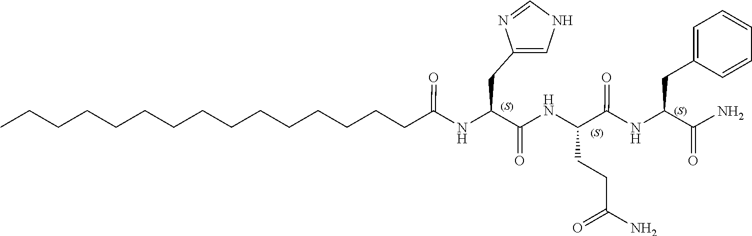 |
| 53 (701.51) | 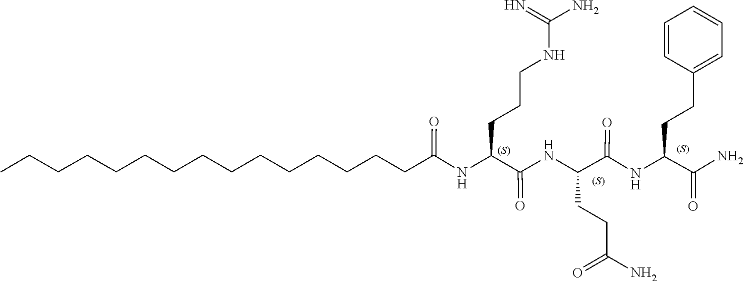 |
| 54 (758.53) | 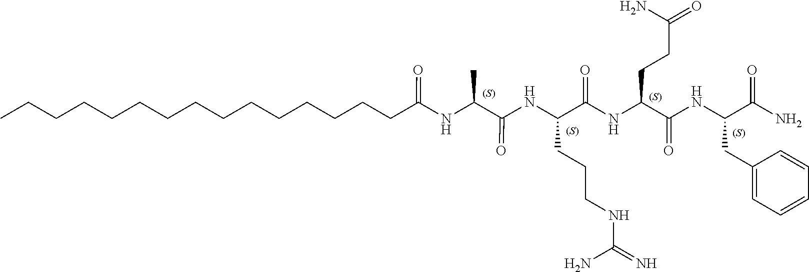 |
| 55 (697.47) | 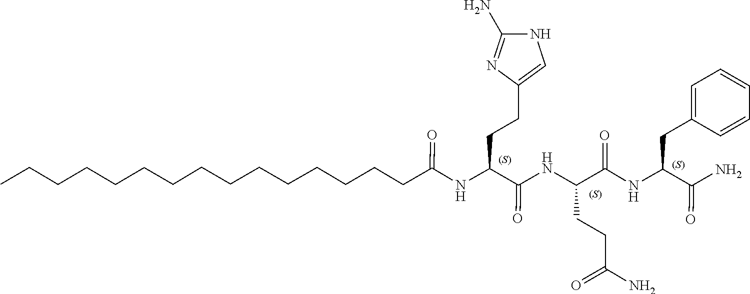 |
| 56 () | 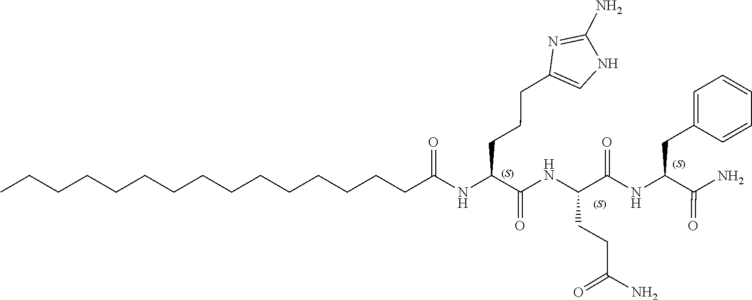 |
| 57 (693.47) | 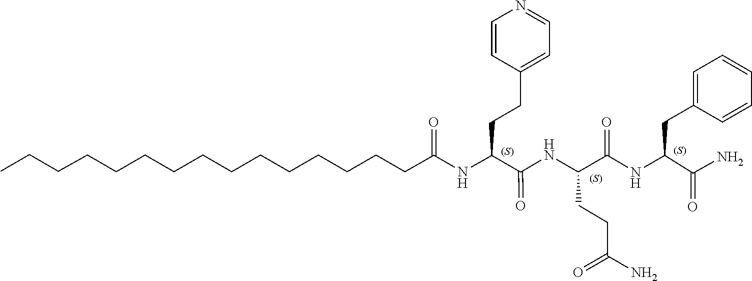 |
| 58 (693.47) | 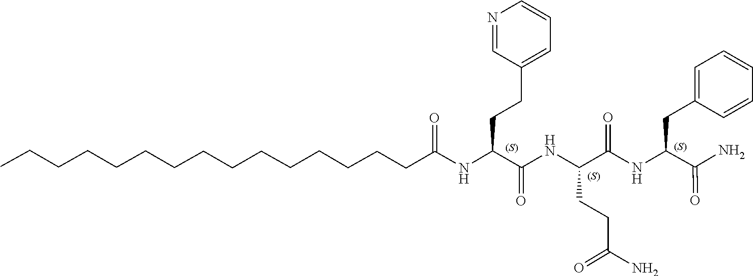 |
| 59 (682.46) | 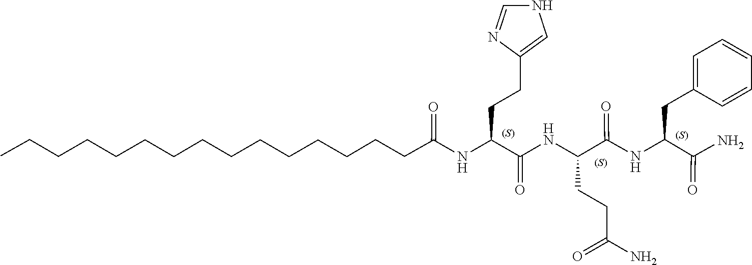 |
| 60 (688.49) | 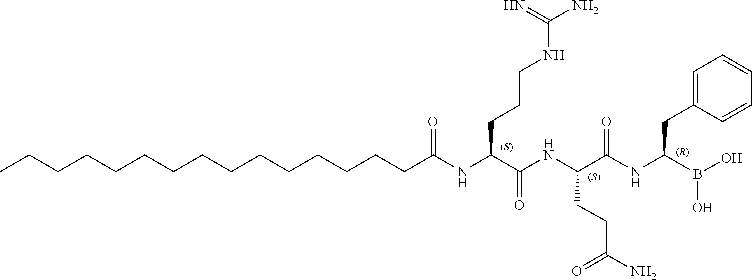 |
| 61 (563.31) | 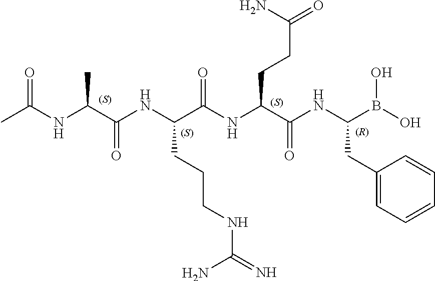 |
| 62 (683.46) | 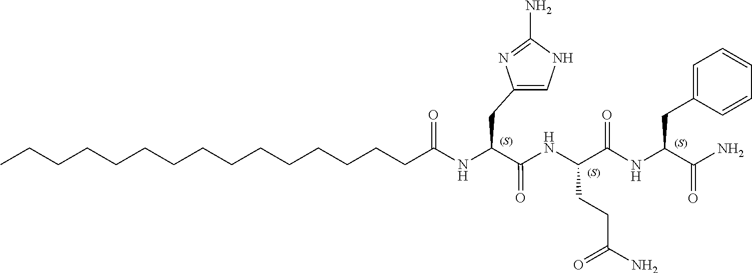 |
| 63 (479.46) | 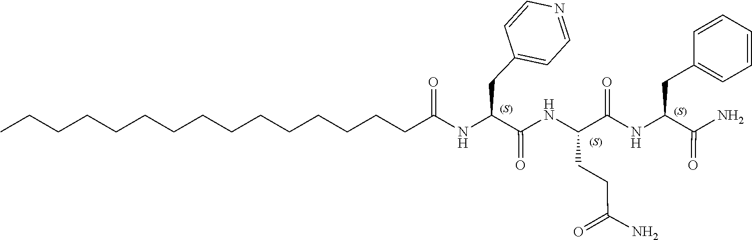 |
| 64 (479.46) | 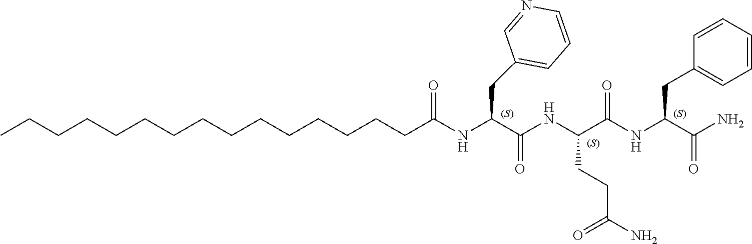 |
| 65 (711.49) | 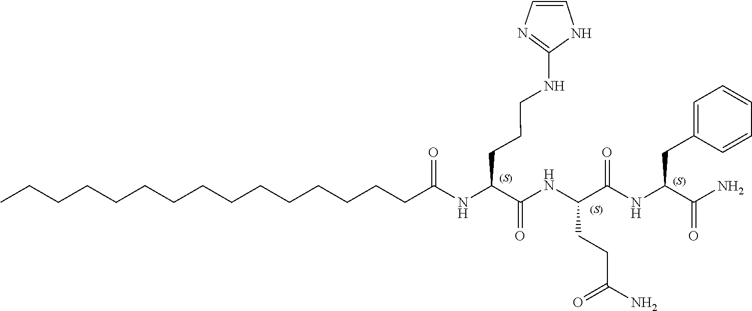 |
| 66 (697.47) | 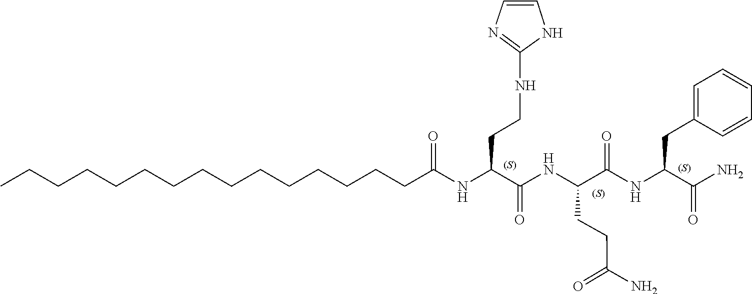 |
| 67 (735.49) | 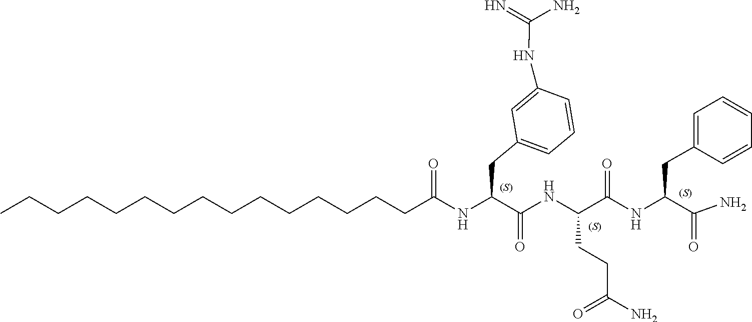 |
| 68 (735.49) | 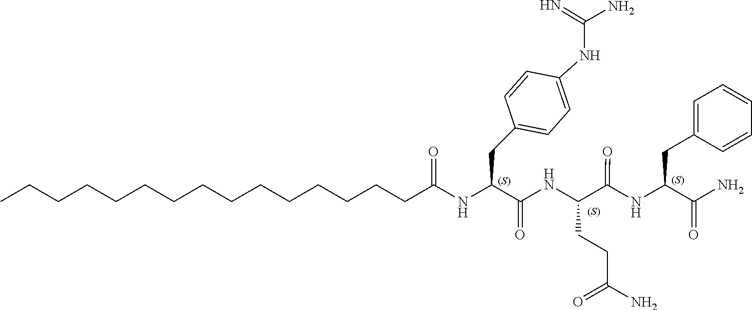 |
| 69 (697.68) | 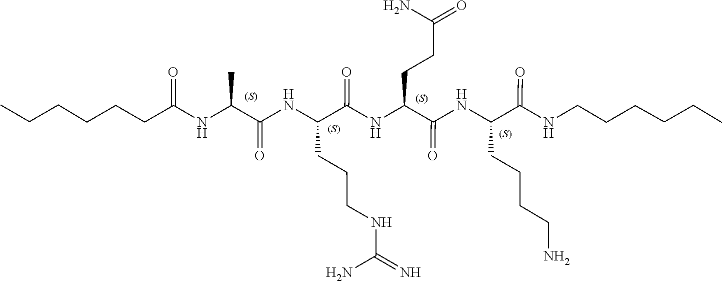 |
| 70 (613.51) | 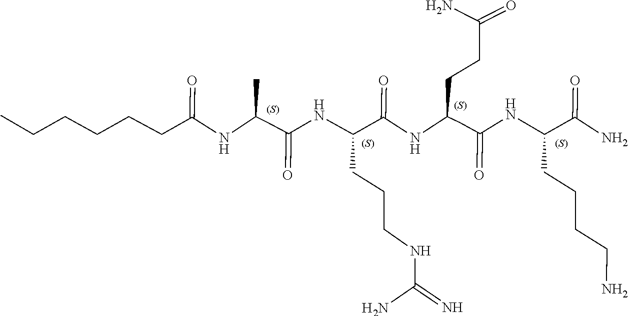 |
| 71 (585.58) | 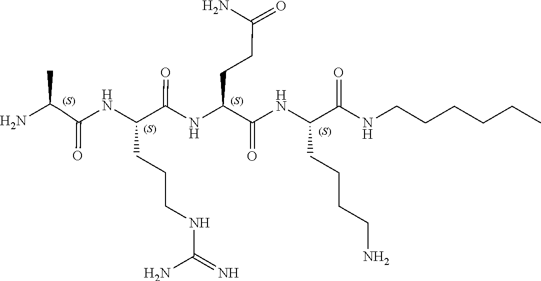 |
| 72 (739.8) | 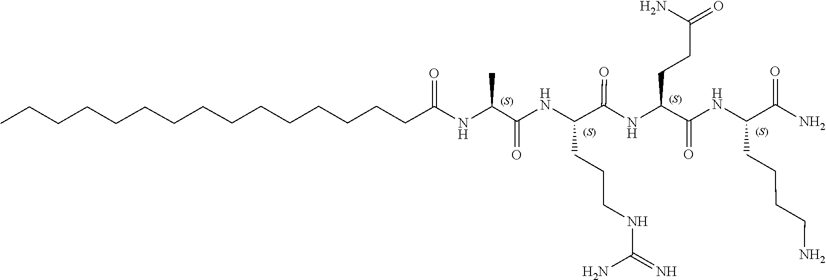 |
| 73 (449.33) | 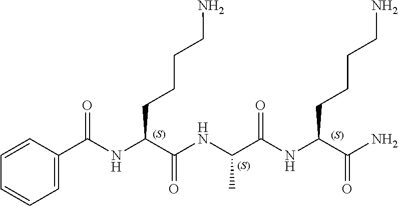 |
| 74 (539.5) | 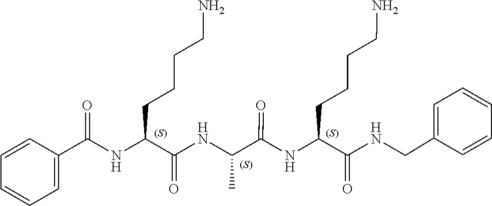 |
| 75 (537.32) | 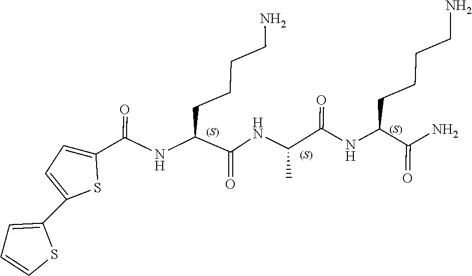 |
| 76 (657.34) | 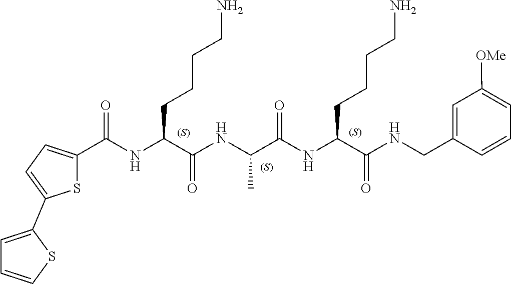 |
| 77 (525.44) | 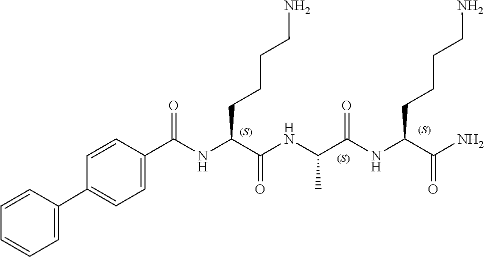 |
| 78 (683.55) | 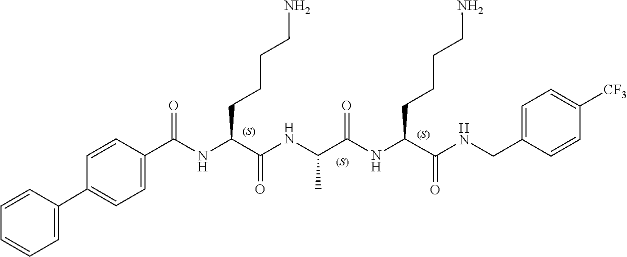 |
| 79 (612.65) | 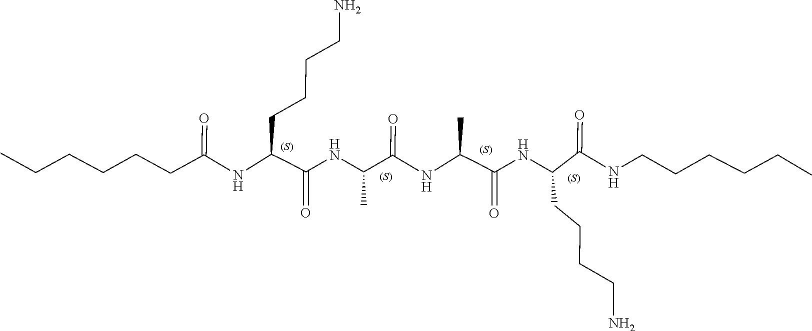 |
| 80 (528.4) | 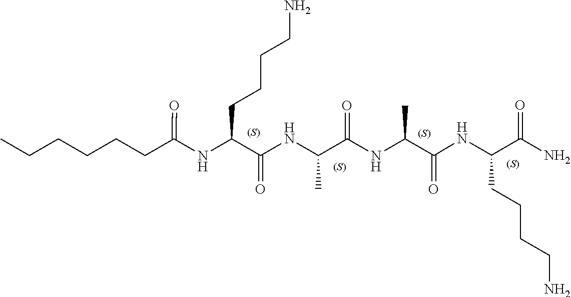 |
| 81 (500.55) | 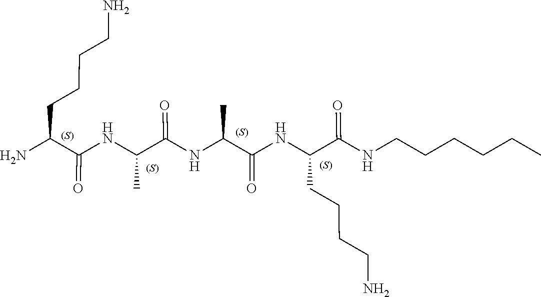 |
| 82 (654.7) | 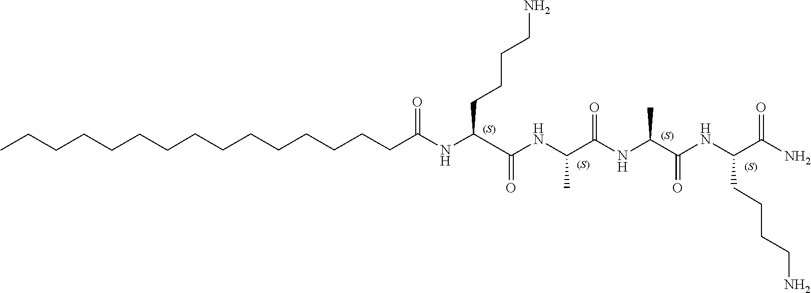 |
| 83 (640.5) | 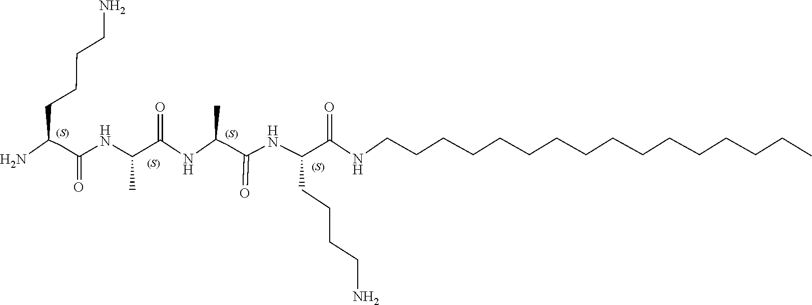 |
| 84 (520.42) | 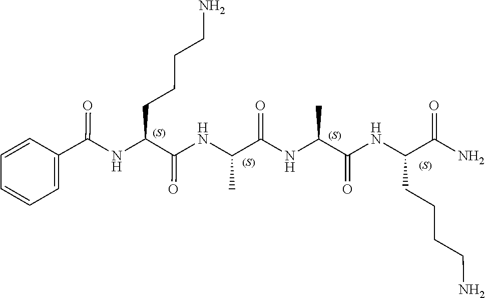 |
| 85 (610.6) | 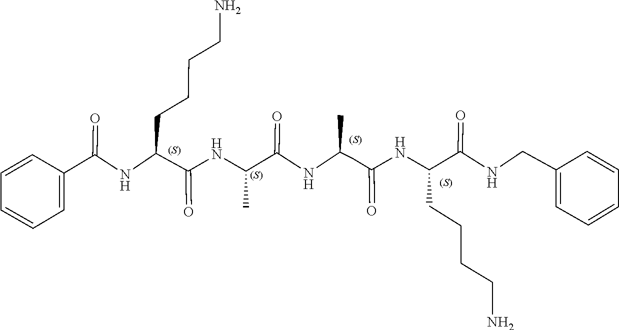 |
| 86 (608.49) | 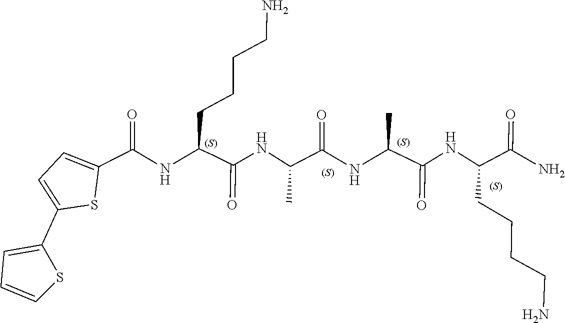 |
| 87 (728.51) | 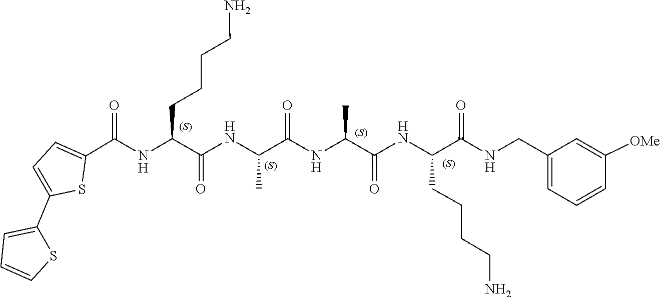 |
| 88 (596.54) | 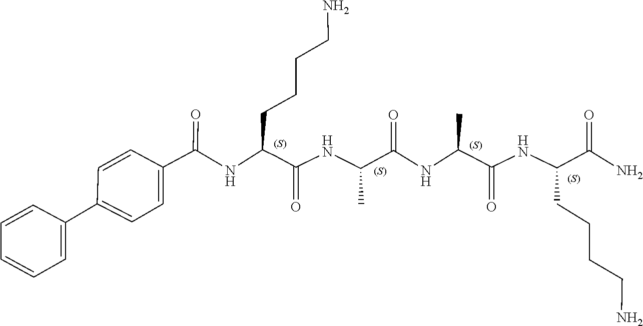 |
| 89 (754.66) | 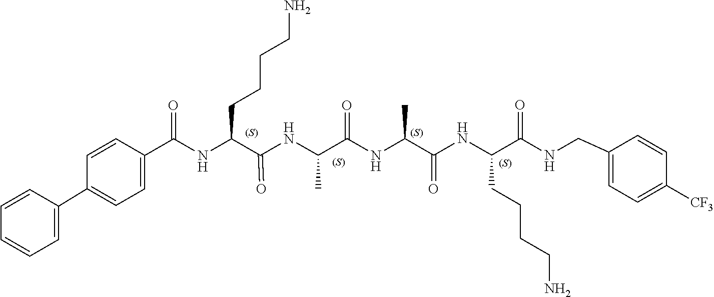 |
| 90 (687.2) | 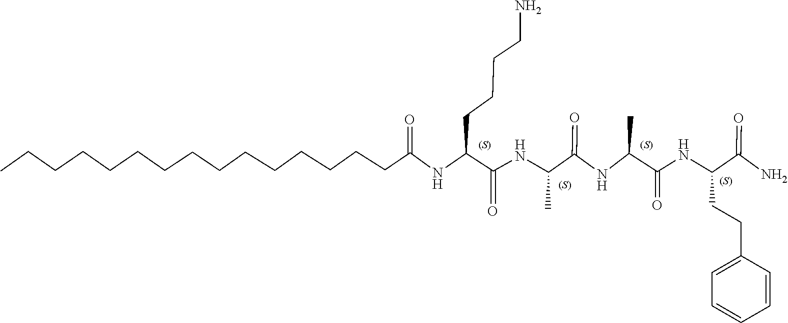 |
| 91 (687.2) | 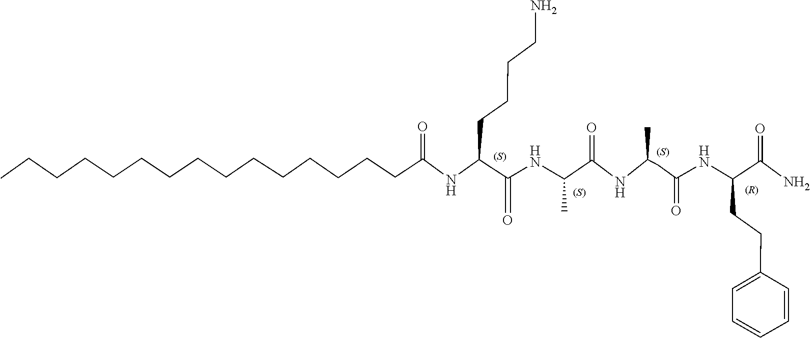 |
| 92 (712.74) | 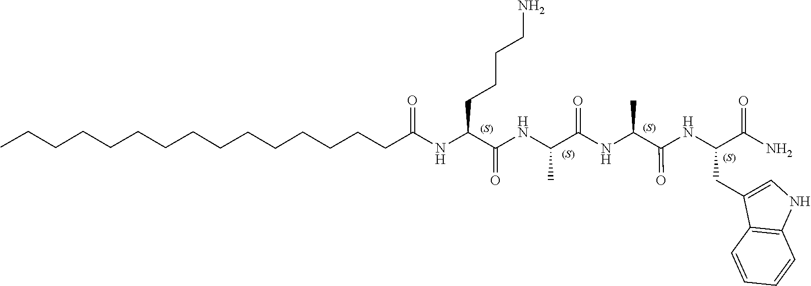 |
| 93 (712.71) | 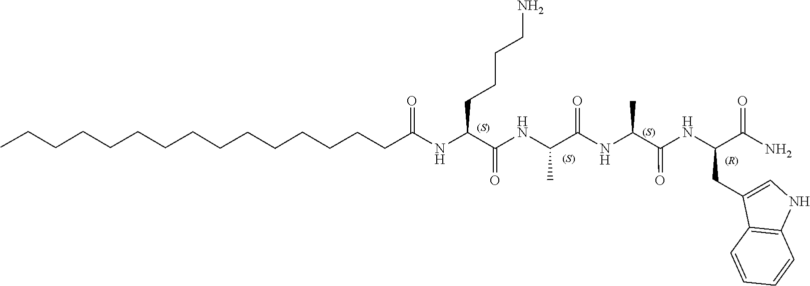 |
| 94 (541.45) | 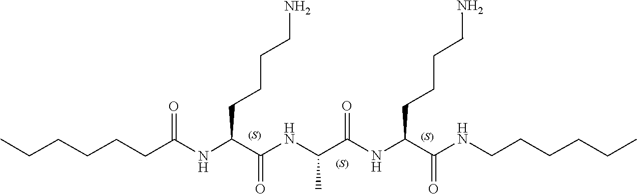 |
| 95 (457.38) | 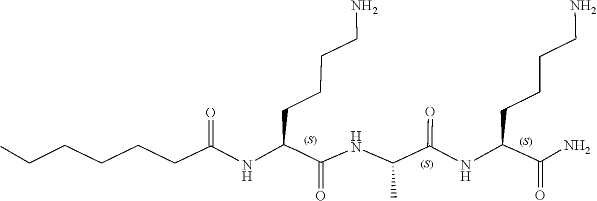 |
| 96 (429.32) | 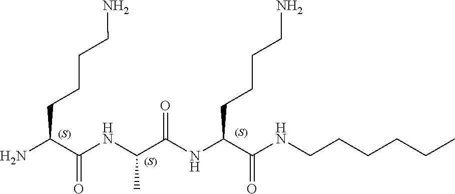 |
| 97 (583.6) | 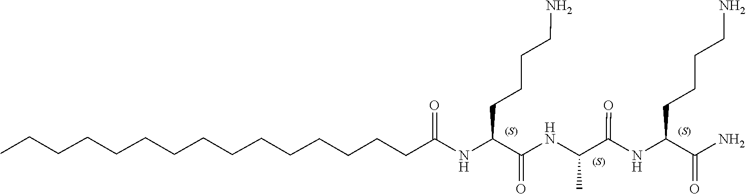 |
| 98 (569.54) | 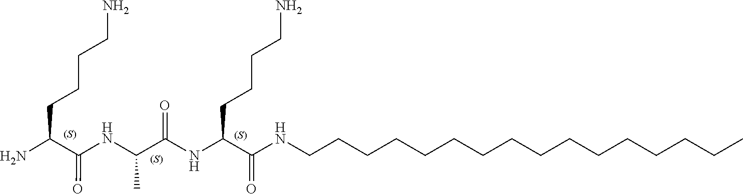 |
| 99 () | 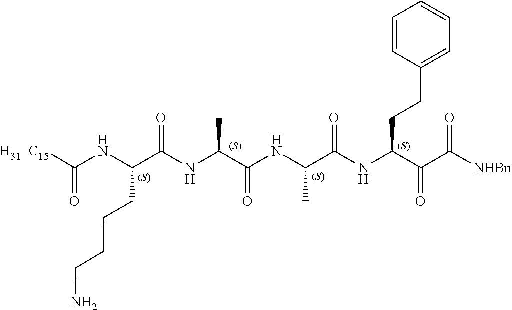 |
| 100 () | 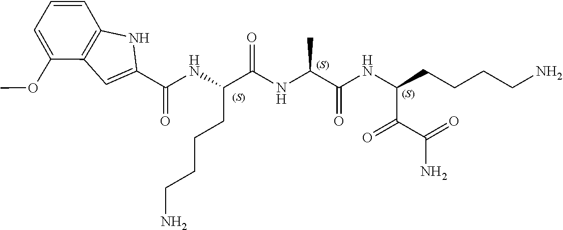 |
| 101 () | 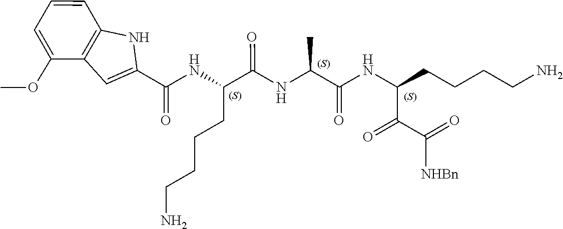 |
| 102 () | 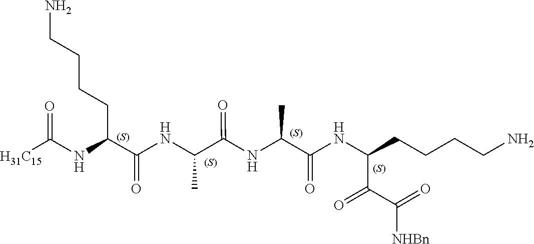 |
| 103 () | 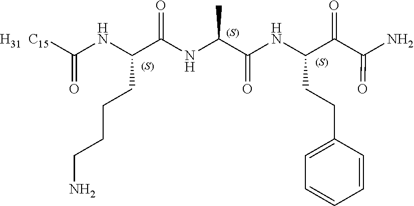 |
| 104 () | 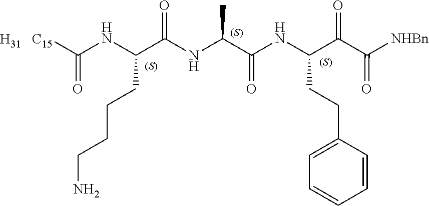 |
| 105 () | 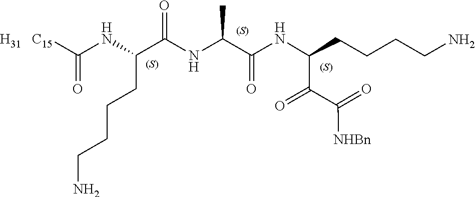 |
| 106 () | 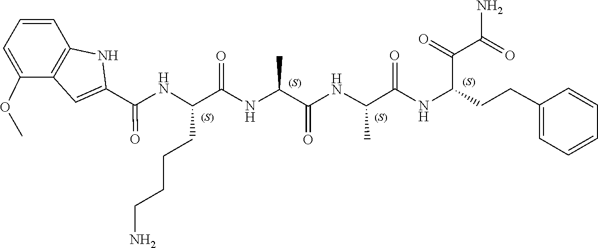 |
| 107 () | 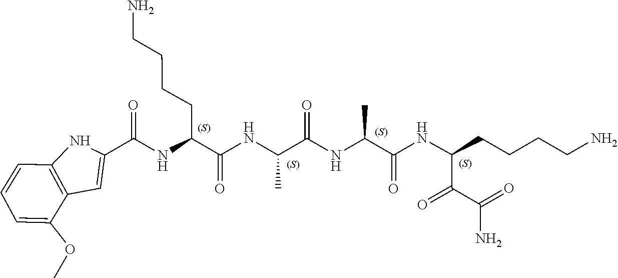 |
| 108 () | 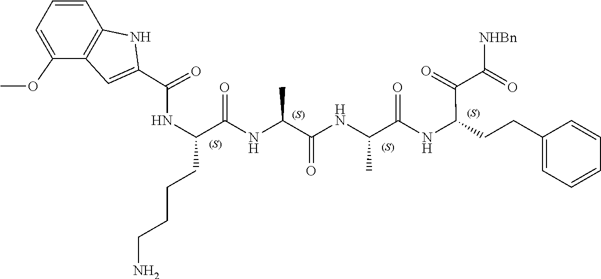 |
| 109 () | 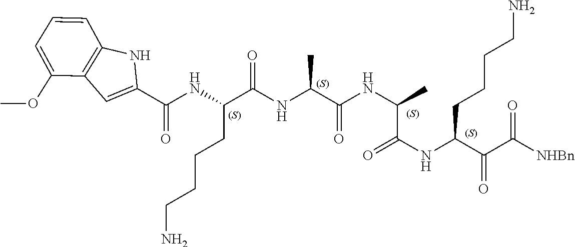 |
| 110 (534.44) | 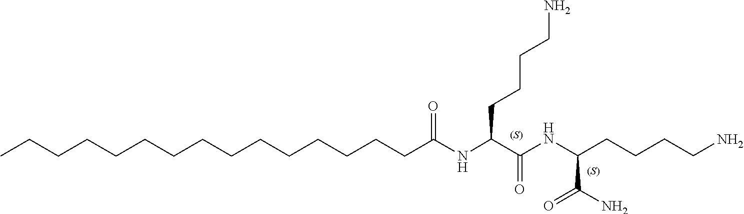 |
| 111 (540.46) | 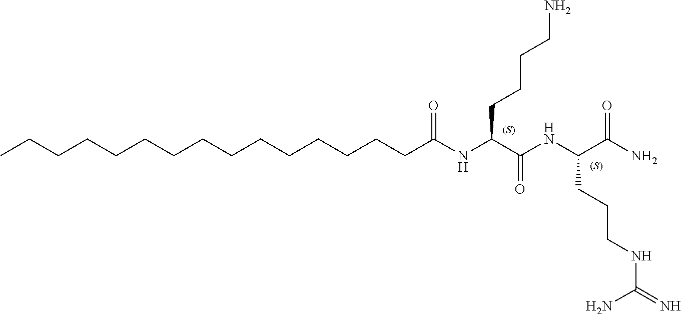 |
| 112 (540.46) | 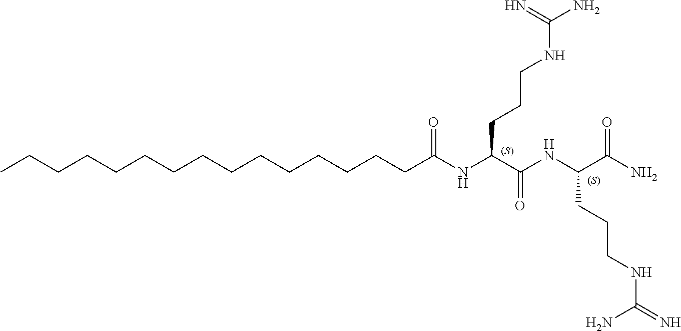 |
| 113 (654.53) | 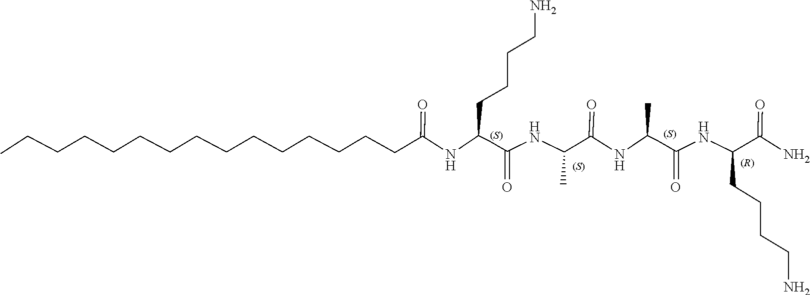 |
| 114 (654.53) | 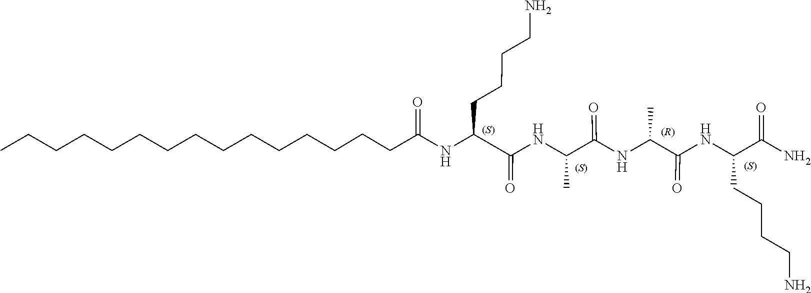 |
| 115 (654.53) | 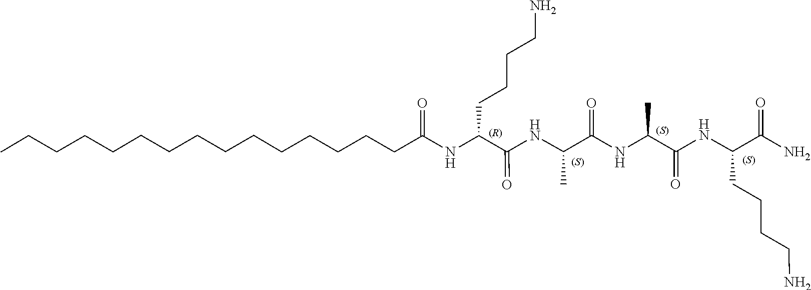 |
| 116 (699.82) | 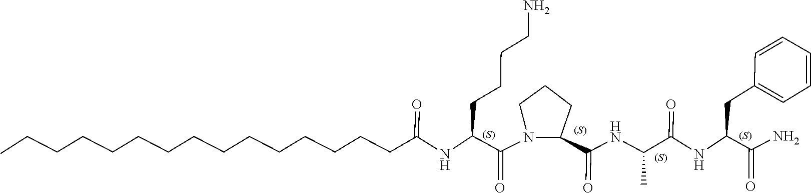 |
| 117 (713.72) | 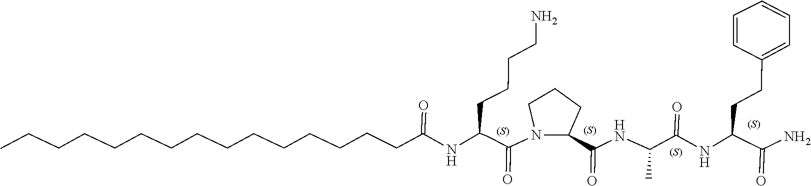 |
| 118 (713.94) | 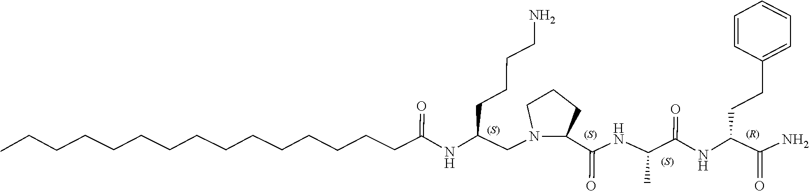 |
| 119 (680.3) | 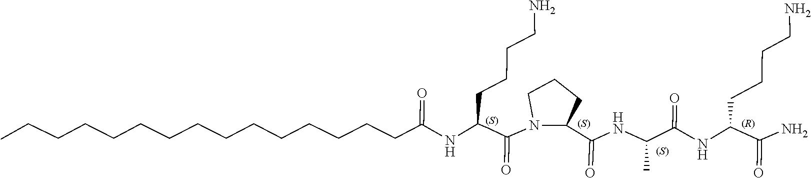 |
| 120 (738.2) | 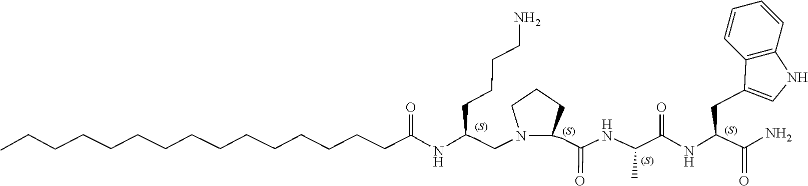 |
| 121 (631.49) | 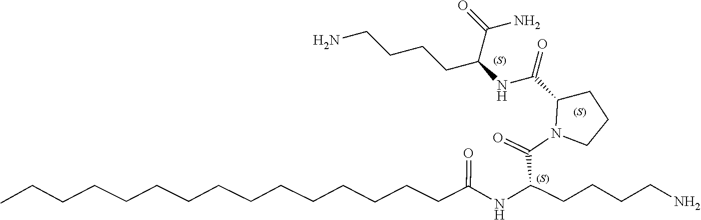 |
| 122 (738.63) | 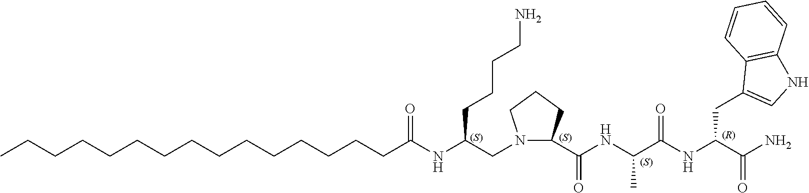 |
| 123 (699.72) | 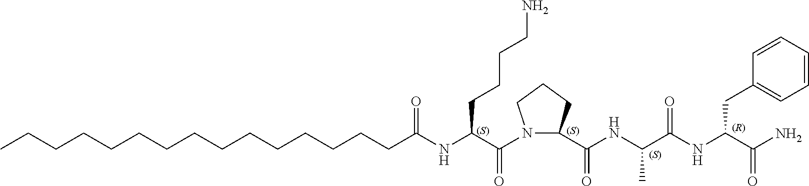 |
| 124 (624.2) | 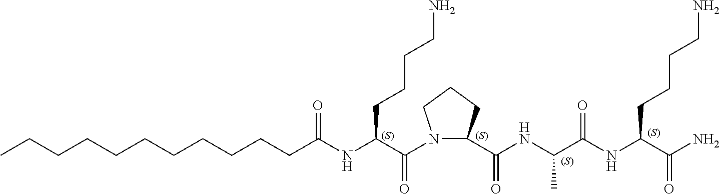 |
| 125 (736.3) | 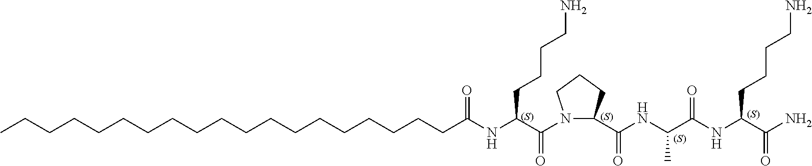 |
| 126 (681.3) | 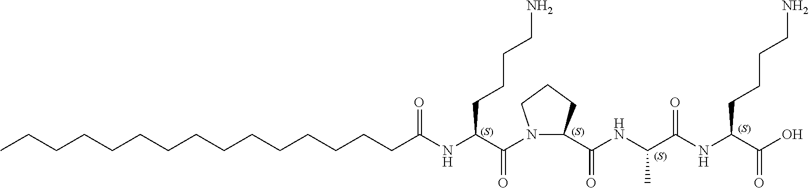 |
| 127 (784.68) | 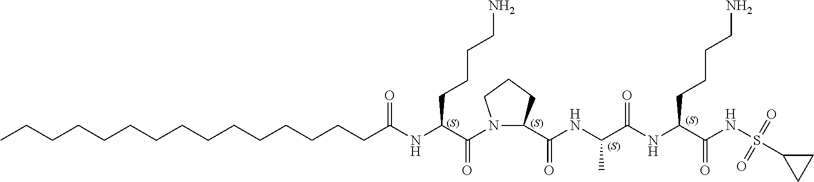 |
| 128 (741.3) | 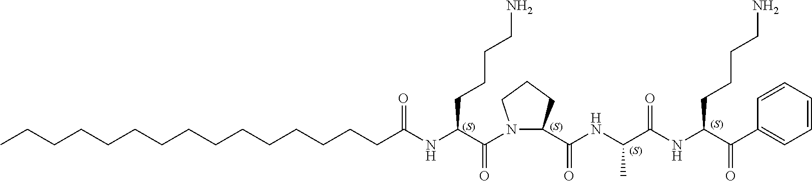 |
| 129 (791.54) | 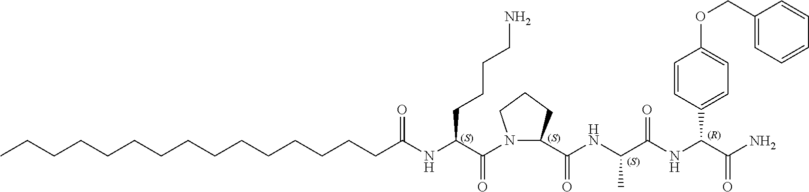 |
| 130 (791.54) | 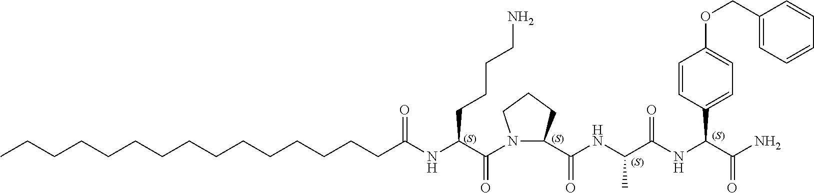 |
| 131 (598.2) | 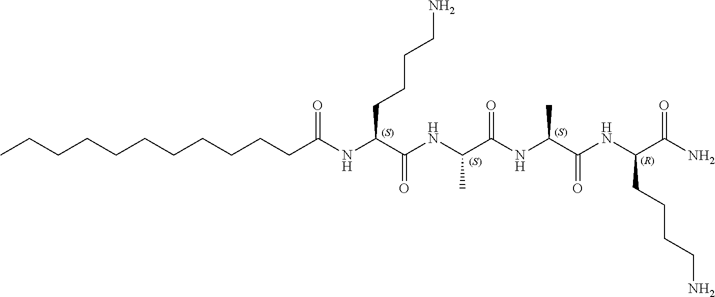 |
| 132 (710.3) | 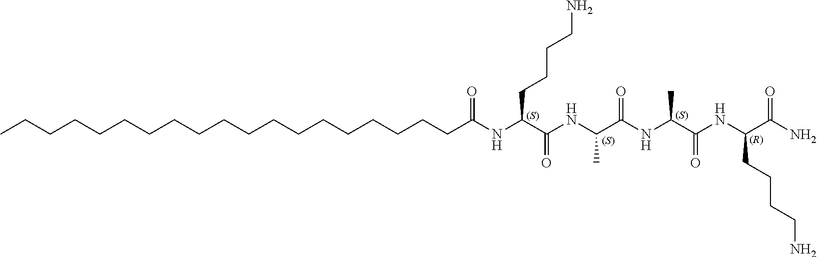 |
| 133 (702.52) | 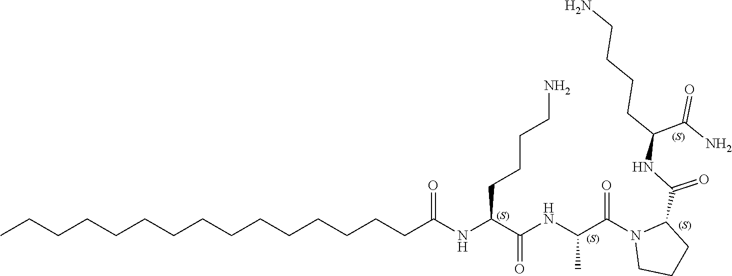 |
| 134 (655.3) | 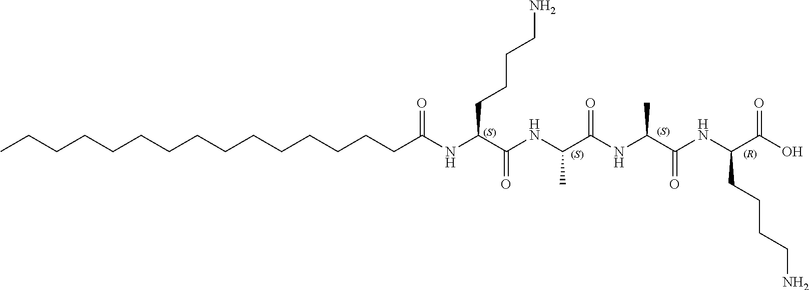 |
| 135 (758.4) | 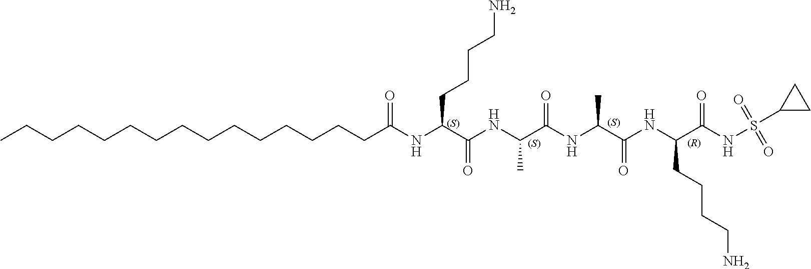 |
| 136 (715.5) | 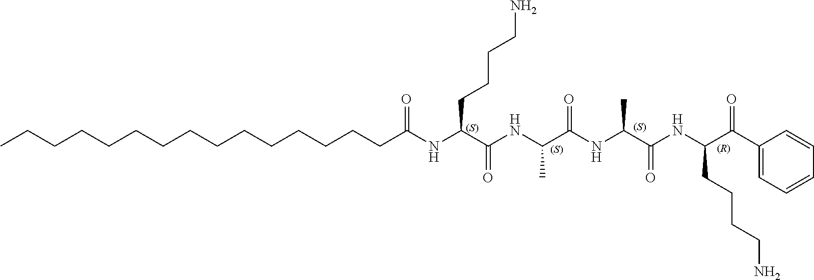 |
| 137 (715.5) | 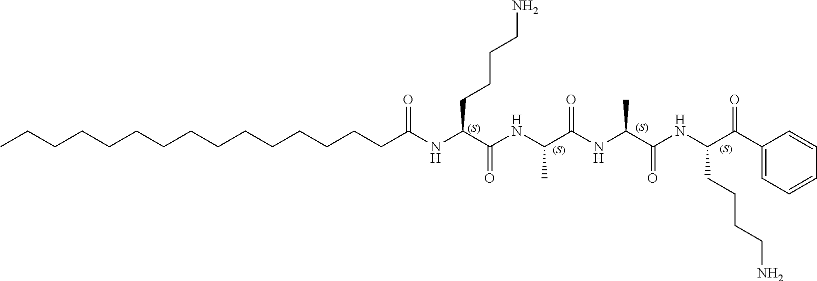 |
| 138 (765.53) | 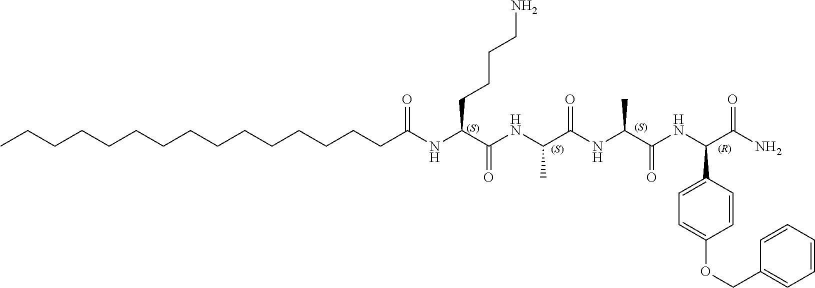 |
| 139 (765.53) | 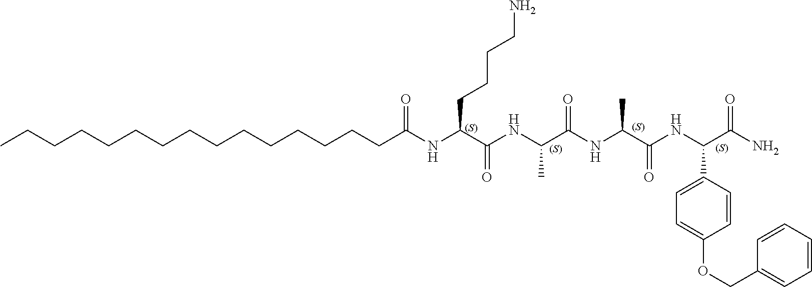 |
| 140 (552.53) | 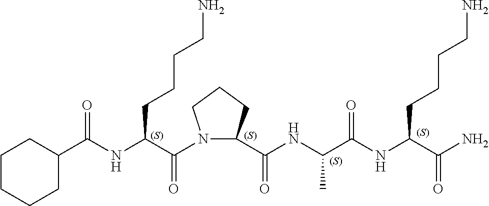 |
| 141 (604.58) | 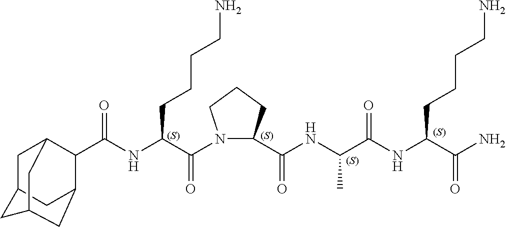 |
| 142 (526.59) | 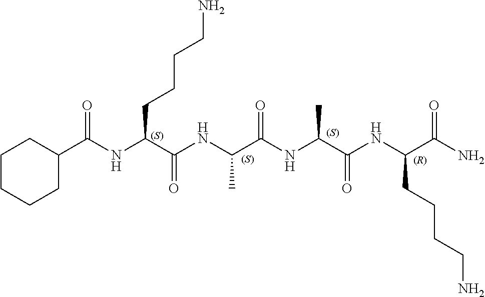 |
| 143 (578.69) | 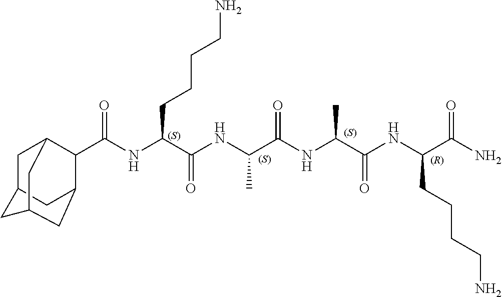 |
| 144 (484.32) | 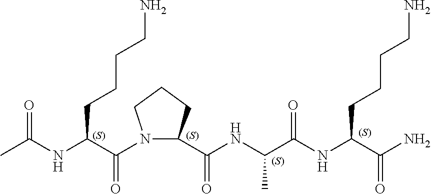 |
| 145 (458.31) | 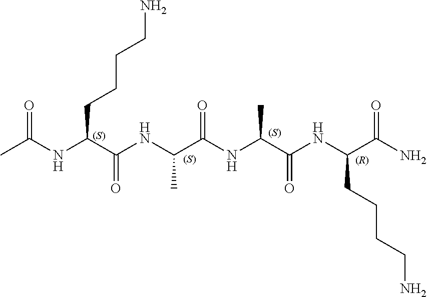 |
| 146 (605.47) | 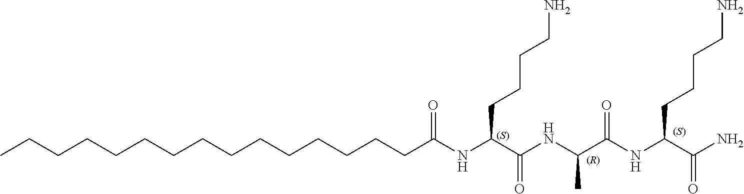 |
| 147 (597.47) | 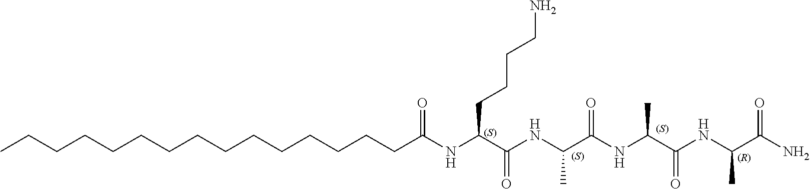 |
| 148 (597.47) | 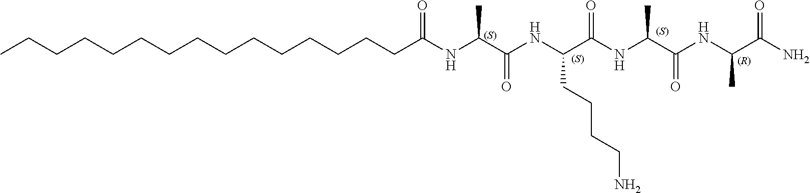 |
| 149 (597.47) | 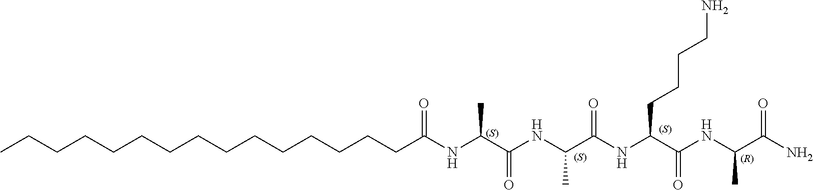 |
| 150 (597.47) | 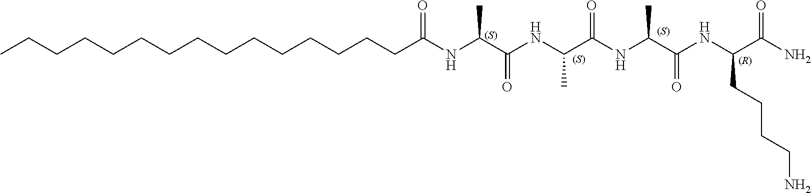 |
| 151 (626.5) | 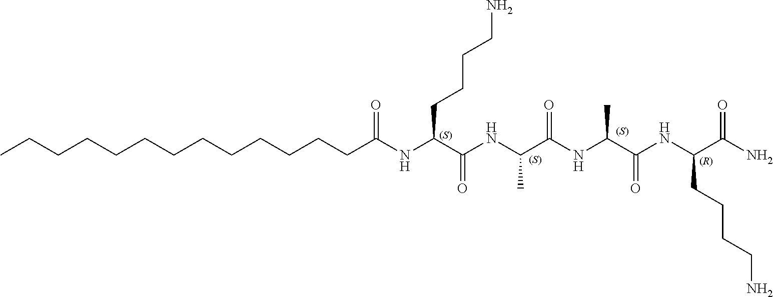 |
| 152 (541.41) | 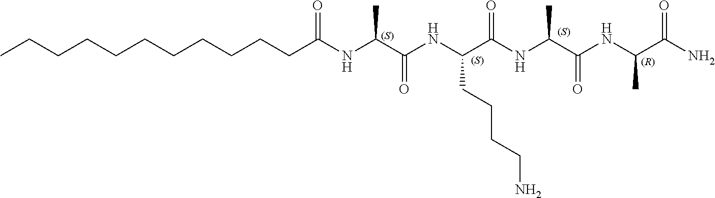 |
| 153 (513.38) | 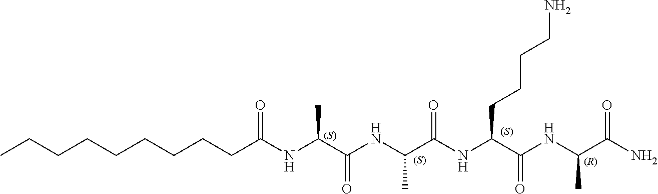 |
| 154 (485.34) | 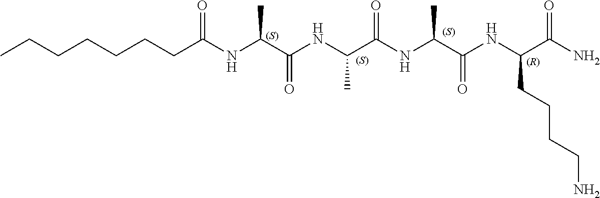 |
| 155 (570.43) | 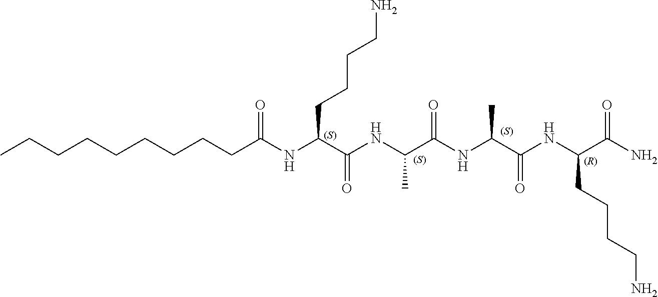 |
| 156 (542.4) | 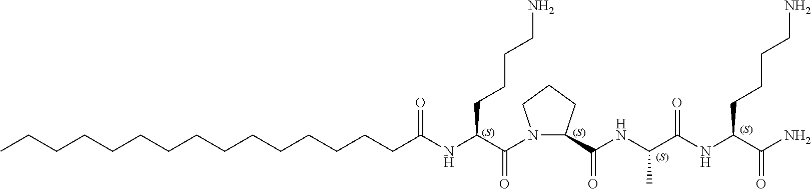 |
| 157 (702.53) | 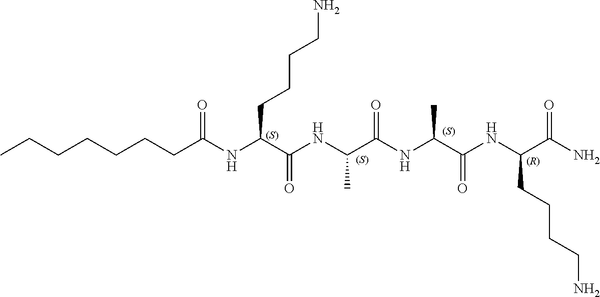 |
| 158 (654.53) | 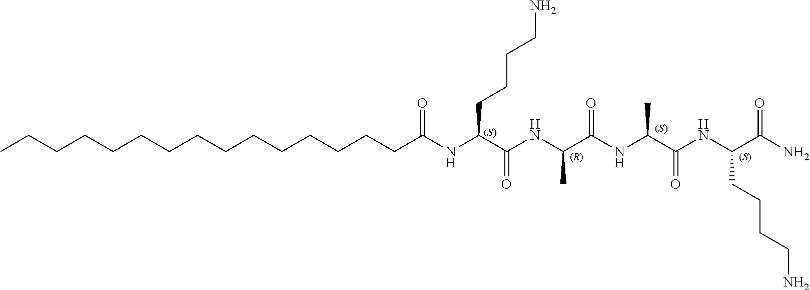 |
| 159 (583.49) | 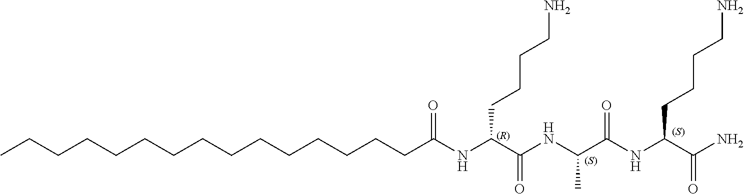 |
| 160 (583.49) | 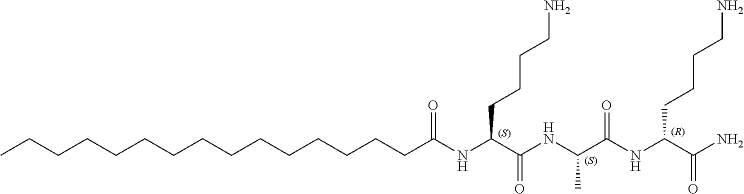 |
| 161 (540.46) | 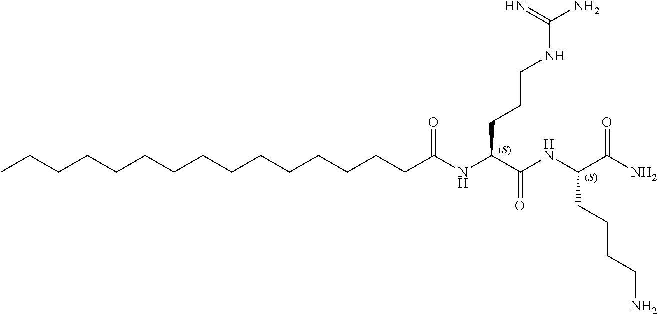 |
| 162 (521.42) | 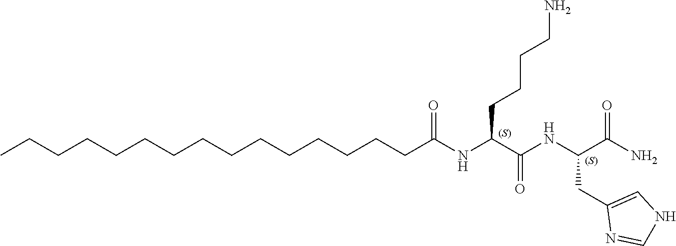 |
| 163 (689.72) | 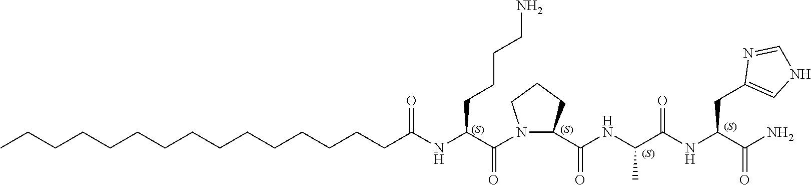 |
| 164 (689.2) | 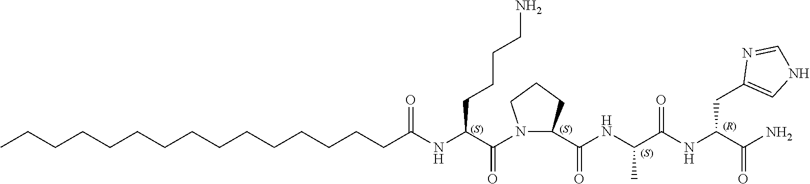 |
| 165 (703.52) | 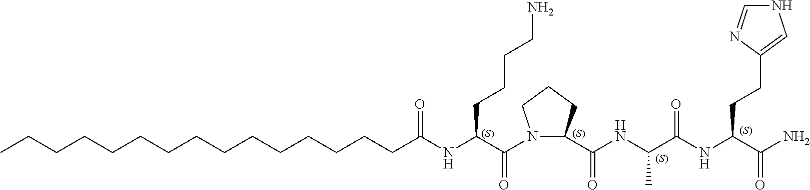 |
| 166 (703.52) | 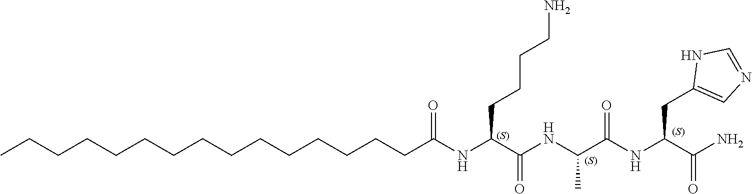 |
| 167 (592.46) | 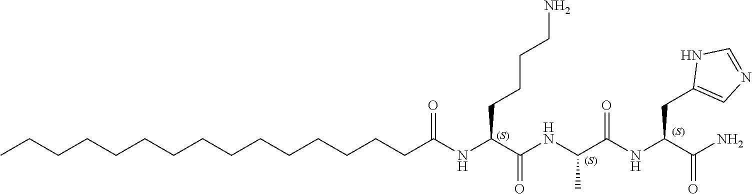 |
| 168 (663.49) | 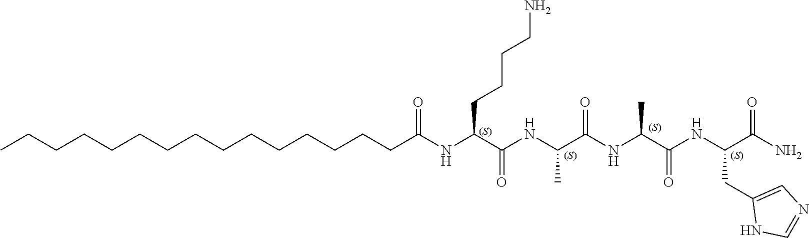 |
| 169 (623.4) | 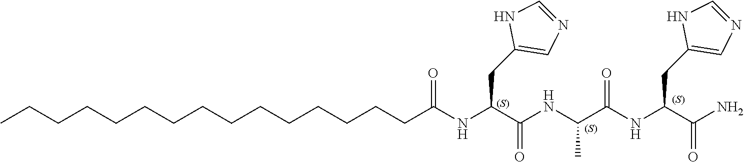 |
| 170 (549.42) | 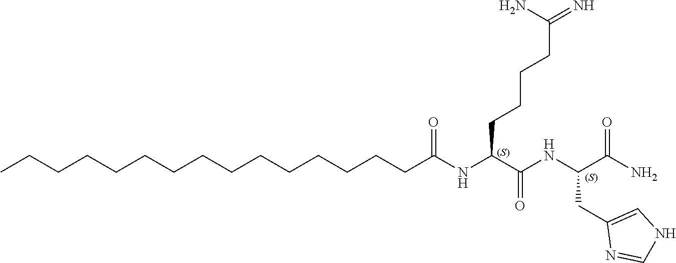 |
| 171 (663.2) | 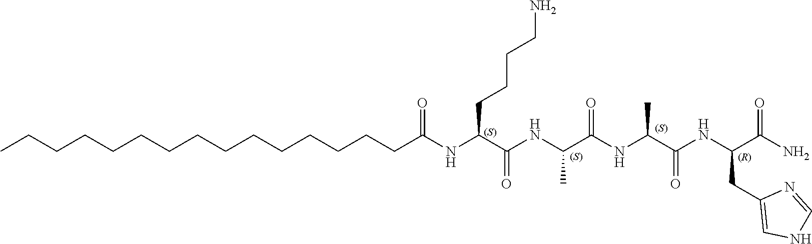 |
| 172 (677.5) | 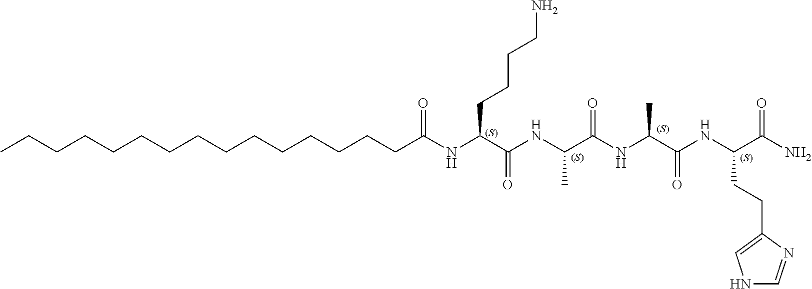 |
| 173 (677.5) | 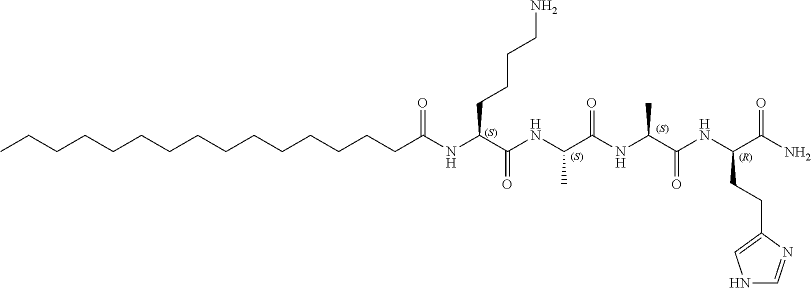 |
| 174 (644.9) | 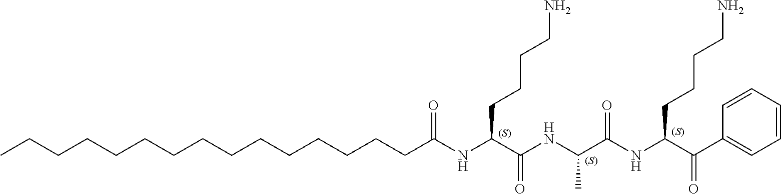 |
| 175 (583.2) | 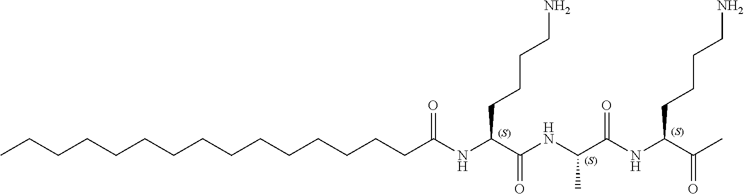 |
| 176 (593.48) | 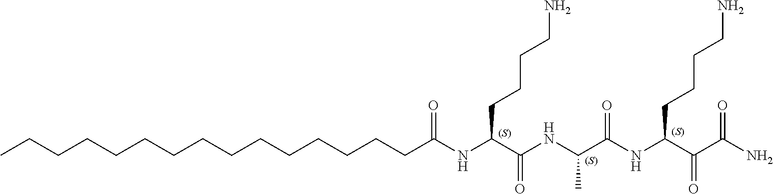 |
| 177 (698.48) | 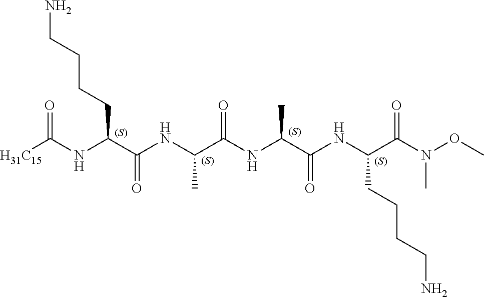 |
| 178 (687.4) | 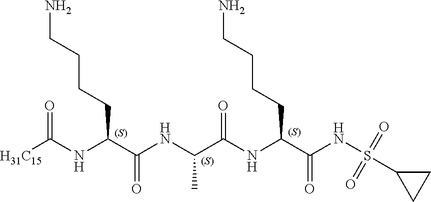 |
| 179 (687.4) | 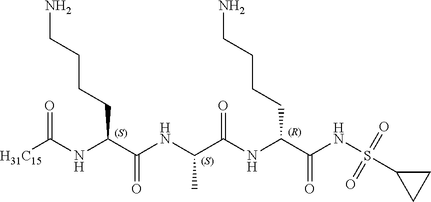 |
| 180 (621.48) | 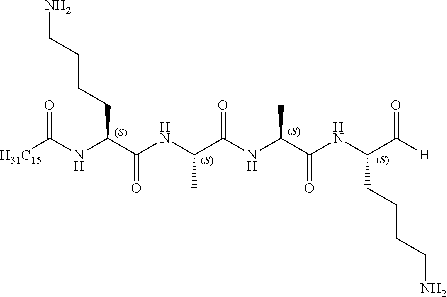 |
| 181 (621.48) | 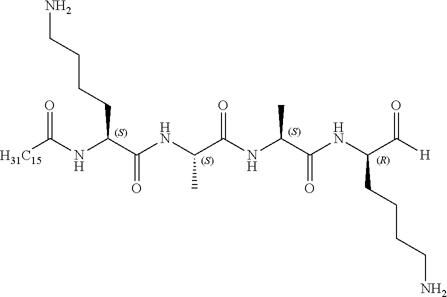 |
| 182 (566.48) | 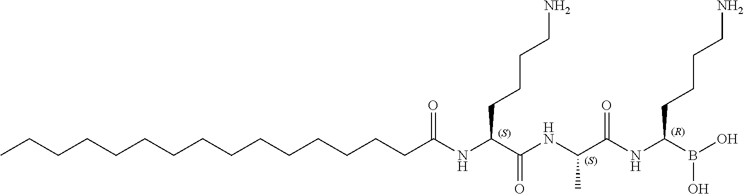 |
| 183 (677.51) | 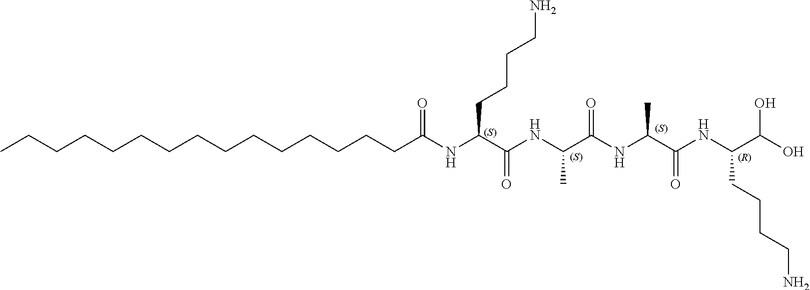 |
| 184 (611.52) | 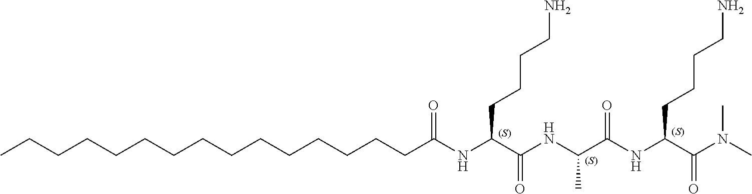 |
| 185 (627.52) | 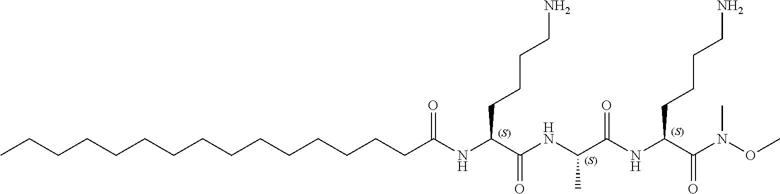 |
| 186 (590.46) | 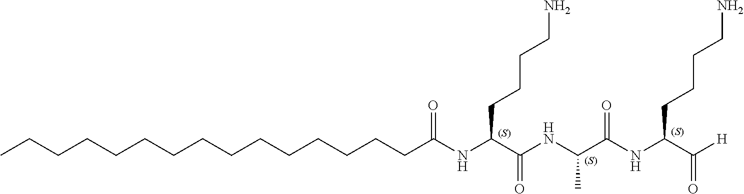 |
| 187 (682.28) | 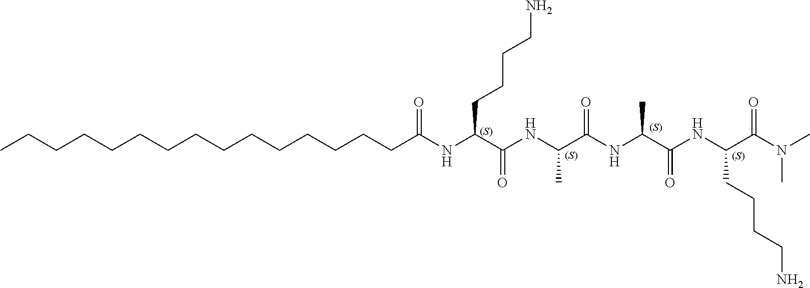 |
| 188 (584.24) | 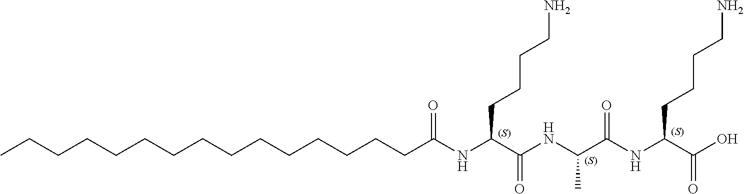 |
| 189 (655.28) | 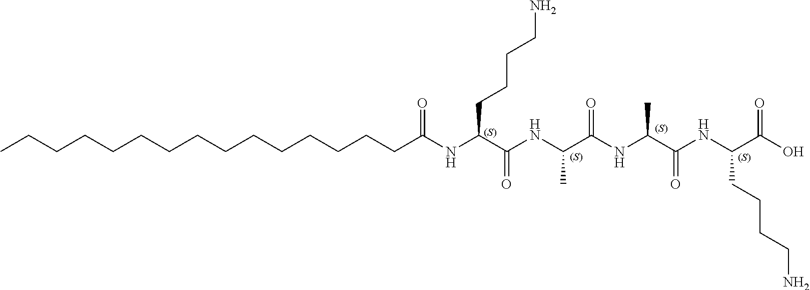 |
| 190 (602.4) | 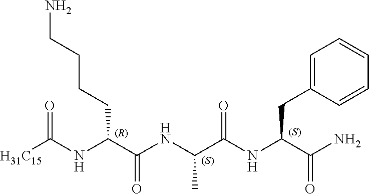 |
| 191 (769.84) | 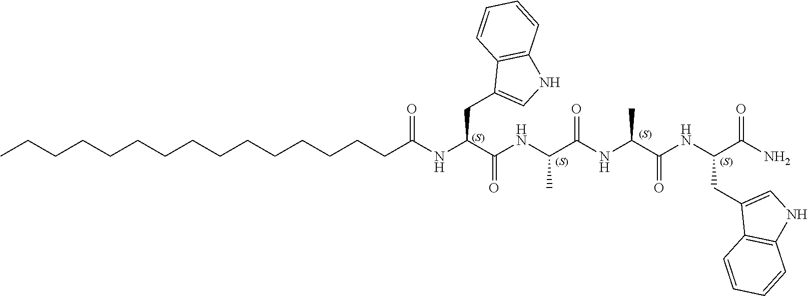 |
| 192 (602.28) | 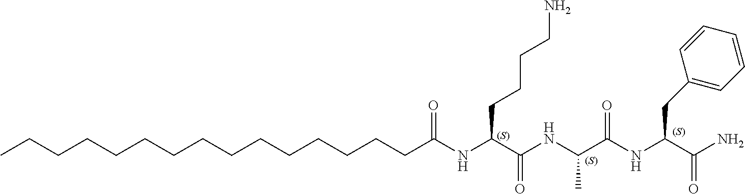 |
| 193 (673.48) | 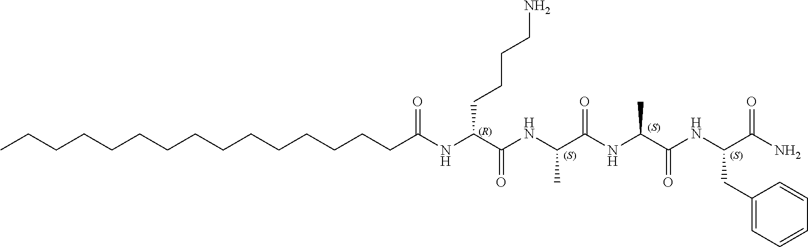 |
| 194 (621.08) | 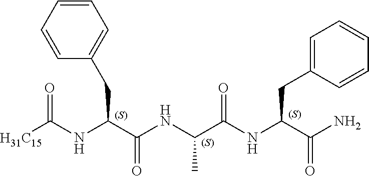 |
| 195 (692.2) | 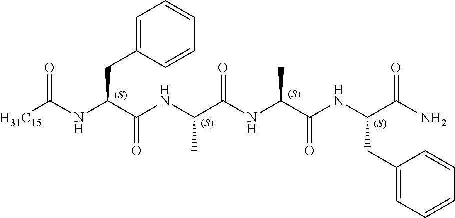 |
| 196 (738.48) | 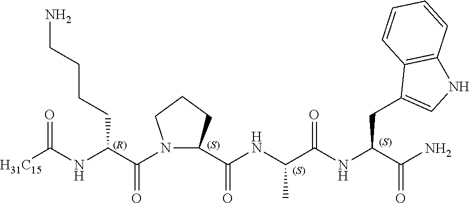 |

### Viral Proteases

Compounds of the invention are suitable as protease inhibitors, advantageously as inhibitors of viral proteases, particularly of flaviviral proteases. Flavivirus is a genus of the family Flaviviridae. This genus includes the West Nile virus, dengue virus, Tick-borne Encephalitis Virus, Japanese Encephalitis virus, Yellow Fever Virus, and several other viruses that may cause encephalitis.

Dengue virus and its various strains and isolates are members of the genus Flavivirus. The genus Flavivirus is a genera of the Flaviviridae family and includes the viral groups of Yellow Fever virus group, Tick-borne encephalitis virus group, Rio Bravo Group, Japanese encephalitis Group, Tyuleniy Group, Ntaya Group, Uganda S Group, Dengue Group, and Modoc Group. Members of the Flavivirus genus may produce a wide variety of disease states, such as fever, arthralgia, rash, hemorrhagic fever, and/or encephalitis. The outcome of infection is influenced by both the virus and host-specific factors, such as age, sex, genetic susceptibility, and/or pre-exposure to the same or a related agent. Some of the various diseases associated with members of the genus Flavivirus are yellow fever; dengue fever; and West Nile, Japanese, and St. Louis encephalitis.

The existence of a trypsin-like serine protease domain in the N-terminal region of the flaviviral NS3 proteins was originally predicted by sequence comparisons between cellular and virus-encoded proteases. The NS2B—NS3 endopeptidases of the Flavivirus genus, which at present comprises at least 68 known members, are now commonly designated as flavivirin (EC 3.4.21.91). The dengue virus 69 kDa NS3 protein is a multifunctional protein with a serine protease domain located within the N-terminal 167 amino acid residues and activities of a nucleoside triphosphatase (NTPase) and RNA helicase in the C-terminal moiety. A catalytic triad consisting of residues His51, Asp75 and Ser135 was identified by site-directed mutagenesis experiments and replacement of the catalytic serine by alanine resulted in an enzymatically inactive NS3 protein. The NS3 protease is an essential component for maturation of the virus and viable virus was never recovered from infectious cDNA clones carrying mutations in the NS3 sequence that abolished protease activity. Interaction of the helicase portion of NS3 with the viral RNA-dependent RNA polymerase NS5 may promote the association of the viral replicase complex to the membranes of the ER.

The DENV NS3 is a serine protease, as well as an RNA helicase and RTPase/NTPase. The protease domain consists of six β-strands arranged into two β-barrels formed by residues 1-180 of the protein. The catalytic triad (His-51, Asp-75 and Ser-135), is found between these two β-barrels, and its activity is dependent on the presence of the NS2B cofactor. This cofactor wraps around the NS3 protease domain and becomes part of the active site. The remaining NS3 residues (180-618), form the three subdomains of the DENV helicase. A six-stranded parallel β-sheet surrounded by four α-helices make up subdomains I and II, and subdomain III is composed of 4 α-helices surrounded by three shorter α-helices and two antiparallel β-strands

The presence of a small activating protein or co-factor is a prerequisite for optimal activity of the flaviviral NS3 proteases with their natural polyprotein substrates. Although the dengue virus NS3 protease exhibits NS2B-independent activity with model substrates for serine proteases, enzymatic cleavage of dibasic peptides is markedly enhanced with the NS2B—NS3 co-complex and the presence of the NS2B activation sequence is important for the cleavage of polyprotein substrates in vitro. The initial characterization of the co-factor requirement for the dengue virus NS3 protease had revealed that the minimal region necessary for protease activation was located in a 40-residue hydrophilic segment of NS2B.

A typical enzyme of interest in the present invention is a serine protease from viruses of the flaviviridae family (especially the flavivirus genus), such as dengue protease or West Nile protease. For example, dengue proteases from the four different dengue virus serotypes have all been well known and characterized in the art. See, e.g., Chambers et al., Proc. Nat. Acad. Sci. USA 87:8898-902, 1990; Chambers et al., J. Virol. 67:6797-807, 1993, Ryan et al. J. Gen. Virology 79:947-959, 1998; Murthy et al., J Biol Chem 274:5573-5580, 1999; Murthy et al., J Mol Biol 301:759-767, 2000; Brinkworth et al., J Gen Virol 80, 1167-1177, 1999 (Den2), and Leung et al., J Biol Chem 276:45762-71, 2001 (Den2). The virus-encoded dengue protease comprises the amino-terminal 180 amino acids of NS3 (NS3pro) of the polyprotein. It is responsible for cleavage both in cis and in trans to generate viral proteins that are essential for viral replication and maturation of infectious dengue virions. In addition to its protease activity, the carboxyl-terminal region of NS3 encodes both nucleoside triphosphatase and helicase activities (see, e.g., Li et al., J. Virol. 73:3108-3116, 1999). Similarly, other flaviviruses (e.g., West Nile virus and yellow fever virus) and their proteases have also been well characterized in the art. See, e.g., Anderson et al., Ann N Y Acad. Sci. 951:328-31, 2001; Nall et al., J Biol Chem. 79:48535-42, 2004; J Biol Chem. 280:2896-903, 2005; Hillyer et al., Histochem Cell Biol. 117:431-40, 2002; Chambers et al., J Gen Virol. 86:1403-13, 2005; Scaramozzino et al., Biochem Biophys Res Commun. 294:16-22, 2002; and Bessaud et al., Virus Res. 120:79-90, 2006. The viral genome of the Flavivirus genus is translated as a single polyprotein and is subsequently cleaved into mature proteins.

Compounds of the invention are preferably inhibitors of viral protease, preferably of flaviviral protease, more preferably of NS3 proteases such as flaviviral NS3 proteases, most preferably of flavivirin NS2B—NS3 endopeptidases such as dengue NS2B—NS3 protease. Throughout this document preferred proteases to be inhibited are viral protease, preferably flaviviral protease, more preferably NS3 proteases such as flaviviral NS3 proteases, most preferably of flavivirin NS2B—NS3 endopeptidase such as dengue NS2B—NS3 protease.

Compounds of the invention can additionally be useful for being specific. Preferably the compounds are specific protease inhibitors. Preferably the compounds do not inhibit or do not strongly inhibit other proteases such as host proteases or proteases of the subject to be treated. More preferably the compounds do not inhibit or do not strongly inhibit other serine proteases such as trypsin. As used herein, compounds that do not strongly inhibit a protease preferably inhibit it with a Ki of 200, 300, 400, 500 UM or greater, or do not detectably inhibit it.

In embodiments, the compounds according to the invention inhibit their target proteases at least 10% more than that they inhibit trypsin, preferably human trypsin, under the same experimental conditions. More preferably this is 20%, 30%, 40%, 50%, 60%, 70%, 80%, 90%, 95%, 96%, 97%, 98%, 99%, 100%, 150%, 200%, 250%, 300%, 350%, 400%, 450%, or 500% more, or even more. In other embodiments the compounds according to the invention inhibit trypsin, preferably human trypsin, at most 90% as compared to their inhibition of a viral protease, under the same experimental conditions. More preferably at most 80%, 70%, 60%, 50%, 40%, 30%, 20%, 15%, 10%, 9%, 8%, 7%, 6%, 5%, 4%, 3%, 2%, 1%, 0.5%, or less. Methods for determining inhibition are known in the art, and are exemplified in the examples.

Therapeutic compounds and compositions of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

### Methods and Uses of the Compounds

Because the compounds of the invention are good inhibitors of viral proteases, they can be used in related methods. The invention provides a method for inhibiting a viral protease, the method comprising the step of contacting the viral protease with a compound according to the invention, or with a composition according to the invention. This method is preferably for the treatment of a disease or condition associated with viral infection or a condition related to a viral infection, and comprises administration to the subject of an effective dose of a compound or composition according to the invention. Preferably the viral protease is a flaviviral protease, more preferably it is a dengue virus protease, a West Nile virus protease, or a tick-borne encephalitis virus protease. In some embodiments it is a West Nile virus protease. In some embodiments it is a tick-borne encephalitis virus protease. Most preferably it is a dengue virus protease. In some embodiments it is a dengue virus protease or a tick-borne encephalitis virus protease. In some embodiments it is a dengue virus protease or a West Nile virus protease. In some embodiments it is a West Nile virus protease or a tick-borne encephalitis virus protease.

In one embodiment, the use is provided of either a compound according to the invention, or with a composition according to the invention. Said use is preferably for the treatment of a disease or condition associated with viral infection or a condition related to a viral infection, and comprises administration to the subject of an effective dose of a compound or composition according to the invention. Further features and definitions are preferably as defined elsewhere herein, particularly for diseases or conditions to be treated. The methods and uses described here can be in vitro, in vivo, or ex vivo.

### Medical Use of the Compounds

In another aspect, the invention provides the compound or composition according to the invention for use as a medicament. The medicament is preferably for use in the treatment of a viral infection or a condition related to a viral infection. The medicament is very suitable for use in a method of treating, preventing, or delaying a viral infection or a condition related to a viral infection in a subject in need thereof, the method comprising the step of administering to the subject an effective amount of a compound or composition according to the invention.

Preferred viral infections are flaviviral infections. Flavivirus is a genus of the family Flaviviridae. This genus includes the West Nile virus, dengue virus, Tick-borne Encephalitis Virus, Japanese Encephalitis virus, Yellow Fever Virus, and several other viruses that may cause encephalitis. In certain aspects, the flavivirus infection may be a Dengue virus infection or a West Nile virus infection. Additional flaviviruses that can be treated or prevented include other mosquito-borne flaviviruses, such as Japanese encephalitis, Murray Valley encephalitis, St. Louis encephalitis, Kunjin, Rocio encephalitis, and Ilheus viruses; tick-borne flaviviruses, such as Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, Apoi, and Hypr viruses.

The compounds of the present invention can inhibit flaviviral NS3 proteases and therefore can have applications in treating or preventing diseases caused by these viruses. For example, they are useful in the treatment of West Nile encephalitis, yellow fever, Japanese encephalitis, dengue fever, dengue hemorrhagic fever, and dengue shock syndrome associated with the different dengue virus serotypes. In some embodiments the treatment is of serotype DENV-1, serotype DENV-2, serotype DENV-3, or serotype DENV-4. In some embodiments the treatment is of serotype DENV-1. In preferred embodiments the treatment is of serotype DENV-2. In some embodiments the treatment is of serotype DENV-3. In some embodiments the treatment is of serotype DENV-4. In preferred embodiments the treatment is of infection by Dengue virus or of infection by West Nile Virus, more preferably by DENV-2 or by West Nile Virus.

The flaviviral protease inhibitors or prodrugs of the present invention can be used alone or in combination with any known antiviral drugs to treat these infections.

The methods described herein are useful for both prophylactic and therapeutic treatment of viral infections such as Flavivirus infections. For prophylactic use, a therapeutically effective amount of one or more compounds as described herein is administered to a subject prior to exposure (e.g., before or when traveling to a location where Flavivirus infections are possible), during a period of potential exposure to Flavivirus infections, or after a period of potential exposure to Flavivirus infections. Prophylactic administration can occur for several days to weeks prior to potential exposure, during a period of potential exposure, and for a period of time, e.g., several days to weeks, after potential exposure. Therapeutic treatment involves administering to a subject a therapeutically effective amount of one or more compounds or compositions according to the invention.

The term “treat” or “treatment” or “treating” refers to the medical management of a patient with the intent to cure, ameliorate, stabilize, or prevent a disease, pathological condition, or disorder. In certain aspect treating reduces viral load, ameliorate symptoms, delays progression of disease, etc. This term includes active treatment that includes treatment directed specifically toward the improvement of a disease, pathological condition, or disorder, and also includes causal treatment, that is, treatment directed toward removal of the cause of the associated disease, pathological condition, or disorder. In addition, this term includes supportive treatment including treatment employed to supplement another specific therapy directed toward the improvement of the associated disease, pathological condition, or disorder.

As used herein the terms treatment, treat, or treating refer to a method of reducing or delaying one or more symptoms of viral infections such as a Flavivirus infection. Thus in the disclosed method, treatment can refer to a 10%, 20%, 30%, 40%, 50%, 60%, 70%, 80%, 90%, or 100% reduction in the severity or progression of one or more symptoms of the disease or condition. For example, a method for treating a disease is considered to be a treatment if there is a 10% reduction in one or more symptoms or signs of the Flavivirus infection in a subject as compared to a control. As used herein, control refers to the untreated condition. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Examples of symptoms are fever and fatigue.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder. For example, the method is considered to be a prevention if there is a reduction or delay in onset, incidence, severity, or recurrence of a Flavivirus infection. The reduction or delay in onset, incidence, severity, or recurrence of a Flavivirus infection can be a 10%, 20%>, 30%, 40%, 50%, 60%, 70%, 80%, 90%, 100%, or any percent reduction in between 10% and 100% as compared to native or control levels.

As used herein the terms treatment, treat, or treating refer to a method of reducing or delaying one or more symptoms of a Flavivirus infection. Thus in the disclosed method, treatment can refer to a 10%, 20%, 30%, 40%, 50%, 60%, 70%, 80%, 90%, or 100% reduction in the severity or progression of one or more symptoms of the disease or condition. For example, a method for treating a disease is considered to be a treatment if there is a 10% reduction in one or more symptoms or signs of the Flavivirus infection in a subject as compared to a control. As used herein, control refers to the untreated condition. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

### Compositions, Formulation, and Administration

In a further aspect, the invention provides a composition comprising a pharmaceutically acceptable excipient and a compound according to the invention, preferably wherein the composition is a pharmaceutical composition. Such a composition is referred to herein as a composition according to the invention. Preferred compositions according to the invention are pharmaceutical compositions and are for use in treatment as defined elsewhere herein. In preferred embodiments, the composition according to the invention is formulated for oral, sublingual, parenteral, intravascular, intravenous, subcutaneous, inhaled, or transdermal administration; preferably for oral administration. More features and definitions of administration methods are provided below.

Oral drug administration is a commonly used route for many drugs. Compounds of the invention can be seen as peptidomimetics, for which intravenous injections and intramuscular injections each are clinically commonly used administration routes. Surprisingly, subcutaneous delivery (another well-accepted administration method) was found to improve the bioavailability and pharmacokinetic behaviour of compounds of the invention. Preclinical data (see Example 18) show that subcutaneous administration of compounds such as compounds 113 and 43 has great efficacy, and that the compounds show improved pharmacokinetic behaviour when administered subcutaneously. This can enable prophylactic and therapeutic use, for example in the case of dengue infection, in comparison to other routes of intake such as oral intake.

The increased efficiency allows subcutaneous drug delivery to achieve additional advantages for the three main target populations related to dengue, which are (1) therapeutic use for early treatment of dengue; this first group are the patients that have recently been infected by the virus and need effective treatment as soon as possible and preferentially with a fast onset of action, to immediately suppress the viral load built-up and associated disease symptoms, thereby reducing the risk of progression to severe disease and progression of the virus to others through cross-infection. The two other groups comprise healthy subjects that are at risk of infection and therefore require access to prophylactic use: (2) prophylactic use for subjects at risk of contracting dengue, and (3) prophylactic use for subjects visiting regions where dengue is endemic.

To control dengue epidemics (e.g., outbreak management), it is important that all target populations have access to treatment. In preferred embodiments, the compound for use or the composition for use is for therapeutic use for early treatment of dengue, for prophylactic use for subjects at risk of contracting dengue, or for prophylactic use for subjects visiting regions where dengue is endemic. In some embodiments the compound for use or the composition for use is for therapeutic use for early treatment of dengue. In some embodiments the compound for use or the composition for use is for prophylactic use, preferably for subjects at risk of contracting dengue, or for subjects visiting regions where dengue is endemic. In some embodiments the compound for use or the composition for use is for prophylactic use for subjects at risk of contracting dengue. In some embodiments the compound for use or the composition for use is for prophylactic use for subjects visiting regions where dengue is endemic.

Subcutaneous administration was found to have specific dengue-relevant advantages for efficient systemic delivery and patient/traveller compliance and convenience. Preclinical pharmacokinetic data (see Example 18) showed slow elimination of the compounds, indicating that a single dose subcutaneous administration can be sufficient for one or two weeks of treatment in a therapeutic and prophylactic setting. Based on preclinical data, a single dose subcutaneous administration can be sufficient for one or two weeks of therapeutic and/or prophylactic treatment with compounds according to the invention. This has relevant advantages for dengue patient compliance and convenience since patients with severe dengue may not be able to take oral drugs. This approach also yields advantages for visitors to areas where there is an increased risk of infection, such as travellers who visit risk areas, who need only a (bi) weekly shot (similar to a vaccination) instead of an oral (twice) daily prophylaxis pill.

Subcutaneous administration can be either via direct injection, via autoinjectors, or via patches. Preferably it is via direct injection or via patches, more preferably via direct injection.

Vector-mediated transmission of DENV is initiated when a blood-feeding female Aedes mosquito injects saliva, together with the virus, into the skin of its mammalian host. The cells that first encounter the infection are skin resident macrophages, dendritic cells and keratinocytes (Martina, Koraka, and Osterhaus. 2009. Clinical Microbiology Reviews 22 (4)). These infected cells migrate to the lymph nodes where macrophages and monocytes are exposed to the virus and get infected. The DENV infection progresses to viremia (primary viremia; virus in blood) due to the presence of the virus leaving the draining and remote lymph nodes. Infection of DENV is observed in the spleen, kidneys, lungs, and liver, although spleen and liver are the key target organs. Specifically, the macrophages in these organs are the major sites of viral replication (Martina et al., 2009). The secondary viremia occurs when the virus leaves the organs, which can be detected as early as 24-48 h (4-7 days after infection) before the onset of clinical symptoms (such as fever) and can last up to 10-12 days. High concentration of virus in the blood is related to the risk to develop severe disease (haemorrhage and shock). Low to moderate concentration of virus in the blood is associated with mild disease (Martina et al., 2009).

Following subcutaneous administration, molecules reach the systemic circulation via either the blood capillaries or the lymphatic system. Data showed that efficacy against DENV increased substantially when compounds were administered via subcutaneous injection over other routes of administration. The compounds reached more than three times higher concentrations in the main target sites of dengue (liver and spleen) when compared to blood plasma. Accordingly, in preferred embodiments the compound for use or composition for use is for use in a method of targeting a protease inhibitor to a target organ of a subject, the method comprising injected, preferably subcutaneous administration of the protease inhibitor. Injected administration can be intravenous, intramuscular, or subcutaneous. Preferred targeted organs are liver, kidney, lungs, and spleen, more preferably liver, kidney, and spleen, still more preferably liver and spleen, most preferably liver. In some embodiments the target organs are liver and kidney. In some embodiments the target organ is spleen. Preferably the protease inhibitor is a compound according to the invention, such as compound 113 or compound 43. Preferably the protease inhibitor is a DENV protease inhibitor.

The invention also provides combinations of compounds according to the invention with further measures known for treating or ameliorating diseases or conditions related to a viral infection. In preferred embodiments of such combinations is provided a combination of a compound according to the invention and an antiviral agent. Antiviral agents are widely known. In another preferred combination, the compound according to the invention is combined with an anti-inflammatory agent. In some preferred combinations the compound may be combined with clinical management, for example involving physical therapy, aerobic exercise, respiratory function therapy, or orthopedic interventions.

Antiviral agents are known in the art and a skilled can select a suitable antiviral agent in light of this disclosure. Examples of suitable antiviral agents are abacavir, acyclovir, adefovir, amantadine, ampligen, amprenavir, umifenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, penciclovir, peramivir, pleconaril, podophyllotoxin, raltegravir, remdesivir, ribavirin, rilpivirine, rilpivirine, rimantadine, ritonavir, squinavir, simeprevir, sofosbuvir, stavudine, taribavirin, telaprevir, telbivudine, tenofovir alafenamide, tenofovir disoproxil, tipranavir, trifluridine, trizivir, tromantadine, truvada, umifenovir, valaciclovir, valganciclovir, vicriviroc, vidarabine, zalcitabine, zanamivir, and zidovudine.

Anti-inflammatory agents are known in the art and a skilled can select a suitable anti-inflammatory agent in light of this disclosure. Examples of suitable antiviral agents are nonsteroidal anti-inflammatory drugs (NSAIDs), and corticosteroids. Examples of NSAIDs are pyrazolones (such as aminophenazone ampyrone azapropazone clofezone difenamizole famprofazone feprazone kebuzone metamizole mofebutazone morazonen ifenazone oxyphenbutazone phenazone phenylbutazone propyphenazone sulfinpyrazone and suxibuzone), salicylates (such as acetylsalicylic acid, aloxiprin, benorylate, carbasalate calcium, diflunisal, dipyrocetyl, ethenzamide, guacetisal, magnesium salicylate, methyl salicylate, salsalate, salicin, salicylamide, salicylic acid, and sodium salicylate), acetic acid derivatives (such as aceclofenac, acemetacin, alclofenac, amfenac, bendazac, bromfenac, bumadizone, bufexamac, diclofenac, difenpiramide, etodolac, felbinac, fenclozic acid, fentiazac, indomethacin, indometacin farnesyl, isoxepac, ketorolac, lonazolac, oxametacin, prodolic acid, proglumetacin, sulindac, tiopinac, tolmetin, and zomepirac), oxicams (such as ampiroxicam, droxicam, isoxicam, lornoxicam, meloxicam, piroxicam, and tenoxicam), propionic acid derivatives (profens, such as alminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, suprofen, tarenflurbil, tepoxalin, tiaprofenic acid, vedaprofen, zaltoprofen, and naproxcinod), N-arylanthranilic acids (fenamates, such as azapropazone, clonixin, etofenamate, floctafenine, flufenamic acid, flunixin, glafenine, meclofenamic acid, mefenamic acid, morniflumate, niflumic acid, tolfenamic acid, and flutiazin) coxibs (such as apricoxib, celecoxib, cimicoxib, deracoxib, etoricoxib, firocoxib, lumiracoxib, mavacoxib, parecoxib, robenacoxib, rofecoxib, and valdecoxib), aminopropionitrile, benzydamine chondroitin sulfate, diacerein, fluproquazone. Glucosamine, glycosaminoglycan, hyperforin, nabumetone, nimesulide, oxaceprol, proquazone, superoxide dismutase/orgotein, and tenidap. Examples of corticosteroids are hydrocortisone, hydrocortisone acetate, cortisone acetate, tixocortol pivalate, prednisolone, methylprednisolone, prednisone, amcinonide, budesonide, desonide, fluocinolone acetonide, fluocinonide, halcinonide, triamcinolone acetonide, beclometasone, betamethasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

The compositions comprising the compounds as described above, can be prepared as a medicinal or cosmetic preparation or in various other media, such as foods for humans or animals, including medical foods and dietary supplements. A “medical food” is a product that is intended for the specific dietary management of a disease or condition for which distinctive nutritional requirements exist. By way of example medical foods may include vitamin and mineral formulations fed through a feeding tube (referred to as enteral administration). A “dietary supplement” shall mean a product that is intended to supplement the human diet and is typically provided in the form of a pill, capsule, tablet or like formulation. By way of example a dietary supplement may include one or more of the following ingredients: vitamins, minerals, herbs, botanicals; amino acids, dietary substances intended to supplement the diet by increasing total dietary intake, and concentrates, metabolites, constituents, extracts or combinations of any of the foregoing. Dietary supplements may also be incorporated into food, including food bars, beverages, powders, cereals, cooked foods, food additives and candies; or other functional foods designed to promote health or to prevent or halt the progression of a treatment of a viral infection or a condition related to a viral infection.

The subject compounds and compositions may be compounded with other physiologically acceptable materials that can be ingested including foods. In addition, or alternatively, the compositions as described herein may be administered orally in combination with (the separate) administration of food.

The compositions or compound according to the invention may be administered alone or in combination with other pharmaceutical or cosmetic agents and can be combined with a physiologically acceptable carrier thereof. In particular, the compounds described herein can be formulated as pharmaceutical or cosmetic compositions by formulation with additives such as pharmaceutically or physiologically acceptable excipients carriers, and vehicles. Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003), 21st edition (2005) and 22nd edition (2012).

Compositions for use according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in a conventional manner using one or more physiologically acceptable carriers comprising excipients and auxiliaries that facilitate processing of the active compounds into preparations which can be used pharmaceutically. Proper formulation is dependent on the route of administration chosen.

For injection, the compounds and compositions for use according to the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Oral and parenteral administration may be used where the compounds and compositions for use are formulated by combining them with pharmaceutically acceptable carriers well known in the art, or by using them as a food additive. Such strategies enable the compounds and compositions for use according to the invention to be formulated as tablets, pills, dragées, capsules, liquids, gels, syrups, slurries, suspensions and the like, for oral ingestion by a subject to be treated. Preparations or pharmacological preparations for oral use may be made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Additionally, coformulations may be made with uptake enhancers known in the art.

Dragée cores are provided with suitable coatings. For this purpose, concentrated sugar solutions may be used, which may optionally contain gum arabic, talc, PVP, carbopol gel, polyethylene glycol, and/or titanium dioxide, lacquer solution, and suitable organic solvents or solvent mixtures. Polymethacrylates can be used to provide pH-responsive release profiles so as to pass the stomach. Dyestuffs or pigments may be added to the tablets or dragée coatings for identification or to characterize different combinations of active compound doses.

Compounds and compositions which can be administered orally include push-fit capsules made of gelatin, as well as soft, sealed capsules made of gelatin and a plasticizer, such as glycerol or sorbitol. The push-fit capsules may contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compounds and compositions for use according to the invention may be administered in the form of tablets or lozenges formulated in a conventional manner. The compounds and compositions for use according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents.

Compositions for parenteral administration include aqueous solutions of the compositions in water soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-free water, before use.

The compositions for use according to the invention may also be formulated in rectal compositions such as suppositories or retention enemas, e.g., containing conventional suppository bases such as cocoa butter or other glycerides.

In addition to the formulations described previously, the compounds and compositions for use according to the invention may also be formulated as a depot preparation. Such long acting formulations may be administered by implantation (for example subcutaneously or intramuscularly) or by intramuscular injection. Thus, for example, they may be formulated with suitable polymeric or hydrophobic materials (for example as an emulsion in an acceptable oil), or as part of a solid or semi-solid implant that may or may not be auto-degrading in the body, or ion exchange resins, or one or more components of the composition can be formulated as sparingly soluble derivatives, for example, as a sparingly soluble salt. Examples of suitable polymeric materials are known to the person skilled in the art and include PLGA and polylactones such as polycaproic acid.

The compositions for use according to the invention also may comprise suitable solid or gel phase carriers or excipients. Examples of such carriers or excipients include calcium carbonate, calcium phosphate, various sugars, starches, cellulose derivatives, gelatin, and polymers such as polyethylene glycols.

The compositions for use according to the invention may also be comprised in a transdermal patch. Preferred transdermal patches for use according to the invention are selected from single-layer drug-in-adhesive patch, or multi-layer drug-in-adhesive patch, or reservoir patch, or matrix patch, or vapour patch.

Compositions for use according to the invention include compounds and compositions wherein the active ingredients are contained in an amount effective to achieve their intended purposes. More specifically, a therapeutically effective amount means an amount of compound effective to prevent, stabilize, alleviate, revert, or ameliorate causes or symptoms of disease, or prolong the survival, mobility, or independence of the subject being treated. Determination of a therapeutically effective amount is within the capability of those skilled in the art, especially in light of the detailed disclosure provided herein. For any compounds and compositions used in the invention, the therapeutically effective amount or dose can be estimated initially from cell culture assays, for example as exemplified herein. Dosage may vary within this range depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics” Ch. 1 p. 1). The amount of compound and compositions administered will, of course, be dependent on the subject being treated, on the subject's weight, the severity of the affliction, the manner of administration and the judgment of the prescribing physician.

A composition for use according to the invention may be supplied such that a compound for use according to the invention and one or more of the other components as defined herein are in the same container, either in solution, in suspension, or in powder form. A composition for use according to the invention may also be provided with all components provided separately from one another, for example to be mixed with one another prior to administration, or for separate or sequential administration. Various packaging options are possible and known to the ones skilled in the art, depending, among others, on the route and mechanism of administration. In light of the methods of administration described above, the invention provides a compound for use according to the invention, or a composition for use according to the invention, characterized in that it is administered orally, sublingually, intravascularly, intravenously, subcutaneously, transdermally, or optionally by inhalation; preferably orally; more preferably subcutaneously.

An “effective amount” of a compound or composition is an amount which, when administered to a subject, is sufficient to reduce or eliminate either one or more symptoms of a disease, or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations.

The “effective amount” of that may be combined with the carrier materials to produce a single dosage form will vary depending upon the host to which the active ingredient is administered and the particular mode of administration. The unit dosage chosen is usually fabricated and administered to provide a desired final concentration of the compound in the blood.

The effective amount (i.e. the effective total daily dose), preferably for adults, is herein defined as a total daily dose of about 0.01 to 2000 mg, or about 0.01 to 1000 mg, or about 0.01 to 500 mg, or about 5 to 1000 mg, or about 20 to 800 mg, or about 30 to 800 mg or about 30 to 700 mg, or about 20 to 700 mg or about 20 to 600 mg, or about 30 to 600 mg, or about 30 to 500 mg, about 30 to 450 mg or about 30 to 400 mg, or about 30 to 350 mg or about 30 to 300 mg or about 50 to 600 mg, or about 50 to 500 mg, or about 50 to 450 mg, or about 50 to 400 mg or about 50 to 300 mg, or about 50 to 250 mg, or about 100 to 250 mg or about 150 to 250 mg. In the most preferred embodiment, the effective amount is about 200 mg. In preferred embodiments, the invention provides a compound for use according to the invention, or a composition for use according to the invention, characterized in that it is administered to a subject in an amount ranging from 0.1 to 1500 mg/day, preferably from 0.1 to 1000 mg/day, more preferably from 0.1 to 400 mg/day, still more preferably from 0.25 to 150 mg/day, such as about 100 mg/day.

Alternatively, the effective amount of the compound, preferably for adults, preferably is administered per kg body weight. The total daily dose, preferably for adults, is therefore about 0.05 to about 40 mg/kg, about 0.1 to about 20 mg/kg, about 0.2 mg/kg to about 15 mg/kg, or about 0.3 mg/kg to about 15 mg/kg or about 0.4 mg/kg to about 15 mg/kg or about 0.5 mg/kg to about 14 mg/kg or about 0.3 mg/kg to about 14 mg/kg or about 0.3 mg/kg to about 13 mg/kg or about 0.5 mg/kg to about 13 mg/kg or about 0.5 mg/kg to about 11 mg/kg.

The total daily dose for children is preferably at most 200 mg. More preferably the total daily dose is about 0.1 to 200 mg, about 1 to 200 mg, about 5 to 200 mg about 20 to 200 mg about 40 to 200 mg, or about 50 to 200 mg. Preferably, the total daily dose for children is about 0.1 to 150 mg, about 1 to 150 mg, about 5 to 150 mg about 10 to 150 mg about 40 to 150 mg, or about 50 to 150 mg. More preferably, the total daily dose is about 5 to 100 mg, about 10 to 100 mg, about 20 to 100 mg about 30 to 100 mg about 40 to 100 mg, or about 50 to 100 mg. Even more preferably, the total daily dose is about 5 to 75 mg, about 10 to 75 mg, about 20 to 75 mg about 30 to 75 mg about 40 to 75 mg, or about 50 to 75 mg.

Alternative examples of dosages which can be used are an effective amount of the compounds for use according to the invention within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight.

The NS2B—NS3 protease inhibitor can be given in a single dose, or multiple doses. Continuous administration also may be applied where appropriate. The dose of a therapeutic composition via continuous perfusion may be equivalent to that given by a single or multiple injections, adjusted over a period of time during which the perfusion occurs. The amount of NS2B—NS3 protease inhibitor administered may be dependent on the subject being treated, the subject's weight, the manner of administration, and the judgment of the physician. Treatment regimens may vary as well, and often depend on the type and location of the damage or symptoms, disease progression, and health and age of the patient.

In some embodiments, an NS2B—NS3 protease inhibitor may be administered to a patient systemically or by local injection. Systemic administration can be by intravenous or intraperitoneal delivery. The NS2B—NS3 protease inhibitor can be administered to reach a circulating level of about 2 to 20 mg/ml in blood, or a dose of about 100-300 mg can be delivered to a patient. Compounds or compositions for use according to the present invention may be administered in a single daily dose, or the total daily dosage may be administered in divided dosage of two, three or four times daily.

In a preferred embodiment of the invention, “subject”, “individual”, or “patient” is understood to be an individual organism, preferably a vertebrate, more preferably a mammal, even more preferably a primate and most preferably a human. A subject is preferably a subject who has been diagnosed with a disease or condition as described herein, or who is suspected of suffering from a disease or condition as described herein, or who resides or has resided in an area where a disease or condition as described herein is prevalent, more preferably the subject has been diagnosed.

Compounds of the invention were found to not inhibit cytochrome p450. Accordingly, the invention is suitable for treating subjects who have comedication that is known to inhibit cytochrome p450. A skilled person knows which drugs inhibit cytochrome p450, and a subject may be aware of the fact and can inform the treating physician.

In a further preferred embodiment of the invention, the human is an adult, e.g. a person that is 18 years or older. In addition, it is herein understood that the average weight of an adult person is 62 kg, although the average weight is known to vary between countries. In another embodiment of the invention the average weight of an adult person is therefore between about 50-90 kg. It is herein understood that the effective dose as defined herein is not confined to subjects having an average weight. Preferably, the subject has a BMI (Body Mass Index) between 18.0 to 40.0 kg/m2, and more preferably a BMI between 18.0 to 30.0 kg/m2.

Alternatively, the subject to be treated is a child, e.g. a person that is 17 years or younger. In addition, the subject to be treated may be a person between birth and puberty or between puberty and adulthood. It is herein understood that puberty starts for females at the age of 10-11 years and for males at the age of 11-12 year. Furthermore, the subject to be treated may be a neonate (first 28 days after birth), an infant (0-1 year), a toddler (1-3 years), a preschooler (3-5 years); a school-aged child (5-12 years) or an adolescent (13-18 years).

To maintain an effective range during treatment, the compound or composition may be administered once a day, or once every two, three, four, or five days. However preferably, the compound may be administered at least once a day. Hence in a preferred embodiment, the invention pertains to a compound for use according to the invention, or a composition for use according to the invention, characterized in that it is administered to a subject 4, 3, 2, or 1 times per day or less, preferably 1 time per day. The total daily dose may be administered as a single daily dose. Alternatively, the compound is administered at least twice daily. Hence, the compound as defined herein may be administered once, twice, three, four or five times a day. As such, the total daily dose may be divided over the several doses (units) resulting in the administration of the total daily dose as defined herein. In a preferred embodiment, the compound is administered twice daily. It is further understood that the terms “twice daily”, “bid” and “bis in die” can be used interchangeable herein.

In preferred embodiments the compound or composition is administered once every week, more preferably once every 8, 9, 10, 11, 12, 13, or 14 days, most preferably once every 14 days. In other embodiments the compound or composition is administered once every 15, 16, 17, 18, 19, 20, or 21 days, such as once every 21 days.

In a preferred embodiment, the total daily dose is divided over several doses per day. These separate doses may differ in amount. For example, for each total daily dose, the first dose may have a larger amount of the compound than the second dose or vice versa. However preferably, the compound is administered in similar or equal doses. Therefore, in a most preferred embodiment, the compound is administered twice daily in two similar or equal doses.

In a further preferred embodiment of the invention, the total daily dose of the compound as defined herein above is administered in at least two separate doses. The interval between the administration of the at least two separate doses is at least about 0.5, 1, 2, 3, 4, 5, 6, 7, 8, 9, 10, 11 or 12 hours, preferably the interval between the at least two separate doses is at least about 4, 5, 6, 7, 8, 9, 10, 11 or 12 hours and more preferably the interval between the at least two separate doses is at least about 8, 9, 10, 11 or 12 hours.

Also provided herein are kits, suitable for and for treating a disease or condition as described herein. A kit can include any of the compounds or compositions described herein. A kit can further include one or more additional agents, such as a protease inhibitor or an antiviral agent as described elsewhere herein. A kit can additionally include directions for use of the kit (e.g., instructions for treating a disease or condition as described elsewhere herein in a subject), a container, a means for administration, or a means for pain relief.

### General Definitions

When a structural formula or chemical name is understood by the skilled person to have chiral centers, yet no chirality is indicated, for each chiral center individual reference is made to all three of either the racemic mixture (having any enantiomeric excess), the pure R enantiomer, and the pure S enantiomer. Whenever a fragment of a molecule, often referred to as a moiety, is represented, a dotted or wavy line indicates which bond links it to the entirety of the molecule; alternately, an asterisk (*) indicates where the represented moiety is linked to the rest of the molecule. This asterisk does not imply an atom, and neither does a bond that is crossed by a dotted or wavy line convey information about which atom is at the non-moiety side of the bond. All this is known in the art, and is routine practice.

Compounds and compounds for use provided in this invention can be optionally substituted. Suitable optional substitutions are replacement of—H by a halogen. Preferred halogens are F, Cl, Br, and I. Further suitable optional substitutions are substitution of one or more-H by —NH₂, —OH, ═O, alkyl, alkoxy, haloalkyl, haloalkoxy, alkene, haloalkene, alkyne, haloalkyn, and cycloalkyl. Alkyl groups have the general formula CₙH₂ₙ₊₁ and may alternately be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of alkyl groups include methyl, ethyl, propyl, 2-propyl, t-butyl, 1-hexyl, 1-dodecyl, etc. Throughout this application, the valency of atoms should always be fulfilled, and H can be added or removed as required.

Unless stated otherwise, —H may optionally be replaced by one or more substituents independently selected from the group consisting of C1-C12 alkyl groups, C2-C12 alkenyl groups, C2-C12 alkynyl groups, C3-C12 cycloalkyl groups, C5-C12 cycloalkenyl groups, C8-C12 cycloalkynyl groups, C1-C12 alkoxy groups, C2-C12 alkenyloxy groups, C2-C12 alkynyloxy groups, C3-C12 cycloalkyloxy groups, halogens, amino groups, oxo and silyl groups, wherein the silyl groups can be represented by the formula (R²)₃Si—, wherein R² is independently selected from the group consisting of C1-C12 alkyl groups, C2-C12 alkenyl groups, C2-C12 alkynyl groups, C3-C12 cycloalkyl groups, C1-C12 alkoxy groups, C2-C12 alkenyloxy groups, C2-C12 alkynyloxy groups and C3-C12 cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S. Preferably, these optional substitutions comprise no more than twenty atoms, more preferably no more than fifteen atoms.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase or inhibition of a parameter to be assessed can mean a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter. Such terms can include, but do not necessarily include, complete elimination.

The use of a compound or composition as a medicament as described in this document can also be interpreted as the use of said compound or composition in the manufacture of a medicament. Similarly, whenever a compound or composition is used for as a medicament, it can also be used for the manufacture of a medicament, or in a method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 10% of the value, or more or less 1% of the value.

All patent and literature references cited in the present specification are hereby incorporated by reference in their entirety.

In the context of this invention, a cell or a sample can be a cell or a sample from a sample obtained from a subject. Such an obtained sample can be a sample that has been previously obtained from a subject. Such a sample can be obtained from a human subject. Such a sample can be obtained from a non-human subject.

A salt is preferably a pharmaceutically acceptable salt. A salt is preferably a base addition salt wherein a cationic counterion is present. Examples of suitable salts are non-metallic salts such as ammonia salts, and metallic salts such as sodium salts and potassium salts. A skilled person can select suitable salt forms, and their means of production are well known (see e.g. “Occurrence of pharmaceutically acceptable anions and cations in the Cambridge Structural Database” Haynes et al., DOI: 10.1002/jps.20441). A salt can also be an acid addition salt. Acid addition salts are known in the art and examples are HCl salts and acetic acid salts. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002). These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. {See S. M. Barge et al., J. Pharm. Sci. (1977)₆₆, 1).

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Herein, boldface can be used in variables to assist the reader; it does not imply further definition. As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds. It is contemplated that any embodiment of a method or composition described herein can be implemented with respect to any other method or composition described herein. The examples below are intended to further illustrate certain aspects of the methods and compositions described herein, and are not intended to limit the scope of the claims.

The following are numbered embodiments of the invention.

1. A compound of general formula (0) or a salt thereof:

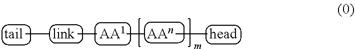

wherein
link is absent or is a linking moiety with a length of 1, 2, 3, 4, 5, or 6 atoms, wherein the atoms are selected from carbon, nitrogen, oxygen, and sulphur, wherein the linking moiety is optionally unsaturated, wherein each atom in the linking moiety is optionally substituted with halogen or with an oxygen atom (either as an —OH moiety or as an ═O moiety) or with a sulphur atom (either as a —SH moiety or as an ═S moiety) or with a nitrogen moiety (either as an —NH₂ moiety or as an ═NH moiety), wherein link connects tail to the N-terminus of AA¹, or wherein link and the N-terminal amine of AA¹ together represent such a linking moiety,
tail is H, C1-24alkyl, —O—C1-24alkyl, 5-20-membered (hetero)aryl, or 3-20-membered (hetero)cycloalkyl, wherein tail is optionally unsaturated, wherein tail is optionally substituted with halogen, C1-3 (halo)alkyl, or C1-3 (halo)alkoxyl;
head is —H, -h1, —O-h1, —C(O)-h1, —C(O)—N(H)h1, —N(h2) h1, or head is -C1-24alkyl, —(NH)₀₋₁-5-20-membered (hetero)aryl, or —(NH)₀₋₁-3-20-membered (hetero)cycloalkyl, wherein head is optionally unsaturated, wherein head is optionally substituted with halogen, C1-3 (halo)alkyl, or C1-3 (halo)alkoxyl;
h1 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], —C₁₋₄alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h1 is optionally substituted with halogen, C1-3 (halo)alkyl, or C1-3 (halo)alkoxyl;
h2 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], —C₁₋₄alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h2 is optionally substituted with halogen, C1-3 (halo)alkyl, or C1-3 (halo)alkoxyl;
AA¹ is an amino acid residue that is connected to its neighbouring AAⁿ via an amide bond to which it contributes a donor carboxylic acid, and that via its amine is connected to link;
AAⁿ is for each instance independently an amino acid residue, wherein the carbonyl moiety in the residue adjacent to head can instead together with head be replaced by —B(OH)₂ or a C1-6alkyl ester thereof, —P(O)(OH)₂ or a C1-6alkyl ester thereof, —S(O)₂—C1, or 5-12 membered (hetero)aryl that is optionally substituted with halogen, C1-3 (halo)alkyl, or C1-3 (halo)alkoxy;
m is 1, 2, 3, 4, or 5.
   2. A compound according to embodiment 1, wherein link has a length of 1 or 2 atoms.
   3. A compound according to embodiment 1 or 2, wherein link has a length of 1 atom.
   4. A compound according to any one of embodiments 1-3, wherein link has at least one carbon atom.
   5. A compound according to any one of embodiments 1-4, wherein link has at most one nitrogen, oxygen, or sulphur atom.
   6. A compound according to any one of embodiments 1-5, wherein link is —C(O)—, —C(═S)—, —C(═NH)—, —(CH₂)₁₋₆—, —O—(CH₂)₂₋₄—C(O)—, —O—(CH₂)₂₋₄—C(═S)—, —O—(CH₂)₂₋₄—C(═NH)—, —S—(CH₂)₂₋₄—C(O)—, —S—(CH₂)₂₋₄—C(═S)—, —S—(CH₂)₂₋₄—C(═NH)—, —NH—(CH₂)₂₋₄—C(O)—, —NH—(CH₂)₂₋₄—C(═S)—, or —NH—(CH₂)₂₋₄—C(═NH)—.
   7. A compound according to any one of embodiments 1-6, wherein link is —C(O)—, —C(═S)—, —C(═NH)—, —NH—(CH₂)₂₋₄—C(O)—, —NH—(CH₂)₂₋₄—C(═S)—, or —NH—(CH₂)₂₋₄—C(═NH)—.
   8. A compound according to any one of embodiments 1-7, wherein link is —C(O)—, —C(═S)—, or —C(═NH)—.
   9. A compound according to any one of embodiments 1-8, wherein link is —C(O)—.
   10. A compound according to any one of embodiments 1-9 or a salt thereof, wherein the compound is of general formula (1):

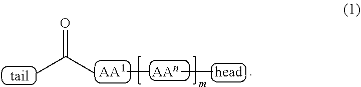

11. The compound according to any one of embodiments 1-10, wherein m is 1, 2, or 3.
12. The compound according to any one of embodiments 1-11, wherein m is 2 or 3.
13. The compound according to any one of embodiments 1-12, wherein m is 2.
14. The compound according to any one of embodiments 1-12, wherein m is 3.
15. The compound according to any one of embodiments 1-14, wherein tail is
C1-20alkyl such as methyl or ethyl or hexanyl or heptanyl or nonanyl or undecanyl or tridecanyl or pentadecanyl or nonadecanyl,
—O—C1-16alkyl such as —O-butyl,
3-12-membered (hetero)cycloalkyl such as adamantanyl or cyclohexyl, or
5-12-membered (hetero)aryl such as phenyl or biphenyl or bithiophene or substituted indole such as methoxyindole.
   16. The compound according to any one of embodiments 1-15, wherein head is
H or —OH;
C(O)—N(H)h1 such as —C(O) NH-Bn or —C(O)—NH₂;
5-10-membered (hetero)aryl such as phenyl,
C1-4alkyl such as methyl
   —N(h2) h1 such as —N(H)h1 such as —NH₂, —NH(C1-6alkyl) such as —NH (propyl) or —NH (hexyl) or —NH (octyl) or —NH (hexadecyl), —NH—S(O)₂—C1-6 (cyclo)alkyl such as —NH—S(O)₂-cyclopropyl, —NH—CH₂-(5-6-membered aryl) such as —NH—CH₂-methoxyphenyl or —NH—CH₂-trifluoromethylphenyl or —NH-benzyl; or such as —N(C1-6alkyl)₂ such as —N(CH₃)₂, or —N(C1-6alkyl)(C1-6alkoxyl) such as —N(CH₃)(OCH₃);
   or the carbonyl moiety of the AAⁿ adjacent to head together with head is replaced by —B(OH)₂ or together forms optionally substituted 5-membered heteroaryl such as 5-methyl-4-aza-oxazol-2-yl.
   17. The compound according to any one of embodiments 1-16, wherein amino acid residues are represented by optionally substituted-NH—C(sc1)(sc2)-C(O)—or by optionally substituted and optionally unsaturated proline or pipecolic acid, wherein
sc1 is for each instance independently H or -C1-3 (halo)alkyl or —CH₂-(halo)phenyl,
sc2 is for each instance independently H or optionally substituted and optionally unsaturated C2-6 (halo)alkyl-N(sc1)₂, C1-6 (halo)alkyl, 5-10-membered (hetero)aryl, C₁-4 (halo)alkyl-[5-10-membered (hetero)aryl], C2-6 (halo)alkyl-N(sc1) C(N (sc1)₂)(═Nsc1), C1-6 (halo)alkyl-C(O)—N(sc1)₂, or C1-4 (halo)alkyl-[3-10-membered (hetero)cycloalkyl]
wherein optional substitutions are preferably halogen, C1-3 (halo)alkyl, C1-3 (halo)alkoxy, guanidinyl, or optionally unsaturated and optionally (halo)methylated-(O)₀₋₁—(CH₂)₀₋₁-5-6-membered (hetero)cycloalkyl such as phenyl or benzyl or imidazolyl or —O-pyridinyl or methyl-dihydropyrrolyl.
   18. The compound according to any one of embodiments 1-17, wherein amino acid residues are optionally substituted and optionally unsaturated alanine, cysteine, aspartic acid, glutamic acid, phenylalanine, glycine, histidine, isoleucine, lysine, leucine, methionine, asparagine, ornithine, pyrrolysine, proline, pipecolic acid, glutamine, arginine, serine, threonine, selenocysteine, valine, tryptophan, or tyrosine, or homo-analogues or nor-analogues thereof.
   19. The compound according to any one of embodiments 1-18, wherein amino acid residues are optionally substituted and optionally unsaturated lysine, ornithine, arginine, histidine, phenylalanine, alanine, glutamine, tryptophan, proline, or valine, or homo-analogues or nor-analogues thereof such as homophenylalanine, homohistidine, pipecolic acid, and phenylglycine.
   20. The compound according to any one of embodiments 1-19, wherein AA¹ is lysine, arginine, phenylalanine, histidine, alanine, valine, leucine, or tryptophan.
   20. The compound according to any one of embodiments 1-19, wherein AA¹ is lysine, arginine, or alanine.
   21. The compound according to any one of embodiments 1-20, wherein AA¹ is lysine.
   22. The compound according to any one of embodiments 1-20, wherein AA¹ is arginine.
   23. The compound according to any one of embodiments 1-20, wherein AA¹ is alanine.
   24 The compound according to any one of embodiments 1-23, wherein m is 2 and AA¹ and the two instances of AAⁿ together form a tripeptide represented by KAK, KAH, KAF, KA-homoPhe, KAA, KPA, KPK, FAF, HAH, ROK, RQ-homoPhe, RNF, RAF, or RQF.
   25. The compound according to any one of embodiments 1-23, wherein m is 3 and AA¹ and the three instances of AAⁿ together form a tetrapeptide represented by

### KAAA, KAAF, KAAH, KAAK, KAAW, KAA-homoHis, KAA-homoPhe, KAA-phenylGly, KAA-(4-OBn-phenylGly),

### KPAF, KPAH, KPAK, KAPK, KPAW, KPA-homoHis, KPA-homoPhe, KPA-phenylGly, KPA-(4-OBn-phenylGly),

### AAAK, AAKA, AKAA, AKAK, APAK, A-(4-O-(4-Py)-Pro)-AK, AVAK, AKKK, LKAK, LKKK, VKAK,

KPHK, KPH-homoPhe, K-(pipecolic acid)-AK, K-(pipecolic acid)-HK, KP-phenylglycine-K, K-(pipecolic acid)-phenylglycine-K,

### ARQK, ARQF, ARRK, FKKK, RARK, FAAF, or WAAW.

26. The compound according to any one of embodiments 1-25, wherein tail comprises at least 8 carbon atoms.
27. The compound according to any one of embodiments 1-26, wherein tail is pentadecyl.
28. The compound according to any one of embodiments 1-27, wherein head comprises at most 1-10 total of carbon atoms and heteroatoms, or the carbonyl moiety of the AAⁿ adjacent to head together with head is replaced by —B(OH)₂ or together forms optionally methylated 5-membered heteroaryl such as 5-methyl-4-aza-oxazol-2-yl.
29. The compound according to any one of embodiments 1-28, wherein head comprises at most 1-10 total of carbon atoms and heteroatoms.
30. The compound according to any one of embodiments 1-29, wherein head comprises at most 1-8 total of carbon atoms and heteroatoms.
31. The compound according to any one of embodiments 1-30, wherein head comprises at most 1-7 total of carbon atoms and heteroatoms.
32. The compound according to any one of embodiments 1-27, wherein head is —NH₂.
33. The compound according to any one of embodiments 1-32, wherein it is any one of compounds 1-196.
34. A composition comprising a pharmaceutically acceptable excipient and a compound as defined in any one of embodiments 1-33.
35. The composition according to embodiment 34, wherein the composition is a pharmaceutical composition.
36. A compound according to any one of embodiments 1-33, for use as a medicament.
37. A composition according to any one of embodiments 34-35, for use as a medicament.
38. A composition or compound for use according to embodiment 36 or 37, wherein the medicament is for use in the treatment of a viral infection or a condition related to a viral infection.
39. Method for inhibiting a viral protease, the method comprising the step of contacting the viral protease with a compound as defined in any one of embodiments 1-33.
40. Method for inhibiting a viral protease, the method comprising the step of contacting the viral protease with a composition as defined in embodiment 34 or 35.
41. The method according to embodiment 39 or 40, wherein the viral protease is a flaviviral protease.
42. The method according to embodiment 39 or 40, wherein the viral protease is a dengue virus protease, a West Nile virus protease, or a tick-borne encephalitis virus protease.
43. The method according to embodiment 39 or 40, wherein the viral protease is a dengue virus protease.
44. The method according to embodiment 39 or 40, wherein the viral protease is a West Nile virus protease.
45. The method according to embodiment 39 or 40, wherein the viral protease is a tick-borne encephalitis virus protease.
46. Method of treating, preventing, or delaying a viral infection or a condition related to a viral infection in a subject in need thereof, the method comprising the step of administering to the subject an effective amount of a compound as defined in any one of embodiments 1-33, or a composition as defined in embodiment 34 or 35.
47. The compound according to any one of embodiments 1-33, wherein
tail comprises at least 8 carbon atoms, preferably it is pentadecyl; and
head comprises at most 1-10 total of carbon atoms and heteroatoms, preferably 1-8, more preferably 1-7, or the carbonyl moiety of the AAⁿ adjacent to head together with head is replaced by —B(OH)₂ or together forms optionally methylated 5-membered heteroaryl such as 5-methyl-4-aza-oxazol-2-yl; and
head is —NH₂.

### DESCRIPTION OF DRAWINGS

FIG. 1-Body weights of experimental groups of mice following treatment. Error bars are Mean±SEM)

FIG. 2-Spleen weights in experimental groups of mice following treatment. Error bars are Mean±SD. * p<0.05 significantly different from vehicle control (G1).

FIG. 3-viral load (plaque assay) in plasma of experimental groups of mice following treatment. Error bars are Mean±SEM. * p<0.05 significantly different from vehicle control (G1).

FIG. 4-plasma concentrations of TNF-alpha, IL-6, and IL-12 in plasma of experimental groups of mice at the end of treatment. Error bars are mean±SEM. * p<0.05 significantly different from vehicle control (G1).

FIG. 5A-multidose PK-profiles of compound 113 in mice after intravenous (IV), intraperitoneal (IP) and subcutaneous (SC) administration to mice (dosed twice per day for 5 days, mean of 3 mice per group).

FIG. 5B-viral load (plaque assay) in plasma of mice following treatment with vehicle or with compound 113 (20 mg/kg, b.i.d. IP and SC starting at 5h post-infection). Error bars are Mean±SEM.

FIG. 5C-tissue distribution after intravenous injection in rats, measured by ratio concentration tissue over plasma.

FIG. 5D-multidose PK-profiles of compound 43 in mice after intravenous (IV), intraperitoneal (IP) and subcutaneous (SC) administration to mice (dosed twice per day for 5 days, mean of 3 mice per group).

### EXAMPLES

### Abbreviations

AA Amino Acid
Ac Acetyl
ACOH Acetic acid
Ala Alanine
AM Amino mehtyl
BB Building Block
Bn Benzyl
Boc Tert-butyloxycarbonyl
Bs Brosyl
calcd calculated
Cbz Carboxybenzylcarbonyl
CDI Carbonyl Diimidazole
CTC-Cl 3-Chlorotrityl chloride
Cyp Cyclopropyl
DBU 1,8-Diazabicyclo[5.4.0]undec-7-ene
DCM Dichloromethane
DEAD Diethyl azodicarboxylate
DIC Diisopropylcarbodiimide
DIPEA Diisopropylethylamine
DMAP 4-(N,N-dimethyl)aminopyridine
DMF Dimethylformamide
DMP Dess-Martin Periodinane
ESI Electron Spray Ionization
Fmoc Fluorenylmethoxycarbonyl
HATU HexafluorophosphateAzabenzotriazole Tetramethyl Uronium
hHis Homo-Histidine
HMDS bis(trimethylsilyl)amide
HOBt Hydroxybenzotriazole
Hpg Hydroxyphenylglycine
HRMS High-resolution mass spectrometry
Hyp Hydroxyproline
IPA Isopropan -2-ol
KHMDS Potassium bis(trimethylsilyl)amide
LDA Lithium diisopropylamide
LRMS Liquid-phase Peptide synthesis
LPPS Low resolution mass spectrometry
MBHA 4-Methylbenzhydrylamine
Mmt 4-Methyltrityl
MS Mass Spectrometry
Oxd Oxadiazole
Oxyma Ethyl cyano (hydroxyimino)acetate
PDA Poly diode array
Ph Phenyl
PHTI Phthalimide
Pin (1S,2S,3R,5S)-2,6,6-trimethylbicyclo[3.1.1]heptane-2,3-diol
Pip. Plperidine
PPh3 Triphenylphosphine
Pro Proline
quant. Quantitative
rac Racemate
R_{f} Retardation factor
RP-HPLC Reversed Phase-HPLC
RT Room temperature
SFC Supercritical Fluid Chromatography
SPPS Solid-phase peptide synthesis
SOC Synthetic Organic Chemistry
Su Succinimidyl
TEA Triethylamine
TFA Trifluoroacetic acid
TFE Trifluoroethanol
THF Tetrahydrofuran
TIC Total Ion Chromatogram
TIPS Triisopropylsilane
TLC Thin-Layer Chromatography
TMS Tetramethylsilane
TsCl Tosylchloride
V Volumes
wt weight

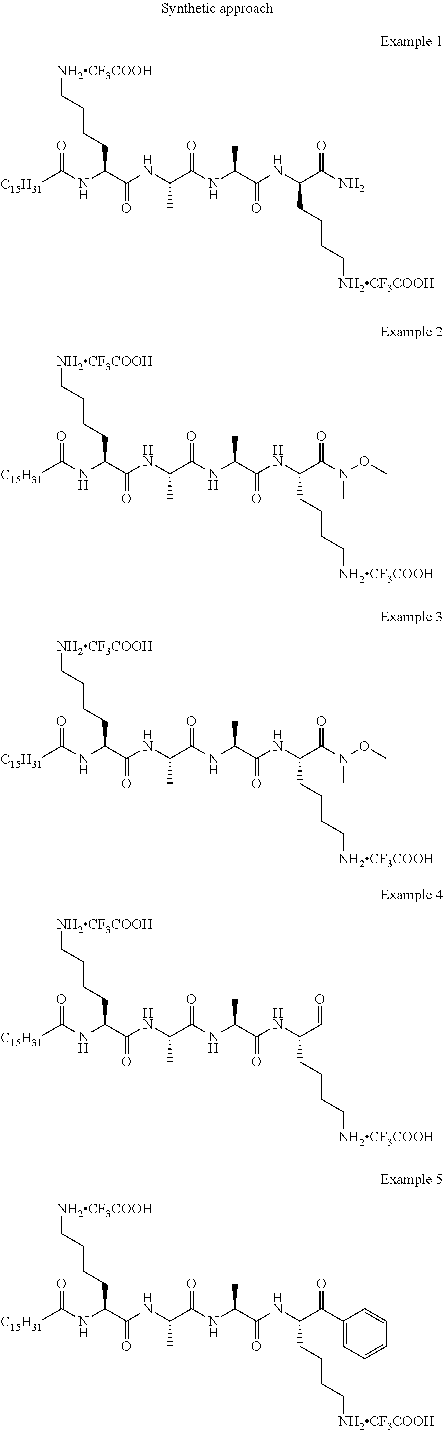

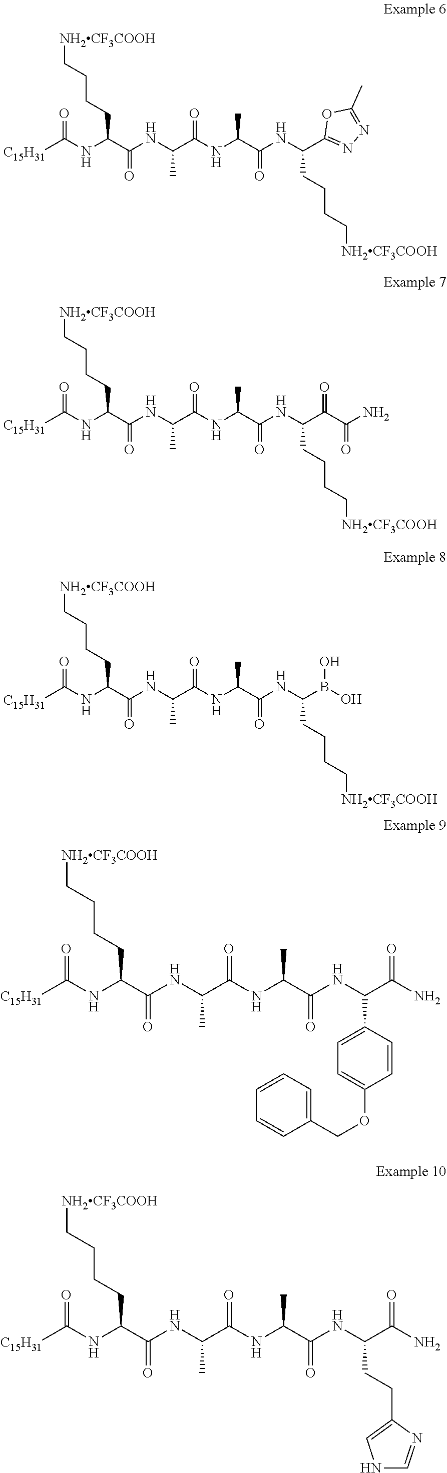

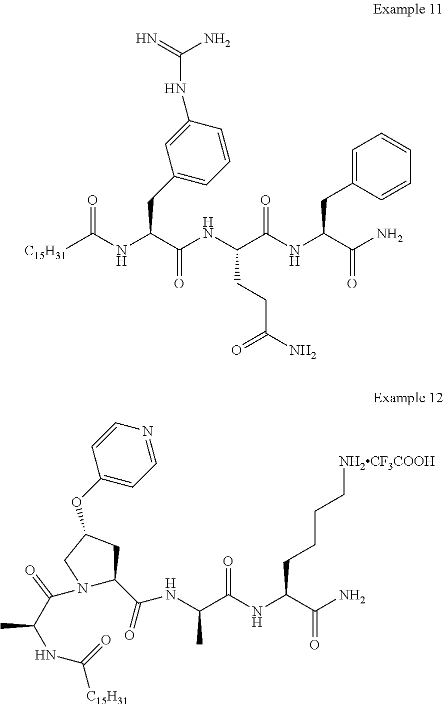

The scheme on the previous pages shows compound examples covering compound classes according to synthetic procedures. It describes the compounds of the invention classified by their synthetic accessibility: compounds can be grouped as a group with 1, a group with 2, 3, 4, a group with 5, 6, 7, 8, a group with 9, 10, 11, and a group with 12. Different examples in the same group required same synthetic strategy but different building blocks. Similar compounds can be synthetized accordingly. Building blocks and intermediates can be either purchased commercially or obtained following well established chemistry. All compounds were obtained after global deprotection and purification via preparative HPLC.

As shown in Scheme 1 (next page), peptides primary amides as Example 1 were prepared via SPPS, using Rink amide MBHA resin and commercially available Fmoc-AAs (1-3) Compounds according to examples 2, 3, and 4 were obtained via late stage derivatizations of the same peptide precursor Intermediate 1, which was synthesized side-chain protected via SPPS, using CTC-Cl resin (Barlos' Resin)(4).

Compounds according to Examples 5-8 were obtained by joining via amide coupling side-chain protected peptide intermediate 2 with the appropriate building block 1-4. Intermediate 2 was synthesized side-chain protected via SPPS, using CTC-Cl resin (Barlos' Resin)(4). Building Blocks 1 and 2 are prepared from the appropriately protected Lys precursor, adapting literature procedures reported for other amino acids (ketone (5, 6) in Scheme 9, hydroxyamide (7) in Scheme 11) or commonly found in the synthesis of pharmaceuticals (oxadiazole (6-10) in Scheme 10). Building block 4 was obtained via Matteson homologation (11), adapting known literature procedures (Scheme 12). (12, 13) To obtain compounds according to examples 7 (Scheme 11) and 8 (Scheme 12), prior to global deprotection, the appropriate functional group interconversion was performed, respectively to oxidize the hydroxyamide to alpha-ketoamide, (14) and convert the bromide to an amino group. (12)

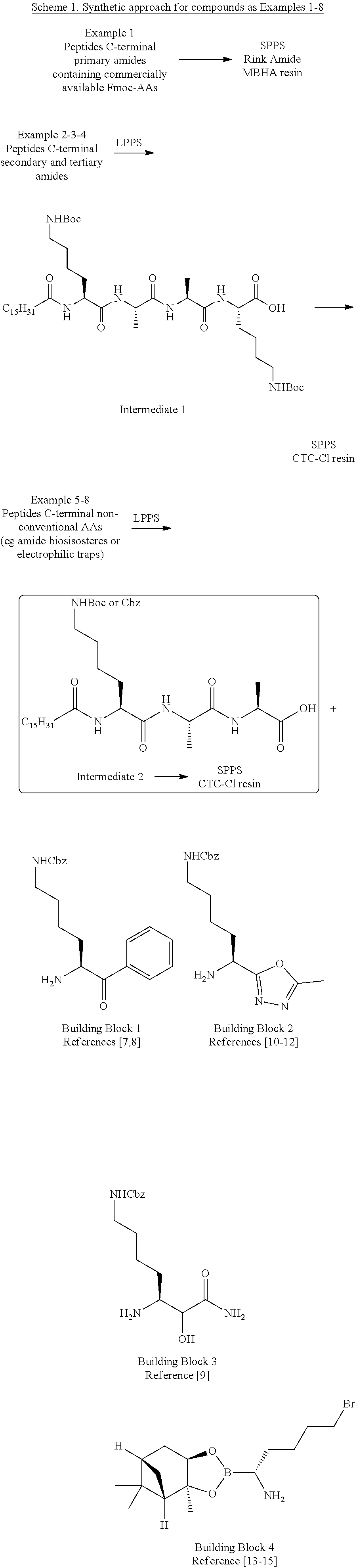

Example compounds 9-11 can be obtained via SPPS using Rink-amide resin, however their synthesis required the ad-hoc preparation of Fmoc-AAs as Building Blocks 5-7, whose synthesis is detailed in Schemes 13-15. BB5 was prepared adapting the procedure described by Behnam and coworkers (15, 16) as detailed in Scheme 13. BB6 was prepared adapting the procedure of Bloemhoff and coworkers (17) as detailed in Scheme 14. BB7 was prepared adapting the procedure described by Elliot and coworkers (18) as detailed in Scheme 15.

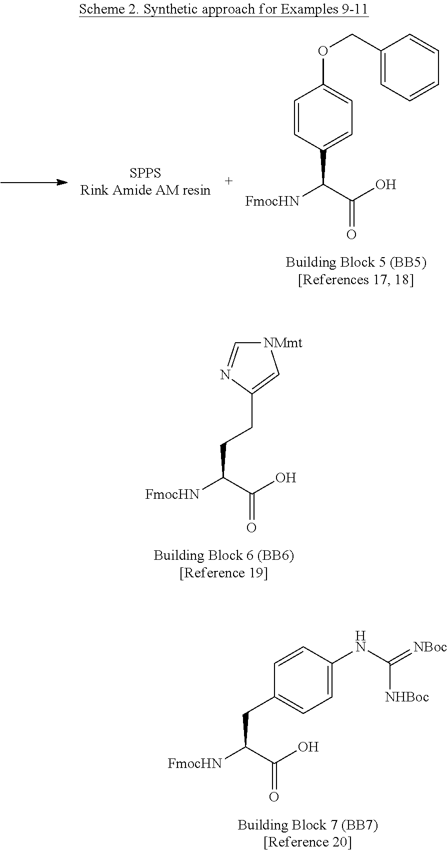

Compounds according to Example 12 (Scheme 16) were synthesized via LPPS (19) joining pre-assembled intermediate 3 (i3), and intermediate 4 (i4). i4 was prepared via LPPS, from BB8 and BB9. Boc-protected AA BB8 was obtained via Mitsunobu displacement (20) from commercially available amino- and carboxyl-protected commercially available Hyp (BB8a)(21). BB9 was prepared via LPPS from commercially available AAs.

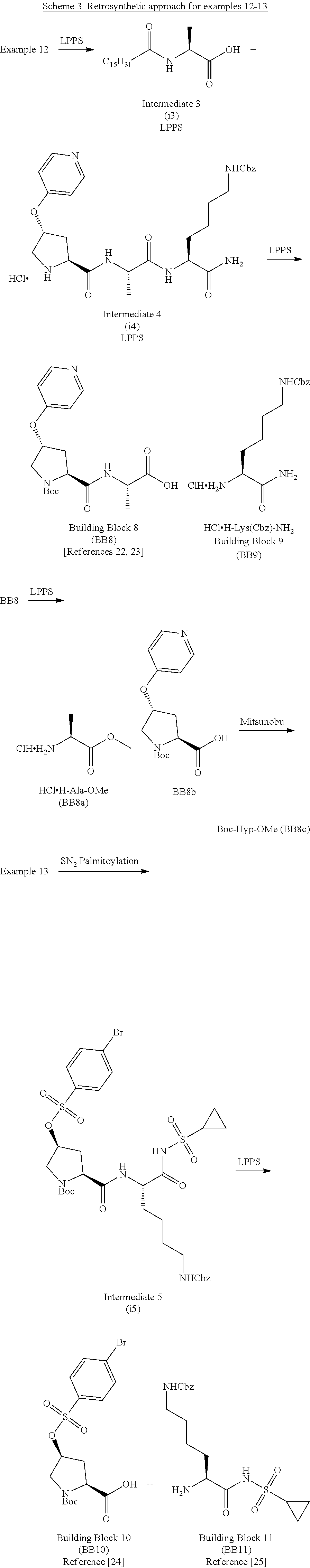

### General Experimental

All peptides were purified via RP-HPLC(unless stated otherwise) using a Shimadzu LC-20A Prominence system (Shimadzu, 's Hertogenbosch, The Netherlands) equipped with a C18 Gemini-NX column, 150×21.20 mm, particle size 10 μm (Phenomenex, Utrecht, The Netherlands), a pre-column guard and UV detection at 215 and 254 nm. RP-HPLC elution was performed with 0.1% TFA in a MeCN/milliQ H2O solution (isocratic 10% MeCN in H2O over 5 min, gradient from 10 to 100% MeCN in H2O over 20 min, with a solvent flow rate of 10.0 mL/min, unless stated otherwise). Chemicals were purchased from Fluorochem, VWR, Fischer Scientific or Sigma-Aldrich and used as received, unless stated otherwise. Dry DCM and THF as were obtained extra-pure from commercial vendors and used as ultra-pure after purification with MBraun Solvent Purification System (MB SPS). Reactions were magnetically stirred and carried out under inert atmosphere of dry nitrogen or argon, unless stated otherwise. Standard syringe techniques were applied for the transfer of dry solvents and air- or moisture-sensitive reagents. Reactions were followed either via analytical LCMS or TLC. R_{f} values are obtained using thin layer chromatography (TLC) on silica gel-coated plates (Merck 60 F254) with the indicated solvent mixture. Detection was performed with UV-light, and/or by charring at ˜150° C. after dipping into a solution of either KMnO₄ (7.5 g/L), K₂CO₃ (50 g/L) and 10% NaOH (6.25 ML/L) in H2O, or (NH₄)₆MO₇O₂₄. 4H₂O (25 g/L) and (NH₄)₄Ce(SO₄)₄ ·2H₂O (10 g/L) in 10% H₂SO₄. Alternatively, iodine staining was performed burying the plate in 12 adsorbed on SiO₂ for 30 seconds. LCMS spectrograms were recorded on a Thermo Finnigan LCQ-Fleet ion trap mass spectrometer (ESI-IT-MS) coupled to a Shimadzu analytical HPLC [LC-20AD (pump) and SPD-M30A (photodiode array detector)], equipped with a Gemini C18 110A column, 50 mm×2 mm, particle size 3 μm (Phenomenex, Utrecht, The Netherlands), eluting with 0.1% formic acid in a MeOH/milliQ H₂O solution (isocratic 5% MeOH in H₂O over 5 min, gradient from to 95% MeOH in H₂O over 20 min, with a solvent flow rate of 1.0 mL/min). NMR spectra were recorded using either a Bruker Avance 400 (400 MHZ) or a Bruker Avance III (500 MHZ) spectrometer, in MeOH (d4), CDCl₃, or D₂O solutions, unless stated otherwise. Chemical shifts are given in ppm with respect to residual non-deuterated solvents or TMS as internal standard for CDCl₃. Coupling constants are reported as J-values in Hz. The following abbreviations are used to explain multiplicities: s=singlet, d=doublet, t=triplet, q=quartet, dd=doublet of doublets, dp=doublet of quintets, ddd=doublet of doublet of doublets, dtd=doublet of triplet of doublets, td=triplet of doublets, m=multiplet. Flash column chromatography was carried out using ACROS silica gel (0.035-0.070 mm, and ca 6 nm pore diameter). High resolution mass spectra (HRMS) were recorded on a JEOL AccuToF CS JMS-T100CS (ESI-HRMS). Regular MS (ESI-MS) measurements were recorded on Thermo Finnigan LCQ-Q Advantage Max.

### General Information SPPS

Fmoc deprotection. The resin was swollen with DCM (10 mL/gram of resin, 1 min) and DMF (2×10 mL/gram of resin, 1 min) and treated with 20% pip in DMF (10 mL/gram of resin) and left to shake for 20 minutes. The suspension was filtered and the resin was washed with DMF (2×10 mL/gram of resin, 1 min) and treated with a second portion of 20% pip in DMF (10 mL/gram of resin) and left to shake for 10 minutes. The suspension was then filtered and the resin was washed with DMF (3×10 mL/gram of resin, 1 min), DCM (3×10 mL/gram of resin, 1 min) and MeOH (3×10 mL/gram of resin, 1 min). Deprotection efficiency was determined by means of Kaiser or chloranil tests (for Pro deprotection).

Loading of the first amino acid (Rink Amide MBHA/AM resin). The resin was swollen with DCM (1 min) and DMF (2×1 min). Fmoc-amino acid (3 equiv) and HOBt (3 equiv) were dissolved in DMF (10 mL/gram of resin) and the resulting solution was added to the resin. Next DIPCDI (3 equiv) was added and the reactor was left to shake for 16 h. The suspension was then filtered and the resin was washed with DMF (3×1 min), DCM (3×1 min), before treating with a capping solution of acetic anhydride/pyridine (3:2, 10 mL/gram of resin) for 20 minutes. The suspension was then filtered and the resin was washed with DMF (3×1 min), DCM (3×1 min) and MeOH (3×1 min).

### Coupling Efficiency was Determined by Means of a Kaiser Test

Loading of the first amino acid (CTC-Cl Barlos' resin). The Fmoc-amino acid (3 equiv) was dissolved in dry DCM (10 mL/gram of resin) and collidine (3 equiv), and the resulting solution was added to resin. The reactor was left shaking overnight, and the suspension was filtered before treating with a capping solution of 5% DIPEA in MeOH (10 mL/gram of resin) for 15 minutes. The suspension was then filtered and the resin was washed with DMF (3×1 min), DCM (3×1 min) and MeOH (3× 1 min).

Peptide coupling. The resin was swollen with DCM (1 min) and DMF (2×1 min). Fmoc-amino acid (3 equiv) and HOBt (3 equiv) were dissolved in DMF (10 mL/gram of resin) and the resulting solution was added to the resin. Next DIPCDI (3 equiv) was added and left to shake for 3 h. The suspension was then filtered and the resin was washed with DMF (3×1 min), DCM (3×1 min) and MeOH (3× 1 min). Coupling efficiency was determined by means of a Kaiser or chloranil tests (for couplings on Pro).

Coupling to palmitic acid. The resin was swollen with DCM (1 min) and DMF (2×1 min). Palmitic acid (3 equiv) and HATU (2.9 equiv) were dissolved in DCM/DMF (1:1, 10 mL/gram of resin) and the resulting solution was added to the resin. Next DIPEA (3 equiv) was added and the reactor was left to shake for 3 h. The suspension was then filtered and the resin was washed with DMF (3×1 min), DCM (3×1 min) and MeOH (3×1 min). Coupling efficiency was determined by means of Kaiser or chloranil tests (for couplings on Pro).

Peptide cleavage (Rink Amide MBHA/AM resin). The peptidyl-resin was washed with DCM (3×1 min) and dried under nitrogen. The resin was treated with a cleavage solution (95% TFA, 2.5% TIPS, 2.5% H₂O, 5 mL) and left to shake for 2 h (unless stated otherwise). The mixture was filtered and the resin was washed with DCM (3×1 min), filtrates were collected, combined and volatiles were removed in vacuo. The crude residue was triturated in dry Et₂O and after centrifuge the precipitate was collected by decantation. Solvent leftovers were removed under high-vacuum.

Purification. The crude material was dissolved in minimal amount of MeOH (unless stated otherwise), filtered through a 0.20 μm syringe filter, and purified using preparative RP-HPLC (isocratic 20% MeCN in H₂O over 5 min, gradient from 20 to 80% MeCN in H₂O over 15 min, with a solvent flow rate of 10.0 mL/min, at 30° C.). All fractions containing product were combined, concentrated to 5 mL in vacuo, prior to lyophilization (unless stated otherwise) to afford the pure materials.

Peptide cleavage (CTC-Cl Barlos' resin). The peptidyl-resin was washed with DCM (3×1 min) and dried under nitrogen before being suspended in a cleavage solution of AcOH/TFE/DCM (1:1:8, 10 mL/g of resin) and shaken for 1 h, then the resin was washed with DCM (3×1 min), collecting all the filtrates. The cleavage process was repeated three time in total, then the filtrates were combined and reduced to 10 mL in vacuo before lyophilization. The resulting white powder was suspended in dry Et₂O and after centrifuge the precipitate was collected by decantation. Solvent leftovers were removed under high-vacuum.

### Example 1

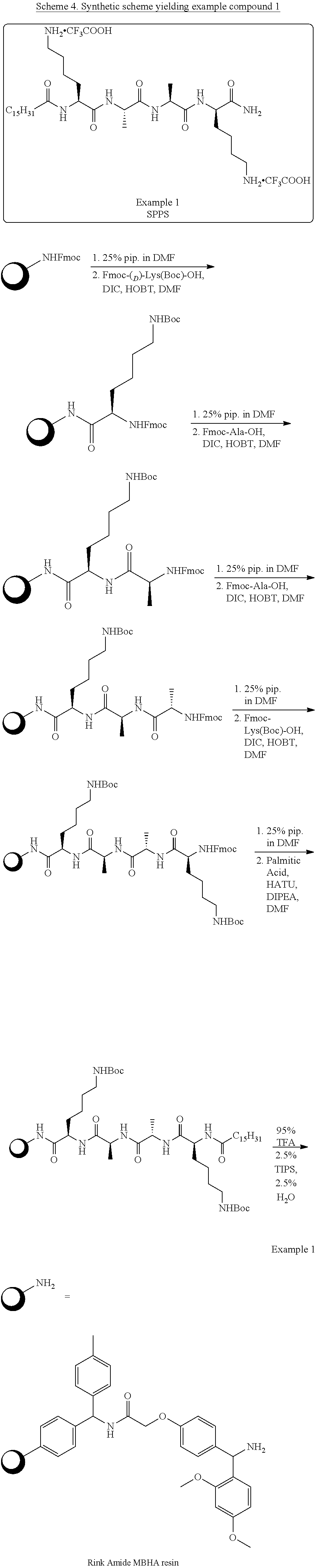

N—(S)-6-amino-1-(((S)-1-(((S)-1-(((R)-1,6-diamino-1-oxohexan -2-yl)amino)-1-oxopropan -2-yl)amino)-1-oxopropan -2-yl)amino)-1-oxohexan -2-yl) palmitamide bis-TFA [Palmitoyl-Lys-Ala-Ala-D-Lys-NH₂ bis-TFA salt (Example 1)]. was prepared according the general procedure for SPPS using 700 mg Rink amide resin (0.26 mmol). The crude peptide was purified by preparative HPLC, all fractions containing the product were combined and concentrated in vacuo to 5 mL, prior to lyophilization, to afford peptide Example 1 (140 mg) as a white powder.

HRMS (ESI-MS m/z): mass calcd for C₃₄H₆₇N₇O₅ [M+H]⁺, 654.5276, found 654.5268.

Compounds made following the synthesis of example 1:11, 12, 13, 14, 15, 16, 17, 33, 35, 37, 40, 43, 44, 47, 49, 50, 51, 52, 53, 54, 57, 58, 63, 64, 70, 72, 73, 75, 77, 80, 82, 84, 86, 88, 90, 91, 92, 93, 95, 97, 110, 111, 112, 113, 114, 115, 116, 117, 118, 119, 120, 121, 122, 123, 124, 125, 131, 132, 133, 140, 141, 142, 143, 144, 145, 146, 147, 148, 149, 150, 151, 152, 153, 154, 155, 156, 157, 158, 159, 160, 161, 162, 163, 164, 167, 168, 169, 170, 171, 190, 191, 192, 193, 194, 195, 196

### Example 2

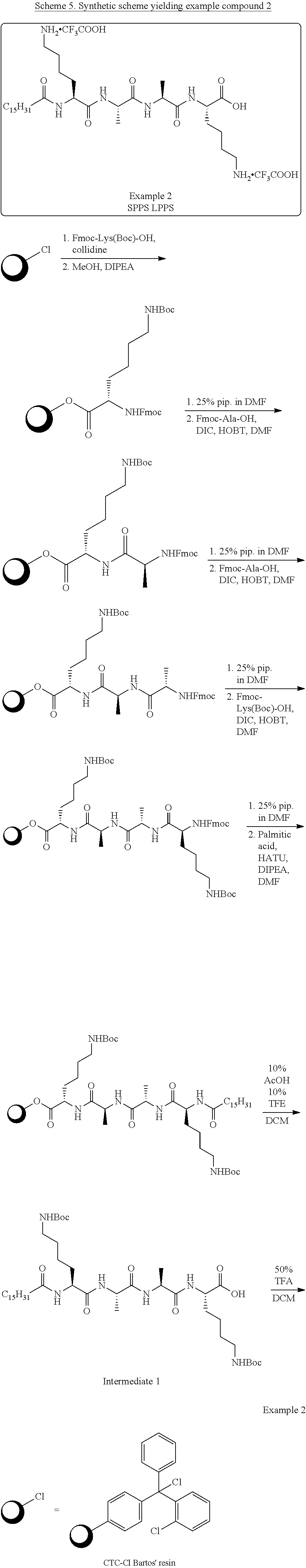

N⁶-(tert-butoxycarbonyl)-N²-N⁶-(tert-butoxycarbonyl)-N²-palmitoyl-L-lysyl-L-alanyl-L-alanyl-L-lysine[Palmitoyl-Lys(Boc)-Ala-Ala-Lys(Boc)-OH(Intermediate 1)]. was prepared according the general procedure for SPPS using 2.5 g CTC-Cl resin (3.3 mmol), to recover Intermediate 1 (1.7 g, 60%) as a white powder. The crude peptide was used as such in the next synthetic step.

LCMS (ESI-MS m/z): mass calcd for C₄₄H₈₃N₆O₁₀ [M+H]⁺, 855.62; found 854.96.

Palmitoyl-L-lysyl-L-alanyl-L-alanyl-L-lysine bis-TFA [Palmitoyl-Lys-Ala-Ala-Lys-OH bis-TFA salt (Example 2)]. To a DCM (2.0 mL) solution of Intermediate 1 (110 mg, 0.12 mmol, 1.0 equiv), TFA (2.0 mL) was added, and the resulting mixture was left stirring 2 h at RT. The reaction mixture was concentrated in vacuo and the crude residue was dissolved in H₂O, filtered through a 0.20 μm syringe filter, and purified using preparative RP-HPLC. All fractions containing product were combined, concentrated to 5 mL in vacuo, prior to lyophilization, to recover Example 2 (73 mg, 66%) as a white powder.

LCMS (ESI-MS m/z): mass calcd for C₃₆H₇₂N₇O₅ [M+H]⁺, 655.51, found 655.28.

Compounds made following the synthesis of example 2:45, 46, 134, 188, 189.

### Example 3

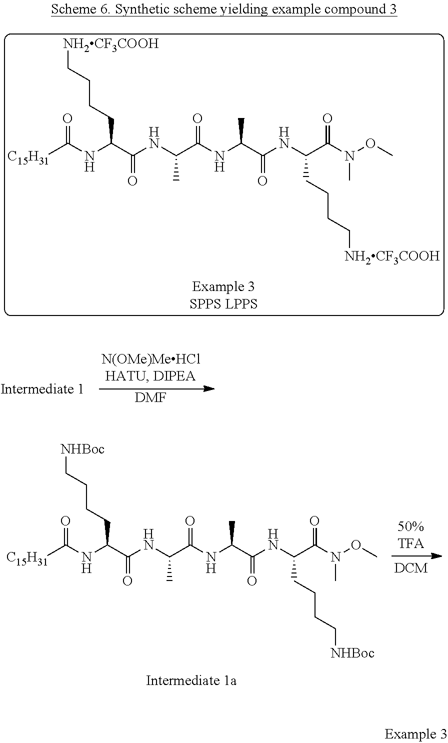

Tert-butyl ((10S,13S,16S,19S)-19-(methoxy(methyl)carbamoyl)-2,2,13,16-tetramethyl-4,11,14,17-tetraoxo-10-palmitamido-3-oxa-5,12,15,18-tetraazatricosan -23-yl)carbamate[Palmitoyl-Lys(Boc)-Ala-Ala-Lys(Boc)-N(OMe)Me (Intermediate 1a)]. To a DMF solution (5.0 mL) of Intermediate 1 (300 mg, 0.35 mmol, 1.0 equiv) NH(OMe)Me hydrochloride was added at 0° C., (41 mg, 0.42 mmol, 1.2 equiv), followed by HATU (150 mg, 0.38 mmol, 1.1 equiv) and DIPEA (180 μL, 1.0 mmol, 3.0 equiv). The resulting mixture was left warming up at RT over 16 h, 10 before it was quenched by pouring over 50 mL of ice and 10% aqueous citric acid. The so-formed solid was filtered, washed with water (2×20 mL) and dried in vacuo. The crude residue was purified via flash column chromatography (SiO₂, MeOH/DCM, from 1:19 to 1:20). Fractions containing product were combined and concentrated in vacuo, the residue was dissolved in dioxane and lyophilized to recover intermediate 1a (290 mg, 93%) as a white powder. LCMS (ESI-MS m/z): mass calcd for C₄₆H₈₈N₇O₁₀ [M+H]⁺, 898.66; found 898.12. N-((5S,8S,11S,14S)-18-amino-5-(4-aminobutyl)-3,8,11-trimethyl-4,7,10,13-tetraoxo-2-oxa-3,6,9,12-tetraazaoctadecane -14-yl) palmitamide bis-TFA [Palmitoyl-Lys-Ala-Ala-Lys-N(OMe)Me bis-TFA salt (Example 3)]. To a DCM (2.0 mL) solution of Intermediate 1a (107 mg, 0.12 mmol, 1.0 equiv), TFA (2.0 mL) was added, and the resulting mixture was left stirring 2 h at room temperature. The reaction mixture was concentrated in vacuo and the crude residue was dissolved in H₂O, filtered through a 0.20 μm syringe filter, and purified using preparative RP-HPLC(isocratic 20% MeCN in H₂O over 5 min, gradient from 20 to 100% MeCN in H₂O over 15 min). All fractions containing product were combined, concentrated to 5 mL in vacuo, prior to lyophilization, to recover Example 3 (83 mg, 75%) as a white powder.

LCMS (ESI-MS m/z): mass calcd for C₃₆H₇₂N₇O₅ [M+H]⁺, 698.55, found 698.48.

Compounds made following the synthesis of example 3:18, 19, 20, 21, 22, 23, 24, 25, 26, 27, 28, 29, 30, 31, 32, 34, 36, 38, 69, 71, 74, 76, 78, 79, 81, 83, 85, 87, 89, 94, 96, 98, 126, 177, 184, 185, 187,

### Example 4

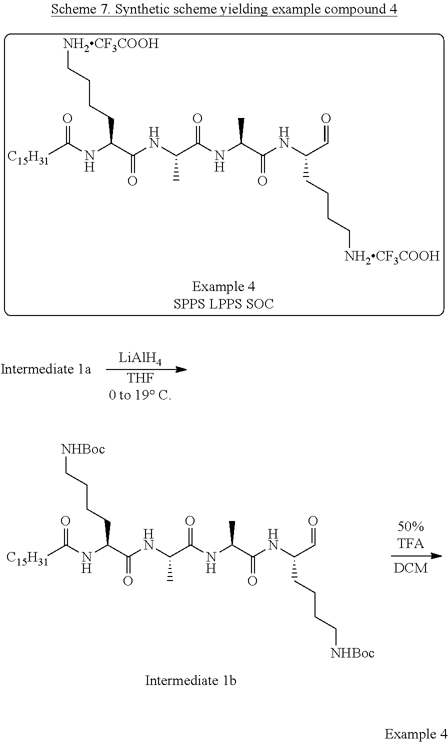

Tert-butyl ((10S,13S,16S,19S)-10-formyl-2,2,13,16-tetramethyl-4,12,15,18-tetraoxo-19-palmitamido-3-oxa-5,11,14,17-tetraazatricosan -23-yl)carbamate[Palmitoyl-Lys(Boc)-Ala-Ala-Lys(Boc)-H (Intermediate 1b)]. To a THF solution (10 mL) of Intermediate 1a (206 mg, 0.23 mmol, 1.0 equiv), LiAlH₄ (36.0 mg, 0.95 mmol, 4.1 equiv) was added at 0° C., and the resulting mixture was left stirring for 0.5 h. Next, the reaction mixture was quenched by the addition of saturated aqueous NH₄Cl (20 mL) and diluted with EtOAc (40 mL). After phase separation the organic layer was diluted with DCM (3.0 mL), IPA (3.0 mL) and MeOH (3.0 mL), washed with saturated aqueous potassium tartrate (3×20 mL), brine (2×10 mL), dried over sodium sulfate, and concentrated in vacuo to recover a crude residue, which was purified via flash column chromatography (SiO₂, MeOH/CHCl₃, from 1:49 to 1:9). Fractions containing product were combined and concentrated in vacuo. The residue was dissolved in dioxane and lyophilized to recover intermediate 1b (50 mg, 26%) as a white powder.

LCMS (ESI-MS m/z): mass calcd for C₃₆H₇₂N₇O₅ [M+H]⁺, 839.62, found 839.16.

N—((S)-6-amino-1-(((S)-1-(((S)-1-(((S)-6-amino-1-oxohexan -2-yl)amino)-1-oxopropan -2-yl)amino)-1-oxopropan -2-yl)amino)-1-oxohexan -2-yl) palmitamide bis-TFA [Palmitoyl-Lys-Ala-Ala-Lys-H bis-TFA salt (Example 4)]. To a DCM (2.0 mL) solution of Intermediate 1b (110 mg, 0.12 mmol, 1.0 equiv), TFA (2.0 mL) was added, and the resulting mixture was left stirring 2 h at RT. The reaction mixture was concentrated in vacuo and the crude residue was dissolved in H₂O, filtered through a 0.20 μm syringe filter, and purified using preparative RP-HPLC. All fractions containing product were combined, concentrated to 5 mL in vacuo, prior to lyophilization, to recover Example 4 (41 mg, 33%) as a white powder.

LCMS (ESI-MS m/z): mass calcd for C₃₄H₆₅N₆O₄ [M−H₂O+H]⁺, 621.51; found 621.48.

Compounds made following the synthesis of example 4:180, 181, 186.

### Intermediate 2a and 2b

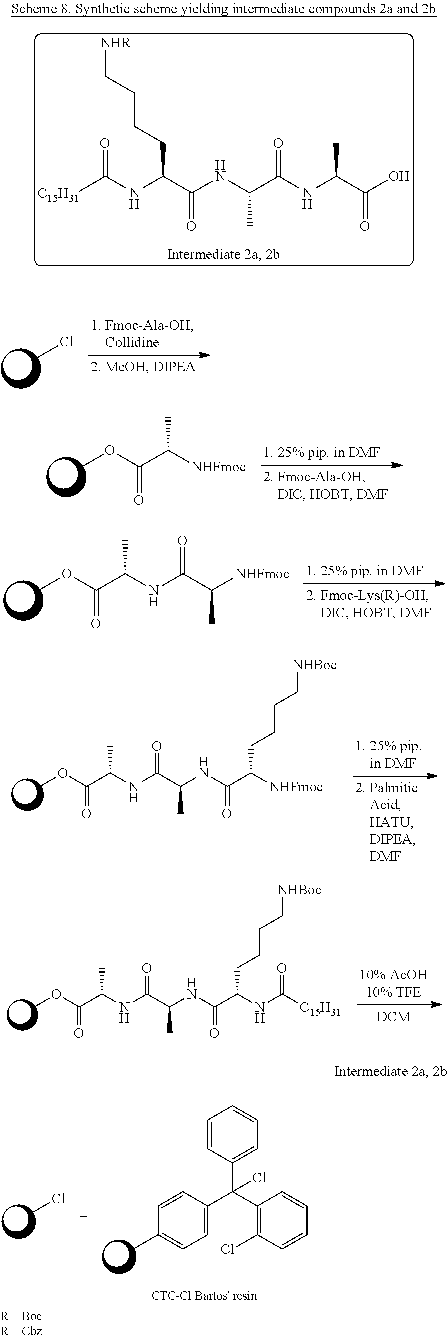

N6-(tert-butoxycarbonyl)-N2-palmitoyl-L-lysyl-L-alanyl-L-alanine[Palmitoyl-Lys(Boc)-Ala-Ala-OH(Intermediate 2a)]. was prepared according the general procedure for SPPS using 0.50 g CTC-Cl resin (0.85 mmol), to recover Intermediate 2a (340 mg, 63%) as a white powder. The crude peptide was used as such in the next synthetic step.

MS (ESI-MS m/z): mass calcd for C₃₃H₆₃N₄O₇ [M+H]⁺, calcd 627.5; found 627.1.

N6-(benzyloxy)carbonyl)-N2-palmitoyl-L-lysyl-L-alanyl-L-alanine[Palmitoyl-Lys (Cbz)-Ala-Ala-OH(Intermediate 2b)]. was prepared according the general procedure for SPPS using 0.50 g CTC-Cl resin (0.85 mmol), to recover intermediate 2b (320 mg, 60%) as a white powder. The crude peptide was used as such in the next synthetic step.

LCMS (ESI-MS m/z): mass calcd for C₃₆H₆₁N₄O₇ [M+H]⁺, calcd 861.45; found 661.00.

### Example 5

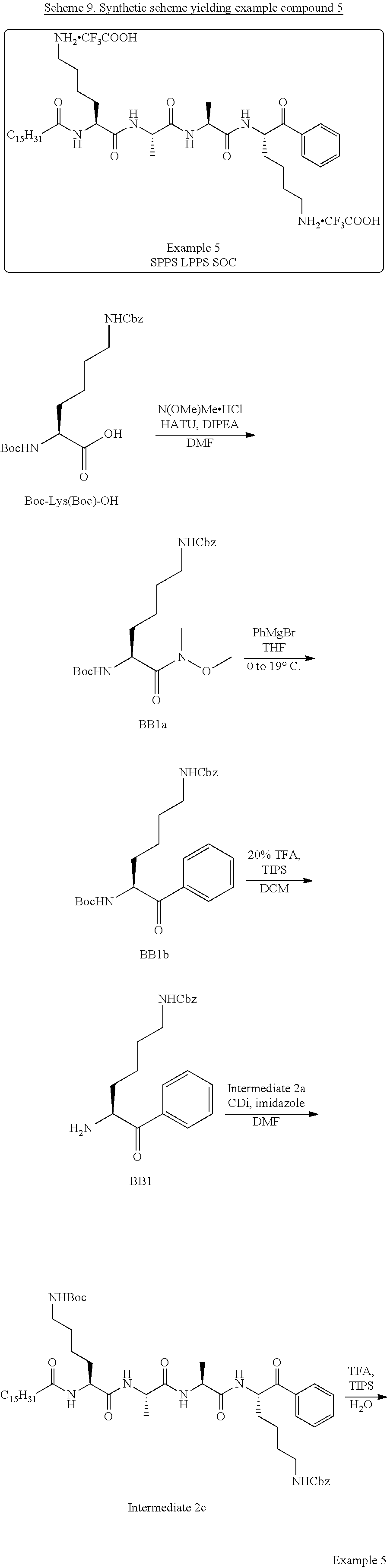

Benzyl tart-butyl (6-(methoxy(methyl)amino)-6-oxohexane-1,6-diyl)(S)-dicarbamate[Boc-Lys (Cbz)-NOMe)Me (BB1a]. To a DMF solution (20.0 ml) of Boc-Lys (Cbz)-OH (2.0 g, 5.3 mmol, 1.0 equiv), NH(OMe)Me hydrochloride (760 mg, 7.9 mmol, 1.5 equiv) was added at 0° C., followed by HATU (3.0 g, 7.9 mmol, 1.1 equiv) and DIPEA (4.7 mL, 16 mmol, 3.0 equiv). The resulting mixture was left stirring over 10 minutes, before it was quenched by diluting with H₂O (50 mL). The aqueous layer was extracted with EtOAc (3×100 mL), and the combined organic layers were dried over Na₂SO₄ and concentrated in vacuo. The crude residue was purified via flash column chromatography (SiO₂, EtOAc/pet-ether, 1:4), to recover Weinreb amide BB1a (1.4 g, 64%) as a light yellow oil.

R_{f}=0.7 (SiO₂, EtOAc)

LCMS (ESI-MS m/z): mass calcd for C₂₁H₃₄N3O₆ [M+H]⁺, 424.24; found 424.42.

Benzyl tert-butyl (6-oxo-6-phenylhexane-1,5-diyl)(S)-dicarbamate[Boc-Lys (Cbz)-Ph (BB1b)]. To a THF (15 mL) solution of BB1a (1.5 g, 3.3 mmol, 1.0 equiv), 1 M PhMgBr in THF (10.6 mL, 10.6 mmol, 3.0 equiv) was added dropwise over 5 minutes at 0° C. The resulting mixture was warmed to RT and stirred for 2 h, before it was quenched by diluting with saturated aqueous NH₄Cl (30 mL). The aqueous layer was extracted with EtOAc (3×100 mL), and the combined organic layers were dried over Na₂SO₄ and concentrated in vacuo. The crude residue was purified via flash column chromatography (SiO₂, EtOAc/pet-ether, 1:5), to recover BB1b (0.35 g, 22%) as a light yellow oil.

R_{f}=0.5 (SiO₂, EtOAc/pet-ether, 1:1)

LCMS (ESI-MS m/z): mass calcd for C₂₅H₃₃N₂O₅ [M+H]⁺, 441.24; found 441.49.

Benzyl(S)-(5-amino-6-oxo-6-phenylhexyl)carbamate TFA [H-Lys (Cbz)-Ph TFA salt (BB1)]. To a TFA/DCM solution (1:5, 7.0 mL), BB1a (0.35 g, 0.79 mmol, 1.0 equiv) was added at 0° C. The resulting mixture was warmed to RT and stirred for 1 h, before it was concentrated in vacuo, to recover amine hydrochloride BB1 (0.25 g, quant.) as crude material, which was used as such in the next synthetic step.

LCMS (ESI-MS m/z): mass calcd for C₂₀H₂₅N₂O₃ [M+H]⁺, 341.19 found 341.37.

Tert-butyl ((9S, 12S,15S, 18S)-9-benzoyl-12,15-dimethyl-3,11,14,17-tetraoxo-18-palmitamido-1-phenyl-2-oxa-4,10,13,16-tetraazadocosan -22-yl)carbamate[Palmitoyl-Lys(Boc)-Ala-Ala-Lys (Cbz)-Ph (Intermediate 2c)]. To a DMF (20 mL) solution of Intermediate 2a (300 mg, 0.47 mmol, 1.0 equiv) was added CDI at 0° C., followed by imidazole (48 mg, 0.71 mmol, 1.5 equiv). The resulting mixture was warmed to RT and stirred for 10 minutes, before BB1 (97 mg, 0.28 mmol, 0.6 equiv) was added. The resulting mixture was stirred for 16 h, before it was quenched by diluting with H₂O (50 mL). The aqueous layer was extracted with EtOAc (3×100 mL), and the combined organic layers were dried over Na₂SO₄ and concentrated in vacuo, to recover Intermediate 2c (0.4 g, quant.) as crude material which was used as such in the next synthetic step.

(ESI-MS m/z): mass calcd for C₅₃H₈₅N₆O₉ [M+H]⁺, 949.64 found 949.65.

N—((S)-6-amino-1-(((S)-1-(((S)-1-(((S)-6-amino-1-oxo-1-phenylhexan -2-yl)amino)-1-oxopropan -2-yl)amino)-1-oxopropan -2-yl)amino)-1-oxohexan -2-yl) palmitamide bis-TFA [Palmitoyl-Lys-Ala-Ala-Lys-Ph bis-TFA salt (Example 5)]. To a TFA/TIPS/H₂O (95:2.5:2.5, 8.0 mL) solution, Intermediate 2c (0.40 g, 0.42 mmol, 1.0 equiv) was added at 0° C. The reaction mixture was heated to 50° C. and stirred for 2 h, before it was concentrated in vacuo, and the crude residue was purified via RP-HPLC. All fractions containing product were combined, concentrated to 5 mL in vacuo, prior to lyophilization, to recover Example 5 (31 mg, 8%) as a white powder.

MS (ESI-MS m/z): mass calcd for C₄₀H₇₁N₆O₅ [M+H]⁺, 715.5 found 715.5.

¹H NMR (400 MHZ, DMSO-d₆) δ 8.27 (d, J=8.0 Hz, 1H), 7.98-7.91 (m, 5H), 7.68-7.64 (m, 7H), 7.53 (t, J=8.0 Hz, 2H), 5.30-5.21 (m, 1H), 4.29-4.19 (m, 3H), 2.74 (t, J=7.6 Hz, 4H), 2.51 (t, J=2.0 Hz, 4H), 2.10 (t, J=7.6 Hz, 2H), 1.83-1.70 (m, 1H), 1.65-1.39 (m, 9H), 1.41-1.14 (m, 34H), 0.85 (t, J=6.4 Hz, 3H).

Compounds made following the synthesis of example 5:42, 128, 136, 137, 174, 175

### Example 6

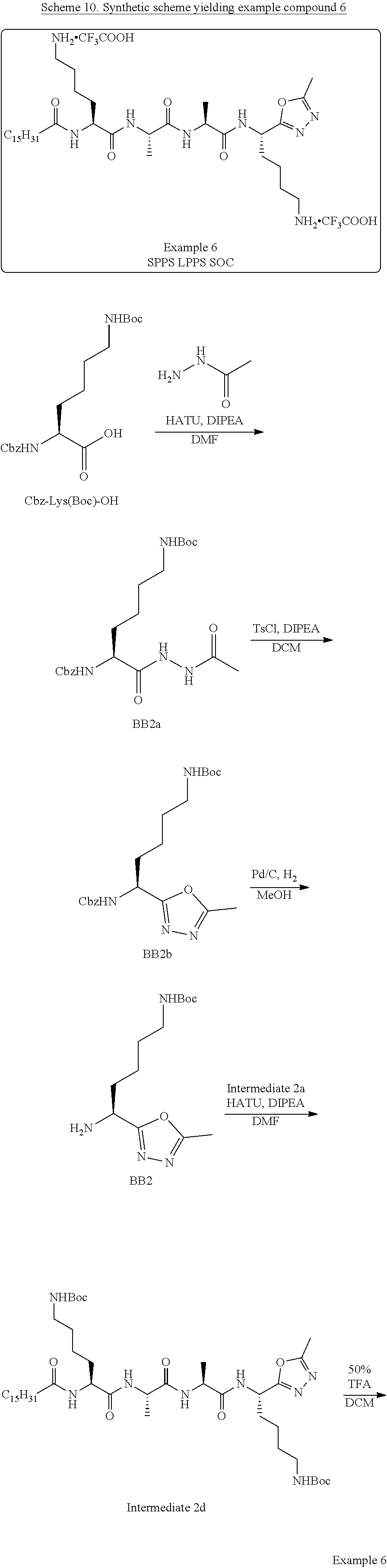

Benzyl ter-butyl (6-(2-acetylhydrazineyl)-6-oxohexane-1,5-diyl (S)-dicarbamate[Cbz-Lys(Boc)-NH—NH—Ac (BB2a]. To a DMF solution (13 ml) of CBz-Lys(Boc)-OH (500 mg, 1.3 mmol, 1.0 equiv) Acetyl Hydrazide (130 mg, 1.7 mmol, 1.3 equiv) was added, followed by HATU (550 mg, 1.4 mmol, 1.1 equiv) and DIPEA (080 μL, 1.7 mmol, 1.3 equiv). The resulting mixture was left stirring over 16 h, before it was quenched by diluting with H₂O (200 mL). The aqueous layer was extracted with EtOAc (2×100 mL), and the combined organic layers were washed with brine (2×100 mL), dried over Na₂SO₄ and concentrated in vacuo. The crude residue was purified via flash column chromatography (SiO₂, MeOH/DCM, from 0:100 to 1:9) to recover BB2a (500 mg, 86%) as transparent crystals.

R_{f}=0.35 (SiO₂, MeOH/DCM, 1:9)

HRMS (ESI-MS m/z): mass calcd for C₂₁H₃₂N₄NaO₆ [M+Na]⁺, 459.22195, found 459.22252. Benzyl tert-butyl (1-(5-methyl-1,3,4-oxadiazol-2-yl)pentane-1,5-diyl)(S)-dicarbamate[Cbz-Lys(Boc)-Oxd-Me (BB2b)]. To a DCM solution (18 mL) of BB2a (850 mg, 1.9 mmol, 1.0 equiv),

TsCl (742 mg, 3.9 mmol, 2.0 equiv) was added, followed by DIPEA (1.6 mL, 12 mmol, 6.3 equiv) and DIPEA (690 μL, 1.7 mmol, 1.3 equiv). The resulting mixture was left stirring over 5 h, before it was quenched by diluting with saturated aqueous NH₄Cl (20 mL). After layer separation, the aqueous layer was extracted with DCM (2×40 mL), and the combined organic layers were washed with brine (2×40 mL), dried over Na₂SO₄ and concentrated in vacuo. The crude residue was purified via flash column chromatography (SiO₂, MeOH/DCM, from 0:100 to 1:9) to recover BB2b (590 mg, 72%) as a yellow oil.

R_{f}=0.57 (SiO₂, MeOH/DCM, 1:9)

HRMS (ESI-MS m/z): mass calcd for C₂₁H₃₀N₄NaO₅ [M+Na]⁺, 441.21139, found 441.21214. Tert-butyl(S)-(5-amino-5-(5-methyl-1,3,4-oxadiazol-2-yl)pentyl)carbamate[H-Lys(Boc)-Oxd-Me (BB2)]. To a MeOH (18 mL) solution of Compound BB2b (580 mg, 1.4 mmol, 1.0 equiv) was added 10% Pd on Carbon (150 mg, 0.14 mmol, 0.1 equiv) followed by Hydrogen gas by means of a balloon. The resulting mixture was left stirring for 2 h before hydrogen was purged and backfilled with Ar, before filtering over Celite®, washing with MeOH (20 mL). The filtrate was concentrated in vacuo to recover amine BB2 (390 mg, 98%) as a yellow oil.

R_{f}=0.32 (SiO₂, MeOH/DCM, 1:9)

HRMS (ESI-MS m/z): mass calcd for C₁₂H₂₄N₄NaO₃ [M+Na]⁺, 307.17461, found 307.17739. Tert-butyl ((10S, 13S, 16S, 19S)-2,2,13,16-tetramethyl-10-(5-methyl-1,3,4-oxadiazol-2-yl)-4,12,15,18-tetraoxo-19-palmitamido-3-oxa-5,11,14,17-tetraazatricosan -23-yl)carbamate[Palmitoyl-Lys(Boc)-Ala-Ala-Lys(Boc)-Oxd-Me (Intermediate 2d)]. To a DMF (5.0 mL) solution of amine BB2 (140 mg, 0.50 mmol, 1.0 equiv) acid intermediate 2a (350 mg, 0.55 mmol, 1.1 equiv) was added at 0° C., followed by HATU (210 mg, 0.55 mmol, 1.1 equiv) and DIPEA (0.33 mL, 1.9 mmol, 3.5 equiv). The resulting mixture was left stirring 2 h, before it was quenched by diluting with H₂O (150 mL) at 0° C. The so-formed precipitate was filtered, washed with H₂O (15 mL) and recovered dissolving in MeOH (30 mL). The methanolic solution was concentrated in vacuo, and the crude residue was purified via flash column chromatography (SiO₂, MeOH/DCM, from 0:100 to 1:9) to recover peptide intermediate 2d (110 mg, 24%) as a white amorphous solid.

R_{f}=0.36 (SiO₂, MeOH/DCM, 1:9)

HRMS (ESI-MS m/z): mass calcd for C₄₆H₈₄N₈O₉ [M+H]⁺, 893.64395, found 893.84690.

N—(S)-6-amino-1-(((S)-1-(((S)-1-(((S)-5-amino-1-(5-methyl-1,3,4-oxadiazol-2-yl)pentyl)amino)-1-oxopropan -2-yl)amino)-1-oxopropan -2-yl)amino)-1-oxohexan -2-yl) palmitamide bis-TFA [Palmitoyl-Lys-Ala-Ala-Lys-Oxd-Me bis-TFA salt (Example 6)]. To a DCM (2.0 mL) solution of Intermediate 2d (108 mg, 0.12 mmol, 1.0 equiv), TFA (2.0 mL) was added, and the resulting mixture was left stirring 2 h at RT. The reaction mixture was concentrated in vacuo and the crude residue was dissolved in H₂O, filtered through a 0.20 μm syringe filter, and purified using preparative RP-HPLC. All fractions containing product were combined, concentrated to 5 mL in vacuo, prior to lyophilization, to recover Example 6 (13 mg, 12%) as a white powder.

HRMS (ESI-MS m/z): mass calcd for C₃₆H₆₈N₈O₅ [M+H]⁺, 693.53909, found 693.53875.

Compounds made following the synthesis of example 6:1, 2, 3, 4.

### Example 7

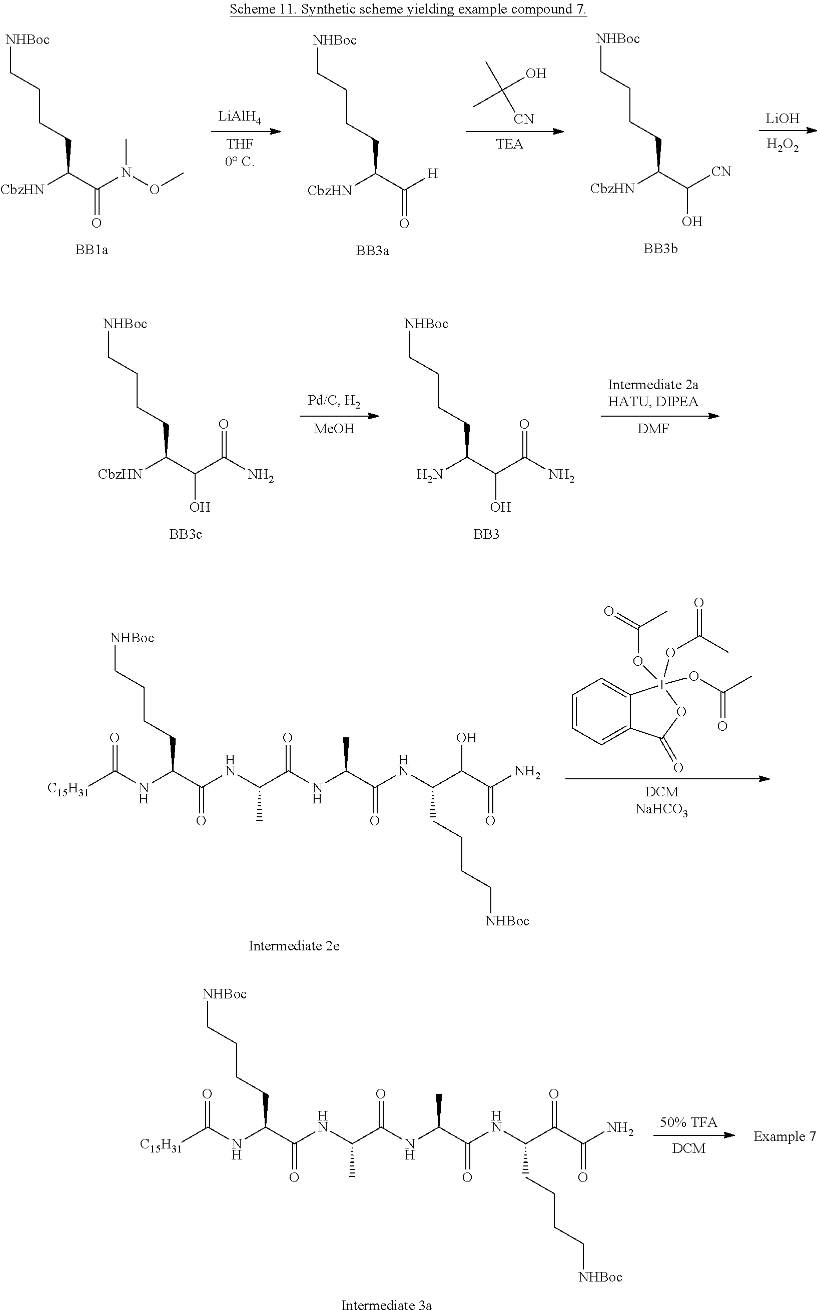

Benzyl tert-butyl (6-oxohexane-1,5-diyl)(S)-dicarbamate[Cbz-Lys(Boc)-H (BB3a)]. To a dry THE solution (50 mL) of BB1a (2.2 g, 5.2 mmol, 1.0 equiv), LiAlH₄ (390 mg, 10.4 mmol, 2.0 equiv) was added at 0° C., and the resulting mixture was left stirring for 2 h. Next, the reaction mixture was quenched by the addition of 1 M aqueous KHSO₄ (20 mL), before THF was concentrated in vacuo. The aqueous residue was diluted with 1 M aqueous HCl (20 mL) and Et₂O (50 mL). After phase separation, the aqueous layer was extracted further with Et₂O (3×50 mL), and the 10 combined organic layers were washed with 1 M aqueous HCl (3×20 mL), saturated aqueous NaHCO₃ (3×20 mL), brine (3×20 mL), dried over MgSO4 and concentrated in vacuo, to obtain crude aldehyde BB3a (1.7 g, 89%) as a yellow oil. The crude material was used as such in the next synthetic step. Spectral and analytical data are in accordance to those published before by Lindgren and coworkers (24)

Benzyl tert-butyl ((5S)-6-cyano-6-hydroxyhexane-1,5-diyl)dicarbamate (BB3b). To a DCM solution (5 mL) of aldehyde BB3a (240 mg, 0.66 mmol, 1.0 equiv), acetone cyanohydrine (0.30 mL, 3.3 mmol, 5.0 equiv) was added, followed by TEA (0.91 mL, 6.6 mmol, 10 equiv), and the resulting mixture was left stirring for 16 h, at RT. Next, the reaction mixture was concentrated in vacuo, and the residue was dissolved in EtOAc (20 mL), and the organic layer was washed with H₂O (3×10 mL), saturated aqueous NaHCO₃ (10 mL), brine (10 mL), dried over MgSO4 and concentrated in vacuo. The crude residue was purified via flash column chromatography (SiO₂, MeOH/DCM, from 0:100 to 1:9) to recover cyanohydrine BB3b (250 mg, 96%) as a yellow oil.

R_{f}=0.51 (SiO₂, MeOH/DCM, 1:9).

¹H NMR (400 MHZ, CDCl₃) δ 7.36 (s, 5H), 5.43 (s, 1H), 5.13 (s, 2H), 4.58 (bs, 1H), 4.57 (d, J=16.3 Hz, 1H), 3.92 (s, 1H), 3.82 (s, 1H), 3.12 (m, 2H), 1.50 (m, 15H).

Benzyl tert-butyl ((5S)-7-amino-6-hydroxy-7-oxoheptane-1,5-diyl)dicarbamate (BB3c). To a MeOH solution (30 mL) of cyanohydrine BB3b (1.8 g, 4.6 mmol, 1.0 equiv), LiOH (130 mg, 5.5 mmol, 1.2 equiv) was added at 0° C., followed by H₂O₂ (10 mL, 115 mmol, 25 equiv), and the resulting mixture was left stirring for 4 h. Next, the reaction mixture was was quenched by diluting with 5% aqueous Na₂S₂O₃ (30 mL), and the aqueous layer was extracted with DCM (3×100 mL). The combined organic layers were dried over MgSO4 and concentrated in vacuo, the crude residue was purified via flash column chromatography (SiO₂, MeOH/DCM, from 0:100 to 1:9) to recover hydroxyamide BB3c (710 mg, 44%) as a white solid.

R_{f}=0.47 (SiO₂, MeOH/DCM, 1:9).

¹H NMR (400 MHZ, CDCl₃) õ 7.33-7.13 (m, 5H), 6.75 (s, 1H), 5.56 (s, 1H), 5.04 (s, 1H), 4.96 (d, J=7.6 Hz, OH), 4.24 (s, 0.5H), 4.14 (s, 0.5H), 3.78 (s, 1H), 2.68 (m, 2H), 2.18-1,85 (m, 2H), 1.45 (s, 5H), 1.43 (s, 4H).

Tert-butyl ((5S)-5,7-diamino-6-hydroxy-7-oxoheptyl)carbamate (BB3). To a MeOH (22 mL) solution of Cbz-protected hydroxyamide BB3c (920 mg, 2.2 mmol, 1.0 equiv), 10% Pd/C(230 mg, 0.22 mmol, 0.1 equiv) was added, and the mixture was left to stir in H₂ atmosphere (1.0 atm) over 1 h. Next, the reaction mixture was filtered over Celite®, washing the pad with MeOH (20 mL), the combined filtrate was concentrated in vacuo, and the residue was dissolved in dioxane (10 mL), prior to lyophilization, to recover free amine BB3 (610 mg, 99%) as a white solid. The crude material was used as such in the next synthetic step.

¹H NMR (400 MHZ, DMSO-d₆) δ 7.20-7.13 (2 br s, 2H), 6.75 (t, 1H), 5.37 (bs, 1H), 3.71 (d, J=4.1 Hz, 1H), 3.65 (d, J=3.2 Hz, 1H), 2.89 (m, 2H), 2.79 (m, 1H), 1.84-1.69 (2 bs, 2H), 1.37 (s, 13H), 1.27-1.09 (m, 2H).

Tert-butyl ((10S, 13S, 16S,19S)-19-(2-amino-1-hydroxy-2-oxoethyl)-2,2,13,16-tetramethyl-4,11,14,17-tetraoxo-10-palmitamido-3-oxa-5,12,15,18-tetraazatricosan -23-yl)carbamate[Palmitoyl-Lys(Boc)-Ala-Ala-Lys(Boc)-CH—OH—C═O—NH₂ (Intermediate 2e)]. To a DMF (5.0 mL) solution of amine BB3 (100 mg, 0.36 mmol, 1.0 equiv) acid intermediate 2a (250 mg, 0.40 mmol, 1.1 equiv) was added at 0° C., followed by HATU (140 mg, 0.40 mmol, 1.1 equiv) and DIPEA (0.22 mL, 1.3 mmol, 3.5 equiv). The resulting mixture was left stirring 4 h, before it was quenched by diluting with H₂O (50 mL) at 0° C. The so-formed precipitate was filtered, washed with H₂O (15 mL) and recovered dissolving in MeOH (30 mL). The methanolic solution was concentrated in vacuo, and the crude residue was purified via flash column chromatography (SiO₂, MeOH/DCM, from 0:100 to 1:9) to recover peptide intermediate 2e (83 mg, 26%) as a white amorphous solid.

R_{f}=0.41 (SiO₂, MeOH/DCM, 1:9)

HRMS (ESI-MS m/z): mass calcd for C₄₅H₈₅N₇NaO₁₀ [M+Na]⁺, 906.62556; found, 906.62468. Tert-butyl ((10S,13S,16S, 19S)-19-(2-amino-2-oxoacetyl)-2,2,13,16-tetramethyl-4,11,14,17-tetraoxo-10-palmitamido-3-oxa-5,12,15,18-tetraazatricosan -23-yl)carbamate[Palmitoyl-Lys(Boc)-Ala-Ala-Lys(Boc)-C═O—NH₂ (Intermediate 3a)]. To a DCM (8 mL) solution of Intermediate 2e (83 mg, 0.094 mmol, 1.0 equiv), DMP (130 mg, 0.28 mmol, 3.0 equiv) was added and the resulting mixture was left stirring for 12 h. Next, the reaction mixture was was quenched by diluting with saturated aqueous NaHCO₃ (6 mL) and 10% aqueous Na₂S₂O₃ (6 mL), and the resulting mixture was stirred for 0.33 h, before diluting with DCM (20 mL) and stirring additional 0.33 h. After layer separation, the organic layer was collected, washed with H₂O (2×40 mL), dried over MgSO4 and concentrated in vacuo, to recover Intermediate 3a (82 mg, 99%) as a white solid. The crude material was used as such in the next synthetic step.

HRMS (ESI-MS m/z): mass calcd for C₄₅H₈₃N₇NaO₁₀ [M+Na]⁺, 904.60991; found, 904.61264. N—((S)-6-amino-1-(((S)-1-(((S)-1-(((S)-1,7-diamino-1,2-dioxoheptan -3-yl)amino)-1-oxopropan -2-yl)amino)-1-oxopropan -2-yl)amino)-1-oxohexan -2-yl) palmitamide bis-TFA [Palmitoyl-Lys-Ala-Ala-Lys-C═O—NH₂ bis-TFA salt (Example 7)]. To a DCM (2.0 mL) solution of Intermediate 2d (83 mg, 0.094 mmol, 1.0 equiv), TFA (2.0 mL) was added, and the resulting mixture was left stirring 2 h at RT. The reaction mixture was concentrated in vacuo and the crude residue was dissolved in H₂O, filtered through a 0.20 μm syringe filter, and purified using preparative RP-HPLC. All fractions containing product were combined, concentrated to 5 mL in vacuo, prior to lyophilization, to recover Example 7 (29 mg, 34%) as a white powder.

HRMS (ESI-MS m/z): mass calcd for C₃₅H₆₆N₇O₅ [M−H₂O+H]⁺, 664.51199, found 664.51246. Compounds made following the synthesis of example 7:5, 6, 9, 10, 176.

### Example 8

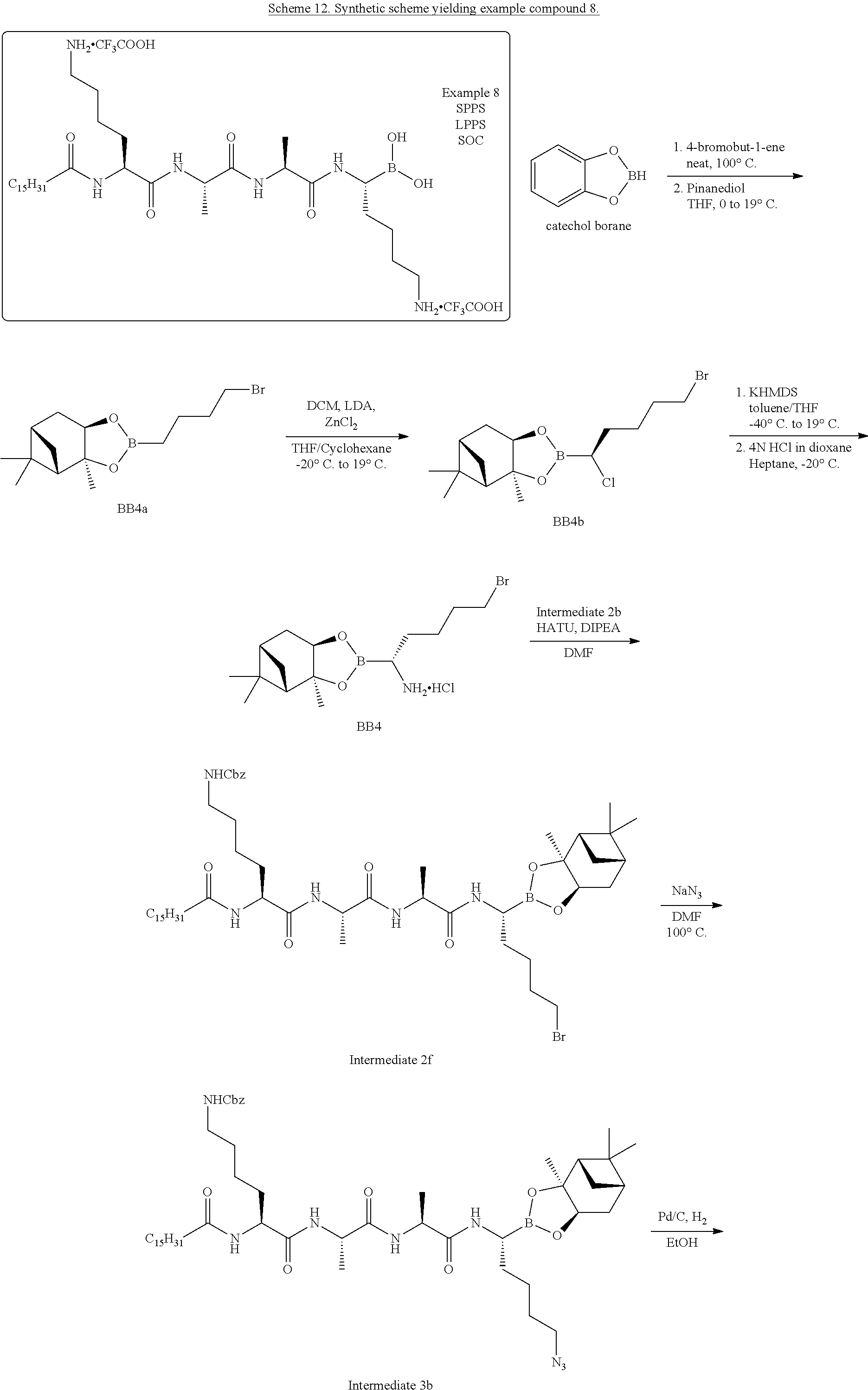

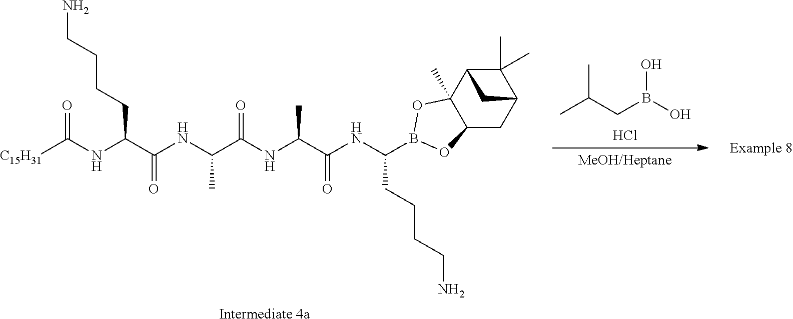

(3aS,4S,6S,7aR)-2-(4-bromobutyl)-3a,5,5-trimethylhexahydro-4,6-methanobenzo[d][1,3,2]dioxaborole (BB4a). Catechol Borane (2.3 mL, 21 mmol, 1.2 equiv) was added to 4-bromobut-1-ene (2.2 mL, 22 mmol, 1.3 equiv) and the resulting mixture was heated to 100° C. for 3 h. After cooling to RT, the reaction mixture was added dropwise to a dry THF (20 mL) solution of (1S,2S,3R,5S)-2,6,6-trimethylbicyclo[3.1.1]heptane-2,3-diol (3.0 g, 18 mmol, 1.0 equiv) at 0° C., and the resulting mixture was warmed to RT and stirred for 16 h. Next, the reaction mixture was concentrated in vacuo and the crude residue was purified via flash column chromatography (SiO₂. Heptane/EtOAc, from 99:1 to 9:1) to recover boronic ester BB4 (4.5 g, 81%) as a transparent oil.

¹H NMR (400 MHZ, CDCl₃) δ 4.26 (dd, J=8.7, 2.0 Hz, 1H), 3.41 (t, J=6.9 Hz, 2H), 2.34 (ddt, J=14.5, 8.7, 2.4 Hz, 1H), 2.22 (dtd, J=10.8, 6.1, 2.2 Hz, 1H), 2.08-2.00 (m, 1H), 1.95-1.79 (m, 4H), 1.63-1.51 (m, 3H), 1.38 (s, 3H), 1.29 (s, 3H), 1.09 (d, J=10.9 Hz, 1H), 0.84 (s, 5H).

(3aS,4S,6S,7aR)-2-((S)-5-bromo-1-chloropentyl)-3a,5,5-trimethylhexahydro-4,6-methanobenzo[d][1,3,2]dioxaborole (BB4b). To a THF/cyclohexane (2:1, 30 mL) solution of boronic ester BB4 (2.7 g, 8.7 mmol, 1.0 equiv) DCM (0.73 mL, 11 mmol, 1.3 equiv) was added at −20° C. Next, a 1M THF solution of LDA (11 mL, 11 mmol, 1.3 equiv) was added dropwise over 0.5 h, followed by a cold (−20° C.)₁M THF solution of ZnCl2 (14 mL, 14 mmol, 1.6 equiv). The reaction mixture was warmed to RT and stirred for 16 h, before it was concentrated in vacuo. The crude residue was purified via flash column chromatography (SiO₂. Heptane/EtOAc, from 9:1 to 20:3) to recover chlorohomologation product BB4b (3.2 g, 58%) as a transparent oil.

¹H NMR (400 MHZ, CDCl₃) δ 4.37 (dd, J=8.8, 2.0 Hz, 1H), 3.51-3.38 (m, 3H), 2.42-2.31 (m, 1H), 2.30-2.19 (m, 1H), 2.10 (d, J=5.0 Hz, 1H), 1.97-1.79 (m, 5H), 1.55 (d, J=0.7 Hz, 3H), 1.42 (s, 3H), 1.29 (d, J=3.8 Hz, 3H), 1.17 (d, J=11.0 Hz, 1H), 0.85 (s, 3H).

(R)-5-bromo-1-((3aS,4S,6S,7aR)-3a,5,5-trimethylhexahydro-4,6-methanobenzo[d][1,3,2]dioxaborol-2-yl)pentan -1-amine hydrochloride (BB4). To a dry THF (13 mL) solution of chlorohomologation product BB4b (1.9 g, 5.3 mmol, 1.0 equiv) a 0.5 M toluene solution of KHMDS (13 mL, 6.4 mmol, 1.2 equiv) was added at −40° C., and the resulting mixture was stirred overnight. Next, the reaction mixture was concentrated in vacuo and the crude residue was suspended in heptane (15 mL), before being filtered through a CELITE® pad, washing with heptane (15 mL). The filtrates were combined and concentrated in vacuo, and the residue was re-dissolved in heptane (15 mL), before it was diluted by the dropwise addition of a 4 M 1,4-dioxane solution of HCl (3.0 mL, 12 mmol, 2.1 equiv) at −20° C. The resulting mixture was stored at −20° C. for 16 h, before it was concentrated in vacuo, and the crude residue was triturated in heptane (15 mL). The solids were separated via decantation, washed with cold heptane (15 mL) and dried in vacuo, to recover amine hydrochloride BB4 (750 mg, 37%) as a white solid.

¹H NMR (400 MHZ, cdcl₃) δ 8.28 (s, 3H), 4.46-4.35 (m, 1H), 3.42 (td, J=6.9, 1.8 Hz, 2H), 2.96 (d, J=5.8 Hz, 1H), 2.40-2.18 (m, 2H), 2.10-2.03 (m, 1H), 1.95-1.80 (m, 5H), 1.80-1.56 (m, 3H), 1.43 (s, 3H), 1.29 (s, 3H), 1.16 (d, J=11.1 Hz, 1H), 0.83 (s, 3H).

Benzyl ((S)-6-(((S)-1-(((S)-1-(((R)-5-bromo-1-(3aS,4S,6S,7aR)-3a,5,5-trimethylhexahydro-4,6-methanobenzo[d][1,3,2]dioxaborol-2-yl)pentyl)amino)-1-oxopropan -2-yl)amino)-1-oxopropan -2-yl)amino)-6-oxo-5-palmitamidohexyl)carbamate[Palmitoyl-Lys (Cbz)-Ala-Ala-5-bromo-1-BO₂Pin-pentylamide (Intermediate 2f)].To a DMF (8 mL) solution of amine BB4 (170 mg, 0.44 mmol, 1.0 equiv) acid intermediate 2b (290 mg, 0.44 mmol, 1.0 equiv) was added at 0° C., followed by HATU (180 mg, 0.48 mmol, 1.1 equiv) and DIPEA (0.17 mL, 0.96 mmol, 2.2 equiv). The resulting mixture was left stirring 2 h, before it was quenched by diluting with H₂O (80 mL) at 0° C. The so-formed precipitate was filtered, washed with H₂O (15 mL) and recovered dissolving in MeOH (30 mL). The methanolic solution was concentrated in vacuo, the crude residue was purified via flash column chromatography (SiO₂, CHCl₃/MeCN, from 7:3 to 1:4) to recover peptide intermediate 2f (152 mg, 35%) as a white amorphous solid.

R_{f}=0.3 (SiO₂, CHCl₃/MeCN, 1:4)

LCMS (ESI-MS m/z): mass calcd for C₅₁H₈₆BBrN₅O₈ [M+H]⁺, 986.57; found, 986.88.

Benzyl ((S)-6-(((S)-1-(((S)-1-(((R)-5-azido-1-((3aS,4S,6S,7aR)-3a,5,5-trimethylhexahydro-4,6-methanobenzo[d][1,3,2]dioxaborol-2-yl)pentyl)amino)-1-oxopropan -2-yl)amino)-1-oxopropan -2-yl)amino)-6-oxo-5-palmitamidohexyl)carbamate[Palmitoyl-Lys (Cbz)-Ala-Ala-5-azido-1-BO₂Pin-pentylamide (Intermediate 3b)]. To a DMF (1.5 mL) solution of intermediate2 f (150 mg, 0.15 mmol, 1.0 equiv), NaN₃ (9.7 mg, 0.15 mmol, 1.0 equiv) was added, and the resulting mixture was heated to 100° C. and stirred for 1 h. The reaction mixture was quenched by diluting with H₂O (20 mL) at 0° C., and the so-formed precipitate was filtered, washed with H₂O (10 mL) and recovered dissolving in MeOH (15 mL). The methanolic solution was concentrated in vacuo, to recover azide intermediate 3b (130 mg, 91%) as a white amorphous solid. The crude material was used as such in the next synthetic step.

LCMS (ESI-MS m/z): mass calcd for C₅₁H₈₆BN₈O₈ [M+H]⁺, 949.67; found, 949.32.

N—((S)-6-amino-1-(((S)-1-(((S)-1-(((R)-5-amino-1-((3aS,4S,6S,7aR)-3a,5,5-trimethylhexahydro-4,6-methanobenzo[d][1,3,2]dioxaborol-2-yl)pentyl)amino)-1-oxopropan -2-yl)amino)-1-oxopropan -2-yl)amino)-1-oxohexan -2-yl) palmitamide[Palmitoyl-Lys-Ala-Ala-5-amine-1-BO₂Pin-pentylamide (Intermediate 4a)]. To a MeOH (10 mL) solution of intermediate 3b (130 mg, 0.13 mmol, 1.0 equiv) 10% wt Pd/C(29 mg, 0.027 mmol, 0.2 equiv) was added, followed by H₂ (balloon). The resulting mixture was left stirring for 1.5 h, before a 4 N 1,4-dioxane solution of HCl (0.067 mL, 0.27 mmol, 2.0 equiv) was added, and the resulting mixture was stirred for 0.5h. Next, the reaction mixture was filtered through a CELITE® pad, washing with MeOH (15 mL). The filtrates were combined and concentrated in vacuo to recover intermediate 4a (111 mg, 95%) as a white amorphous solid. The crude material was used as such in the next synthetic step.

LCMS (ESI-MS m/z): mass calcd for C₄₃H₈₂BN₆O₆ [M+H]⁺, 789.64; found, 789.40.

((5R,8S,11S,14S)-1-amino-14-(4-aminobutyl)-8,11-dimethyl-7,10,13,16-tetraoxo-6,9,12,15-tetraazahentriacontan -5-yl) boronic acid bis-TFA [Palmitoyl-Lys-Ala-Ala-5-amine-1-B(OH)₂-pentylamide (Example 8)]. To a Heptane/MeOH (1:1, 10 mL) mixture of intermediate 4a (100 mg, 0.12 mmol, 1.0 equiv) isobutyl boronic acid (71 mg, 0.70 mmol, 6.0 equiv) was added, followed by a 1 M aqueous HCl (0.46 mL, 0.46 mmol, 6.0 equiv). The resulting mixture was stirred for 16 h, before it was diluted with heptane (5 mL) and MeOH (5 mL). After layer separation, the heptane layer was further extracted with MeOH (5 mL), and the methanolic layers were combined, washed with heptane (2×10 mL), and concentrated in vacuo. The crude residue was dissolved in H₂O, filtered through a 0.20 μm syringe filter, and purified using preparative RP-HPLC(isocratic 30% MeCN in H₂O over 5 min, gradient from 30 to 100% MeCN in H₂O over 30 min). All fractions containing product were combined, concentrated to 5 mL in vacuo, prior to lyophilization, to recover Example 8 (16 mg, 15%) as a white powder.

HRMS (ESI-MS m/z): mass calcd for C₃₃H₆₇BN₆NaO₆ [M+Na]⁺, 677.51074; found, 677.51419. Compounds made following the synthesis of example 8:39, 41, 48, 60, 61, 182, 183.

### Example 9

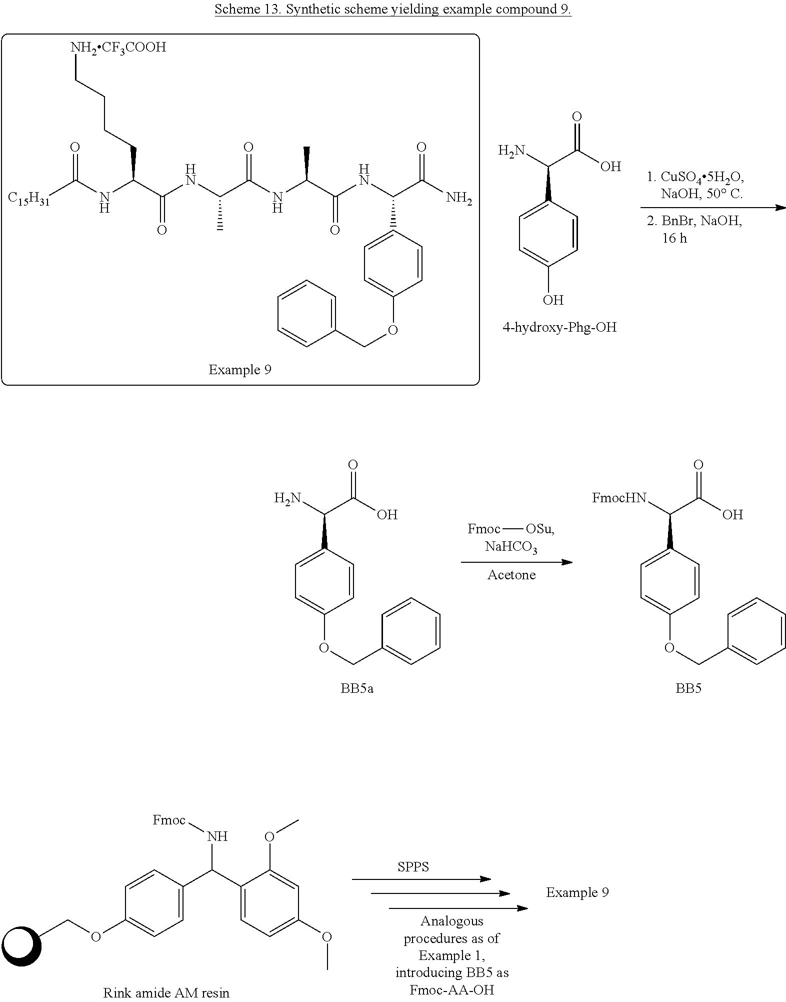

(R)-2-amino-2-(4-(benzyloxy)phenyl) acetic acid (BB5a). To a H₂O (20 mL) solution of CuSO₄ (1.8 g, 36 mmol, 3.0 equiv), a H₂O (20 mL) solution of 1 N aqueous NaOH (24 mL, 24 mmol, 2.0 equiv) and H-(4-hydroxy) Phg-OH (2.0 g, 12 mmol, 1.0 equiv) was added at 50° C. The resulting mixture was stirred at that temperature for 0.5 h, before it was cooled to 0° C. and stirred further 0.25 h. The so-formed precipitate was filtered, washed with H₂O (20 mL) and dried. Next, the solid was take up in MeOH (40 mL), and the resulting suspension was diluted with 1 N aqueous NaOH (12 mL, 12 mmol, 1.0 equiv), followed by the addition of BnBr (2.2 g, 12 mmol, 1.1 equiv). The resulting mixture was stirred for 16 h at RT, before the so-formed precipitate was filtered, washed with H₂O (100 mL) and triturated in 1 N aqueous HCl (250 mL). The precipitated was filtered, washed with H₂O (100 mL), Et₂O (100 mL) and dried in vacuo, to recover AA BB5a (700 mg, 23%), as an off-white solid.

LCMS (ESI-MS m/z): mass calcd for C15H16NO3 [M+H]⁺, 258.11; found, 258.34.

(R)-2-((((9H-fluoren-9-yl)methoxy)carbonyl)amino)-2-(4-(benzyloxy)phenyl) acetic acid [Fmoc-(OBn)-Hpg-OH(BB5)]. To a H₂O (14 mL) solution of BB5a (0.70 g, 2.7 mmol, 1.0 equiv),

NaHCO₃ (0.46 g, 5.4 mmol, 2.0 equiv) was added, followed by a acetone (14 mL) solution of Fmoc-OSu (1.0 g, 2.9 mmol, 1.1 equiv). The resulting mixture was stirred at RT for 16 h, before it was quenched by the addition of saturated aqueous KHSO₄ and the aqueous layer was extracted with EtOAc (3×100 mL). The combined organic layer were washed with brine (100 mL), dried over NA₂SO₄ and concentrated in vacuo. The crude residue was purified via flash column chromatography (SiO₂, DCM/MeOH, from 19:1 to 9:1), to recover Fmoc-(OBn)-Hpg-OH BB5 (500 mg, 39%) as an off-white solid.

R_{f}=0.5 (SiO₂, MeOH/DCM, 1:9)

LCMS (ESI-MS m/z): mass calcd for C30H₂₆NO5 [M+H]⁺, 480.18; found, 480.36.

N—((S)-6-amino-1-(((S)-1-(((S)-1-(((S)-2-amino-1-(4-(benzyloxy)phenyl)-2-oxoethyl)amino)-1-oxopropan -2-yl)amino)-1-oxopropan -2-yl)amino)-1-oxohexan -2-yl) palmitamide TFA [Palmitoyl-Lys-Ala-Ala-(OBn) Hpg-NH₂ TFA salt (Example 9)]. was prepared according the general procedure for SPPS using 1.5 g Rink amide AM resin (0.90 mmol). The crude peptide was purified by preparative RP-HPLC, all fractions containing the product were combined and concentrated in vacuo to 5 mL, prior to lyophilization, to afford peptide Example 9 (19 mg) as a white powder.

HRMS (ESI-MS m/z): mass calcd for C₄₃H₆₉N₆O₆ [M+H]⁺, 765.5273; found, 765.5255. Compounds made following the synthesis of example 9:129, 130, 138, 139.

### Example 10

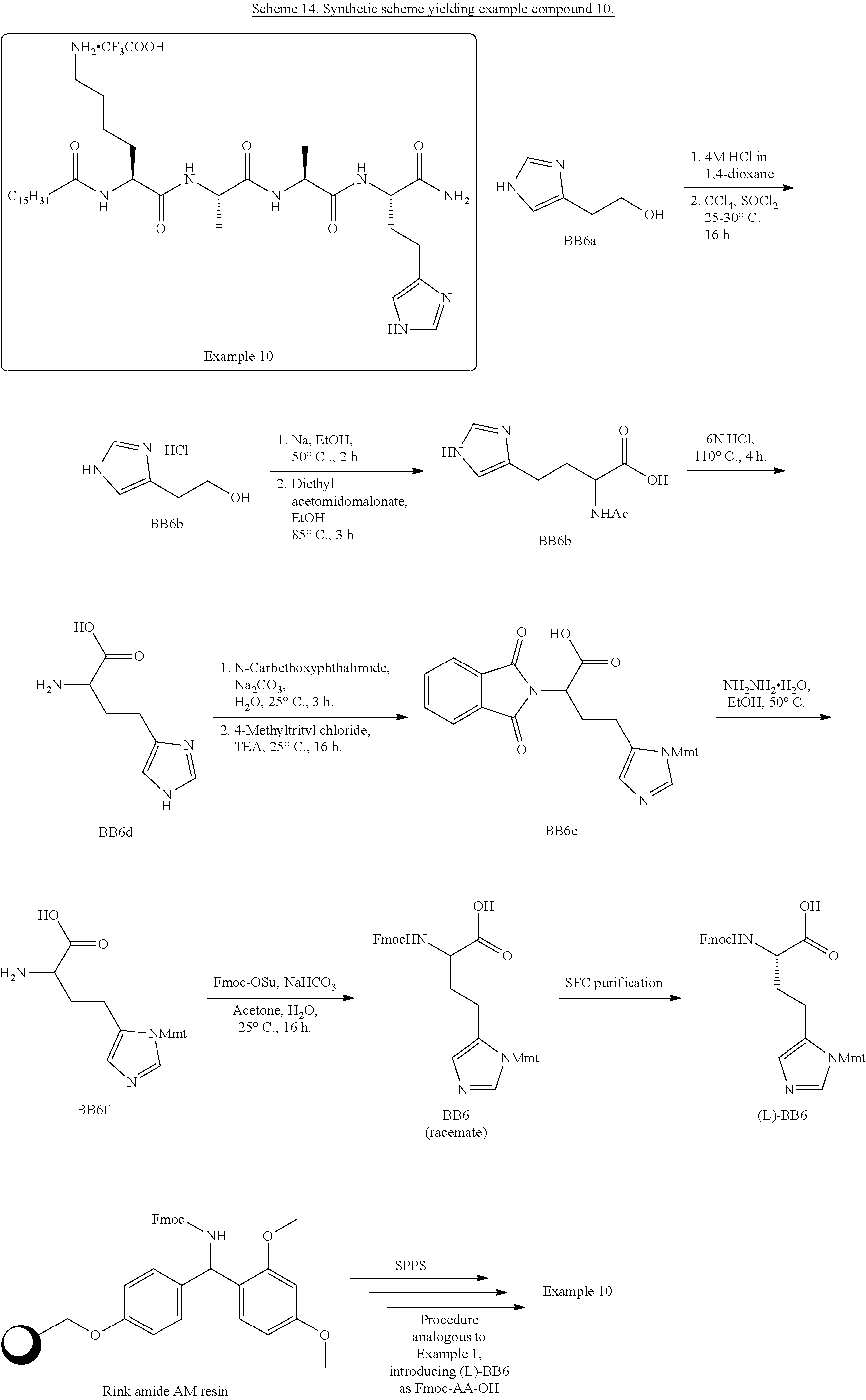

4-(2-chloroethyl)-1H-imidazole (BB6b). To a 4 M 1,4-dioxane solution of HCl (75 mL), 2-(1H-imidazol-4-yl)ethan -1-ol (BB6a)(15 g, 130 mmol, 1.0 equiv) was added, and the resulting mixture was stirred for 0.5 at RT. Next, the reaction mixture was concentrated in vacuo, and the residue was co-evaporated with toluene (2×50 mL), before it was dissolved in CCl₄ (75 mL) and cooled to 0° C. Then, SOCl₂ (75 mL) was added, and the resulting mixture was stirred at RT for 16 h, before being warmed to 90° C. and stirred at that temperature for 0.5 h. After cooling to RT, the reaction mixture was diluted with benzene (150 mL), and the so-formed precipitate was washed with Et₂O (100 mL), to recover crude chloride BB6b (15 g, 67%), which was used as such in the next synthetic step.

LCMS (ESI-MS m/z): mass calcd for C₆H₈ClN₂ [M+H]⁺, 131.04; found, 131.05.

2-Acetamido-4-(1H-imidazol-4-yl) butanoic acid [Ac-hHis-OH(BB6c)]. To a EtOH (200 mL) solution of chloride BB6b (20 g, 150 mmol, 1.0 equiv) was added a EtOH (175 mL) solution of Na (8.8 g, 380 mmol, 2.5 equiv) dropwise at 0° C., over the course of 10 minutes. To the resulting mixture, a EtOH (25 mL) solution of Diethyl acetamidomalonate (50 g, 230 mmol, 1.5 equiv) was added dropwise over 10 minutes, before the reaction mixture was heated to 85° C. and stirred for 5 h. After cooling to RT, the reaction mixture was quenched by the addition of 1 N aqueous HCl (200 mL), and concentrated in vacuo. The crude residue was washed with pet-ether (300 mL) to recover crude Ac-hHis-OH BB6c (30 g, quant.) as a brown gum, which was used as such in the next synthetic step.

LCMS (ESI-MS m/z): mass calcd for C₉H₁₃N₃O₃ [M+H]⁺, 212.10; found, 212.25.

2-Amino-4-(1H-imidazol-4-yl) butanoic acid [H-hHis-OH(BB6d)]. To 6 N aqueous HCl (300 mL), Ac-hHis-OH BB6c (30 g, 120 mmol, 1.0 equiv) was added, and the resulting mixture was heated to 110° C., stirring for 5 h. Next, the reaction mixture was quenched by the addition of 2 M aqueous NaOH (1.0 L), and concentrated in vacuo, and the crude residue was purified via preparative RP-HPLC, to recover H-hHis-OH BB6d (5.5 g, 27%) as an off-white solid. LCMS (ESI-MS m/z): mass calcd for C₇H₁₂N₃O₂ [M+H]⁺, 170.09; found, 170.12.

2-(1,3-dioxoisoindolin-2-yl)-4-(1H-imidazol-5-yl) butanoic acid [PHTI-hHis-OH(Not isolated)]. To a H₂O (55 mL) solution of H-hHis-OH BB6d (4.5 g, 27 mmol, 1.0 equiv), Na₂CO₃ (2.8 g, 27 mmol, 1.0 equiv) was added, followed by N-carbethoxyphthalimide (5.8 g, 27 g, 1.0 equiv). The resulting mixture was stirred at RT for 3, before it was quenched by the addition of 1 N aqueous HCl (75 mL) and concentrated in vacuo. The crude residue was suspended in MeOH (100 mL), filtered, and the filtrate was concentrated in vacuo, to recover crude PTHI-hHis-OH (8.1 g, quant.) as a brown solid, which was used as such in the next synthetic step.

LCMS (ESI-MS m/z): mass calcd for C₁₅H₁₄N₃O₄ [M+H]⁺, 300.10; found, 300.21.

2-(1,3-dioxoisoindolin-2-yl)-4-(1-(diphenyl(p-tolyl)methyl)-1H-imidazol-5-yl) butanoic acid [PHTI-hHis (Mmt)-OH(BB6e)]. To a DMF/DCM (1:2, 90 mL) solution of crude PHTI-hHis-OH (8.1 g, 27 mmol, 1.0 equiv), TEA (15 mL, 110 mmol, 4.0 equiv) was added at 0° C., followed by Mmt-Cl (15 g, 53 mmol, 2.0 equiv). The resulting mixture was warmed to RT and stirred for 16 h, before it was quenched by the addition of saturated aqueous KHSO₄ (200 mL). The aqueous layer was extracted with EtOAc (3×200 mL), and the combined organic layers were dried over Na₂SO₄ and concentrated in vacuo, the crude residue was purified via flash column chromatography (SiO₂, MeOH/DCM, from 1:19 to 1:9) to recover PHTI-hHis (Mmt)-OH BB6e (2.4 g, 16%) as an off-white solid.

LCMS (ESI-MS m/z): mass calcd for C₃₅H₃₀N₃O₄ [M+H]⁺, 556.22; found, 556.49.

2-Amino-4-(1-(diphenyl(p-tolyl)methyl)-1H-imidazol-5-yl) butanoic acid [H-hHis (Mmt)-OH(BB6f)]. To a EtOH (40 mL) solution of PTHI-hHis (Mmt)-OH BB6e (2.0 g, 3.6 mmol, 1.0 equiv), hydrazine hydrate (0.34 mL, 7.2 mmol, 2.0 equiv) was added, and the resulting mixture was stirred for 3 h at RT. Next, the so-formed solids were filtered, and the filtrate was concentrated in vacuo, to recover crude H-hHis (Mmt)-OH BB6f (2.0 g, quant) as an off-white solid, which was used as such in the next synthetic step.

LCMS (ESI-MS m/z): mass calcd for C₂₇H₂₈N₃O₂ [M+H]⁺, 426.22; found, 426.46 2-((((9H-fluoren-9-yl)methoxy)carbonyl)amino)-4-(1-{diphenyl(p-tolyl)methyl)-1H-imidazol-5-yl) butanoic acid [Fmoc-(+/−) hHis (Mmt)-OH (rac-BB6)]. To a H₂O (20 mL) solution of H-hHis (Mmt)-OH BB6f (2.0 g, 4.7 mmol, 1.0 equiv), NaHCO₃ (0.78 g, 9.3 mmol, 2.0 equiv) was added, before the reaction mixture was cooled to 0° C., and an acetone (20 mL) solution of Fmoc-OSu (2.3 g, 7.0 mmol, 1.5 equiv) was added drop-wise. The resulting mixture was warmed to RT and stirred for 16 h, before it was quenched by the addition of saturated aqueous KHSO₄ The aqueous layer was extracted with EtOAc (3×200 mL), the combined organic layers were dried over Na₂SO₄ and it was concentrated in vacuo. The crude residue was purified via flash column chromatography (SiO₂, MeOH/DCM+1% AcOH, from 1:9 to 3:15) to recover Fmoc-(+/−) hHis (Mmt)-OH rac-BB6 (1.5 g, 57%) as a light-yellow gum.

LCMS (ESI-MS m/z): mass calcd for C₄₂H₃₈N₃O₄ [M+H]⁺, 648.29; found, 648.47

(S)-2-((((9H-fluoren-9-yl)methoxy)carbonyl)amino)-4-(1-(diphenyl(p-tolyl)methyl)-1H-imidazol-5-yl) butanoic acid [Fmoc-(S)-hHis (Mmt)-OH(L-BB6)]. Fmoc-(+/−) hHis (Mmt)-OH (rac-BB6) was purified by chiral SFC, to recover two sets of fractions. The set of fractions eluting at first showed negative optical rotation value [ ]_{D}²⁵=−31.16, therefore the structure was assigned to be the D-enantiomer. Concentration in vacuo of the secondly eluting fraction allowed to recover Fmoc-deprotected L-BB6, which was re-submitted to Fmoc-protection as in the synthesis of rac-BB6, to recover Fmoc-(+) hHis (Mmt)-OH L-BB6 (400 mg, 27%) as a white powder.

LCMS (ESI-MS m/z): mass calcd for C₄₂H₃₈N₃O₄ [M+H]⁺, 648.29; found, 648.47

N—((S)-6-amino-1-(((S)-1-(((S)-1-(((S)-1-amino-4-(1H-imidazol-4-yl)-1-oxobutan -2-yl)amino)-1-oxopropan -2-yl)amino)-1-oxopropan -2-yl)amino)-1-oxohexan -2-yl) palmitamide bis-TFA [Palmitoyl-Lys-Ala-Ala-hHis-NH₂ bis-TFA salt (Example 10)]. was prepared according the general procedure for SPPS using 0.4 g Rink amide AM resin (0.90 mmol). The crude peptide was purified by preparative RP-HPLC, all fractions containing the product were combined and concentrated in vacuo to 5 mL, prior to lyophilization, to afford peptide Example 10 (8.2 mg, 6%) as a white powder.

HRMS (ESI-MS m/z): mass calcd for C₃₅H₆₅N₈O₅ [M+H]⁺, 677.5072; found, 677.5044. Compounds made following the synthesis of example 10:59, 165, 166, 172, 173.

### Example 11

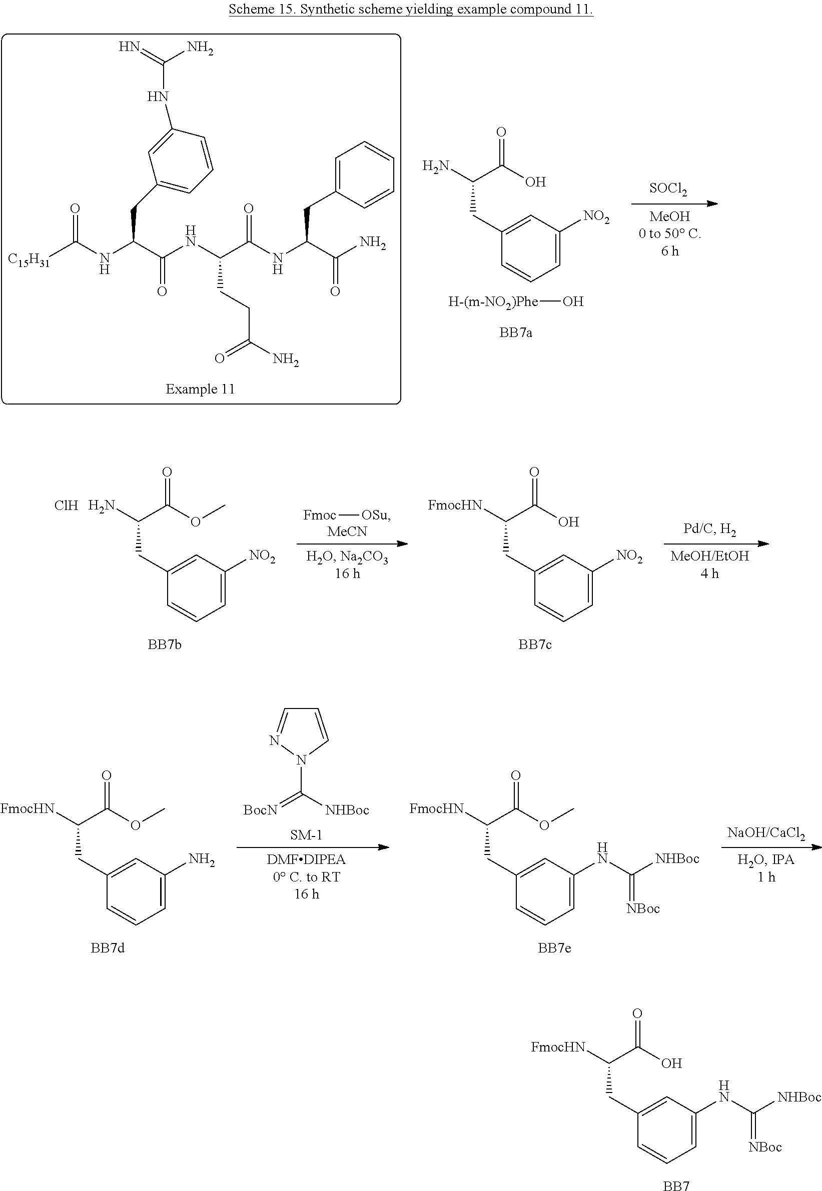

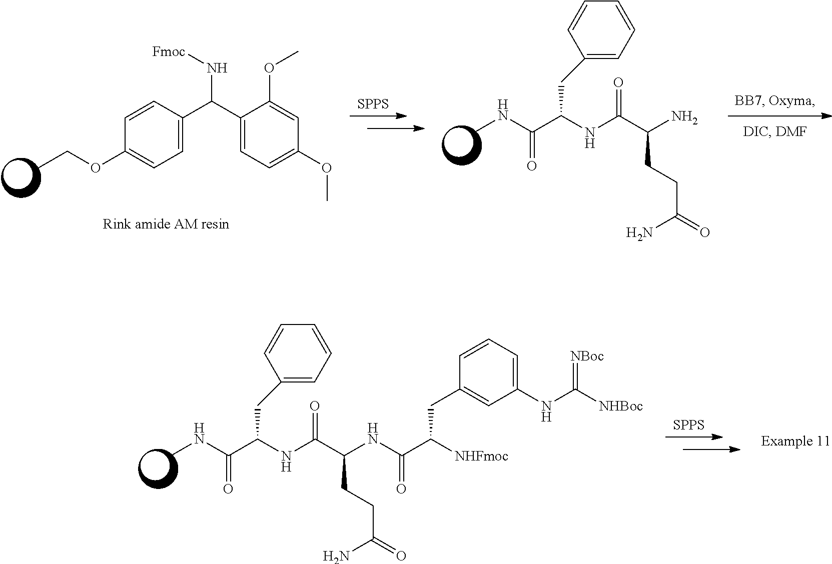

Methyl(S)-2-amino-3-(3-nitrophenyl) propanoate hydrochloride (BB7b). To a MeOH (80 mL) solution of H-(m-NO2) Phe-OH BB7a (4.0 g, 19 mmol, 1.0 equiv), SOCl₂ (2.3 mL, 32 mmol, 1.7 equiv) was added at 0° C. The resulting mixture was warmed to RT, then heated to 50° C., and stirred for 6 h, before being concentrated in vacuo. The crude residue was triturated in Et₂O (100 mL), to recover hydrochloride BB7b (4.0 g, 94%) as an off white solid.

LCMS (ESI-MS m/z): mass calcd for C₁₀H₁₃N₂O₄ [M+H]⁺, 225.09; found, 225.06.

methyl(S)-2-((((9H-fluoren-9-yl)methoxy)carbonyl)amino)-3-(3-nitrophenyl) propanoate[Fmoc-(m-NO2)-Phe-OMe (BB7c)]. To a H₂O (50 mL) solution of hydrochloride BB7b (5.0 g, 22 mmo, 1.0 equiv), Na₂CO₃ (4.7 g, 44 mmol, 2.0 equiv) was added, followed by a MeCN (50 mL) solution of Fmoc-OSu (11 g, 33 mmol, 1.5 equiv). The resulting mixture was stirred for 16 h at RT, before it was diluted with H₂O (50 mL), and extracted with EtOAc (3×100 mL). The combined organic layer was washed with brine (100 mL), dried over Na₂SO₄, and concentrated in vacuo. The crude residue was purified via flash column chromatography (SiO₂, EtOAc/pet-ether, 1:4) to recover Fmoc-(m-NO2)-Phe-OMe BB7c (3.2 g, 33%) as an off-white solid.

R_{f}=0.6 (SiO₂, EtOAc/pet-ether, 3:2).

LCMS (ESI-MS m/z): mass calcd for C₂₅H₂₃N₂O₆ [M+H]⁺, 447.16; found 447.18.

Methyl(S)-2-((((9H-fluoren-9-yl)methoxy)carbonyl)amino)-3-(3-aminophenyl) propanoate[Fmoc-(m-NH₂)-Phe-OMe (BB7d)]. To a MeOH/EtOH (1:1, 64 mL) solution of Fmoc-(m-NO2)-Phe-OMe BB7c (3.2 g, 7.2 mmol, 1.0 equiv), 10% wt Pd/C(0.32 g, 10% wt) was added, followed by H₂ (balloon). The resulting mixture was stirred at RT for 4 h, before it was filtered over a CELITE® pad, washing with MeOH (20 mL). The filtrate was collected and concentrated in vacuo, to recover amine BB7d (2.9 g, 97%) as a brown oil.

LCMS (ESI-MS m/z): mass calcd for C₂₅H₂₅N₂O₄ [M+H]⁺, 417.18; found, 417.14.

Methyl (S,Z)-2-((((9H-fluoren-9-yl)methoxy)carbonyl)amino)-3-(3-(2,3-bis(tert-butoxycarbonyl) guanidino)phenyl) propanoate[Fmoc-[m-(bis-Boc) guanidyl]-Phe-OMe (BB7e)]. To a DMF (60 mL) solution of amine BB7d (3.0 g, 7.2 mmol, 1.0 equiv), DIPEA (3.8 mL, 22 mmol, 3.0 equiv) was added at 0° C., followed by SM-1 (4.0 g, 13 mmol, 2.0 equiv) and DMAP (87 mg, 0.72 mmol, 0.1 equiv). The resulting mixture was warmed to RT an stirred for 16h, before in was quenched by the addition of H₂O (45 mL). The aqueous layer was extracted with EtOAc (3× 60 mL), and the combined organic layer was washed with brine (60 mL), dried over Na₂SO₄ and concentrated in vacuo. The crude residue was purified via flash column chromatography (SiO₂, EtOAc/pet-ether, from 1:9 to 1:5) to recover bis-boc-guanidyl BB7e (1.6 g, 34%) as a pale yellow liquid.

LCMS (ESI-MS m/z): mass calcd for C₃₆H₄₃N₄O₈ [M+H]⁺, 659.31; found, 659.26.

(S,Z)-2-((((9H-fluoren-9-yl)methoxy)carbonyl)amino)-3-(3-(2,3-bis(tert-butoxycarbonyl) guanidino)phenyl) propanoic acid [Fmoc-[m-(bis-Boc) guanidyl]-Phe-OH(BB7)]. To a H₂O (7.5 mL) solution of NaOH (97 mg, 2.4 mmol, 1.0 equiv), 0.8 M aqueous CaCl₂) (1.6 mL) was added at 0° C., followed by an IPA (16 mL) solution of methyl ester BB7e (1.6 g, 1.4 mL, 1.0 equiv). The resulting mixture was warmed to RT and stirred for 1 h, before it was quenched by the addition of saturated aqueous KHSO₄ (until pH 4). The aqueous layer was extracted with MeOH/DCM (1:9, 3×50 mL), and the combined organic layer was washed with brine (50 mL), dried over Na₂SO₄ and concentrated in vacuo. The crude residue was purified via flash column chromatography (SiO₂, EtOAc/pet-ether, from 7:3 to 4:1) to recover Fmoc-AA-OH BB7 (1.6 g, 34%) as a pale yellow liquid.

R_{f}=0.3 (SiO₂, DCM/MeOH, 9:1).

LCMS (ESI-MS m/z): mass calcd for C₃₅H₄₁N₄O₈ [M+H]⁺, 645.29; found, 645.22.

(S)—N₁—((S)-1-amino-1-oxo-3-phenylpropan -2-yl)-2-((S)-3-(3-guanidinophenyl)-2-palmitamidopropanamido)pentanediamide bis-TFA [Palmitoyl-(m-guanidyl) Phe-GIn-Phe-NH₂ bis-TFA salt (Example 11)]. was prepared according the general procedure for SPPS using 1.0 g Rink amide AM resin (0.90 mmol), ad exception of the amide coupling of BB7, which was performed using DIC and Oxyma. The crude peptide was purified by preparative RP-HPLC, all fractions containing the product were combined and concentrated in vacuo to 5 mL, prior to lyophilization, to afford peptide Example 11 (33 mg, 12%) as a white powder.

HRMS (ESI-MS m/z): mass calcd for C₄₀H₆₃N₈O₅ [M+H]⁺, 735.4916; found, 735.4905.

Compounds made following the synthesis of example 11:67, 68.

### Example 12

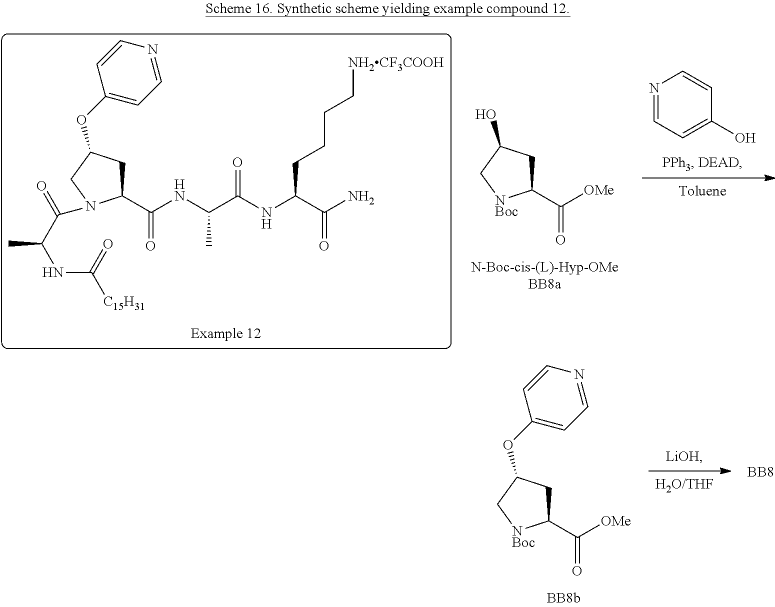

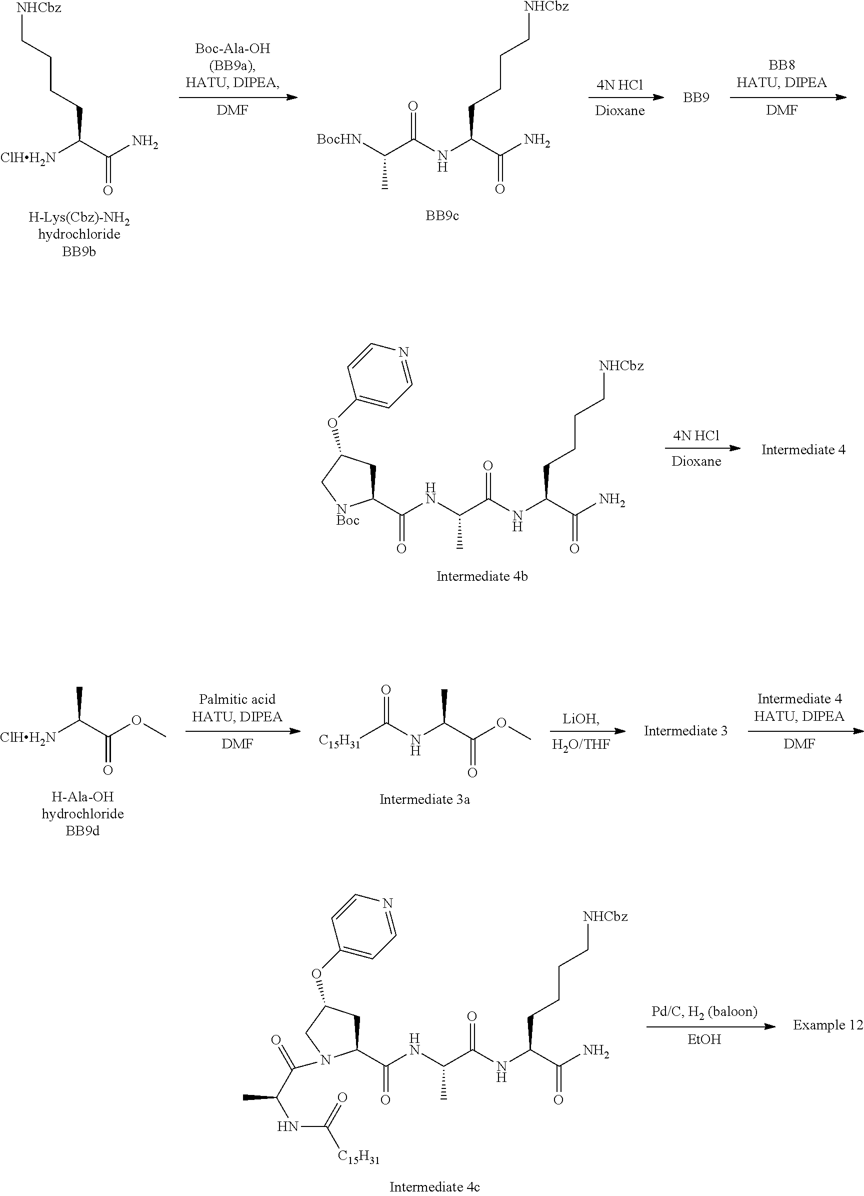

1-(Tort-butyl)₂-methyl (2S,4R)-4-(pyridin-4-yloxy) pyrrolidine-1,2-dicarboxylate [Boc-trans-(O-pyridinyl) Hyp-OMe (BB8b)]. To a toluene (38 mL) solution of BB8a (1.0 g, 4.1 mmol, 1.0 equiv), 4-hydroxypyridine (580 mg, 6.1 equiv, 1.5 equiv) was added at 0° C., followed by a 40% toluene solution of DEAD (2.4 mL, 6.1 mmol, 1.5 equiv). The resulting mixture was warmed to RT and stirred for 24 h, before it was concentrated in vacuo, and the crude residue was purified via filtration over a silica plug. The plug was washed with Et₂O (100 mL) to remove the impurities, before the product was stripped down by washing the plug with DCM/MeOH (9:1, 200 mL). The DCM/MeOH fraction was concentrated in vacuo, to recover Mitsunobu product BB8b (910 mg, 70%) as a white amorphous solid.

R_{f}=0.60 (SiO₂, DCM/MeOH, 9:1).

¹H NMR (400 MHZ, CDCl₃) δ 8.47-8.39 (m, 2H), 6.75-6.67 (m, 2H), 4.96 (m, 1H), 4.57*(t, J=5.6 Hz, 0.4H), 4.45 (dd, J=8.9, 2.8 Hz, 0.6H), 3.81-3.63 (m, 5H), 2.61-2.46 (m, 2H), 1.48*(s, 4H), 1.44 (s, 5H)(*=minor rotamer).

(2S,4R)-1-(tert-butoxycarbonyl)-4-(pyridin-4-yloxy) pyrrolidine-2-carboxylic acid [Boc-trans-(O-pyridinyl) Hyp-OH(BB8)]. To a THF (15 mL) solution of methyl ester BB8b (900 mg, 2.8 mmol, 1.0 equiv), 1.0 M aqueous LiOH (15 mL, 15 mmol, 5.3 equiv) at 0° C. The resulting mixture was stirred for 2 h, before it was quenched by the addition of 1 M aqueous HCl (until pH=1), and it concentrated in vacuo to half its volume. The aqueous layer was extracted with IPA/CHCl₃ (1:3, 3×30 mL), the combined organic layers were dried over Na₂SO₄, and concentrated in vacuo. The crude residue was purified via flash column chromatography (SiO₂, DCM/MeOH/AcOH, from 46:4:0 to 45:4:1) to recover carboxylic acid BB8 (0.15 g, 17%) as a white solid. R_{f}=0.43 (SiO₂, DCM/MeOH/AcOH, 45:4:1).

¹H NMR (400 MHZ, DMSO-d₆) δ 8.35-8.30 (m, 2H), 6.93-6.87 (m, 2H), 5.06-4.98 (m, 1H), 4.15-4.02 (m, 1H), 3.68-3.54 (m, 1H), 3.50-3.41 (m, 1H), 2.36-2.12 (m, 2H), 1.30*(s, 4H), 1.28 (s, 5H)(*=minor rotamer).

Tert-butyl ((S)-1-(((S)-1-amino-6-(((benzyloxy)carbonyl)amino)-1-oxohexan -2-yl)amino)-1-oxopropan -2-yl)carbamate[Boc-Ala-Lys (Cbz) NH₂ (BB9c)]. To a DCM (25 mL) solution of amine hydrochloride H-Lys (Cbz)-NH₂ BB9b (730 mg, 2.3 mmol, 1.0 equiv), carboxylic acid Boc-Ala-OH BB9a (480 mg, 2.5 mmol, 1.1 equiv) was added at 0° C., followed by HATU (950 mg, 2.5 mmol, 1.1 equiv) and DIPEA (1.4 mL, 8.0 mmol, 3.5 equiv). The resulting mixture was left stirring 2 h, before it was quenched by diluting with 15% wt aqueous citric acid (30 mL) at 0° C. After layer separation, the aqueous layer was further extracted with DCM (3×30 mL), and the combined organic layers were washed with saturated aqueous NaHCO₃ (30 mL), H₂O (30 mL) and brine (30 mL). The organic layer was dried over Na₂SO₄, and concentrated in vacuo, to recover crude dipeptide BB9c (1.1 g, 97) as an off-white solid, which was used as such in the next synthetic step.

¹H NMR (400 MHZ, DMSO-d₆) õ 7.59 (d, J=8.2 Hz, 1H), 7.36-7.20 (m, 5H), 7.29 (d, J=2.3 Hz, 1H), (m, 1H), 7.00-6.93 (m, 2H), 4.95 (s, 2H), 4.11 (m, 1H), 3.89 (m, 1H), 2.91 (q, J=6.6 Hz, 2H), 1.65-1.53 (m, 1H), 1.51-1.38 (m, 1H), 1.32 (s, 11H), 1.25-1.15 (m, 2H), 1.12 (d, J=6.5 Hz, 3H).

Benzyl ((S)-6-amino-5-((S)-2-aminopropanamido)-6-oxohexyl)carbamate HCl [H-Ala-Lys (Cbz) NH₂ HCl salt (BB9)]. To a 1,4-dioxane (8 mL) solution of dipeptide BB9c (1.0 g, 2.2 mmol, 1.0 equiv), a 4 N 1,4-dioxane solution of HCl (16 mL, 2V) was added at 0° C., and the resulting mixture was stirred for 1 h. The reaction mixture was concentrated in vacuo, and the crude residue was triturated in Et₂O (3×30 mL), before the so-formed solids were dissolved in DCM (50 mL). The DCM solution was concentrated in vacuo, to recover amine hydrochloride BB9 (880 mg, 90%) as transparent crystals.

¹H NMR (400 MHZ, DMSO-d₆) δ 8.53 (d, J=8.1 Hz, 1H), 8.32-8.21 (m, 3H), 7.50-7.40 (m, 1H), 7.30-7.29 (m, 1H), 7.37-7.19 (m, 5H), 7.02-6.96 (m, 1H), 4.95 (s, 1H), 4.18-4.10 (m, 1H), 3.89-3.78 (m, 1H), 2.97-2.87 (m, 2H), 1.74-1.45 (m, 2H), 1.31 (d, J=6.9 Hz, 3H), 1.39-1.16 (m, 4H).

Tert-butyl (2S,4R)-2-(((S)-1-(((S)-1-amino-6-(((benzyloxy)carbonyl)amino)-1-oxohexan -2-yl)amino)-1-oxopropan -2-yl)carbamoyl)-4-(pyridin-4-yloxy) pyrrolidine-1-carboxylate [Boc-trans-(O-pyridinyl) Hyp-Ala-Lys (Cbz) NH₂ (Intermediate 4b)]. To a DMF (7.0 mL) solution of amine hydrochloride BB9 (190 mg, 0.49 mmol, 1.0 equiv), carboxylic acid BB9 (150 mg, 0.49 mmol, 1.0 equiv) was added at 0° C., followed by HATU (200 mg, 0.53 mmol, 1.1 equiv) and DIPEA (0.34 mL, 1.9 mmol, 4.0 equiv). The resulting mixture was left stirring 3 h, before it was quenched by diluting with H₂O (80 mL) at 0° C. The aqueous layer was extracted with IPA/CHCl₃ (1:3, 3×100 mL), and the combined organic layers were dried over Na₂SO₄ and concentrated in vacuo. The crude residue was purified via flash column chromatography (SiO₂, DCM/MeOH, from 1:0 to 9:1) to recover tripeptide intermediate 4b (0.30 g, 95%) as a white powder. Benzyl ((S)-6-amino-6-oxo-5-((S)-2-((2S,4R)-4-(pyridin-4-yloxy) pyrrolidine-2-carboxamido) propanamido) hexyl)carbamate HCl [H-trans-(O-pyridinyl) Hyp-Ala-Lys (Cbz)-NH₂ HCl salt (Intermediate 4)]. To a 1,4-dioxane (2.5 mL) solution of dipeptide BB9c (0.3 g, 0.42 mmol, 1.0 equiv), a 4 N 1,4-dioxane solution of HCl (5 mL) was added at 0° C., and the resulting mixture was stirred for 1 h. The reaction mixture was concentrated in vacuo, and the crude residue was triturated in Et₂O (3×30 mL), before the so-formed solids were dissolved in DCM (50 mL). The DCM solution was concentrated in vacuo, to recover amine hydrochloride Intermediate 4 (280 mg, 95%) as a white powder. Methyl palmitoyl-L-alaninate [Palmitoyl-Ala-OMe (Intermediate 3a)]. To a DCM (35 mL) solution of amine hydrochloride H-Ala-OMe BB9d (550 mg, 3.9 mmol, 1.0 equiv), palmitic acid (1.0 g, 3.9 mmol, 1.0 equiv) was added at 0° C., followed by HATU (1.6 g, 4.3 mmol, 1.1 equiv) and DIPEA (2.1 mL, 12 mmol, 3.0 equiv). The resulting mixture was left stirring 2 h, before it was quenched by diluting with 15% wt aqueous citric acid (30 mL) at 0° C. After layer separation, the aqueous layer was further extracted with DCM (3×30 mL), and the combined organic layers were washed with saturated aqueous NaHCO₃ (30 mL), H₂O (30 mL) and brine (30 mL). The organic layer was dried over Na₂SO₄, and concentrated in vacuo, to recover crude dipeptide BB9c (1.1 g, 97%) as an off-white solid, which was used as such in the next synthetic step.

¹H NMR (400 MHZ, CDCl₃) δ 6.07 (d, J=7.4 Hz, 1H), 4.61 (p, J=7.2 Hz, 1H), 3.75 (s, 3H), 2.25-2.16 (m, 2H), 1.69-1.57 (m, 2H), 1.40 (d, J=7.1 Hz, 3H), 1.25 (m, 24H), 0.88 (t, J=7.2, 3H). Palmitoyl-L-alanine[Palmitoyl-Ala-OH(Intermediate 3)]. To a THF (15 mL) solution of methyl ester BB8b (840 mg, 2.5 mmol, 1.0 equiv), 1.0 M aqueous LiOH (13 mL, 13 mmol, 5.2 equiv) at 0° C. The resulting mixture was stirred for 2 h, before it was quenched by the addition of 1 M aqueous HCl (until pH=1), and it concentrated in vacuo to half its volume. The aqueous layer was extracted with CHCl₃ (3×20 mL), the combined organic layers were dried over Na₂SO₄, and concentrated in vacuo, to recover crude carboxylic acid BB8 (0.72 g, 90%) as a white amorphous solid, which was used as such in the next synthetic step.

¹H NMR (400 MHZ, CDCl₃) δ 6.25 (d, J=7.1 Hz, 1H), 4.58 (p, J=7.1 Hz, 1H), 2.27-2.18 (m, 2H), 1.69-1.56 (m, 2H), 1.45 (d, J=7.1 Hz, 3H), 1.33-1.23 (m, 24H), 0.88 (d, J=6.6 Hz, 3H). Benzyl ((S)-6-amino-6-oxo-5-((S)-2-((2S,4R)-1-(palmitoyl-L-alanyl)-4-(pyridin-4-yloxy) pyrrolidine-2-carboxamido) propanamido) hexyl)carbamate[Palmitoyl-Ala-trans-(O-pyridinyl) Hyp-Ala-Lys (Cbz) NH₂ (Intermediate 4c)]. To a DMF (6.0 mL) solution of amine hydrochloride BB9 (300 mg, 0.52 mmol, 1.0 equiv), carboxylic acid BB8 (290 mg, 0.88 mmol, 1.7 equiv) was added at 0° C., followed by HATU (300 mg, 0.78 mmol, 1.5 equiv) and DIPEA (0.32 mL, 0.78 mmol, 3.5 equiv). The resulting mixture was left stirring 24 h, before it was quenched by diluting with H₂O (60 mL) at 0° C. The so-formed precipitate was filtered, washed with H₂O (30 mL) and recovered dissolving in MeOH (30 mL). The methanolic solution was concentrated in vacuo, to recover crude peptide Intermediate 4c (350 mg, 79%) as an off-white solid, which was used as such in the next synthetic step.

(2S,4R)—N—((S)-1-(((S)-1,6-diamino-1-oxohexan -2-yl)amino)-1-oxopropan -2-yl)-1-(palmitoyl-L-alanyl)-4-(pyridin-4-yloxy) pyrrolidine-2-carboxamide TFA [Palmitoyl-Ala-trans-(O-pyridinyl) Hyp-Ala-Lys-NH₂ TFA salt (Example 12)].

To a MeOH (5.0 mL) solution of intermediate 4c (350 mg, 0.41 mmol, 1.0 equiv)₁₀% wt Pd/C (44 mg, 0.041 mmol, 0.1 equiv) was added, followed by H₂ (balloon). The resulting mixture was left stirring for 16 h, before a 4 N 1,4-dioxane solution of HCl (0.067 mL, 0.27 mmol, 2.0 equiv) was added, and the resulting mixture was stirred for 0.5h. Next, the reaction mixture was filtered through a CELITE® pad, washing with MeOH (15 mL). The filtrates were combined and concentrated in vacuo, and the crude residue was dissolved in MeOH (10 mL), filtered through a 0.20 μm syringe filter, and purified using preparative RP-HPLC(isocratic 30% MeCN in H₂O over 5 min, gradient from 35 to 45% MeCN in H₂O over 20 min, isocratic 45% over 7 min, with a solvent flow rate of 10.0 mL/min, at 30° C., Rt.=25 min). All fractions containing product were combined, concentrated to 5 mL in vacuo, prior to lyophilization, to recover Example 12 (12 mg, 3.5%) as a white powder.

LCMS (ESI-MS m/z): mass calcd for C₃₈H₆₆N₇O₆ [M+H]⁺, 716.51; found, 716.60.

Compounds made following the synthesis of example 12:7, 8.

### Example 13

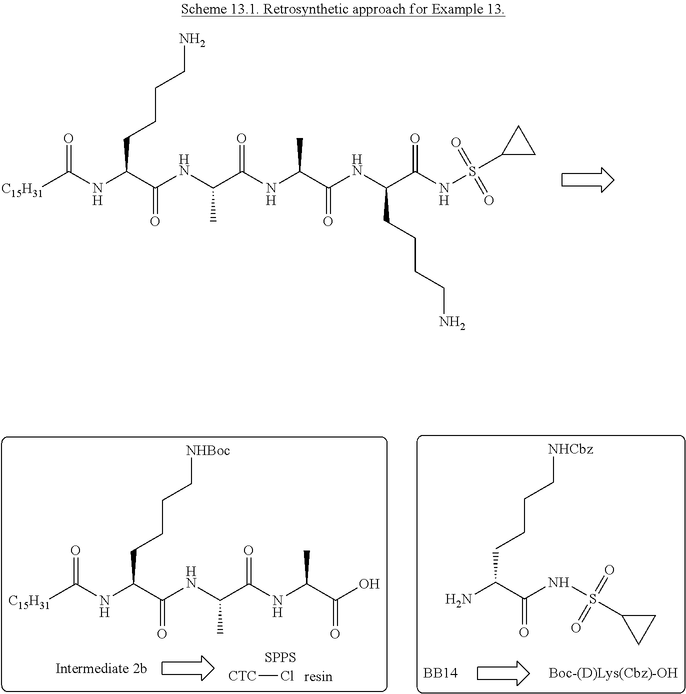

Lipopeptides where a sulfonamide moiety was installed on C-terminus, yielding compounds such as Example 13, were obtained analogously to examples 5-8, i.e. via CDI mediated coupling of side-chain protected intermediate 2b (whose synthesis was discussed earlier in the document) with a Lysine derivative such as BB14. BB14 was synthesized after HATU coupling of commercially available Boc-(D) Lys (Cbz)-OH with cyclopropyl sulfonamide.

Compounds made according to the following procedures: 127, 135, 178, 179

### Example 13. Experimental Procedures

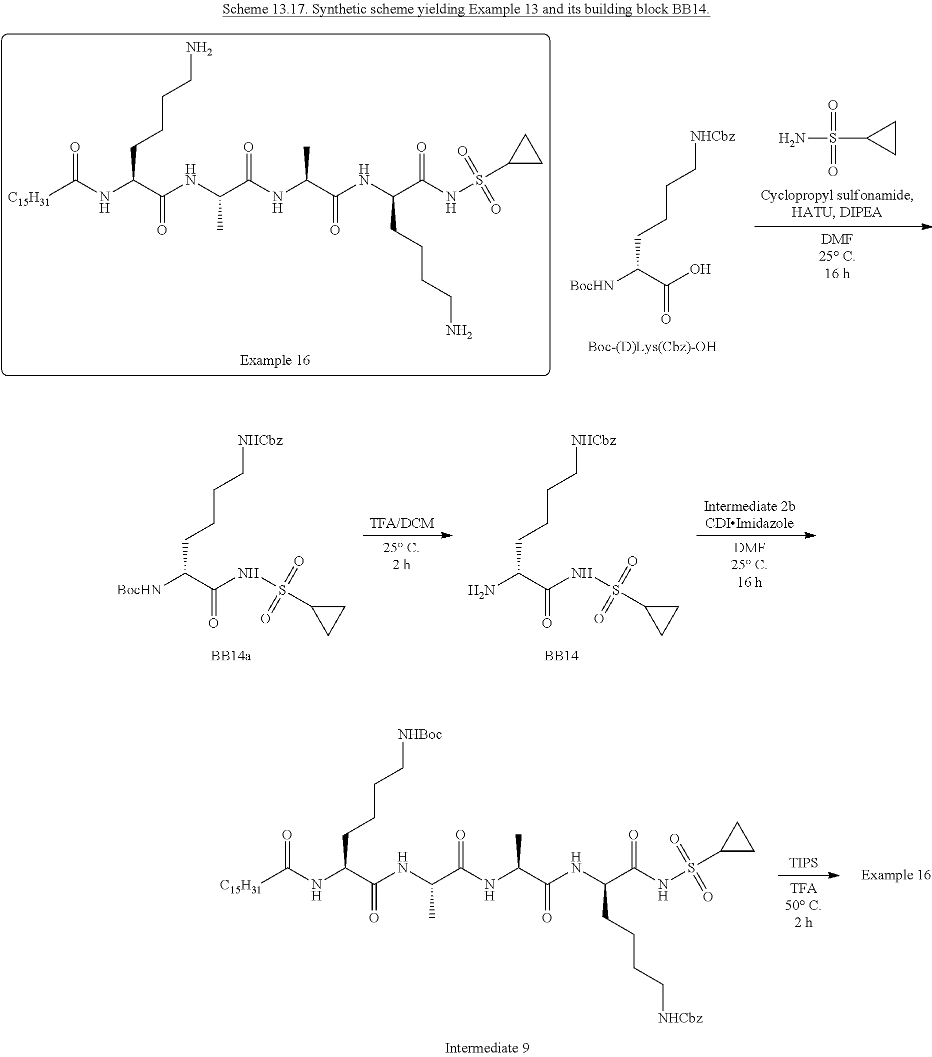

Benzyl tert-butyl (6-(cyclopropanesulfonamido)-6-oxohexane-1,5-diyl)(R)-dicarbamate (BB14a). To a DMF (20 mL) solution of Boc-(D) Lys (Cbz)-OH (1.0 g, 2.6 mmol, 1.0 equiv), HATU (1.3 g, 3.2 mmol, 1.2 equiv) was added at 0° C., followed by DIPEA (1.1 mL, 6.4 mmol, 2.5 equiv). The resulting mixture was left stirring for 10 minutes, before cyclopropyl sulfonamide (0.39 g, 3.2 mmol, 1.2 equiv) was added. The resulting mixture was warmed to 25° C. and left stirring for 16 h, before it was quenched by the addition of water (50 mL), and extracted with EtOAc (3×100 mL).

The combined organic layer was dried over Na₂SO₄ and concentrated in vacuo, to recover BB14a (0.9 g, 73%) as crude material, which was used as such in the next synthetic step. R_{f}=0.6 (SiO₂, EtOAc).

LCMS (ESI-MS m/z): mass calcd for C₂₂H₃₄N₃O₇S [M+H]⁺, 484.21; found 484.45.

Benzyl (R)-(5-amino-6-(cyclopropanesulfonamido)-6-oxohexyl)carbamate TFA (BB14). To a TFA/DMF (8 mL, 1:4) solution, BB14a (0.90 g, 1.9 mmol, 1.0 equiv) was added at 0° C. The resulting mixture was warmed to to 25° C. and left stirring for 16 h, before it was concentrated in vacuo. The crude residue was triturated in Et₂O (50 mL), and the solid was decanted, to recover BB14 (0.8g, quant.) as an off-white solid, which was used as such in the next synthetic step.

Tert-butyl ((9R,12S,15S,18S)-9-((cyclopropylsulfonyl)carbamoyl)-12,15-dimethyl-3,11,14,17-tetraoxo-18-palmitamido-1-phenyl-2-oxa-4,10,13,16-tetraazadocosan -22-yl)carbamate (Intermediate 9). To a DMF (8 mL) solution of Intermediate 2b (0.40 g, 0.63 mmol, 1.0 equiv), CDI (0.15 g, 0.95 mmol, 1.5 equiv) was added, followed by imidazole (65 mg, 0.95 mmol, 1.5 equiv). The resulting mixture was left stirring for 10 minutes, before BB14 (0.15 g, 0.38 mmol, 0.6 equiv) was added. The resulting mixture was left stirring for 16 h, before it was quenched by the addition of water (50 mL), and extracted with EtOAc (3×100 mL). The combined organic layer was dried over Na₂SO₄ and concentrated in vacuo, to recover Intermediate 9 (0.5 g, 73%) as crude material, which was used as such in the next synthetic step.

LCMS (ESI-MS m/z): mass calcd for C₅₀H₈₆N₇O₁₁S [M+H]⁺, 992.61; found 992.78

N—((S)-6-amino-1-(((S)-1-(((S)-1-(((R)-6-amino-1-(cyclopropanesulfonamido)-1-oxohexan -2-yl)amino)-1-oxopropan -2-yl)amino)-1-oxopropan -2-yl)amino)-1-oxohexan -2-yl) palmitamide (Example 13). To a TFA/TIPS (95:5, 10 mL) solution, Intermediate 9 (0.50 g, 0.50 mmol, 1.0 equiv) was added at 0° C. The reaction mixture was heated to 50° C. and stirred for 2 h, before it was concentrated in vacuo, and the crude residue was purified via RP-HPLC. All fractions containing product were combined, concentrated to 5 mL in vacuo, prior to lyophilization, to recover Example 13 (28 mg, 6%) as a white powder.

LCMS (ESI-MS m/z): mass calcd for C₃₇H₇₂N₇₀₇S [M+H]⁺, 758.5; found 758.4

### Example 14

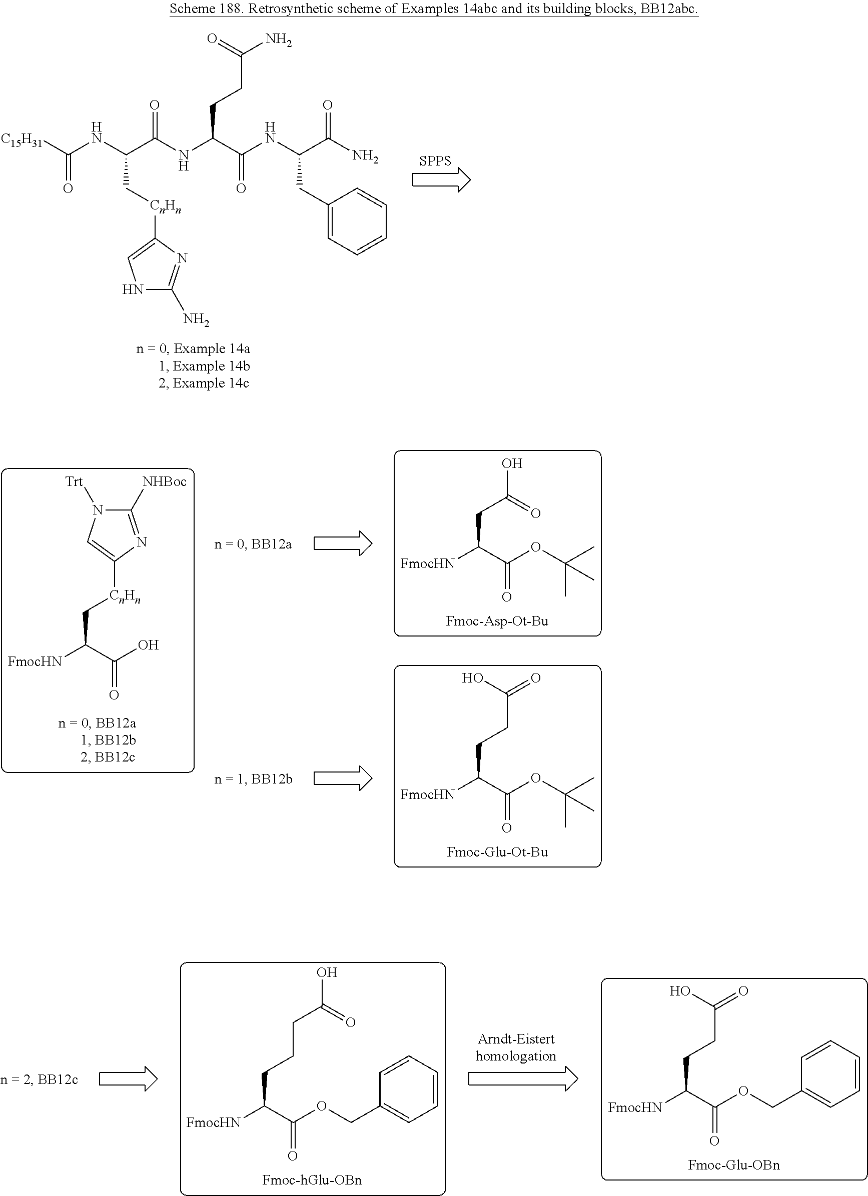

Examples 14a, b and c are palmitoylated tripeptide amides, characterized by a non-natural amino acid in the third position from the C-terminus. The latter amino acid is analogous to Ala, whereas the methyl side-chain is functionalized with a 2-aminoimidazole moiety (through C-4 of the imidazole ring), either directly (n=0, as in Example 14a), or spaced apart by one (n=1,

Example 14b) or two (n=2, Example 14c) methylene groups. Examples 14a, b and c were synthesized via standard SPPS, using Fmoc-amino acids and analogous conditions as before for standard SPPS with Rink AM resin. Non-natural N-Fmoc protected aminoacids as BB12abc, where synthesized via ad hoc synthesis (detailed in the experimental) respectively from Fmoc-Asp-OtBu, Fmoc-Glu-OtBu and Fmoc-hGlu-OBn. In order to be deployed in SPPS, BB12abc had the two basic nitrogens of the 2-aminoimidazole side-chain respectively Boc and Trt protected. Whilst Fmoc-Asp-OtBu and Fmoc-Glu-OtBu are commercially available, Fmoc-hGlu-OBn was synthesized from Fmoc-Glu-OBn via Arndt-Eistert homologation and protecting groups interconversion, as detailed in the experimental section. The carboxylate side chain of Fmoc-Asp-tBu, Fmoc-Glu-OtBu and Fmoc-hGlu-OBn could be converted into the 2-aminoimidazole through the same sequence of transformations, therefore in the experimental section are reported only the synthesis of BB14b as an example, and the synthesis of Fmoc-hGlu-OBn, which is not commercially available.

### Example 14.1 Experimental procedure-Synthesis of Fmoc-hGlu-OBn

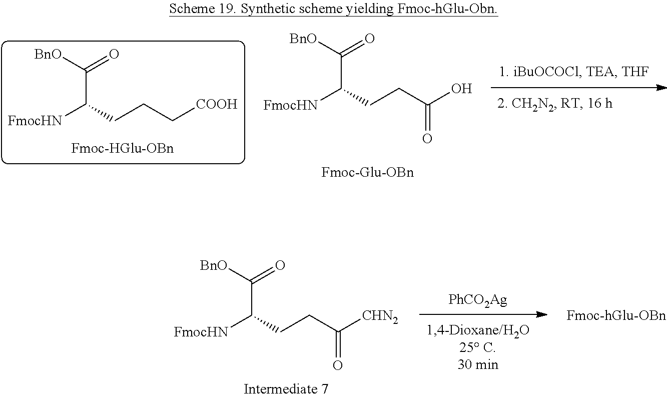

(S)-5-((((9H-fluoren-9-yl)methoxy)carbonyl)amino)-6-(benzyloxy)-2,6-dioxohexane-1-diazonium (Intermediate 7). To a THF (300 mL) solution of Fmoc-Glu-OBn (30 g, 65 mmol, 1.0 equiv), TEA (27 mL, 200 mmol, 3.0 equiv) was added at 0° C., followed by isobutyl chloroformate (17 mL, 130 mmol, 2.0 equiv). The resulting mixture was stirred at 0° C. for 30 min and the so-formed solids were filtered and washed with THF (50 mL). The filtrates were combined in a round-bottom flask and a ether (150 mL) solution of freshly prepared diazomethane (excess) was added. The reaction mixture was stirred at 0° C. for 1 h, before it was warmed to 25° C. and stirred additional 2 h. The reaction mixture was quenched by the addition of AcOH (2.0 mL), diluted with water (500 mL), and extracted with EtOAc (3×500 mL). The combined organic layer was washed with saturated aqueous NaHCO₃ (500 mL), dried over Na₂SO₄ and concentrated in vacuo. The crude residue was purified via flash column chromatography (SiO₂, EtOAc,/pet-ether, from 0:1 to 1:4) to recover intermediate 7 (12 g, 38%) as a light-green solid.

R_{f}=0.3 (SiO₂, EtOAc/pet-ether, 2:3).

LCMS (ESI-MS m/z): mass calcd for C₂₈H₂₇N₃O₅ [M+H]⁺, 485.19; found, 484.31.

(S)-5-((((9H-fluoren-9-yl)methoxy)carbonyl)amino)-6-(benzyloxy)-6-oxohexanoic acid (Fmoc-hGlu-OBn). To a dioxane/water (1.2 L, 5:1) solution of intermediate 7 (12 g, 25 mmol, 1.0 equiv), PhCO₂Ag (0.56 g, 2.4 mmol, 0.10 equiv) was added. The resulting mixture was sonicated at 25° C. for 30 min, before the reaction was quenched by the addition of 1 N aqueous HCl (1.0 mL), and extracted with EtOAc (3×500 mL). The combined organic layer was dried over Na₂SO₄ and concentrated in vacuo. The combined organic layer was washed with saturated aqueous NaHCO₃ (500 mL), dried over Na₂SO₄ and concentrated in vacuo. The crude residue was purified via flash column chromatography (SiO₂, EtOAc,/pet-ether, from 0:1 to 3:2) to recover Fmoc-hGlu-OBn (10 g, 87%) as a white solid.

R_{f}=0.4 (SiO₂, EtOAc).

LCMS (ESI-MS m/z): mass calcd for C₂₈H₂₈NO6 [M+H]⁺, 474.19; found, 474.35.

### Example 14.2 Experimental procedure-Synthesis of BB12b and Example 14b

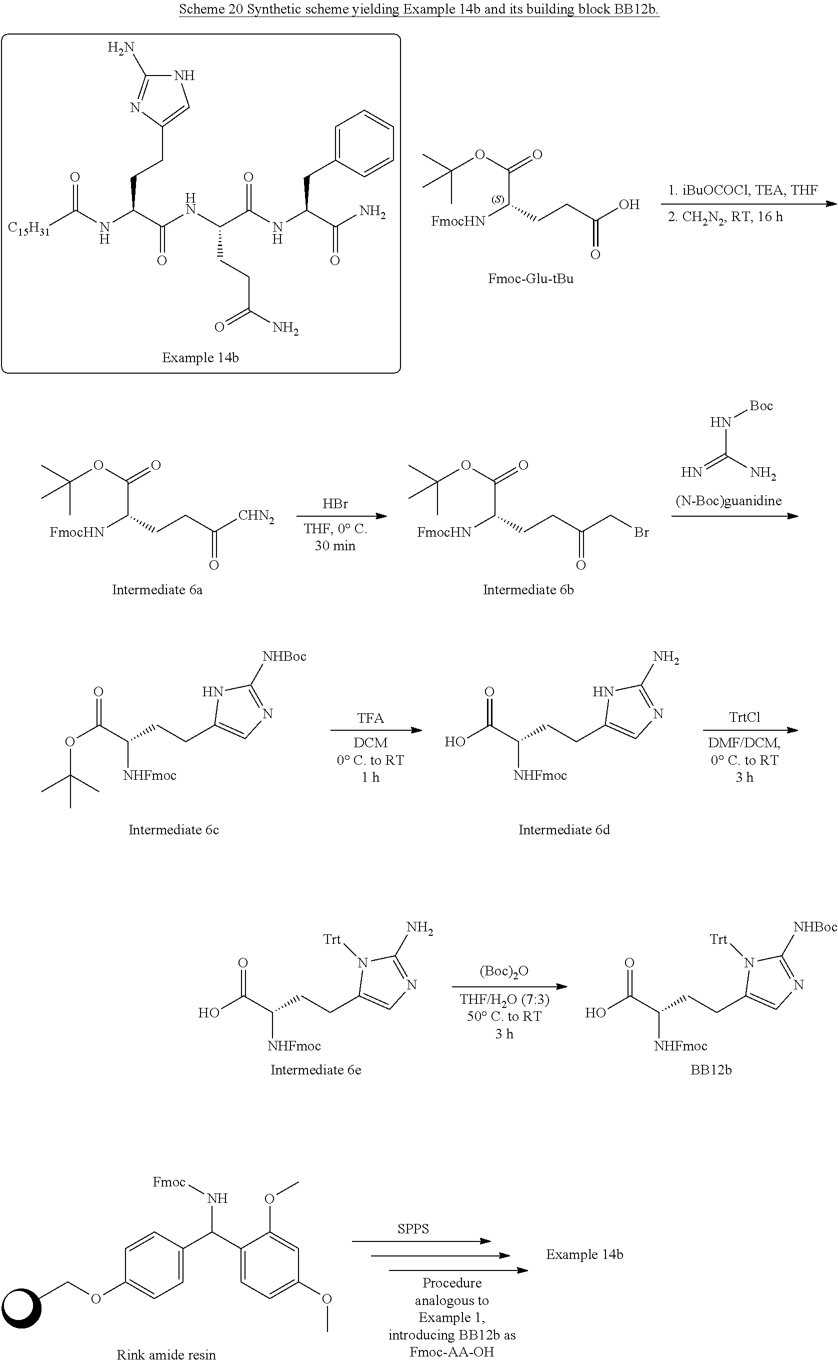

(S)-5-((((9H-fluoren-9-yl)methoxy)carbonyl)amino)-6-(tert-butoxy)-2,6-dioxohexane-1-diazonium (Intermediate 6a). To a THF (500 mL) solution of Fmoc-Glu-tBu (50 g, 120 mmol, 1.0 equiv), TEA (37 mL, 290 mmol, 2.5 equiv) was added at −10° C., followed by isobutyl chloroformate (30 mL, 230 mmol, 2.0 equiv). The resulting mixture was stirred at 0° C. for 15 min and the so-formed solids were filtered and washed with THF (50 mL). The filtrates were combined in a round-bottom flask and a ether (200 mL) solution of freshly prepared diazomethane (excess) was added. The reaction mixture was stirred at 25° C. for 2 h, before it was quenched by the addition of AcOH (2.0 mL) and diluted with water (500 mL). The resulting mixture was extracted with EtOAc (3×500 mL), and the combined organic layer was washed with saturated aqueous NaHCO₃ (500 mL), dried over Na₂SO₄ and concentrated in vacuo. The crude residue was purified via flash column chromatography (SiO₂, EtOAc,/pet-ether, from 0:1 to 1:4) to recover intermediate 6a (26 g, 51%) as a yellow liquid.

R_{f}=0.6 (SiO₂, EtOAc/pet-ether, 2:3).

¹H NMR (400 MHZ, DMSO-d₆), δ: 7.86 (d, J=6.8 Hz, 2H), 7.72 (d, J=7.6 Hz, 2H), 7.78 (d, J=8.0 Hz, 1H), 7.42 (t, J=7.6 Hz, 2H), 7.33 (t, J=7.6 Hz, 2H), 6.07 (bs, 1H), 4.32-4.19 (m, 3H), 3.90-3.85 (m, 1H), 2.47-2.32 (m, 2H), 2.00-1.89 (m, 1H), 1.83-1.73 (m, 1H), 1.39 (s, 9H).

tert-butyl(S)-2-((((9H-fluoren-9-yl)methoxy)carbonyl)amino)-6-bromo-5-oxohexanoate (Intermediate 6b). To a THF (200 mL) solution of intermediate 6a (20 g, 44 mmol, 1.0 equiv), 33% HBr in AcOH (12 mL, 49 mmol, 1.1 equiv) was added at 0° C. The resulting mixture was stirred for 30 min at 0° C., before it was diluted with water (50 mL) and it was extracted with EtOAc (3×350 mL). The combined organic layer was washed with brine (500 mL), dried over Na₂SO₄ and concentrated in vacuo. The crude residue was purified via flash column chromatography (SiO₂, EtOAc,/pet-ether, from 0:1 to 5:17) to recover intermediate 6b (12 g, 55%) as a pale yellow liquid.

R_{f}=0.6 (SiO₂, EtOAc/pet-ether, 2:3).

¹H NMR (400 MHZ, DMSO-d₆), δ: 7.90 (d, J=7.6 Hz, 2H), 7.83-7.62 (m, 3H), 7.42 (t, J=7.2 Hz), 7.37-7.28 (m, 2H), 4.48-4.14 (m, 2H), 3.92-3.84 (m, 1H), 2.73-2.62 (m, 1H), 2.09 (s, 1H), 1.99-1.67 (m, 3H), 1.58-1.47 (m, 1H), 1.39 (s, 9H).

tert-butyl(S)-2-((((9H-fluoren-9-yl)methoxy)carbonyl)amino)-4-(2-((tert-butoxycarbonyl)amino)-1H-imidazol-5-yl) butanoate (Intermediate 6c).

To a DMF (100 mL) solution of intermediate 6b (10 g, 20 mmol, 1.0 equiv), Boc-guanidine (9.5 g, 60 mmol, 3.0 equiv) was added, followed by powdered molecular sieves (10 g). The resulting mixture was stirred at 25° C. for 16 h, before it was quenched by the addition of water (100 mL), and extracted with EtOAc (3×200 mL). The combined organic layer was washed with brine, dried over Na₂SO₄ and concentrated in vacuo. The crude residue was purified via flash column chromatography (SiO₂, EtOAc/pet-ether, from 1:9 to 7:3) to recover intermediate 6c (6.0 g, 53%) as an off-white solid.

R_{f}=0.5 (SiO₂, EtOAc).

LCMS (ESI-MS m/z): mass calcd for C₃₁H₃₉N₄O₆ [M+H]+563.3; found 563.3.

(S)-2-((((9H-fluoren-9-yl)methoxy)carbonyl)amino)-4-(2-amino-1H-imidazol-5-yl) butanoic acid (Intermediate 6d). To a TFA/DCM (100 mL, 2:3) solution, intermediate 6c (5.0 g, 8.9 mmol, 1.0 equiv.) was added at 0° C. The resulting mixture was warmed to 25° C. and stirred for 1 h, before it was concentrated in vacuo. The crude residue was co-evaporated with toluene (5 mL) to recover intermediate 6d (4.6 g, quant.) as a crude material, which was used as such in the next synthetic step.

R_{f}=0.3 (SiO₂, MeOH/DCM, 1:9).

LCMS (ESI-MS m/z): mass calcd for C₂₂H₂₃N₄O₄ [M+H]⁺, 407.2; found 407.4.

(S)-2-((((9H-fluoren-9-yl)methoxy)carbonyl)amino)-4-(2-amino-1-trityl-1H-imidazol-5-yl) butanoic acid (Intermediate 6e). To a DCM/DMF (45 mL, 1:1) solution of intermediate 6d (4.5 g, 11 mmol, 1.0 equiv.), TEA (3.8 mL, 28 mmol, 2.5 equiv.) was added at 0° C., followed by Trt-Cl (4.6 g, 16.1 mmol, 1.5 equiv.). The resulting mixture was warmed to 25° C. and stirred for 3 h, before it was concentrated in vacuo. The residue was diluted with water (45 mL) and extracted with EtOAc (3×100 mL), and the combined organic layer was dried over Na₂SO₄ and concentrated in vacuo. The crude residue was purified via flash column chromatography (SiO₂, MeOH/DCM, from 1:99 to 1:9) to recover intermediate 6e (2.2 g, 38%) as a white solid. R_{f}=0.5 (SiO₂, MeOH/DCM, 1:9).

LCMS (ESI-MS m/z): mass calcd for C₄₁H₃₇N₄O₄ [M+H]⁺, 649.28; found 649.27.

(S)-2-((((9H-fluoren-9-yl)methoxy)carbonyl)amino)-4-(2-((tert-butoxycarbonyl)amino)-1-trityl-1H-imidazol-5-yl) butanoic acid (BB12b). To a THF/water (20 mL, 1:1) solution of intermediate 6d (2.0 g, 3.1 mmol, 2.0 equiv), NaHCO₃ (0.51 g, 6.2 mmol, 2.0 equiv) was added, followed by Boc₂O (1.4 mL, 62 mmol, 20 equiv). The resulting mixture was warmed to 50° C. and stirred for 3 h, before it was quenched by the addition of water (50 mL) and extracted with DCM (2× 50 mL). The combined organic layer was dried over Na₂SO₄ and concentrated in vacuo. The crude residue was purified via flash column chromatography (SiO₂, MeOH/DCM, from 1:99 to 1:9) to recover BB12b (500 mg, 47%) as an off-white solid.

R_{f}=0.7 (SiO₂, MeOH/DCM, 1:9).

LCMS (ESI-MS m/z): mass calcd for C₄₆H₄₅N₄O₆ [M+H]⁺, 749.3; found 749.1.

### Compounds made following the synthesis of BB12b

BB12a, starting from Fmoc-Asp-tBu, (used in the synthesis of Example 14a).
BB12c, starting from Fmoc-hGlu-Bn (used in the synthesis of Example 14c).

(S)—N₁—((S)-1-amino-1-oxo-3-phenylpropan -2-yl)-2-((S)-4-(2-amino-1H-imidazol-4-yl)-2-palmitamidobutanamido)pentanediamide TFA salt (Example 14b) was prepared according the general procedure for SPPS using 0.7 g Rink amide AM resin. The crude peptide was purified by preparative RP-HPLC, all fractions containing the product were combined and concentrated in vacuo to 5 mL, prior to lyophilization, to afford peptide Example 14b (33 mg, 15%) as a white powder.

HRMS (ESI-MS m/z): mass calcd for C₃₇H₆₁N₈O₅ [M+H]⁺, 697.4759; found, 697.4726.

### Compounds Made Following the Synthesis of Example 14b (55)

Example 14a (62).
Example 14c (56).

### Example 15. Synthetic Approach

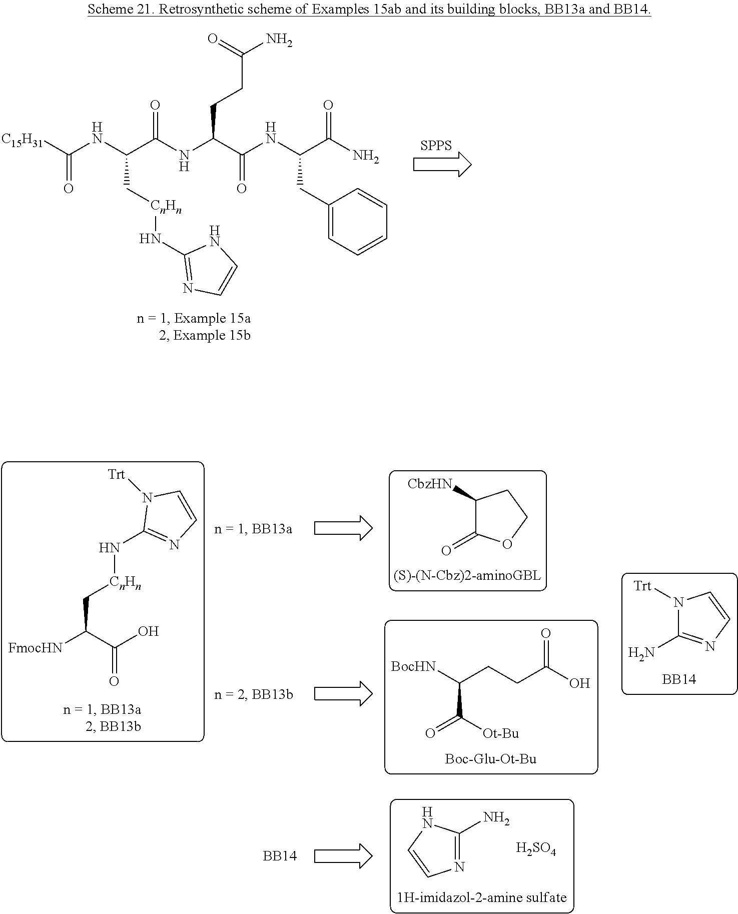

Examples 15a and b are palmitoylated tripeptide amides, characterized by a non-natural amino acid in the third position from the C-terminus. The latter amino acid is analogous to Ala, whereas the methyl side-chain is functionalized with a 2-aminoimidazole moiety (through the 2-amino substituent of the imidazole ring), either spaced apart by one (n=1, Example 15a) or two (n=2, Example 15b) methylene groups. Examples 15a and b were synthesized via standard SPPS, using Fmoc-amino acids and analogous conditions as before for standard SPPS with Rink AM resin. Non-natural N-Fmoc protected aminoacids as BB13ab, where synthesized via ad hoc synthesis (detailed in the experimental) respectively from commercially available(S)—(N-Cbz)₂-aminoGBL or Boc-Glu-OtBu, and the ad hoc synthesized BB14. In order to be deployed in SPPS, BB13ab, other than the alpha-nitrogen Fmoc-protected, had the basic nitrogen of the imidazole ring Trt protected. After several protecting group interconversions, the sidechain of(S)—(N-Cbz)₂-aminoGBL and Boc-Glu-OtBu were converted to aldehyde, prior to reacting with BB14 via reductive amination. BB14 was obtained one protecting group interconversion and Trt protection, from commercially available 1H-imidazol-2-amine sulfate.

### Example 15.1 Experimental Procedure-Synthesis of BB14

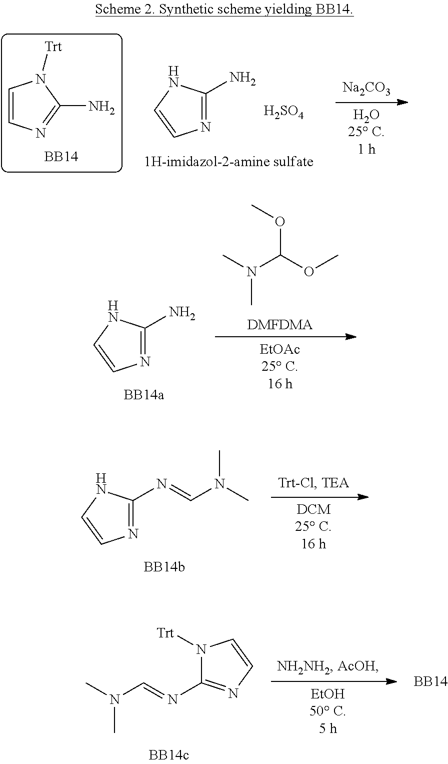

1H-imidazol-2-amine (BB14a). To an aqueous (100 mL) solution of 1H-imidazol-2-amine sulfate (10 g, 38 mmol, 1.0 equiv), Na₂CO₃ (12 g, 110 mmol, 3.0 equiv) was added. The resulting mixture was stirred at 25° C. for 1 h, before it was concentrated in vacuo. The residue was dissolved in EtOH (200 mL), as the resulting mixture was stirred for at 25° C. for 1 h, before filtering over Celite®. The filtrate was concentrated in vacuo, to recover BB14a (5.0 g, quant.) as a crude material, which was used as such in the next synthetic step.

(E)-N′-(1H-imidazol-2-yl)-N,N-dimethylformimidamide (BB14b). A DMFDMA/EtOAc (100 mL, 1:1) solution of crude BB14a (5.0 g, 38 mmol, 1.0 equiv) was stirred at 25° C. for 16 h, before it was concentrated in vacuo, to recover BB14b (10 g, quant.) as a crude material, which was used as such in the next synthetic step.

LCMS (ESI-MS m/z): mass calcd for C₆H₁₁N₄ [M+H]⁺, 139.10; found 138.97.

(E)-N,N-dimethyl-N′-(1-trityl-1H-imidazol-2-yl)formimidamide (BB14c). To a DCM (100 mL) solution of crude BB14b (10 g, 38 mmol, 1.0 equiv), TEA (15 mL, 110 mmol, 2.9 equiv) was added at 0° C., followed by Trt-Cl (10 g, 36 mmol, 0.9 equiv). The resulting mixture was warmed to 25° C. and stirred for 16 h, before it was quenched by the addition of water (100 mL), and extracted with EtOAc (3×200 mL). The combined organic layer was dried over Na₂SO₄ and concentrated in vacuo. The crude residue was purified via flash column chromatography (SiO₂, MeOH/DCM, from 1:99 to 1:9) to recover BB14c (7.0 g, 49%) as a white solid.

R_{f}=0.6 (SiO₂, EtOAc).

LCMS (ESI-MS m/z): mass calcd for C₂₅H₂₅N₄ [M+H]⁺, 381.21; found 381.31.

1-trityl-1H-imidazol-2-amine (BB14). To a EtOH (70 mL) solution of BB14c (7.0 g, 18 mmol, 1.0 equiv), AcOH (5.1 mL, 92 mmol, 5.0 equiv) was added at 0° C., followed by hydrazine hydrate (4.5 mL, 92 mmol, 5.0 equiv). The resulting mixture was warmed to 50° C. and was stirred for 5 h, before it was concentrated in vacuo. The residue was diluted with DCM (100 mL) and 1 N aqueous NaOH (50 mL), and the resulting mixture was stirred for 30 min at 25° C. After layer-separation, the organic layer was dried over Na₂SO₄ and concentrated in vacuo, to recover BB14 (5.5 g, 93%) as a white solid.

R_{f}=0.3 (SiO₂, EtOAc).

LCMS (ESI-MS m/z): mass calcd for C₂₂H₂₀N₃ [M+H]⁺, 326.17; found 326.26.

### Example 15. Experimental Procedure-BB13a and Example 15a

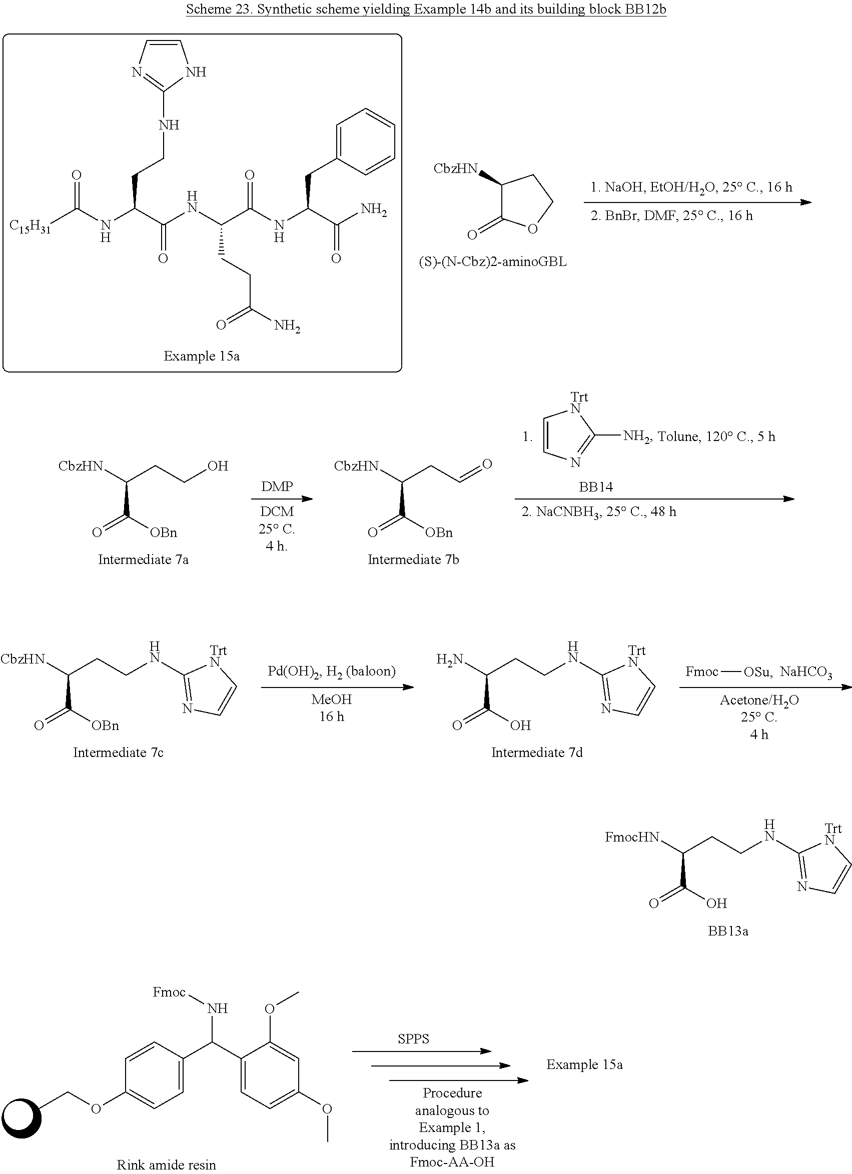

Benzyl ((benzyloxy)carbonyl)-L-homoserinate (Intermediate 7a). To a EtOH (360 mL) solution of(S)—(N-Cbz)₂-aminoGBL (30 g, 120 mmol, 1.0 equiv), an aqueous (50 mL) solution of NaOH (4.7 g, 120 mmol, 1.0 equiv) was added at 0° C. The resulting mixture was warmed to 25° C. and stirred for 16 h, before it was concentrated in vacuo, and the residue was triturated in EtOH (3×100 mL). The so-formed solid was filtered and dissolved in DMF (300 mL), before BnBr (14 mL, 120 mmol, 1.0 equiv) was added at at 0° C. The resulting mixture was warmed to 25° C. and stirred for 16 h, before it was quenched by the addition of water (500 mL), and extracted with EtOAc (3×750 mL). The combined organic layer was dried over Na₂SO₄ and concentrated in vacuo. The crude residue was purified via flash column chromatography (SiO₂, EtOAc/pet-ether, from 0:1 to 3:7) to recover Intermediate 7a (30 g, quant.) as a white solid.

R_{f}=0.4 (SiO₂, EtOAc/pet-ether, 3:2).

LCMS (ESI-MS m/z): mass calcd for C₁₉H₂₂NNaO₅ [M+H]⁺, 366.13; found 366.30. Benzyl(S)-2-(((benzyloxy)carbonyl)amino)-4-oxobutanoate (intermediate 7b). To a DCM (200 mL) solution of Intermediate 7a (10 g, 29 mmol, 1.0 equiv), DMP (18 g, 43 mmol, 1.5 equiv) was added at 0° C. The resulting mixture was warmed to 25° C. and stirred for 4 h, before it was quenched by the addition of 10% aqueous sodium thiosulfate (100 mL) and stirred additional 15 min. The resulting mixture was extracted with DCM (3×250 mL), and the combined organic layer was dried over Na₂SO₄ and concentrated in vacuo. The crude residue was purified via flash column chromatography (SiO₂, EtOAc/pet-ether, from 0:1 to 1:3) to recover Intermediate 7b (7.1 g, 71%) as a white solid.

R_{f}=0.6 (SiO₂, EtOAc/pet-ether, 3:2).

¹H NMR (400 MHZ, CDCl₃), δ: 9.62 (s, 1H), 7.38-7.28 (m, 10H), 5.69 (d, J=7.6 Hz, 1H), 5.20-5.08 (m, 4H), 4.73-4.65 (m, 1H), 3.19-3.04 (m, 2H).

Benzyl(S)-2-(((benzyloxy)carbonyl)amino)-4-((1-trityl-1H-imidazol-2-yl)amino) butanoate (Intermediate 7c). To a toluene (40 mL) solution of Intermediate 7b (2.0 g, 5.9 mmol, 1.0 equiv), BB14 (1.9 g 5.9 mmol, 1.0 equiv) was added. The resulting mixture was heated to 120° C. and stirred for 5 h, before it was cooled to 0° C., and NaCNBH₃ (1.1 g, 18 mmol, 3.0 equiv) was added. The resulting mixture was warmed to 25° C. and stirred for 48 h, before it was quenched by the addition of saturated aqueous ammonium chloride (50 mL), and extracted with EtOAc (3×200 mL). The combined organic layer was dried over Na₂SO₄ and concentrated in vacuo. The crude residue was purified via flash column chromatography (SiO₂, MeOH/DCM, from 1:99 to 3:97) to recover Intermediate 7c (1.2 g, 31%) as a light-yellow gum.

R_{f}=0.4 (SiO₂, MeOH/DCM, 1:19).

LCMS (ESI-MS m/z): mass calcd for C₄₁H₃₉N₄O₄ [M+H]⁺, 651.30; found 651.20.

(S)-2-amino-4-((1-trityl-1H-imidazol-2-yl)amino) butanoic acid (Intermediate 7d). To a MeOH (20 mL) solution of Intermediate 7c (1.0 g, 1.5 mmol, 1.0 equiv), 10% Pd(OH)₂ (0.40 g, 40% wt) was added, and the resulting mixture was stirred under H₂ (balloon) atmosphere for 16 h, before it was filtered through Celite®. The filtrate was concentrated in vacuo to recover intermediate 7d (0.6 g, 91%) as an off-white solid.

LCMS (ESI-MS m/z): mass calcd for C₂₆H₂₇N₄O₂ [M+H]⁺, 427.21; found 427.50.

(S)-2-((((9H-fluoren-9-yl)methoxy)carbonyl)amino)-4-((1-trityl-1H-imidazol-2-yl)amino) butanoic acid (BB13a). To a water/acetone (9 mL, 3:1) solution of Intermediate 7d (0.60 g, 1.4 mmol, 1.0 equiv), NaHCO₃ (0.23 g, 2.8 mmol, 2.0 equiv) was added at 0° C., followed by Fmoc-OSu (0.47 g, 1.4 mmol, 1.0 equiv). The resulting mixture was warmed to 25° C. and stirred for 4 h, before it was quenched by the addition of 10% aqueous citric acid (50 mL), and extracted with EtOAc (3×100 mL). The combined organic layer was dried over Na₂SO₄ and concentrated in vacuo. The crude residue was purified via flash column chromatography (SiO₂, MeOH/DCM, from 1:99 to 1:24) to recover BB13a (0.45 g, 50%) as a white solid.

R_{f}=0.3 (SiO₂, MeOH/DCM, 1:19).

LCMS (ESI-MS m/z): mass calcd for C₄₁H₃₇N₄O₄ [M+H]⁺, 649.28; found 649.20.

(S)-2-((S)-4-((1H-imidazol-2-yl)amino)-2-palmitamidobutanamido)-N₁—((S)-1-amino-1-oxo-3-phenylpropan -2-yl)pentanediamide TFA salt (Example 15a) was prepared according the general procedure for SPPS using 0.7 g Rink amide AM resin. The crude peptide was purified by preparative RP-HPLC, all fractions containing the product were combined and concentrated in vacuo to 5 mL, prior to lyophilization, to afford peptide Example 15a (16 mg, 6.5%) as a white powder.

HRMS (ESI-MS m/z): mass calcd for C₃₇H₆₁N₈O₅ [M+H]⁺, 697.4759; found, 697.4742. Compounds made according to example 15 are 65 and 66

### Example 15. Experimental procedure-BB13b and Example 15b

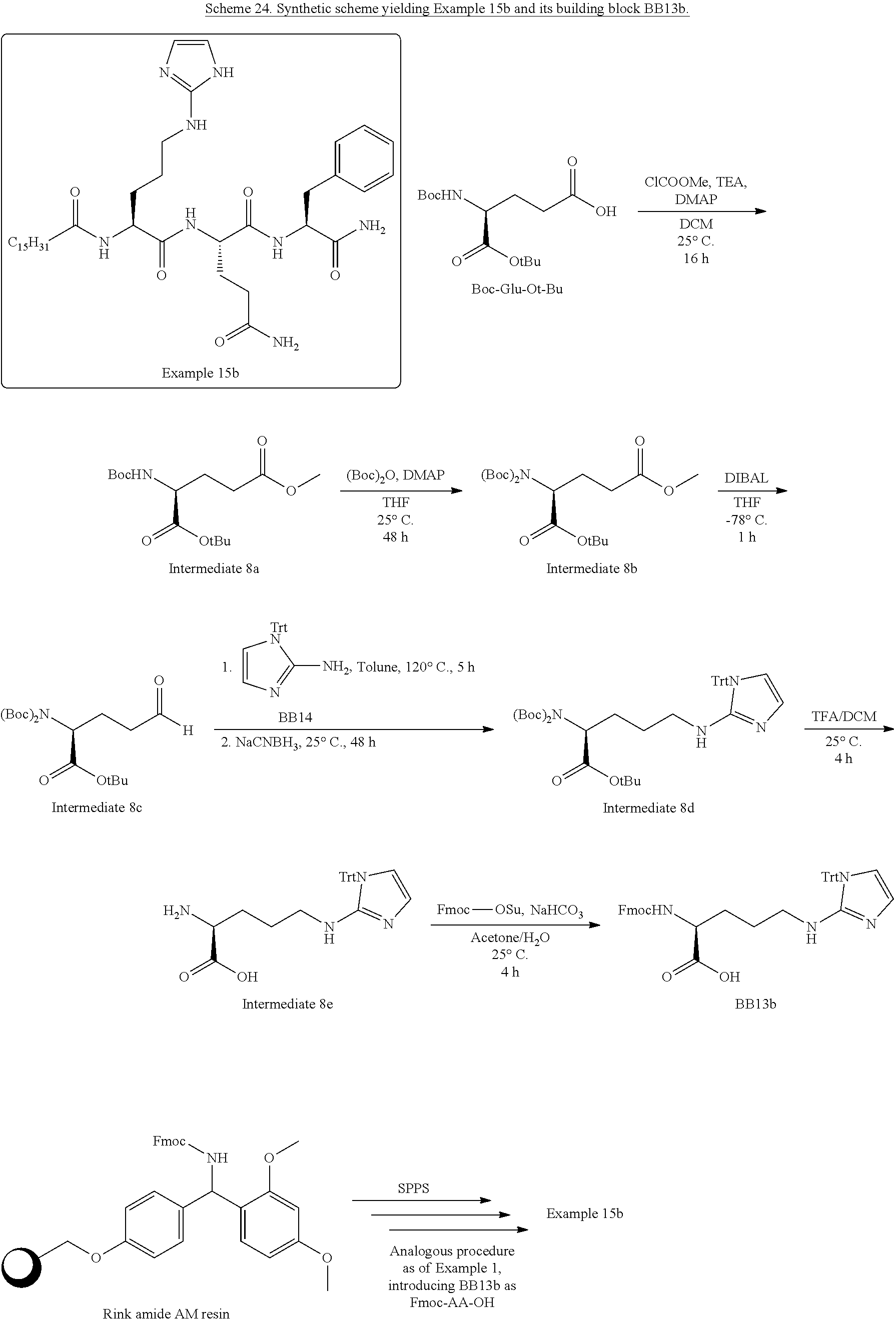

1-(Tert-butyl)₅-methyl (tert-butoxycarbonyl)-L-glutamate (Intermediate 8a). To a DCM (500 mL) solution of Boc-Glu-OtBu (25 g, 82 mmol, 1.0 equiv), TEA (23 mL, 160 mmol, 2.0 equiv) was added at 0° C., followed by DMAP (1.0 g, 8.2 nnol, 0.1 equiv) and CICOOMe (9.4 mL, 120 mmol, 1.5 equiv). The resulting mixture was warmed to 25° C. and stirred for 3 h, before it was quenched by the addition of water (200 mL), and extracted with DCM (3×500 mL). The combined organic layer was dried over Na₂SO₄ and concentrated in vacuo, to recover Intermediate 8a (25 g, quant.) as a crude material, which was used as such in the next synthetic step.

R_{f}=0.5 (SiO₂, EtOAc/pet-ether, 1:4).

LCMS (ESI-MS m/z): mass calcd for C₁₅H₂₈NO6 [M+H]⁺, 318.19; found 318.23.

1-(Tert-butyl)₅-methyl N,N-bis(tert-butoxycarbonyl)- L-glutamate (Intermediate 8b). To a THF (500 mL) solution of Intermediate 8a (25 g, 79 mmol, 1.0 equiv), DMAP (4.8 g, 39 mmol, 1.0 equiv) was added at 0° C., followed by Boc₂O (34 mL, 160 mmol, 2.0 mmol). The resulting mixture was warmed to 25° C. and stirred for 36 h, before with was quenched by the addition of water (300 mL), and extracted with DCM (3×500 mL). The combined organic layer was dried over Na₂SO₄ and concentrated in vacuo. The crude residue was purified via flash column chromatography (SiO₂, EtOAc/pet-ether, from 0:1 to 1:9) to recover Intermediate 8b (19 g, 60%) as a white solid.

R_{f}=0.6 (SiO₂, EtOAc/pet-ether, 1:4).

LCMS (ESI-MS m/z): mass calcd for C₂₀H₃₅NO8 [M+H]⁺, 418.24; found 418.34.

Tert-butyl(S)-2-(bis(tert-butoxycarbonyl)amino)-5-oxopentanoate (Intermediate 8c). To a diethyl ether (100 mL) solution of Intermediate 8b (5.0 g, 12 mmol, 1.0 equiv), a 1 M solution of DIBAL-H in hexane (18 mL, 18 mmol, 1.5 equiv) was added drop-wise, over 5 min, at −78° C. The resulting mixture was quenched by the addition of 10% aqueous sodium potassium tartrate (25 mL), and stirred additional 30 min, before it was extracted with EtOAc (3×100 mL). The combined organic layer was dried over Na₂SO₄ and concentrated in vacuo. The crude residue was purified via flash column chromatography (SiO₂, EtOAc/pet-ether, from 0:1 to 1:4) to recover Intermediate 8c (4.0 g, 86%) as a white solid.

R_{f}=0.4 (SiO₂, EtOAc/pet-ether, 1:4).

¹H NMR (400 MHZ, CDCl₃), δ: 9.80-9.72 (m, 1H), 4.80-4.72 (m, 1H), 2.63-2.38 (m, 4H), 1.58-1.38 (m, 27H).

Tert-butyl(S)-2-(bis(tert-butoxycarbonyl)amino)-5-((1-trityl-1H-imidazol-2-yl)amino)pentanoate (Intermediate 8d). To a toluene (50 mL) solution of Intermediate 8c (2.5 g, 6.4 mmol, 1.0 equiv), BB14 (2.1 g, 6.4 mmol, 1.0 equiv) was added. The resulting mixture was heated to 120° C. and stirred for 5 h, before it was cooled to 0° C., and NaCNBH₃ (0.73 g, 20 mmol, 3.0 equiv) was added. The resulting mixture was warmed to 25° C. and stirred for 48 h, before it was quenched by the addition of saturated aqueous ammonium chloride (50 mL), and extracted with EtOAc (3×200 mL). The combined organic layer was dried over Na₂SO₄ and concentrated in vacuo. The crude residue was purified via flash column chromatography (SiO₂, EtOAc/pet-ether, from 1:4 to 7:3) to recover Intermediate 8d (0.9 g, 20%) as a white solid. R_{f}=0.4 (SiO₂, MeOH/DCM, 1:19).

LCMS (ESI-MS m/z): mass calcd for C₄₁H₅₃N₄O₆ [M+H]⁺, 697.40; found 697.56.

(S)-2-amino-5-((1-trityl-1H-imidazol-2-yl)amino)pentanoic acid (Intermediate 8e). To a TFA/DCM (18 mL, 1:1) solution, Intermediate 8d (0.9 g, 1.3 mmol, 1.0 equiv) was added at 0° C. The resulting mixture was warmed to 25° C. and stirred fot 4 h, before it was concentrated in vacuo.

The crude residue was triturated in diethyl ether (50 mL) to recover crude Intermediate 8e (0.5 g, quant.) as a light-brown solid, which was used as such in the next synthetic step.

LCMS (ESI-MS m/z): mass calcd for C₂₇H₂₉N₄O₂ [M+H]⁺, 441.23; found 441.35.

(S)-2-((((9H-fluoren-9-yl)methoxy)carbonyl)amino)-5-((1-trityl-1H-imidazol-2-yl)amino)pentanoic acid (BB13b). To a water/acetone (7.5 mL, 2:1) solution of intermediate 8e (0.50 g, 1.1.mmol, 1.0 equiv), NaHCO₃ (0.19 g, 2.3 mmol, 2.0 equiv) was added at 0° C., followed by Fmoc-OSu (0.38 g, 1.1 mmol, 1.0 equiv). The resulting mixture was warmed to 25° C. and stirred for 4 h, before it was quenched by the addition of 10% aqueous citric acid (50 mL), and extracted with EtOAc (3×100 mL). The combined organic layer was dried over Na₂SO₄ and concentrated in vacuo. The crude residue was purified via flash column chromatography (SiO₂, MeOH/DCM, from 1:99 to 3:47) to recover BB13b (0.9 g, 20%) as an off-white solid.

R_{f}=0.3 (SiO₂, MeOH/DCM, 1:19).

LCMS (ESI-MS m/z): mass calcd for C₄₂H₃₉N₄O₄ [M+H]⁺, 663.30; found 663.47.

(S)-2-((S)-5-((1H-imidazol-2-yl)amino)-2-palmitamidopentanamido)-N₁—((S)-1-amino-1-oxo-3-phenylpropan -2-yl)pentanediamide TFA (Example 15b) was prepared according the general procedure for SPPS using 0.7 g Rink amide AM resin. The crude peptide was purified by preparative RP-HPLC, all fractions containing the product were combined and concentrated in vacuo to 5 mL, prior to lyophilization, to afford peptide Example 15b (75 mg, 30%) as a white powder.

HRMS (ESI-MS m/z): mass calcd for C₃₈H₆₂N₈O₅ [M+H]⁺, 711.4916; found, 711.4890

### REFERENCES

1. Atherton, E.; Clive, D. L. J.; Sheppard, R. C., Polyamide supports for polypeptide synthesis. Journal of the American Chemical Society 1975, 97 (22), 6584-6585.
2. Albericio, F.; Kneib-Cordonier, N.; Biancalana, S.; Gera, L.; Masada, R. I.; Hudson, D.; Barany, G., Preparation and application of the 5-(4-(9-fluorenylmethyloxycarbonyl)aminomethyl-3,5-dimethoxyphenoxy)-valeric acid (PAL) handle for the solid-phase synthesis of C-terminal peptide amides under mild conditions. The Journal of Organic Chemistry 1990, 55 (12), 3730-3743.
3. Rink, H., Solid-phase synthesis of protected peptide fragments using a trialkoxy-diphenyl-methylester resin. Tetrahedron Letters 1987, 28 (33), 3787-3790.
4. Barlos, K.; Gatos, D.; Kallitsis, J.; Papaphotiu, G.; Sotiriu, P.; Wenqing, Y.; Schäfer, W., Darstellung geschützter peptid-fragmente unter einsatz substituierter triphenylmethyl-harze. Tetrahedron Letters 1989, 30 (30), 3943-3946.
5. Nahm, S.; Weinreb, S. M., N-methoxy-n-methylamides as effective acylating agents. Tetrahedron Letters 1981, 22 (39), 3815-3818.
6. Kwon, H.; Kim, Y.; Park, K.; Choi, S. A.; Son, S.-H.; Byun, Y., Structure-based design, synthesis, and biological evaluation of Leu-Arg dipeptide analogs as novel hepsin inhibitors. Bioorganic & Medicinal Chemistry Letters 2016, 26 (2), 310-314.
7. Venkatraman, S.; Bogen, S. L.; Arasappan, A.; Bennett, F.; Chen, K.; Jao, E.; Liu, Y.-T.; Lovey, R.; Hendrata, S.; Huang, Y.; Pan, W.; Parekh, T.; Pinto, P.; Popov, V.; Pike, R.; Ruan, S.; Santhanam, B.; Vibulbhan, B.; Wu, W.; Yang, W.; Kong, J.; Liang, X.; Wong, J.; Liu, R.; Butkiewicz, N.; Chase, R.; Hart, A.; Agrawal, S.; Ingravallo, P.; Pichardo, J.; Kong, R.; Baroudy, B.; Malcolm, B.; Guo, Z.; Prongay, A.; Madison, V.; Broske, L.; Cui, X.; Cheng, K.-C.; Hsieh, Y.; Brisson, J.-M.; Prelusky, D.; Korfmacher, W.; White, R.; Bogdanowich-Knipp, S.; Pavlovsky, A.; Bradley, P.; Saksena, A. K.; Ganguly, A.; Piwinski, J.; Girijavallabhan, V.; Njoroge, F. G., Discovery of (1R,5S)—N-[3-Amino-1-(cyclobutylmethyl)-2,3-dioxopropyl]-3-[2 (S)-[[(1,1-dimethylethyl)amino]carbonyl]amino]-3,3-dimethyl-1-oxobutyl]-6,6-dimethyl-3-azabicyclo[3.1.0]hexan -2 (S)-carboxamide (SCH 503034), a Selective, Potent, Orally Bioavailable Hepatitis C Virus NS3 Protease Inhibitor: A Potential Therapeutic Agent for the Treatment of Hepatitis C Infection. Journal of Medicinal Chemistry 2006, 49 (20), 6074-6086.
8. Rakesh; Sun, D.; Lee, R. B.; Tangallapally, R. P.; Lee, R. E., Synthesis, optimization and structure-activity relationships of 3,5-disubstituted isoxazolines as new anti-tuberculosis agents. European Journal of Medicinal Chemistry 2009, 44 (2), 460-472.
9. Saitoh, M.; Kunitomo, J.; Kimura, E.; Iwashita, H.; Uno, Y.; Onishi, T.; Uchiyama, N.; Kawamoto, T.; Tanaka, T.; Mol, C. D.; Dougan, D. R.; Textor, G. P.; Snell, G. P.; Takizawa, M.; Itoh, F.; Kori, M., 2-{3-[4-(Alkylsulfinyl)phenyl]-1-benzofuran -5-yl}-5-methyl-1,3,4-oxadiazole Derivatives as Novel Inhibitors of Glycogen Synthase Kinase-3B with Good Brain Permeability. Journal of Medicinal Chemistry 2009, 52 (20), 6270-6286.
10. Dekhane, D. V.; Pawar, S. S.; Gupta, S.; Shingare, M. S.; Patil, C. R.; Thore, S. N., Synthesis and anti-inflammatory activity of some new 4,5-dihydro-1,5-diaryl-1H-pyrazole-3-substituted-heteroazole derivatives. Bioorganic & Medicinal Chemistry Letters 2011, 21 (21), 6527-6532.
11. Matteson, D. S.; Sadhu, K. M., Boronic ester homologation with 99% chiral selectivity and its use in syntheses of the insect pheromones (3S,4S)-4-methyl-3-heptanol and exo-brevicomin. Journal of the American Chemical Society 1983, 105 (7), 2077-2078.
12. Nitsche, C.; Zhang, L.; Weigel, L. F.; Schilz, J.; Graf, D.; Bartenschlager, R.; Hilgenfeld, R.; Klein, C. D., Peptide-Boronic Acid Inhibitors of Flaviviral Proteases: Medicinal Chemistry and Structural Biology. Journal of Medicinal Chemistry 2017, 60 (1), 511-516.
13. Galemmo, R. A.; Fevig, J. M.; Carini, D. J.; Cacciola, J.; Wells, B. L.; Hillyer, G. L.; Buriak, J.; Rossi, K. A.; Stouten, P. F. W.; Alexander, R. S.; Hilmer, R.; Bostrom, L.; Abelman, M. M.; Lee, S.-L.; Weber, P. C.; Kettner, C. A.; Knabb, R. M.; Wexler, R. R., (N-acyl-N-alkyl) glycyl borolysine analogs: A new class of potent thrombin inhibitors. Bioorganic & Medicinal Chemistry Letters 1996, 6 (24), 2913-2918.
14. Dess, D. B.; Martin, J. C., Readily accessible 12-1-5 oxidant for the conversion of primary and secondary alcohols to aldehydes and ketones. The Journal of Organic Chemistry 1983, 48 (22), 4155-4156.
15. Behnam, M. A. M.; Graf, D.; Bartenschlager, R.; Zlotos, D. P.; Klein, C. D., Discovery of Nanomolar Dengue and West Nile Virus Protease Inhibitors Containing a 4-Benzyloxyphenylglycine Residue. Journal of Medicinal Chemistry 2015, 58 (23), 9354-9370.
16. Kühl, N.; Graf, D.; Bock, J.; Behnam, M. A. M.; Leuthold, M.-M.; Klein, C. D., A New Class of Dengue and West Nile Virus Protease Inhibitors with Submicromolar Activity in Reporter Gene DENV-2 Protease and Viral Replication Assays. Journal of Medicinal Chemistry 2020, 63 (15), 8179-8197.
17. Bloemhoff, W.; Kerling, K. E. T., Studies on polypeptides XV Synthesis of L- and D-homohistidine. Recueil des Travaux Chimiques des Pays-Bas 1975, 94 (8), 182-185.
18. Elliott, J. T.; Hoekstra, W. J.; Maryanoff, B. E.; Prestwich, G. D., Photoactivatable peptides based on BMS-197525: A potent antagonist of the human thrombin receptor (PAR-1). Bioorganic & Medicinal Chemistry Letters 1999, 9 (2), 279-284.
19. Albericio, F.; Bofill, J. M.; El-Faham, A.; Kates, S. A., Use of Onium Salt-Based Coupling Reagents in Peptide Synthesis1. The Journal of Organic Chemistry 1998, 63 (26), 9678-9683.
20. Mitsunobu, O., The Use of Diethyl Azodicarboxylate and Triphenylphosphine in Synthesis and Transformation of Natural Products. Synthesis 1981, 1981 (01), 1-28.
21. Bellier, B.; McCort-Tranchepain, I.; Ducos, B.; Danascimento, S.; Meudal, H.; Noble, F.; Garbay, C.; Roques, B. P., Synthesis and Biological Properties of New Constrained CCK-B Antagonists: Discrimination of Two Affinity States of the CCK-B Receptor on Transfected CHO Cells. Journal of Medicinal Chemistry 1997, 40 (24), 3947-3956.
22. Bailey, M. D.; Bordeleau, J.; Garneau, M.; Leblanc, M.; Lemke, C. T.; O'Meara, J.; White, P. W.; Llinas-Brunet, M., Peptide backbone replacement of hepatitis C virus NS3 serine protease C-terminal cleavage product analogs: Discovery of potent succinamide inhibitors. Bioorganic & Medicinal Chemistry Letters 2013, 23 (15), 4447-4452.
23. Lampa, A.; Ehrenberg, A. E.; Gustafsson, S. S.; Vema, A.; Åkerblom, E.; Lindeberg, G.; Karlen, A.; Danielson, U. H.; Sandström, A., Improved P2 phenylglycine-based hepatitis C virus NS3 protease inhibitors with alkenylic prime-side substituents. Bioorganic & Medicinal Chemistry 2010, 18 (14), 5413-5424.
24. Lindgren, C.; Tyagi, M.; Viljanen, J.; Toms, J.; Ge, C.; Zhang, N.; Holmdahl, R.; Kihlberg, J.; Linusson, A., Dynamics Determine Signaling in a Multicomponent System Associated with Rheumatoid Arthritis. Journal of Medicinal Chemistry 2018, 61 (11), 4774-4790.

### Example 16-In Vitro Protease Assays

### 16.1 Materials and Methods

Single dose screening of compounds and/or IC₅₀ determination was performed by a fluorimetric assay for DENV protease as described before (Nitsche and Klein, Fluorimetric and HPLC-Based Dengue Virus Protease Assays Using a FRET Substrate. In Antiviral Methods and Protocols, Gong, E. Y., Ed. Humana Press: Totowa, New Jersey, 2013; pp 221-236-Nitsche et al., J. Med. Chem. 2013, 56 (21), 8389-8403.) The FRET substrate was an internally quenched anthranilamide/nitrotyrosine substrate as described by Steuer et al. (DOI: 10.1177/1087057109344115)(Km=105 μM). By catalytic activity of the protease, the cleaved substrate results in increased fluorescence that can be monitored using a BMG Labtech Fluostar OPTIMA Microtiter fluorescence plate reader (excitation wavelength of 320 nm and a monitored emission wavelength of 405 nm). Dilutions were made starting from 10 mM stock solutions in DMSO and final concentrations were measured in triplicates. The inhibitors were preincubated for 15 min with DENV protease (100 nM) in the assay buffer (50 mM Tris-HCl pH 9.0, ethylene glycol (10% v/v), and 0.0016% Brij 58). The enzymatic reaction was initiated by the addition of the FRET substrate (final concentration 50 μM) to obtain a final assay volume of 100 μL per well. The enzymatic activity was monitored for 15 min and determined as a slope of relative fluorescence units per second (RFU/s) for each concentration. The mean of the triplicates were plotted against the corresponding concentration in CDDVault to determine the IC₅₀-values. Analogous procedures were used to determine the inhibitory potency on WNV (Kühl et al., J. Med. Chem. 2020, 63 (15): 8179-8197) and ZIKA protease (Nitsche et al., ACS Med. Chem. Lett. 2019 Feb. 14; 10 (2): 168-174). For selectivity screening of active candidates, similar biochemical assays with human serine proteases trypsin (Weigel et al., J. Med. Chem. 2015, 58 (19), 7719-7733.) and/or thrombin (Nitsche et al., J. Med. Chem. 2013, 56 (21), 8389-8403) were used.

### 16.2 Inhibition of Dengue

The following compounds showed at least 25% inhibition of DENV at 50 μM: 1-17, 19, 21, 23, 25, 29, 32, 36-45, 48-68, 72, 78, 82, 83, 89-93, 97, 98, 110-123, 125-130, 132-139, 146-152, 156, 158-168, 170-190, 192, 193, 196

The following compounds showed at least 20% inhibition of DENV at 1 μM: 39, 41, 48-51, 114, 125, 129, 132, 136, 137, 148, 167, 172, 173

The following compounds had IC₅₀ of 10 UM or less for DENV: 1, 2, 4, 7, 9-17, 24, 25, 32, 39-41, 43, 44, 48-51, 53, 54, 72, 82, 83, 90, 92, 97, 98, 110-119, 121-123, 125, 128, 129, 132, 133, 134, 136, 137, 139, 146-150, 156, 158-161, 167, 168, 170-177, 180-183, 186, 187, 189, 190, 192, 193, 196

The following compounds had EC₅₀ of 20 μM or less for DENV: 9, 11, 32, 38, 43, 44, 69, 70, 71, 72, 73, 75, 77, 79, 81, 82, 83, 86, 87, 88, 89, 92, 94, 98, 113, 114, 115, 116, 117, 118, 122, 123, 129, 136, 137, 156, 158, 162, 167, 168, 170, 171, 174, 175, 176, 190, 192, 196.

16.3 Inhibition of West Nile virus

The following compounds showed at least 25% inhibition of WNV at 50 μM: 5, 6, 8-11, 13, 16, 17, 43, 44, 48, 90, 92, 115, 116, 117, 118, 122, 123, 125, 129, 132, 136, 137, 167, 168, 170, 171, 174, 175, 176, 180-186, 190, 191, 192, 193, 196

The following compounds showed at least 10% inhibition of WNNV at 1 μM: 132, 137, 186

The following compounds had IC₅₀ of 40 μM or less for WNNV: 5, 6, 8, 9, 10, 11, 13, 16, 17, 39, 41, 43, 44, 48, 90, 92, 113, 114, 115, 116, 117, 118, 122, 123, 125, 129, 132, 136, 137, 156, 167, 168, 170, 171, 174, 175, 176, 180, 181, 182, 183, 186, 189, 190, 193, 196.

The following compounds had EC₅₀ of 25 μM or less for WNNV: 9, 10, 43, 44, 50, 51, 53, 54, 113, 114, 118, 136, 170, 189, 196.

### 16.4 Inhibition of Zika Virus

The following compounds showed at least 65% inhibition of ZIKV at 50 μM: 5, 6, 8, 9, 10, 11, 39, 43, 44, 48, 49, 50, 51, 53, 54, 92, 113, 114, 115, 116, 117, 118, 122, 123, 125, 129, 136, 137, 167, 168, 170, 171, 175, 176, 189, 196.

The following compounds showed at least 10% inhibition of ZIKV at 1 μM: 9, 11, 39, 43, 44, 48, 49, 50, 51, 54, 92, 113, 114, 115, 116, 117, 118, 123, 125, 129, 136, 137, 167, 168, 189.

The following compounds had IC₅₀ of 25 μM or less for ZIKV: 5, 6, 8, 9, 10, 11, 39, 43, 44, 48, 49, 50, 51, 53, 54, 92, 113, 114, 115, 116, 117, 118, 122, 123, 125, 129, 136, 137, 167, 168, 170, 171, 175, 176, 189, 196.

The following compounds had EC₅₀ of 10 μM or less for ZIKV: 44, 49, 50, 51, 53, 54, 113, 114, 118, 136, 137, 170, 189, 196.

### 16.5 Selectivity of Compounds

This data shows the selectivity of the compounds, in low activity with regard to host proteases.

The following compounds showed less than 20% inhibition of human Thrombin at 25 M: 5, 6, 8, 9, 10, 11, 13, 16, 17, 43, 44, 90, 92, 115, 116, 117, 118, 122, 123, 125, 129, 132, 136, 137, 167, 168, 170, 171, 174, 175, 176, 180, 181, 186, 189, 190, 196.

The following compounds showed less than 5% inhibition of human Thrombin at 5 μM: 5, 6, 8, 9, 10, 11, 13, 16, 17, 43, 44, 90, 92, 115, 116, 117, 118, 122, 123, 125, 129, 132, 136, 137, 167, 168, 170, 171, 174, 175, 176, 180, 181, 186, 189, 190, 196.

The following compounds showed less than 20% inhibition of human Trypsin at 25 μM: 5, 6, 8, 11, 13, 16, 17, 43, 44, 90, 92, 114, 115, 116, 117, 118, 122, 123, 125, 129, 132, 136, 137, 168, 170, 171, 174, 175, 181, 189, 190, 196.

The following compounds showed less than 10% inhibition of human Trypsin at 5 μM: 5, 6, 8, 10, 11, 13, 16, 17, 43, 44, 90, 92, 114, 115, 116, 117, 118, 122, 123, 125, 129, 136, 137, 168, 170, 171, 174, 175, 189, 190, 196.

### Example 17-In Vivo Assay

### 17.1 Materials and Methods

The purpose of this study was to determine the efficacy of the compounds on Dengue virus 2 (DENV2, ATCC® VR-1584™) infection in AG129 mice.

### 17.1.1 Animal Welfare and Husbandry

Six groups of AG129 mice, 6-8 weeks old, with 9 mice per group were used to test compounds according to table 3.1.1. Animal room environment was monitored for temperature and relative humidity twice per day. The temperature range was 22° C.±3° C. and humidity range was 30-70%. The animals were provided with 12 hours light and 12 hours darkness. Feed and drinking water was provided ad libitum. Compounds 43 and 113 were tested.

TABLE 3.1.1
Experimental groups

| Group | | | # of |
|---|---|---|---|
| ID | Treatment | Dosing and administration route | animals |
| G1 | Infection Control | Vehicle (IP) | 9 |
| G2 | 43 (Dose1) | 20 mg/kg body weight (I.P, b.i.d) | 9 |
| G3 | 43 (Dose2) | 10 mg/kg body weight (I.P, b.i.d) | 9 |
| G4 | 113 (Dose1) | 20 mg/kg body weight (I.P, b.i.d) | 9 |
| G5 | 113 (Dose2) | 10 mg/kg body weight (I.P, b.i.d) | 9 |
| G6 | Positive control | Ribavirin (100 mg/kg, SC) | 9 |

17.1.2 Virus Production

C6/36 (ATCC(CRL-1660) cell lines were grown in RPMI 1640 medium (Gibco/HiMedia) plus 5% fetal bovine serum at 28° C. Dengue virus type 2 (ATCC®VR-1584™) New Guinea C(NGC, DENV2) was passaged in BHK-21 cell cultures to make sufficient stocks for assay. Virus titer was determined by TCID₅₀ method using BHK-21 cells.

### 17.1.3 Induction of Infection

Twenty-four hours post treatment (Day 0), animals were injected intraperitoneally (IP) with 0.2 ml of virus suspension (˜5×10⁷/animal).

### 17.1.4 Formulations

The vehicle for tested compounds was 5% DMSO+95% (20%) hydroxypropylcyclodextrin. Saline (0.9%) was used as vehicle for Ribavirin. Formulations were prepared daily immediately before dosing.

### 17.1.5 Treatment, Sampling and Clinical Observations

Administration of compounds and vehicle to animals started twenty four hours before infection (Day −1). Animals were treated daily (From Day-1 to day 4) as per the experimental design shown below in table 3.1.5. Animals were monitored for general clinical signs, morbidity and mortality. Body weights were measured daily.

TABLE 3.1.5
treatment and sampling schedule

| DAY | Treatment & sampling details |
|---|---|
| Day −1 | Administration Vehicle/Compounds |
| | Blood sampling (Animal #1, 2, 3) 1 hr post 1^{st} dose |
| Day 0 | Administration Vehicle/Compounds |
| | Blood sample (Animal #4, 5, 6) & Infection |
| Day 1 | Administration Vehicle/Compounds |
| | Blood sample (Animal #7, 8, 9) |
| Day 2 | Administration Vehicle/Compounds |
| | Blood sample (Animal #1, 2, 3) |
| Day 3 | Administration Vehicle/Compounds |
| | Blood sample (Animal #4, 5, 6) |
| Day 4 | Administration Vehicle/Compounds |
| | Blood sample (Animal #7, 8, 9) |
| Day 5 | Terminal Sacrifice |
| | Blood sample & spleen collection (12 hr post last dose) |

### 17.1.6 Blood Collection

Blood was collected on days-1, 0, 1, 2, 3, 4 to 5.

### 17.1.7 Plaque Assay

BHK-21 cells were cultured to approximately 80% confluency in 24-well plates (NUNC, NY, USA). Plasma samples were 10-fold serially diluted in RPMI 1640 (GIBCO). BHK-21 monolayers were infected with 100 μl of each virus dilution. After incubation at 37° C. and 5% CO₂ atmosphere for one hour with rocking at 15 min intervals, the medium was decanted and 1 ml of 1% (w/v)carboxymethyl cellulose in RPMI supplemented with 2% FCS was added to each well. After four days incubation at 37° C. in 5% CO₂, the cells were fixed with 4% paraformaldehyde and stained for 30 min with 200 ml of 1% crystal violet dissolved in 37% formaldehyde. After thorough rinsing with water, plates were dried and the plaques were scored visually.

### 17.1.8 Infection Markers

Cytokine levels (TNF alpha, IL-6 and IL-12) in plasma samples were measured using Enzyme-Linked Immunosorbent Assay (ELISA). Mouse TNF alpha ELISA Kit (Abcam: ab208348), Mouse IL-6 ELISA Kit (Abcam: ab100712), and Mouse IL-12 p40+IL-12 p70 ELISA Kit (Abcam: ab100699) were used to estimate TNF alpha, IL-6 and IL-12, respectively. The assays were performed according to the manufacturer's instructions and was analysed on a TECAN-NanoQuant Plate™ system.

### 17.1.9 Termination

Animals were sacrificed on day 5 post infection with Isoflurane followed by an overdose of CO₂. Spleens were excised, measured and weighed.

### 17.1.10 Data Analysis

The Mean±SD Log10PFU/ml, relative Mean±SD spleen weight, Mean±SD biomarker concentrations were estimated in each group. Significant differences between group means and control were analyzed by one-way ANOVA, followed by a Dunnett's multiple comparison test, using GraphPad Prism 5 at 95% confidence levels. A p value of <0.05 was considered as significant.

### 17.2 Results

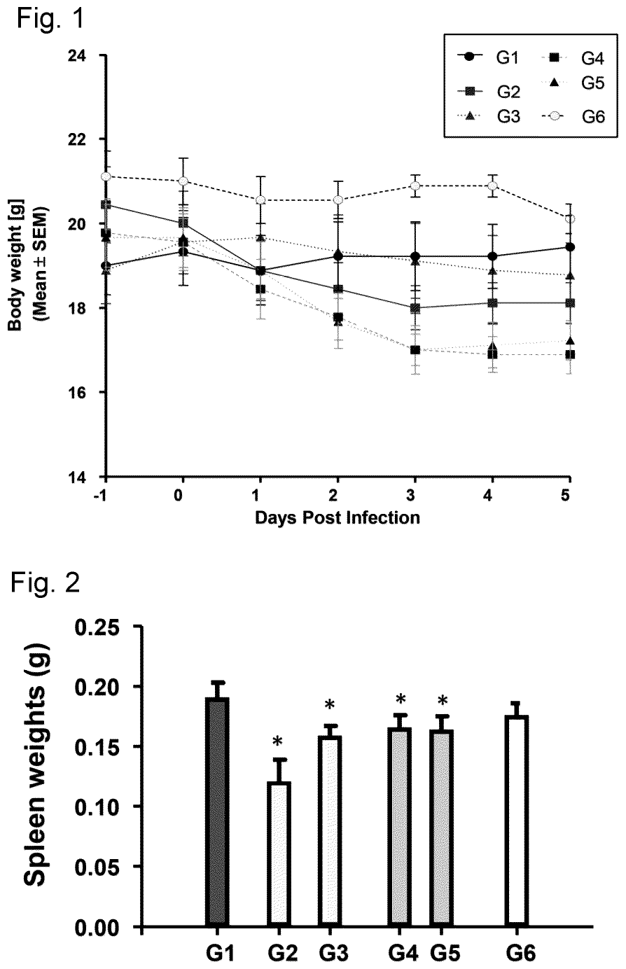

Animals treated with 20 and 10 mg/kg of compounds 43 or 113 were apparently normal. Although there were differences in mean body at the end of treatment (Table 3.2.1 and FIG. 1), they were not statistically significant. 43 (10 mg/kg and 20 mg/kg) and 113 (10 and 20 mg/kg) showed significant reduction in spleen weights (an indication of lowered infection) when compared to the vehicle control (Table 3.2.2 and FIG. 2). No significant reduction was observed with Ribavirin.

TABLE 3.2.1
Body weights (g) of mice at the end of the study (Mean ± SD)

| G1 | G2 | G3 | G4 | G5 | G6 |
|---|---|---|---|---|---|
| (Vehicle | 43 | 43 | 113 | 113 | (Ribavirin |
| Control), | (20 mg/kg) | (10 mg/kg) | (20 mg/kg) | (10 mg/kg) | 100 mg/kg) |
| n = 9 | n = 9 | n = 9 | n = 9 | n = 9 | n = 9 |
| 19.44 ± 2.24 | 18.11 ± 1.45 | 18.78 ± 2.28 | 16.89 ± 1.36 | 17.22 ± 1.39 | 20.11 ± 1.05 |

TABLE 3.2.2
Spleen weights (g) (Mean ± SD)

| G1 | G2 | G3 | G4 | G5 | G6 |
|---|---|---|---|---|---|
| (Vehicle | 43 | 43 | 113 | 113 | (Ribavirin |
| Control), | (20 mg/kg) | (10 mg/kg) | (20 mg/kg) | (10 mg/kg) | 100 mg/kg) |
| n = 9 | n = 9 | n = 9 | n = 9 | n = 9 | n = 9 |
| 0.191 ± 0.012 | 0.121 ± 0.018* | 0.159 ± 0.008* | 0.166 ± 0.010* | 0.164 ± 0.011* | 0.176 ± 0.010 |

*Significantly different from vehicle control (p < 0.05)

### 17.2.3 Viral Reduction in Plasma (Plaque Assay)

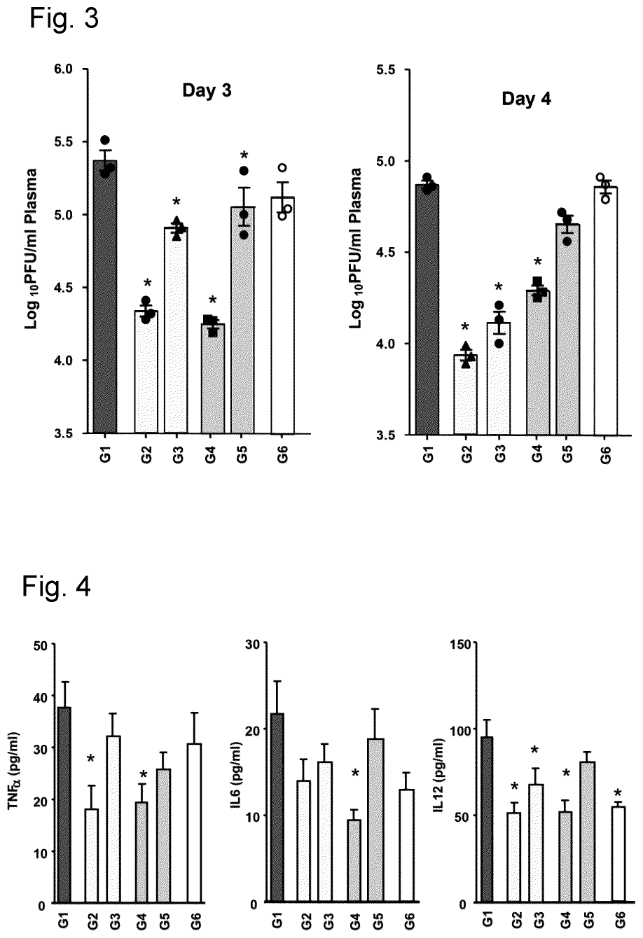

Compound 43 (Intraperitoneal Twice daily, at doses 20 mg/kg and10 mg/kg) showed significant dose dependent anti-viral effect in plasma, determined by Plaque assay, when compared to vehicle control (p<0.05) on day 3 and 4 (Table 3.2.3 and FIG. 3). Compound 113 showed significant dose dependent anti-viral effect in plasma on day 3 (Intraperitoneal Twice daily, at doses 20 mg/kg and10 mg/kg) and 4 (Intraperitoneal Twice daily, at doses 20 mg/kg)(Table 3.2.3 and FIG. 3). Ribavirin (100 mg/kg) did not show significant antiviral activity when compared to vehicle control on both days.

TABLE 3.2.3
Mean + SD Log₁₀PFU/ml Plasma

| Experimental | | | | | | |
|---|---|---|---|---|---|---|
| Group | Day 0 | Day 1 | Day 2 | Day 3 | Day 4 | Day 5 |
| G1 | NA | NA | 3.15 ± 0.43 | 5.37 ± 0.12 | 4.87 ± 0.04 | 3.77 ± 0.10 |
| G2 | NA | NA | 3.48 ± 0.25 | 4.34 ± 0.07* | 3.94 ± 0.05* | 3.06 ± 0.31 |
| G3 | NA | NA | 3.36 ± 0.41 | 4.91 ± 0.06* | 4.11 ± 0.10* | 3.28 ± 0.21 |
| G4 | NA | NA | 3.07 ± 0.28 | 4.25 ± 0.05* | 4.29 ± 0.05* | 3.77 ± 0.11 |
| G5 | Na | NA | 3.79 ± 0.10 | 5.05 ± 0.23* | 4.65 ± 0.08 | 3.66 ± 0.11 |
| G6 | NA | NA | 3.34 ± 0.29 | 5.12 ± 0.18 | 4.86 ± 0.06 | 3.50 ± 0.13 |

### 17.2.4 Biomarkers

Compounds 43 (20 mg/kg) and 113 (20 mg/kg) showed significant reduction in TNF-α levels when compared to the vehicle control (p<0.05). No significant reduction was observed with Ribavirin (p>0.05) when compared to the vehicle control (Table 3.2.4 and FIG. 4).

Compound 113 (20 mg/kg) showed significant reduction in IL-6 levels when compared to the vehicle control (p<0.05). No significant reduction was observed with compound 43 (20 mg/kg, 10 mg/kg), compound 113 (10 mg/kg), and Ribavirin (p>0.05) when compared to the vehicle control (Table 3.2.4 and FIG. 4).

Compound 43 (10 mg/kg, 20 mg/kg), compound 113 (20 mg/kg), and Ribavirin showed significant reduction in IL-12 levels when compared to the vehicle control (p<0.05)(Table 3.2.4 and FIG. 4).

TABLE 3.2.4
Plasma concentrations of TNF-alpha, IL-6 and IL-12 in mouse
plasma after treatment

| | | G2 | G3 | G4 | G5 | |
|---|---|---|---|---|---|---|
| | G1 | 43 | 43 | 113 | 113 | G6 |
| | (Vehicle Control), | (20 mg/kg) | (10 mg/kg) | (20 mg/kg) | (10 mg/kg) | (Ribavirin 100 mg/kg) |
| Cytokine | n = 9 | n = 9 | n = 9 | n = 9 | n = 9 | n = 9 |
| TNF-alpha | 37.85 ± 14.35 | 18.20 ± 13.26* | 32.45 ± 12.30 | 19.55 ± 10.23* | 25.99 ± 9.19 | 31.09 ± 16.91 |
| IL-6 | 21.76 ± 11.21 | 13.92 ± 7.59 | 16.08 ± 6.47 | 9.62 ± 3.02* | 18.92 ± 10.14 | 13.13 ± 5.34 |
| IL-12 | 95.69 ± 28.29 | 51.67 ± 16.83* | 67.27 ± 29.47* | 53.06 ± 16.62* | 81.79 ± 14.30 | 54.56 ± 9.39* |

*Significantly different from vehicle control (p < 0.05)

### Example 18-in vivo assay using subcutaneous administration

### 18.1 Introduction

These studies were largely performed as in Example 17, with a difference found in the administration of compounds of the invention. In Example 17, a comparative control using known antiviral drug ribavirin was administered via subcutaneous administration, and this comparative control was outperformed by the intraperitoneally administered compounds of the invention. In this example the compounds of the invention are administered using subcutaneous administration, and compound 113 was used as exemplary for compounds according to the invention.

### 18.2 Results

Subcutaneous administration showed slow elimination of compound 113 in blood plasma and improved efficacy against dengue virus infection in mice over other routes of administration.

### 18.2.1 Compounds Show Good Pharmacokinetics

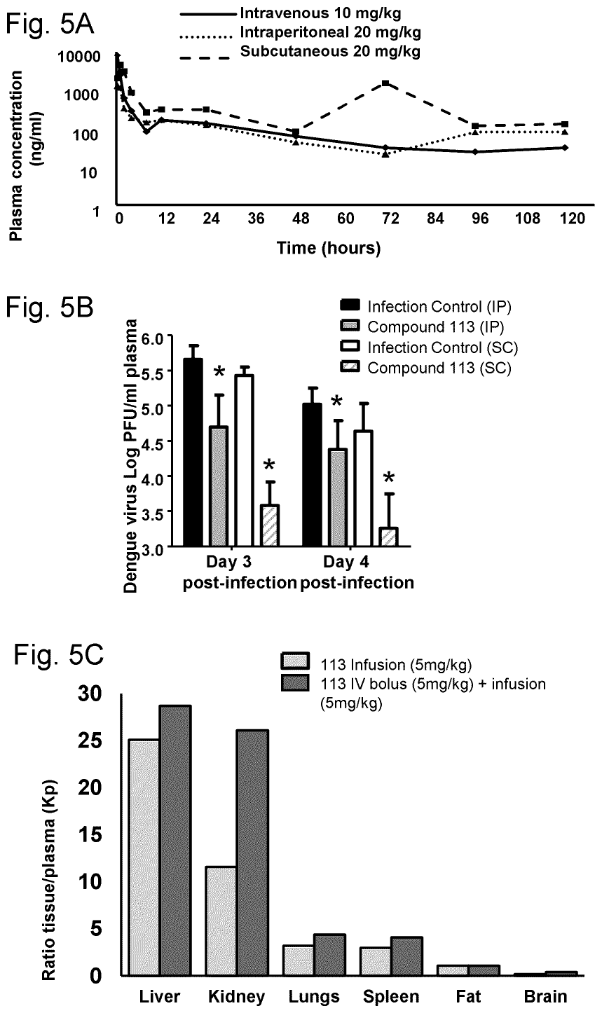

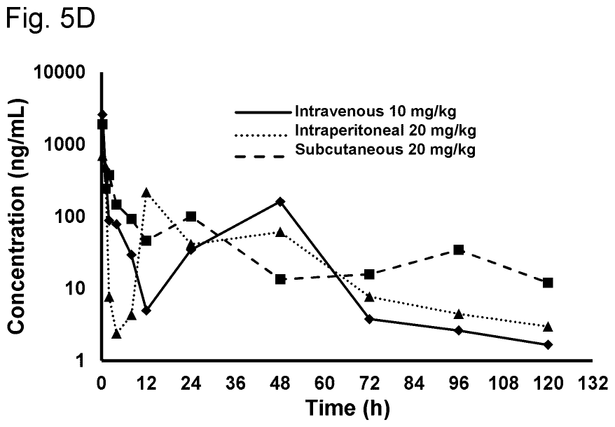

As described in Example 17, compounds of the invention such as compound 113 give potent and specific inhibition of DENV protease in biochemical and cellular infection assays with minimal cytotoxicity. In vivo pharmacokinetic experiments for compound 113 showed reliable pharmacokinetic profiles with limited inter animal variability and dose proportionality following three routes: intravenous, intraperitoneal and subcutaneous (FIG. 5A). Similar data was obtained with compound 43 (FIG. 5D)

Low oral bioavailability of drugs such as peptidomimetics can be due to instability in the gastrointestinal tract, first pass elimination, or low permeability over biological membranes. The first two are not applicable to compounds of the invention: physical and chemical stability was found to be preserved in simulated intestinal fluids containing the appropriate number of metabolizing enzymes (pancreatin/pepsin), while permeability in Caco-2 cells was found to be low. The rate of elimination via systemic administration routes was also found to be slow, in tune with the limited hepatic clearance observed in vitro in both hepatocytes and liver microsomes. Furthermore, in vitro ADMET experiments showed no CYP inhibition/induction liability, indicating a low to absent risk of drug-drug interactions.

### 18.2.2 Subcutaneous Administration Increases Log-Reduction

The experiments described in Example 17 also confirmed excellent efficacy in mice (>92% viral load reduction on day 3 post-infection), with a treatment window up to 48 hours post-infection. It was found that with subcutaneous administration, viral load reduction was improved by more than one additional log-unit (>98% and >99% viral load reduction on day 3 and 4 after infection respectively; FIG. 5B). Via subcutaneous route, measured plasma levels over time were higher than via intraperitoneal administration (FIG. 5A and FIG. 5D).

Compounds 113 and 43 also showed good therapeutic efficacy as evidenced by plaque assays of the compounds in mouse plasma through the treatment period. Results are shown in the table below, where compounds were used at 20 mg/kg (5h post infection). Antiviral activity is much improved after subcutaneous administration, with a longer lasting effect.

| | G1 | G2 | G3 | G4 | G5 | G6 |
|---|---|---|---|---|---|---|
| Administration | IP | | | SC | | |
| Compound | Vehicle | 113 | 43 | Vehicle | 113 | 43 |
| Day 3 | 5.66 ± 0.19 | 4.70 ± 0.75* | 4.32 ± 0.45* | 5.43 ± 0.12 | 3.58 ± 0.34* | 3.43 ± 0.304* |
| Day 4 | 5.02 + 0.23 | 4.23 + 0.52* | 4.38 + 0.41 | 4.64 + 0.39 | 3.26 + 0.49* | 3.34 + 0.40* |

### 18.2.3 Compounds Show Good Organ Targeting

Following administration, molecules reach the systemic circulation via either the blood capillaries or the lymphatic system. Biodistribution in rats showed that compound 113 could reach the main target sites of dengue (liver and spleen, FIG. 5C). These data indicate that subcutaneous administration can enhance delivery to target organs, further reducing viral load.

### 18.3 Conclusion

For dengue there is currently no traveller vaccine or antiviral on the market. For travellers visiting endemic countries, it is currently common practice for other infectious diseases (like hepatitis A or yellow fever) that they are vaccinated by single or repeated injection. In the case of malaria, travellers use daily oral drugs to prevent infection. Both treatment modalities are generally well accepted. Vaccinations are mostly given by (intramuscular) injection and are generally well accepted to obtain protection in this population.

At present there is no dengue prophylaxis on the market. The invention can provide such a prophylaxis or traveller vaccine. Subcutaneous administration was found to have specific dengue-relevant advantages for efficient systemic delivery and patient/traveller compliance and ease. Preclinical pharmacokinetic data showed slow elimination of the compounds, indicating that a single dose subcutaneous administration can be sufficient for one or two weeks of treatment in a therapeutic and prophylactic setting.

1. A compound of general formula (0) or a salt thereof: 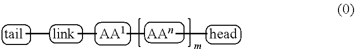
wherein
m is 3 or 2;
link is —C(O)—or is absent;
tail is H or C1-24alkyl;
head is —NH₂, —NH-C1-24alkyl, —C(O)—NH₂, —C(O)—NH-C1-24alkyl, or5-6-membered (hetero)aryl;
AA¹ is an amino acid residue that is connected to its neighbouring AAⁿ via an amide bond to which it contributes a donor carboxylic acid, and that via its amine is connected to link;
AAⁿ is for each instance independently an amino acid residue.

2. The compound according to claim 1, wherein m is 3.

3. The compound according to claim 1, wherein m is 2.

4. The compound according to claim 1, wherein tail is CH₃—(CH₂)₁₄—;

5. The compound according to claim 1, wherein link is —C(O)- and tail is C1-24 alkyl.

6. The compound according to claim 1, wherein link is —C(O)—.

7. The compound according to claim 4, wherein link is —C(O)—.

8. The compound according to claim 1, wherein link is absent and tail is H.

9. The compound according to claim 1, wherein head is —NH₂, —NH—(CH₂)₁₅—CH₃, -phenyl, —CH₃, or —C(O)—NH₂.

10. The compound according to claim 1, wherein head is —NH₂, -phenyl, —CH₃, or —C(O)—NH₂.

11. The compound according to claim 4, wherein head is —NH₂, -phenyl, —CH₃, or —C(O)—NH₂.

12. The compound according to claim 8, wherein head is —NH—C1-24alkyl or —C(O)—NH-C1-24alkyl.

13. The compounds according to claim 1, wherein AA¹ is lysine, arginine, or alanine.

14. The compounds according to claim 1, wherein each AAⁿ is individually selected from alanine, arginine, glutamine, lysine, phenylalanine, histidine, proline, (4-OBn-phenyl)glycine, tryptophan, and homophenylalanine.

15. The compounds according to claim 1, wherein at least one instance of AAⁿ that is not adjacent to head is alanine, glutamine, or histidine.

16. The compounds according to claim 1, wherein the instance of AAⁿ that is adjacent to head is lysine, phenylalanine, homophenylalanine, histidine, tryptophan, or (4-OBn-phenyl)glycine.

17. The compounds according to claim 1, wherein at least one instance of AAⁿ that is not adjacent to head is glutamine.

18. The compounds according to claim 1, wherein the instance of AAⁿ that is adjacent to head is phenylalanine.

19. The compounds according to claim 1, wherein AA¹ and each instance of AAⁿ together form a peptide represented by KAAK, ARQK, KAF, RQ-homoPhe, KAK, RAF, RQF, KAH, KAAH, KPA-(4-OBn-phenylGly), KPAK, KAAW, KPH-homoPhe, KPAF, KPA-homoPhe, or KPAW.

20. The compounds according to claim 1, wherein AA¹ and each instance of AAⁿ together form a peptide represented by KAAK, (D-K)AAK, K (D-A) AK, KA (D-A) K, KAA (D-K), ARQK, KAF, RQ-homoPhe, KAK, RAF, RQF, (D-R) QF, KAH, KAAH, KAA (D-H), KPA-(4-OBn-phenylGly), KPAK, KAAW, KPH-homoPhe, KPAF, KPA (D-F), KPA-homoPhe, KPA-(D-homoPhe), KPAW, (D-K) PAW, or KPA (D-W).

21. The compound according to claim 1, wherein
i. tail is H, link is absent; AA¹ and each instance of AAⁿ together form a peptide represented by ARQK; head is —NH—(CH₂)₁₅—CH₃;
ii. tail is CH₃—(CH₂)₁₄—, link is —C(O)—; AA¹ and each instance of AAⁿ together form a peptide represented by KAAK; head is -phenyl;
iii. tail is CH₃—(CH₂)₁₄—, link is —C(O)—; AA¹ and each instance of AAⁿ together form a peptide represented by KAF; head is —NH₂;
iv. tail is CH₃—(CH₂)₁₄—, link is —C(O)—; AA¹ and each instance of AAⁿ together form a peptide represented by RQ-homoPhe; head is —NH₂;
v. tail is CH₃—(CH₂)₁₄—, link is —C(O)—; AA¹ and each instance of AAⁿ together form a peptide represented by KA (D-A) K; head is —NH₂;
vi. tail is CH₃—(CH₂)₁₄—, link is —C(O)—; AA¹ and each instance of AAⁿ together form a peptide represented by ARQK; head is —NH₂;
vii. tail is CH₃—(CH₂)₁₄—, link is —C(O)—; AA¹ and each instance of AAⁿ together form a peptide represented by KAK; head is -phenyl;
viii. tail is CH₃—(CH₂)₁₄—, link is —C(O)—; AA¹ and each instance of AAⁿ together form a peptide represented by RAF; head is —NH₂;
ix. tail is CH₃—(CH₂)₁₄—, link is —C(O)—; AA¹ and each instance of AAⁿ together form a peptide represented by KAA (D-K); head is —NH₂;
x. tail is CH₃—(CH₂)₁₄—, link is —C(O)—; AA¹ and each instance of AAⁿ together form a peptide represented by (D-R) QF; head is —NH₂;
xi. tail is H, link is absent; AA¹ and each instance of AAⁿ together form a peptide represented by KAK; head is —NH—(CH₂)₁₅—CH₃;
xii. tail is CH₃—(CH₂)₁₄—, link is —C(O)—; AA¹ and each instance of AAⁿ together form a peptide represented by KAAK; head is —NH₂;
xiii. tail is CH₃—(CH₂)₁₄—, link is —C(O)—; AA¹ and each instance of AAⁿ together form a peptide represented by KAH; head is —NH₂;
xiv. tail is CH₃—(CH₂)₁₄—, link is —C(O)—; AA¹ and each instance of AAⁿ together form a peptide represented by KAAK; head is —C(O)—NH₂;
xv. tail is CH₃—(CH₂)₁₄—, link is —C(O)—; AA¹ and each instance of AAⁿ together form a peptide represented by RQF; head is —NH₂;
xvi. tail is H, link is absent; AA¹ and each instance of AAⁿ together form a peptide represented by KAAK; head is —NH—(CH₂)₁₅—CH₃;
xvii. tail is CH₃—(CH₂)₁₄—, link is —C(O)—; AA¹ and each instance of AAⁿ together form a peptide represented by KAK; head is —C(O)—NH₂;
xviii. tail is CH₃—(CH₂)₁₄—, link is —C(O)—; AA¹ and each instance of AAⁿ together form a peptide represented by (D-K)AAK; head is —NH₂;
xix. tail is CH₃—(CH₂)₁₄—, link is —C(O)—; AA¹ and each instance of AAⁿ together form a peptide represented by KAAH; head is —NH₂;
xx. tail is CH₃—(CH₂)₁₄—, link is —C(O)—; AA¹ and each instance of AAⁿ together form a peptide represented by KAA (D-K); head is —C(O)—NH—CH₂-phenyl;
xxi. tail is CH₃—(CH₂)₁₄—, link is —C(O)—; AA¹ and each instance of AAⁿ together form a peptide represented by KPA-(4-OBn-phenylGly); head is —NH₂;
xxii. tail is CH₃—(CH₂)₁₄—, link is —C(O)—; AA¹ and each instance of AAⁿ together form a peptide represented by KAK; head is —CH₃;
xxiii. tail is CH₃—(CH₂)₁₄—, link is —C(O)—; AA¹ and each instance of AAⁿ together form a peptide represented by KPAK; head is —NH₂;
xxiv. tail is CH₃—(CH₂)₁₄—, link is —C(O)—; AA¹ and each instance of AAⁿ together form a peptide represented by K (D-A) AK; head is —NH₂;
xxv. tail is CH₃—(CH₂)₁₄—, link is —C(O)—; AA¹ and each instance of AAⁿ together form a peptide represented by KAA (D-H); head is —NH₂;
xxvi. tail is CH₃—(CH₂)₁₄—, link is —C(O)—; AA¹ and each instance of AAⁿ together form a peptide represented by KAAW; head is —NH₂;
xxvii. tail is CH₃—(CH₂)₁₄—, link is —C(O)—; AA¹ and each instance of AAⁿ together form a peptide represented by KPH-homoPhe; head is —NH₂;
xxviii. tail is CH₃—(CH₂)₁₄—, link is —C(O)—; AA¹ and each instance of AAⁿ together form a peptide represented by KPAF; head is —NH₂;
xxix. tail is CH₃—(CH₂)₁₄—, link is —C(O)—; AA¹ and each instance of AAⁿ together form a peptide represented by KPA-homoPhe; head is —NH₂;
xxx. tail is CH₃—(CH₂)₁₄—, link is —C(O)—; AA¹ and each instance of AAⁿ together form a peptide represented by KPA (D-homoPhe); head is —NH₂;
xxxi. tail is CH₃—(CH₂)₁₄—, link is —C(O)—; AA¹ and each instance of AAⁿ together form a peptide represented by KPA (D-F); head is —NH₂;
xxxii. tail is CH₃—(CH₂)₁₄—, link is —C(O)—; AA¹ and each instance of AAⁿ together form a peptide represented by (D-K) PAW; head is —NH₂; or
xxxiii. tail is CH₃—(CH₂)₁₄—, link is —C(O)—; AA¹ and each instance of AAⁿ together form a peptide represented by KPA (D-W); head is —NH₂.

22. The compound according to claim 1, wherein it is of formula (KAAK-4):

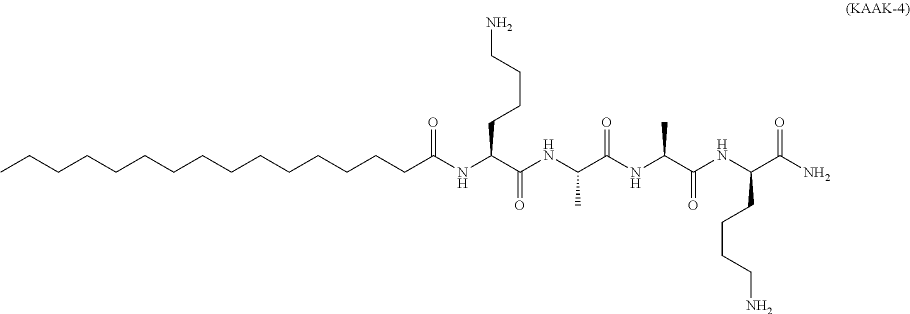 [SMILES: CCCCCCCCCCCCCCCC(=O)N[C@@H](CCCCN)C(=O)N[C@@H](C)C(=O)N[C@@H](C)C(=O)N[C@H](CCCCN)C(N)=O]

23. The compound according to claim 1, wherein it is of formula (RQF-1):

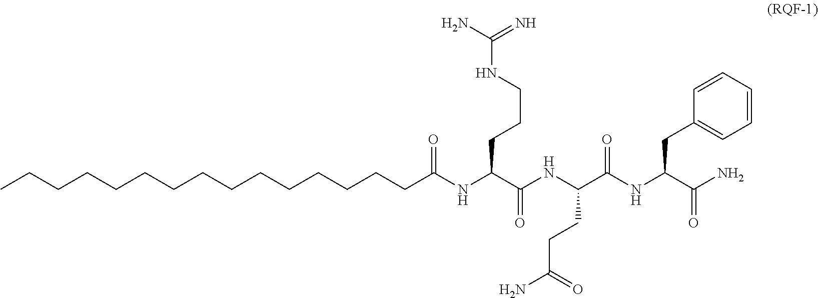 [SMILES: CCCCCCCCCCCCCCCC(=O)N[C@@H](CCCNC(=N)N)C(=O)N[C@@H](CCC(N)=O)C(=O)N[C@@H](Cc1ccccc1)C(N)=O]

24. Method of treating, preventing, or delaying a viral infection or a condition related to a viral infection in a subject in need thereof, the method comprising the step of administering to the subject an effective amount of a compound as defined in claim 1.

25. The method according to claim 24, wherein the viral infection is a dengue viral infection.

26. The method according to claim 24, wherein the compound is of general formula (0) or a salt thereof wherein tail is CH₃—(CH₂)₁₄—, link is —C(O)—; AA¹ and each instance of AAⁿ together form a peptide represented by KAA (D-K); head is —NH₂.

27. A compound of general formula (1) or a salt thereof: 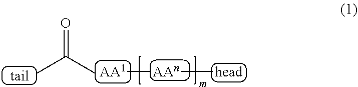
wherein
tail is C1-24alkyl, —O—C1-24alkyl, 5-20-membered (hetero)aryl, or 3-20-membered (hetero)cycloalkyl, wherein tail is optionally unsaturated, wherein tail is optionally substituted with halogen, C1-3 (halo)alkyl, or C1-3 (halo)alkoxyl;
head is —H, -h1, —O-h1, —C(O)-h1, —C(O)—N(H)h1, —N(h2) h1, or head is -C1-24alkyl, —(NH)₀₋₁-5-20-membered (hetero)aryl, or —(NH)₀₋₁-3-20-membered (hetero)cycloalkyl, wherein head is optionally unsaturated, wherein head is optionally substituted with halogen, C1-3 (halo)alkyl, or C1-3 (halo)alkoxyl;
h1 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h1 is optionally substituted with halogen, C1-3 (halo)alkyl, or C1-3 (halo)alkoxyl;
h2 is —H, —OH, —S(O)₀₋₂—OH, C1-8 (halo)alkyl, C1-8 (halo)alkoxyl, —S(O)₀₋₂-C1-8 (halo)alkyl, 3-8-membered (hetero)cycloalkyl, —S(O)₀₋₂-[3-8-membered (hetero)cycloalkyl], -C1-4alkyl-[3-8-membered (hetero)cycloalkyl], 5-6-membered (hetero)aryl, —S(O)₀₋₂-[5-6-membered (hetero)aryl], or C1-4alkyl[5-6-membered (hetero)aryl], wherein h2 is optionally substituted with halogen, C1-3 (halo)alkyl, or C1-3 (halo)alkoxyl;
AA¹ is an amino acid residue that is connected to its neighbouring AAⁿ via an amide bond to which it contributes a donor carboxylic acid, and that is connected to tail via an amide bond of a secondary amide to which it contributes a donor amine;
AAⁿ is for each instance independently an amino acid residue, wherein the carbonyl moiety in the residue adjacent to head can instead together with head be replaced by —B(OH)₂ or a C1-6alkyl ester thereof, —P(O)(OH)₂ or a C1-6alkyl ester thereof, —S(O)₂-halogen, or 5-12 membered (hetero)aryl that is optionally substituted with halogen, C1-3 (halo)alkyl, or C1-3 (halo)alkoxy;
m is 1, 2, 3, 4, or 5.


SEQUENCE LISTING (42 sequences)

SEQ ID NO: 1 - protein, 4 aa, synthetic construct
  REGION 1..4: note = Synthetic tetrapeptide
KAAA

SEQ ID NO: 2 - protein, 4 aa, synthetic construct
  REGION 1..4: note = Synthetic tetrapeptide
KAAF

SEQ ID NO: 3 - protein, 4 aa, synthetic construct
  REGION 1..4: note = Synthetic tetrapeptide
KAAH

SEQ ID NO: 4 - protein, 4 aa, synthetic construct
  REGION 1..4: note = Synthetic tetrapeptide
KAAK

SEQ ID NO: 5 - protein, 4 aa, synthetic construct
  REGION 1..4: note = Synthetic tetrapeptide
KAAW

SEQ ID NO: 6-9 - skipped (4 entries)

SEQ ID NO: 10 - protein, 4 aa, synthetic construct
  REGION 1..4: note = Synthetic tetrapeptide
KPAF

SEQ ID NO: 11 - protein, 4 aa, synthetic construct
  REGION 1..4: note = Synthetic tetrapeptide
KPAH

SEQ ID NO: 12 - protein, 4 aa, synthetic construct
  REGION 1..4: note = Synthetic tetrapeptide
KPAK

SEQ ID NO: 13 - protein, 4 aa, synthetic construct
  REGION 1..4: note = Synthetic tetrapeptide
KAPK

SEQ ID NO: 14 - protein, 4 aa, synthetic construct
  REGION 1..4: note = Synthetic tetrapeptide
KPAW

SEQ ID NO: 15-18 - skipped (4 entries)

SEQ ID NO: 19 - protein, 4 aa, synthetic construct
  REGION 1..4: note = Synthetic tetrapeptide
AAAK

SEQ ID NO: 20 - protein, 4 aa, synthetic construct
  REGION 1..4: note = Synthetic tetrapeptide
AAKA

SEQ ID NO: 21 - protein, 4 aa, synthetic construct
  REGION 1..4: note = Synthetic tetrapeptide
AKAA

SEQ ID NO: 22 - protein, 4 aa, synthetic construct
  REGION 1..4: note = Synthetic tetrapeptide
AKAK

SEQ ID NO: 23 - protein, 4 aa, synthetic construct
  REGION 1..4: note = Synthetic tetrapeptide
APAK

SEQ ID NO: 24 - skipped

SEQ ID NO: 25 - protein, 4 aa, synthetic construct
  REGION 1..4: note = Synthetic tetrapeptide
AVAK

SEQ ID NO: 26 - protein, 4 aa, synthetic construct
  REGION 1..4: note = Synthetic tetrapeptide
AKKK

SEQ ID NO: 27 - protein, 4 aa, synthetic construct
  REGION 1..4: note = Synthetic tetrapeptide
LKAK

SEQ ID NO: 28 - protein, 4 aa, synthetic construct
  REGION 1..4: note = Synthetic tetrapeptide
LKKK

SEQ ID NO: 29 - protein, 4 aa, synthetic construct
  REGION 1..4: note = Synthetic tetrapeptide
VKAK

SEQ ID NO: 30 - protein, 4 aa, synthetic construct
  REGION 1..4: note = Synthetic tetrapeptide
KPHK

SEQ ID NO: 31-35 - skipped (5 entries)

SEQ ID NO: 36 - protein, 4 aa, synthetic construct
  REGION 1..4: note = Synthetic tetrapeptide
ARQK

SEQ ID NO: 37 - protein, 4 aa, synthetic construct
  REGION 1..4: note = Synthetic tetrapeptide
ARQF

SEQ ID NO: 38 - protein, 4 aa, synthetic construct
  REGION 1..4: note = Synthetic tetrapeptide
ARRK

SEQ ID NO: 39 - protein, 4 aa, synthetic construct
  REGION 1..4: note = Synthetic tetrapeptide
FKKK

SEQ ID NO: 40 - protein, 4 aa, synthetic construct
  REGION 1..4: note = Synthetic tetrapeptide
RARK

SEQ ID NO: 41 - protein, 4 aa, synthetic construct
  REGION 1..4: note = Synthetic tetrapeptide
FAAF

SEQ ID NO: 42 - protein, 4 aa, synthetic construct
  REGION 1..4: note = Synthetic tetrapeptide
WAAW